(19) United States
(12) Patent Application Publication
(10) Pub. No.: US 2025/0026753 A1
(43) Pub. Date: Jan. 23, 2025

(54) FUSED TRIAZINONE DERIVATIVES AND METHODS OF USING THE SAME

(71) Applicant: Rgenta Therapeutics, Inc., Woburn, MA (US)

(72) Inventors: Travis T. Wager, Brookline, MA (US); Zhiping Weng, Wellesley, MA (US); Hualin Simon Xi, Lexington, MA (US)

(21) Appl. No.: 18/708,108

(22) PCT Filed: Nov. 11, 2022
(86) PCT No.: PCT/US2022/079748

§ 371 Date: May 7, 2024

Related U.S. Application Data
(60) Provisional application No. 63/278,726, filed on Nov. 12, 2021.

(51) Int. Cl. C07D 471/04; A61K 31/53; A61K 31/551; A61P 35/00; C07D 519/00

(52) U.S. Cl. CPC C07D 471/04; A61K 31/53; A61K 31/551; A61P 35/00; C07D 519/00

(57) ABSTRACT

The present disclosure relates to compounds of Formula (I) or (II) or subformulas thereof: (I) and do their prodrugs, pharmaceutically acceptable salts, pharmaceutical compositions, methods of use, and methods for their preparation. The compounds of the present disclosure may act as small molecule splicing modulator that modulate splicing of mRNA, such as pre-mRNA, encoded genes, and methods of use of the compounds for modulating splicing and treating related diseases and conditions. The compounds disclosed herein may possess activity toward various genetic pathways and are accordingly useful in methods of treatment of the human or animal body.

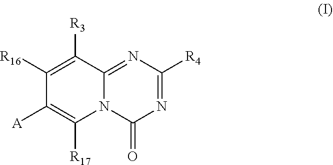

### CROSS REFERENCE TO RELATED APPLICATIONS

This application claims priority to and benefit of U.S. Provisional Application No. 63/278,726, filed Nov. 12, 2021, the contents of which are hereby incorporated by reference in their entirety.

### BACKGROUND

More than 90% of human genes produce multiple mature transcripts via alternative splicing. This process is essential for generating different transcripts in different cell and tissue types, during the developmental process, and in response to internal and external signals. Alternative splicing are prevalent not only for protein-coding genes but also for most other kinds of genes including microRNA genes and long noncoding genes. Splicing is carried out by the spliceosome. Small nuclear RNAs (snRNAs) are key components of the spliceosome. The major spliceosome comprises the U1, U2, U4, U5, and U6 snRNAs, and it catalyzes the removal of ˜95% of human introns, while the remaining introns (called the U12-type of introns) are removed by the minor spliceosome, comprising the U11, U12, U4atac, U5, and U6atac snRNAs. These snRNAs are in complex with their respective protein partners to form the functional unit of small nuclear ribonucleoproteins (snRNPs).

Splicing is a highly regulated process, with the regulation exerted by both cis-elements and trans-factors. The cis-elements that are recognized by the snRNAs include the 5′-splice site, 3′-splice site, and the branchpoint, each of these associating with a sequence motif that is recognized by a component of the spliceosome. In addition, there are intronic splicing enhancers (ISE), intronic splicing silencer (ISS), exonic splicing enhancer (ESE), and exonic splicing enhancer (ESS), which are recognized by a myriad of trans-factors commonly known as RNA-binding proteins (RBPs). Some of these RBPs directly bind to the cis-elements in a sequencing-specific way, while other RBPs recognize RNA structures (e.g., RNA duplex or unpaired loop region), yet others function via protein-protein interaction. There are ˜1600 RBPs annotated in the human genome, and they are expressed in a cell-type-specific manner and form an extensive regulatory network for splicing regulation.

Dysregulation of splicing is implicated in roughly half of human diseases. Some diseases are caused by mutations in the spliceosome components or RBPs, while others by mutations in the cis-elements such as splice sites, branchpoint, or the various splicing enhancers and silencers. Although current approaches to treating these diseases, such as CRISPR-based genome editing, virus-aided gene therapy, or a variety of oligonucleotide-based technologies, continue to improve, they still suffer major technical and clinical challenges. In particular, oligonucleotide-based therapeutics show unfavorable pharmacokinetics, can not be orally administered, and can not be delivered effectively to many tissues, especially the brain. Small-molecule drugs have excellent pharmacokinetics, effective delivery, and bioavailability, and have only recently become available for modulating RNA splicing. Yet, the currently molecules come from a few limited chemical series. Thus, there is a great need to develop additional small molecule splicing modulators (SMSMs).

### SUMMARY

Here we describe a series of novel small molecule splicing modulators (SMSMs), which can be used to treat a wide variety of diseases, including cancers. These SMSMs target regions of a primary RNA transcript that are cis-elements, such as splice sites, branch points, splicing enhancers, or splicing silencers. These regions may contain unpaired nucleotides in an RNA duplex, called bulges. The bulges may be naturally occurring or caused by diseases. When the SMSMs come into contact with the RNA transcript, it may be bound by the spliceosome or the other trans-factors, most notably RNA-binding proteins (RBPs). The SMSMs reported herein may cause an alteration in the sequence or abundance of the mature transcript, which may, in turn, cause a difference in the sequence or abundance of the functional protein should the transcript be protein-coding or the sequence or abundance of the functional RNA should the transcript be non-coding.

In some aspects, the present disclosure provides, inter alia, a compound of Formula (I).

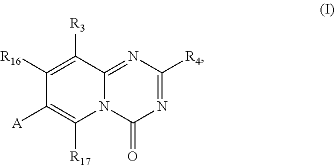

or a pharmaceutically acceptable salt, solvate, or prodrug thereof, wherein:
A is saturated or partially unsaturated mono- or bi-cyclic 4- to 9-membered heterocycloalkyl or NR₁R₂, wherein the heterocycloalkyl comprises 1 or 2 nitrogen ring atoms and is optionally substituted with 1, 2, 3, or 4 R₆;
R₁ is heterocycloalkyl comprising 1 nitrogen ring atom, optionally substituted with 1, 2, 3, or 4 R₆;
R₂ is hydrogen, C₁₋₇alkyl, or C₃₋₈cycloalkyl;
R₃ is H, halo, C₁₋₇alkyl, OR₅, N(R₅)₂, C₃₋₈cycloalkyl, or heterocycloalkyl;
R₄ is aryl or bicyclic 9-membered heteroaryl comprising 2, 3, or 4 heteroatoms independently selected from N, O, and S, wherein R₄ is optionally substituted with 1, 2, or 3 R₇;
each R₅ is independently C₁₋₇alkyl, C₃₋₈cycloalkyl, or heterocycloalkyl;
each R₆ is independently selected from the group consisting of halogen, hydroxy, cyano, —COOH, —C(O)—C₁-C₆alkyl, —C(O)O—C₁-C₆alkyl, C₁-C₇alkyl, C₁-C₈heteroalkyl, C₁₋₇alkoxy-heterocycloalkyl, C₂-C₆alkenyl, C₂-C₆alkynyl, C₁-C₆alkoxy, —(CH₂)₀₋₂—C₃-C₈cycloalkyl, 4-7-membered monocyclic heterocycloalkyl, NH₂, NH(C₁-C₆alkyl), N(C₁-C₆alkyl)₂, —NHC(O)—C₁-C₆alkyl, —N(C₁-C₆alkyl)-C(O)—C₁-C₆alkyl, —C(O)—NH₂, —C(O)—NH(C₁-C₆alkyl), and —C(O)—N(C₁-C₆alkyl)₂, wherein the alkyl, alkenyl, alkynyl, and alkoxy are optionally substituted with one or more halogen, hydroxyl or NH₂, and wherein the cycloalkyl and heterocycloalkyl are optionally substituted with one or more halogen, hydroxyl, C₁-C₆alkyl, C₁-C₆heteroalkyl, C₁-C₆alkoxy, or NH₂; or
two R₆ on the same carbon can be taken together as keto (═O); or
two R₆ together form C₁₋₇alkylene;
each R₇ is independently halo, cyano, C₁₋₇alkyl, C₁₋₇haloalkyl, C₁₋₇alkoxy, C₁₋₇ haloalkoxy, or C₃₋₈cycloalkyl, wherein the C₁₋₇alkyl is optionally substituted with OH;
R₁₆ is H, halo, C₁₋₇alkyl, OR₅, N(R₅)₂, C₃₋₈cycloalkyl, or heterocycloalkyl; and.
R₁₇ is H, halo, C₁₋₇alkyl, OR₅, N(R₅)₂, C₃₋₈cycloalkyl, or heterocycloalkyl.

In some aspects, the present disclosure provides a compound obtainable by, or obtained by, a method for preparing a compound as described here (e.g., a method comprising one or more steps described in herein).

In some aspects, the present disclosure provides a pharmaceutical composition comprising a compound of the present disclosure, or a pharmaceutically acceptable salt, solvate, or prodrug thereof, and a pharmaceutically acceptable diluent or carrier.

In some aspects, the present disclosure provides an intermediate as described herein, being suitable for use in a method for preparing a compound as described herein (e.g., the intermediate is selected from the intermediates described herein).

In some aspects, the present disclosure provides a method of treating or preventing a disease or disorder disclosed herein in a subject in need thereof, comprising administering to the subject a therapeutically effective amount of a compound of the present disclosure or a pharmaceutically acceptable salt, solvate, or prodrug thereof, or a pharmaceutical composition of the present disclosure.

In some aspects, the present disclosure provides a method of treating a disease or disorder disclosed herein in a subject in need thereof, comprising administering to the subject a therapeutically effective amount of a compound of the present disclosure or a pharmaceutically acceptable salt, solvate, or prodrug thereof, or a pharmaceutical composition of the present disclosure.

In some aspects, the present disclosure provides a compound of the present disclosure or a pharmaceutically acceptable salt, solvate, or prodrug thereof for use in treating or preventing a disease or disorder disclosed herein.

In some aspects, the present disclosure provides a compound of the present disclosure or a pharmaceutically acceptable salt, solvate, or prodrug thereof for use in treating a disease or disorder disclosed herein.

In some aspects, the present disclosure provides use of a compound of the present disclosure or a pharmaceutically acceptable salt, solvate, or prodrug thereof for treating or preventing a disease or disorder disclosed herein.

In some aspects, the present disclosure provides use of a compound of the present disclosure or a pharmaceutically acceptable salt, solvate, or prodrug thereof for treating a disease or disorder disclosed herein.

In some aspects, the present disclosure provides use of a compound of the present disclosure or a pharmaceutically acceptable salt, solvate, or prodrug thereof in the manufacture of a medicament for treating or preventing a disease or disorder disclosed herein.

In some aspects, the present disclosure provides use of a compound of the present disclosure or a pharmaceutically acceptable salt, solvate, or prodrug thereof in the manufacture of a medicament for treating a disease or disorder disclosed herein.

In some aspects, the present disclosure provides a method of preparing a compound of the present disclosure.

In some aspects, the present disclosure provides a method of preparing a compound, comprising one or more steps described herein.

Unless otherwise defined, all technical and scientific terms used herein have the same meaning as commonly understood by one of ordinary skill in the art to which this disclosure belongs. Although methods and materials similar or equivalent to those described herein can be used in the practice or testing of the present disclosure, suitable methods and materials are described below. All publications, patent applications, patents, and other references mentioned herein are incorporated by reference in their entirety. In the case of conflict, the present specification, including definitions, will control. In addition, the materials, methods, and examples are illustrative only and are not intended to be limiting.

Other features and advantages of the disclosure will be apparent from the following detailed description and claims.

### DETAILED DESCRIPTION

Compounds described herein are generally designed to treat diseases and disorders disclosed herein.

### Definitions

Unless otherwise stated, the following terms used in the specification and claims have the following meanings set out below.

As used herein, “alkyl”, “C₁, C₂, C₃, C₄, C₅, C₆, or C₇ alkyl” or “C₁-C₇ alkyl” is intended to include C₁, C₂, C₃, C₄, C₅, C₆, or C₇ straight chain (linear) saturated aliphatic hydrocarbon groups and C₃, C₄, C₅, C₆, or C₇ branched saturated aliphatic hydrocarbon groups. For example, C₁-C₇ alkyl is intended to include C₁, C₂, C₃, C₄, C₅, C₆, and C₇ alkyl groups. Examples of alkyl include, moieties having from one to six carbon atoms, such as, but not limited to, methyl, ethyl, n-propyl, i-propyl, n-butyl, s-butyl, t-butyl, n-pentyl, i-pentyl, or n-hexyl. In some embodiments, a straight chain or branched alkyl has six or fewer carbon atoms (e.g., C₁-C₆ for straight chain, C₃-C₆ for branched chain), and in another embodiment, a straight chain or branched alkyl has four or fewer carbon atoms.

As used herein, “alkenyl” is intended to include straight-chain or branched hydrocarbon groups having from 2 to 6 carbon atoms, one or more carbon-carbon double bonds, and no triple bonds (“C₂-C₆alkenyl”). The one or more carbon-carbon double bonds can be internal (such as in 2-butenyl) or terminal (such as in 1-butenyl). Examples of C₂-C₆alkenyl groups include ethenyl (C₂), 1-propenyl (C₃), 2-propenyl (C₃), 1-butenyl (C₄), 2-butenyl (C₄), butadienyl (C₄), and the like.

As used herein, “alkynyl” is intended to include straight-chain or branched hydrocarbon groups having from 2 to 6 carbon atoms, one or more carbon-carbon triple bonds, and optionally one or more double bonds (“C₂-C₆ alkynyl”). The one or more carbon-carbon triple bonds can be internal (such as in 2-butynyl) or terminal (such as in 1-butynyl). Examples of C₂-C₄alkynyl groups include, without limitation, ethynyl (C₂), 1-propynyl (C₃), 2-propynyl (C₃), 1-butynyl (C₄), 2-butynyl (C₄), and the like.

As used herein, the term “optionally substituted alkyl” refers to unsubstituted alkyl or alkyl having designated substituents replacing one or more hydrogen atoms on one or more carbons of the hydrocarbon backbone. Such substituents can include, for example, alkyl, alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, amino (including alkylamino, dialkylamino, arylamino, diarylamino and alkylarylamino), acylamino (including alkylcarbonylamino, arylcarbonylamino, carbamoyl and ureido), amidino, imino, sulphhydryl, alkylthio, arylthio, thiocarboxylate, sulphates, alkylsulphinyl, sulphonato, sulphamoyl, sulphonamido, nitro, trifluoromethyl, cyano, azido, heterocycloalkyl, alkylaryl, or an aromatic or heteroaromatic moiety.

Other optionally substituted moieties (such as optionally substituted cycloalkyl, heterocycloalkyl, aryl, or heteroaryl) include both the unsubstituted moieties and the moieties having one or more of the designated substituents. For example, substituted heterocycloalkyl includes those substituted with one or more alkyl groups, such as 2,2,6,6-tetramethyl-piperidinyl and 2,2,6,6-tetramethyl-1,2,3,6-tetrahydropyridinyl.

As used herein, “heteroalkyl”, “C₁, C₂, C₃, C₄, C₅, C₆, C₇ or C₈ heteroalkyl” or “C₁-C₈ heteroalkyl” is intended to include C₁, C₂, C₃, C₄, C₅, C₆, C₇, or C₈ straight chain (linear) saturated aliphatic hydrocarbon groups and C₃, C₄, C₅, C₆, C₇, or C₈ branched saturated aliphatic hydrocarbon groups, in which at least one of the carbons has been replaced with N, O, or S. The heteroatom will be bonded to any required hydrogens to complete the heteroatom's valence (e.g., a CH₂ may be replaced with an “O” or a “NH”, a CH may be replaced with an N, etc.)). Such substituents can include, for example, —O—CH(CH₃)₂, —CH₂—N(CH₃)—CH₂CH₂OCH₃, —S—CH₂CH₂—O—CH₂CH₃, and so forth.

As used herein, “alkylene”, “C₁, C₂, C₃, C₄, C₅, C₆, or C₇ alkylene” or “C₁-C₇ alkylene” is a bivalent branched or straight alkyl group which has two open valences to connect the alkylene with two other groups. Examples of alkylene include —CH₂—, —CH₂CH₂—, —CH₂CH₂CH₂—, and —CH(CH₂CH₃)—CH₂—. Where an alkylene connects its open valences to the same atom or to separate atoms which are already part of a chain or ring, the alkylene taken together with the atom or atoms to which it is attached will form a ring.

As used herein, the term “alkoxy” refers to the group —OR where R is alkyl. Particular alkoxy groups are methoxy, ethoxy, n-propoxy, isopropoxy, n-butoxy, tert-butoxy, sec-butoxy, n-pentoxy, n-hexoxy, and 1,2-dimethylbutoxy. Particular alkoxy groups are lower alkoxy, i.e., with between 1 and 7 carbon atoms.

As used herein, the term “cycloalkyl” refers to a saturated or partially unsaturated hydrocarbon monocyclic or polycyclic (e.g., fused, bridged, or spiro rings) system having 3 to 30 carbon atoms (e.g., C₃-C₁₂, C₃-C₁₀, or C₃-C₈). Examples of cycloalkyl include, but are not limited to, cyclopropyl, cyclobutyl, cyclopentyl, cyclohexyl, cycloheptyl, cyclooctyl, cyclopentenyl, cyclohexenyl, cycloheptenyl, 1,2,3,4-tetrahydronaphthalenyl, and adamantyl. In the case of polycyclic cycloalkyl, only one of the rings in the cycloalkyl needs to be non-aromatic.

As used herein, the term “heterocycloalkyl” or “heterocyclyl” refers to a saturated or partially unsaturated 3-8 membered monocyclic (e.g., 4-7 membered monocyclic), 7-12 membered bicyclic (fused, bridged, or spiro rings), or 11-14 membered tricyclic ring system (fused, bridged, or spiro rings) (e.g., a mono- or bi-cyclic 4- to 9-membered heterocycloalkyl) having one or more heteroatoms (such as O, N, S, P, or Se), e.g., 1 or 1-2 or 1-3 or 1-4 or 1-5 or 1-6 heteroatoms, or e.g., 1, 2, 3, 4, 5, or 6 heteroatoms, independently selected from the group consisting of nitrogen, oxygen and sulphur, unless specified otherwise. Examples of heterocycloalkyl groups include, but are not limited to, piperidinyl, piperazinyl, pyrrolidinyl, dioxanyl, tetrahydrofuranyl, isoindolinyl, indolinyl, imidazolidinyl, pyrazolidinyl, oxazolidinyl, isoxazolidinyl, triazolidinyl, oxiranyl, azetidinyl, oxetanyl, thietanyl, 1,2,3,6-tetrahydropyridinyl, tetrahydropyranyl, dihydropyranyl, pyranyl, morpholinyl, tetrahydrothiopyranyl, 1,4-diazepanyl, 1,4-oxazepanyl, 2-oxa-5-azabicyclo[2.2.1]heptanyl, 2,5-diazabicyclo[2.2.1]heptanyl, 2-oxa-6-azaspiro[3.3]heptanyl, 2,6-diazaspiro[3.3]heptanyl, 1,4-dioxa-8-azaspiro[4.5]decanyl, 1,4-dioxaspiro[4.5]decanyl, 1-oxaspiro[4.5]decanyl, 1-azaspiro[4.5]decanyl, 3′H-spiro[cyclohexane-1,1′-isobenzofuran]-yl, 7′H-spiro[cyclohexane-1,5′-furo[3,4-b]pyridin]-yl, 3′H-spiro[cyclohexane-1,1′-furo[3,4-c]pyridin]-yl, 3-azabicyclo[3.1.0]hexanyl, 3-azabicyclo[3.1.0]hexan-3-yl, 1,4,5,6-tetrahydropyrrolo[3,4-c]pyrazolyl, 3,4,5,6,7,8-hexahydropyrido[4,3-d]pyrimidinyl, 4,5,6,7-tetrahydro-1H-pyrazolo[3,4-c]pyridinyl, 5,6,7,8-tetrahydropyrido[4,3-d]pyrimidinyl, 2-azaspiro[3.3]heptanyl, 2-methyl-2-azaspiro[3.3]heptanyl, 2-azaspiro[3.5]nonanyl, 2-methyl-2-azaspiro[3.5]nonanyl, 2-azaspiro[4.5]decanyl, 2-methyl-2-azaspiro[4.5]decanyl, 2-oxa-azaspiro[3.4]octanyl, 2-oxa-azaspiro[3.4]octan-6-yl, and the like. In the case of multicyclic heterocycloalkyl, only one of the rings in the heterocycloalkyl needs to be non-aromatic (e.g., 4,5,6,7-tetrahydrobenzo[c]isoxazolyl).

As used herein, the term “aryl” refers to a radical of a monocyclic or polycyclic (e.g., bicyclic or tricyclic) 4n+2 aromatic ring system (e.g., having 6, 10, or 14 π electrons shared in a cyclic array) having 6-14 ring carbon atoms and zero heteroatoms provided in the aromatic ring system. Examples of aryl groups include, but are not limited to, phenyl, naphthyl and the like. Conveniently, an aryl is phenyl.

As used herein, the term “heteroaryl” is intended to include a stable 5-, 6-, or 7-membered monocyclic or 7-, 8-, 9-, 10-, 11- or 12-membered bicyclic aromatic heterocyclic ring which consists of carbon atoms and one or more heteroatoms, e.g., 1 or 1-2 or 1-3 or 1-4 or 1-5 or 1-6 heteroatoms, or e.g., 1, 2, 3, 4, 5, or 6 heteroatoms, independently selected from the group consisting of nitrogen, oxygen and sulphur. The nitrogen atom may be substituted or unsubstituted (i.e., N or NR wherein R is H or other substituents, as defined). The nitrogen and sulphur heteroatoms may optionally be oxidised (i.e., N→O and S(O)ₚ, where p=1 or 2). It is to be noted that total number of S and O atoms in the aromatic heterocycle is not more than 1. Examples of heteroaryl groups include pyrrole, furan, thiophene, thiazole, isothiazole, imidazole, triazole, tetrazole, pyrazole, oxazole, isoxazole, pyridine, pyrazine, pyridazine, pyrimidine, and the like. Heteroaryl groups can also be fused or bridged with alicyclic or heterocyclic rings, which are not aromatic so as to form a multicyclic system (e.g., 4,5,6,7-tetrahydrobenzo[c]isoxazolyl).

Furthermore, the terms “aryl” and “heteroaryl” include multicyclic aryl and heteroaryl groups, e.g., tricyclic, bicyclic, e.g., naphthalene, benzoxazole, benzodioxazole, benzothiazole, benzoimidazole, benzothiophene, quinoline, isoquinoline, naphthrydine, indole, benzofuran, purine, benzofuran, deazapurine, indolizine.

Bicyclic and tricyclic systems can be edge-fused, spiro-fused, or bridged systems.

The cycloalkyl, heterocycloalkyl, aryl, or heteroaryl ring can be substituted at one or more ring positions (e.g., the ring-forming carbon or heteroatom such as N) with such substituents as described above, for example, alkyl, alkenyl, alkynyl, halogen, hydroxyl, alkoxy, alkylcarbonyloxy, arylcarbonyloxy, alkoxycarbonyloxy, aryloxycarbonyloxy, carboxylate, alkylcarbonyl, alkylaminocarbonyl, aralkylaminocarbonyl, alkenylaminocarbonyl, alkylcarbonyl, arylcarbonyl, aralkylcarbonyl, alkenylcarbonyl, alkoxycarbonyl, aminocarbonyl, alkylthiocarbonyl, phosphate, phosphonato, phosphinato, amino (including alkylamino, dialkylamino, arylamino, diarylamino and alkylarylamino), acylamino (including alkylcarbonylamino, arylcarbonylamino, carbamoyl and ureido), amidino, imino, sulphhydryl, alkylthio, arylthio, thiocarboxylate, sulphates, alkylsulphinyl, sulphonato, sulphamoyl, sulphonamido, nitro, trifluoromethyl, cyano, azido, heterocycloalkyl, alkylaryl, or an aromatic or heteroaromatic moiety. Aryl and heteroaryl groups can also be fused or bridged with alicyclic or heterocyclic rings, which are not aromatic so as to form a multicyclic system (e.g., tetralin, methylenedioxyphenyl such as benzo[d][1,3]dioxole-5-yl).

As used herein, the term “substituted,” means that any one or more hydrogen atoms on the designated atom is replaced with a selection from the indicated groups, provided that the designated atom's normal valency is not exceeded, and that the substitution results in a stable compound. When a substituent is oxo or keto (i.e., ═O), then 2 hydrogen atoms on the atom are replaced. Keto substituents are not present on aromatic moieties. Ring double bonds, as used herein, are double bonds that are formed between two adjacent ring atoms (e.g., C═C, C═N or N═N). “Stable compound” and “stable structure” are meant to indicate a compound that is sufficiently robust to survive isolation to a useful degree of purity from a reaction mixture, and formulation into an efficacious therapeutic agent.

When a bond to a substituent is shown to cross a bond connecting two atoms in a ring, then such substituent may be bonded to any atom in the ring. When a substituent is listed without indicating the atom via which such substituent is bonded to the rest of the compound of a given formula, then such substituent may be bonded via any atom in such formula. Combinations of substituents and/or variables are permissible, but only if such combinations result in stable compounds.

When any variable (e.g., R) occurs more than one time in any constituent or formula for a compound, its definition at each occurrence is independent of its definition at every other occurrence. Thus, for example, if a group is shown to be substituted with 0-2 R moieties, then the group may optionally be substituted with up to two R moieties and R at each occurrence is selected independently from the definition of R. Also, combinations of substituents and/or variables are permissible, but only if such combinations result in stable compounds.

As used herein, the term “hydroxy” or “hydroxyl” includes groups with an —OH or —O⁻.

As used herein, the term “cyano” refers to the group —CN.

As used herein, the term “halo” or “halogen” refers to fluoro, chloro, bromo and iodo.

As used herein, the term “haloalkyl” refers to a branched or unbranched alkyl substituted with one or more halogens. For example, a C₁₋₇haloalkyl is an alkyl group of from one to seven carbons wherein at least one H is substituted by a halogen. Examples of haloalkyl include but are not limited to CFH₂, CF₂H, CF₃, CH₂CF₃, CF₂CF₃, C(F)(CH₃)₂, CH₂CH₂Br, CH(I)CH₂F, and CH₂Cl.

As used herein, the term “haloalkoxy” refers to alkoxy structures that are substituted with one or more halo groups or with combinations thereof. For example, the terms “fluoroalkyl” and “fluoro alkoxy” include haloalkyl and haloalkoxy groups, respectively, in which the halo is fluorine.

As used herein, the term “amino” refers to the radical —NH₂. In certain embodiments as specified herein, one or both hydrogen atoms of —NH₂ may be replaced with a different group, e.g., amino-C₁₋₇alkyl.

As used herein, the term “optionally substituted haloalkyl” refers to unsubstituted haloalkyl having designated substituents replacing one or more hydrogen atoms on one or more hydrocarbon backbone carbon atoms. Such substituents can include, for example, alkyl, alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, amino (including alkylamino, dialkylamino, arylamino, diarylamino and alkylarylamino), acylamino (including alkylcarbonylamino, arylcarbonylamino, carbamoyl and ureido), amidino, imino, sulphhydryl, alkylthio, arylthio, thiocarboxylate, sulphates, alkylsulphinyl, sulphonato, sulphamoyl, sulphonamido, nitro, trifluoromethyl, cyano, azido, heterocycloalkyl, alkylaryl, or an aromatic or heteroaromatic moiety.

As used herein, the expressions “one or more of A, B, or C,” “one or more A, B, or C,” “one or more of A, B, and C,” “one or more A, B, and C,” “selected from the group consisting of A, B, and C”, “selected from A, B, and C”, and the like are used interchangeably and all refer to a selection from a group consisting of A, B, and/or C, i.e., one or more As, one or more Bs, one or more Cs, or any combination thereof, unless indicated otherwise.

It is to be understood that the present disclosure provides methods for the synthesis of the compounds of any of the Formulae described herein. The present disclosure also provides detailed methods for the synthesis of various disclosed compounds of the present disclosure according to the following schemes as well as those shown in the Examples.

It is to be understood that, throughout the description, where compositions are described as having, including, or comprising specific components, it is contemplated that compositions also consist essentially of, or consist of, the recited components. Similarly, where methods or processes are described as having, including, or comprising specific process steps, the processes also consist essentially of, or consist of, the recited processing steps. Further, it should be understood that the order of steps or order for performing certain actions is immaterial so long as the invention remains operable. Moreover, two or more steps or actions can be conducted simultaneously.

It is to be understood that the synthetic processes of the disclosure can tolerate a wide variety of functional groups, therefore various substituted starting materials can be used. The processes generally provide the desired final compound at or near the end of the overall process, although it may be desirable in certain instances to further convert the compound to a pharmaceutically acceptable salt thereof.

It is to be understood that compounds of the present disclosure can be prepared in a variety of ways using commercially available starting materials, compounds known in the literature, or from readily prepared intermediates, by employing standard synthetic methods and procedures either known to those skilled in the art, or which will be apparent to the skilled artisan in light of the teachings herein. Standard synthetic methods and procedures for the preparation of organic molecules and functional group transformations and manipulations can be obtained from the relevant scientific literature or from standard textbooks in the field. Although not limited to any one or several sources, classic texts such as Smith, M. B., March, J., March's Advanced Organic Chemistry: Reactions, Mechanisms, and Structure, 5^{th} edition, John Wiley & Sons: New York, 2001; Greene, T. W., Wuts, P. G. M., Protective Groups in Organic Synthesis, 3^{rd} edition, John Wiley & Sons: New York, 1999; R. Larock, Comprehensive Organic Transformations, VCH Publishers (1989); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995), incorporated by reference herein, are useful and recognised reference textbooks of organic synthesis known to those in the art

One of ordinary skill in the art will note that, during the reaction sequences and synthetic schemes described herein, the order of certain steps may be changed, such as the introduction and removal of protecting groups. One of ordinary skill in the art will recognise that certain groups may require protection from the reaction conditions via the use of protecting groups. Protecting groups may also be used to differentiate similar functional groups in molecules. A list of protecting groups and how to introduce and remove these groups can be found in Greene, T. W., Wuts, P. G. M., Protective Groups in Organic Synthesis, 3^{rd} edition, John Wiley & Sons: New York, 1999.

It is to be understood that, unless otherwise stated, any description of a method of treatment includes use of the compounds to provide such treatment or prophylaxis as is described herein, as well as use of the compounds to prepare a medicament to treat or prevent such condition. It is to be understood that, unless otherwise stated, any description of a method of treatment includes use of the compounds to provide such treatment or prophylaxis as is described herein, as well as use of the compounds to prepare a medicament to treat such condition. The treatment includes treatment of human or non-human animals including rodents and other disease models.

As used herein, the term “subject” is interchangeable with the term “subject in need thereof”, both of which refer to a subject having a disease or having an increased risk of developing the disease. A “subject” includes a mammal. The mammal can be e.g., a human or appropriate non-human mammal, such as primate, mouse, rat, dog, cat, cow, horse, goat, camel, sheep or a pig. The subject can also be a bird or fowl. In one embodiment, the mammal is a human. A subject in need thereof can be one who has been previously diagnosed or identified as having a disease or disorder disclosed herein. A subject in need thereof can also be one who is suffering from a disease or disorder disclosed herein. Alternatively, a subject in need thereof can be one who has an increased risk of developing such disease or disorder relative to the population at large (i.e., a subject who is predisposed to developing such disorder relative to the population at large). A subject in need thereof can have a refractory or resistant a disease or disorder disclosed herein (i.e., a disease or disorder disclosed herein that does not respond or has not yet responded to treatment). The subject may be resistant at start of treatment or may become resistant during treatment. In some embodiments, the subject in need thereof received and failed all known effective therapies for a disease or disorder disclosed herein. In some embodiments, the subject in need thereof received at least one prior therapy.

As used herein, the term “treating” or “treat” describes the management and care of a subject for the purpose of combating a disease, condition, or disorder and includes the administration of a compound of the present disclosure, or a pharmaceutically acceptable salt or solvate thereof, to alleviate the symptoms or complications of a disease, condition or disorder, or to eliminate the disease, condition or disorder. The term “treat” can also include treatment of a cell in vitro or an animal model.

It is to be appreciated that references to “treating” or “treatment” include the alleviation of established symptoms of a condition. “Treating” or “treatment” of a state, disorder or condition therefore includes: (1) delaying the appearance of clinical symptoms of the state, disorder or condition developing in a human that may be afflicted with or predisposed to the state, disorder or condition but does not yet experience or display clinical or subclinical symptoms of the state, disorder or condition, (2) inhibiting the state, disorder or condition, i.e., arresting, reducing or delaying the development of the disease or a relapse thereof (in case of maintenance treatment) or at least one clinical or subclinical symptom thereof, or (3) relieving or attenuating the disease, i.e., causing regression of the state, disorder or condition or at least one of its clinical or subclinical symptoms.

It is to be understood that a compound of the present disclosure, or a pharmaceutically acceptable salt or solvate thereof, can or may also be used to prevent a relevant disease, condition or disorder, or used to identify suitable candidates for such purposes.

As used herein, the term “preventing,” “prevent,” or “protecting against” describes reducing or eliminating the onset of the symptoms or complications of such disease, condition or disorder.

It is to be understood that one skilled in the art may refer to general reference texts for detailed descriptions of known techniques discussed herein or equivalent techniques. These texts include Ausubel et al., Current Protocols in Molecular Biology, John Wiley and Sons, Inc. (2005); Sambrook et al., Molecular Cloning, A Laboratory Manual (3^{rd} edition), Cold Spring Harbor Press, Cold Spring Harbor, New York (2000); Coligan et al., Current Protocols in Immunology, John Wiley & Sons, N.Y.; Enna et al., Current Protocols in Pharmacology, John Wiley & Sons, N.Y.; Fingl et al., The Pharmacological Basis of Therapeutics (1975), Remington's Pharmaceutical Sciences, Mack Publishing Co., Easton, PA, 18^{th} edition (1990). These texts can, of course, also be referred to in making or using an aspect of the disclosure.

It is to be understood that the present disclosure also provides pharmaceutical compositions comprising any compound described herein in combination with at least one pharmaceutically acceptable excipient or carrier.

As used herein, the term “pharmaceutical composition” is a formulation containing the compounds of the present disclosure in a form suitable for administration to a subject. In one embodiment, the pharmaceutical composition is in bulk or in unit dosage form. The unit dosage form is any of a variety of forms, including, for example, a capsule, an IV bag, a tablet, a single pump on an aerosol inhaler or a vial. The quantity of active ingredient (e.g., a formulation of the disclosed compound or salt, hydrate, solvate or isomer thereof) in a unit dose of composition is an effective amount and is varied according to the particular treatment involved. One skilled in the art will appreciate that it is sometimes necessary to make routine variations to the dosage depending on the age and condition of the subject. The dosage will also depend on the route of administration. A variety of routes are contemplated, including oral, pulmonary, rectal, parenteral, transdermal, subcutaneous, intravenous, intramuscular, intraperitoneal, inhalational, buccal, sublingual, intrapleural, intrathecal, intranasal, and the like. Dosage forms for the topical or transdermal administration of a compound of this disclosure include powders, sprays, ointments, pastes, creams, lotions, gels, solutions, patches and inhalants. In one embodiment, the active compound is mixed under sterile conditions with a pharmaceutically acceptable carrier, and with any preservatives, buffers, or propellants that are required.

As used herein, the term “pharmaceutically acceptable” refers to those compounds, anions, cations, materials, compositions, carriers, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, the term “pharmaceutically acceptable excipient” means an excipient that is useful in preparing a pharmaceutical composition that is generally safe, non-toxic and neither biologically nor otherwise undesirable, and includes excipient that is acceptable for veterinary use as well as human pharmaceutical use. A “pharmaceutically acceptable excipient” as used in the specification and claims includes both one and more than one such excipient.

It is to be understood that a pharmaceutical composition of the disclosure is formulated to be compatible with its intended route of administration. Examples of routes of administration include parenteral, e.g., intravenous, intradermal, subcutaneous, oral (e.g., ingestion), inhalation, transdermal (topical), and transmucosal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulphite; chelating agents such as ethylenediaminetetraacetic acid; buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

It is to be understood that a compound or pharmaceutical composition of the disclosure can be administered to a subject in many of the well-known methods currently used for chemotherapeutic treatment. For example, a compound of the disclosure may be injected into the blood stream or body cavities or taken orally or applied through the skin with patches. The dose chosen should be sufficient to constitute effective treatment but not so high as to cause unacceptable side effects. The state of the disease condition (e.g., a disease or disorder disclosed herein) and the health of the subject should preferably be closely monitored during and for a reasonable period after treatment.

As used herein, the term “therapeutically effective amount”, refers to an amount of a pharmaceutical agent to treat, ameliorate, or prevent an identified disease or condition, or to exhibit a detectable therapeutic or inhibitory effect. The effect can be detected by any assay method known in the art. The precise effective amount for a subject will depend upon the subject's body weight, size, and health; the nature and extent of the condition; and the therapeutic or combination of therapeutics selected for administration. Therapeutically effective amounts for a given situation can be determined by routine experimentation that is within the skill and judgment of the clinician.

A “therapeutically effective amount” means the amount of a compound that, when administered to a subject for treating a disease, is sufficient to effect such treatment for the disease. The “therapeutically effective amount” will vary depending on the compound, the disease and its severity and the age, weight, etc., of the subject to be treated.

It is to be understood that, for any compound, the therapeutically effective amount can be estimated initially either in cell culture assays, e.g., of neoplastic cells, or in animal models, usually rats, mice, rabbits, dogs, or pigs. The animal model may also be used to determine the appropriate concentration range and route of administration. Such information can then be used to determine useful doses and routes for administration in humans. Therapeutic/prophylactic efficacy and toxicity may be determined by standard pharmaceutical procedures in cell cultures or experimental animals, e.g., ED₅₀ (the dose therapeutically effective in 50% of the population) and LD₅₀ (the dose lethal to 50% of the population). The dose ratio between toxic and therapeutic effects is the therapeutic index, and it can be expressed as the ratio, LD₅₀/ED₅₀. Pharmaceutical compositions that exhibit large therapeutic indices are preferred. The dosage may vary within this range depending upon the dosage form employed, sensitivity of the subject, and the route of administration.

Dosage and administration are adjusted to provide sufficient levels of the active agent(s) or to maintain the desired effect. Factors which may be taken into account include the severity of the disease state, general health of the subject, age, weight, and gender of the subject, diet, time and frequency of administration, drug combination(s), reaction sensitivities, and tolerance/response to therapy.

The pharmaceutical compositions containing active compounds of the present disclosure may be manufactured in a manner that is generally known, e.g., by means of conventional mixing, dissolving, granulating, dragee-making, levigating, emulsifying, encapsulating, entrapping, or lyophilising processes. Pharmaceutical compositions may be formulated in a conventional manner using one or more pharmaceutically acceptable carriers comprising excipients and/or auxiliaries that facilitate processing of the active compounds into preparations that can be used pharmaceutically. Of course, the appropriate formulation is dependent upon the route of administration chosen.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol and sorbitol, and sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilisation. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

Oral compositions generally include an inert diluent or an edible pharmaceutically acceptable carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser, which contains a suitable propellant, e.g., a gas such as carbon dioxide, or a nebuliser.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The active compounds can be prepared with pharmaceutically acceptable carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the disclosure are dictated by and directly dependent on the unique characteristics of the active compound and the particular therapeutic effect to be achieved.

In therapeutic applications, the dosages of the pharmaceutical compositions used in accordance with the disclosure vary depending on the agent, the age, weight, and clinical condition of the recipient subject, and the experience and judgment of the clinician or practitioner administering the therapy, among other factors affecting the selected dosage. Generally, the dose should be sufficient to result in slowing, and preferably regressing, the symptoms of the disease or disorder disclosed herein and also preferably causing complete regression of the disease or disorder. An effective amount of a pharmaceutical agent is that which provides an objectively identifiable improvement as noted by the clinician or other qualified observer. Improvement in survival and growth indicates regression. As used herein, the term “dosage effective manner” refers to amount of an active compound to produce the desired biological effect in a subject or cell.

It is to be understood that the pharmaceutical compositions can be included in a container, pack, or dispenser together with instructions for administration.

It is to be understood that, for the compounds of the present disclosure being capable of further forming salts, all of these forms are also contemplated within the scope of the claimed disclosure.

As used herein, the term “pharmaceutically acceptable salts” refer to derivatives of the compounds of the present disclosure wherein the parent compound is modified by making acid or base salts thereof. Examples of pharmaceutically acceptable salts include, but are not limited to, mineral or organic acid salts of basic residues such as amines, alkali or organic salts of acidic residues such as carboxylic acids, and the like. The pharmaceutically acceptable salts include the conventional non-toxic salts or the quaternary ammonium salts of the parent compound formed, for example, from non-toxic inorganic or organic acids. For example, such conventional non-toxic salts include, but are not limited to, those derived from inorganic and organic acids selected from 2-acetoxybenzoic, 2-hydroxyethane sulphonic, acetic, ascorbic, benzene sulphonic, benzoic, bicarbonic, carbonic, citric, edetic, ethane disulphonic, 1,2-ethane sulphonic, fumaric, glucoheptonic, gluconic, glutamic, glycolic, glycollyarsanilic, hexylresorcinic, hydrabamic, hydrobromic, hydrochloric, hydroiodic, hydroxymaleic, hydroxynaphthoic, isethionic, lactic, lactobionic, lauryl sulphonic, maleic, malic, mandelic, methane sulphonic, napsylic, nitric, oxalic, pamoic, pantothenic, phenylacetic, phosphoric, polygalacturonic, propionic, salicylic, stearic, subacetic, succinic, sulphamic, sulphanilic, sulphuric, tannic, tartaric, toluene sulphonic, and the commonly occurring amine acids, e.g., glycine, alanine, phenylalanine, arginine, etc.

In some embodiments, the pharmaceutically acceptable salt is a sodium salt, a potassium salt, a calcium salt, a magnesium salt, a diethylamine salt, a choline salt, a meglumine salt, a benzathine salt, a tromethamine salt, an ammonia salt, an arginine salt, or a lysine salt.

Other examples of pharmaceutically acceptable salts include hexanoic acid, cyclopentane propionic acid, pyruvic acid, malonic acid, 3-(4-hydroxybenzoyl)benzoic acid, cinnamic acid, 4-chlorobenzenesulphonic acid, 2-naphthalenesulphonic acid, 4-toluenesulphonic acid, camphorsulphonic acid, 4-methylbicyclo-[2.2.2]-oct-2-ene-1-carboxylic acid, 3-phenylpropionic acid, trimethylacetic acid, tertiary butylacetic acid, muconic acid, and the like. The present disclosure also encompasses salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, tromethamine, N-methylglucamine, and the like. In the salt form, it is understood that the ratio of the compound to the cation or anion of the salt can be 1:1, or any ratio other than 1:1, e.g., 3:1, 2:1, 1:2, or 1:3.

It is to be understood that all references to pharmaceutically acceptable salts include solvent addition forms (solvates) as defined herein, of the same salt.

The compounds, or pharmaceutically acceptable salts thereof, are administered orally, nasally, transdermally, pulmonary, inhalationally, buccally, sublingually, intraperitoneally, subcutaneously, intramuscularly, intravenously, rectally, intrapleurally, intrathecally and parenterally. In one embodiment, the compound is administered orally. One skilled in the art will recognise the advantages of certain routes of administration.

A salt, for example, can be formed between an anion and a positively charged group (e.g., amino) on a substituted compound disclosed herein. Suitable anions include chloride, bromide, iodide, sulphate, bisulphate, sulphamate, nitrate, phosphate, citrate, methanesulphonate, trifluoroacetate, glutamate, glucuronate, glutarate, malate, maleate, succinate, fumarate, tartrate, tosylate, salicylate, lactate, naphthalenesulphonate, and acetate (e.g., trifluoroacetate).

As used herein, the term “pharmaceutically acceptable anion” refers to an anion suitable for forming a pharmaceutically acceptable salt. Likewise, a salt can also be formed between a cation and a negatively charged group (e.g., carboxylate) on a substituted compound disclosed herein. Suitable cations include sodium ion, potassium ion, magnesium ion, calcium ion, and an ammonium cation such as tetramethylammonium ion or diethylamine ion. The substituted compounds disclosed herein also include those salts containing quaternary nitrogen atoms.

It is to be understood that the compounds of the present disclosure, for example, the salts of the compounds, can exist in either hydrated or unhydrated (the anhydrous) form or as solvates with other solvent molecules. Nonlimiting examples of hydrates include monohydrates, dihydrates, etc. Nonlimiting examples of solvates include ethanol solvates, acetone solvates, etc.

As used herein, the term “solvate” means solvent addition forms that contain either stoichiometric or non-stoichiometric amounts of solvent. Some compounds have a tendency to trap a fixed molar ratio of solvent molecules in the crystalline solid state, thus forming a solvate. If the solvent is water the solvate formed is a hydrate; and if the solvent is alcohol, the solvate formed is an alcoholate. Hydrates are formed by the combination of one or more molecules of water with one molecule of the substance in which the water retains its molecular state as H₂O.

As used herein, the term “analog” refers to a chemical compound that is structurally similar to another but differs slightly in composition (as in the replacement of one atom by an atom of a different element or in the presence of a particular functional group, or the replacement of one functional group by another functional group). Thus, an analog is a compound that is similar or comparable in function and appearance, but not in structure or origin to the reference compound.

As used herein, the term “derivative” refers to compounds that have a common core structure and are substituted with various groups as described herein.

As used herein, the term “bioisostere” refers to a compound resulting from the exchange of an atom or of a group of atoms with another, broadly similar, atom or group of atoms. The objective of a bioisosteric replacement is to create a new compound with similar biological properties to the parent compound. The bioisosteric replacement may be physicochemically or topologically based. Examples of carboxylic acid bioisosteres include, but are not limited to, acyl sulphonamides, tetrazoles, sulphonates and phosphonates. See, e.g., Patani and LaVoie, Chem. Rev. 96, 3147-3176, 1996.

It is also to be understood that certain compounds of any one of the Formulae disclosed herein may exist in solvated as well as unsolvated forms such as, for example, hydrated forms. A suitable pharmaceutically acceptable solvate is, for example, a hydrate such as hemi-hydrate, a mono-hydrate, a di-hydrate or a tri-hydrate.

Compounds of any one of the Formulae disclosed herein may exist in a number of different tautomeric forms and references to compounds of Formula (I) and (II) include all such forms. For the avoidance of doubt, where a compound can exist in one of several tautomeric forms, and only one is specifically described or shown, all others are nevertheless embraced by Formula (I) and (II). Examples of tautomeric forms include keto-, enol-, and enolate-forms, as in, for example, the following tautomeric pairs: keto/enol (illustrated below), imine/enamine, amide/imino alcohol, amidine/amidine, nitroso/oxime, thioketone/enethiol, and nitro/aci-nitro.

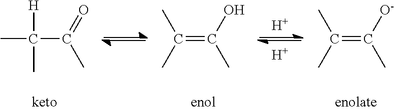

Compounds of any one of the Formulae disclosed herein containing an amine function may also form N-oxides. A reference herein to a compound of Formula (I) and (II) that contains an amine function also includes the N-oxide. Where a compound contains several amine functions, one or more than one nitrogen atom may be oxidised to form an N-oxide. Particular examples of N-oxides are the N-oxides of a tertiary amine or a nitrogen atom of a nitrogen-containing heterocycle. N-oxides can be formed by treatment of the corresponding amine with an oxidising agent such as hydrogen peroxide or a peracid (e.g. a peroxycarboxylic acid), see for example Advanced Organic Chemistry, by Jerry March, 4th Edition, Wiley Interscience, pages. More particularly, N-oxides can be made by the procedure of L. W. Deady (Syn. Comm. 1977, 7, 509-514) in which the amine compound is reacted with meta-chloroperoxybenzoic acid (mCPBA), for example, in an inert solvent such as dichloromethane.

The compounds of any one of the Formulae disclosed herein may be administered in the form of a prodrug which is broken down in the human or animal body to release a compound of the disclosure. A prodrug may be used to alter the physical properties and/or the pharmacokinetic properties of a compound of the disclosure. A prodrug can be formed when the compound of the disclosure contains a suitable group or substituent to which a property-modifying group can be attached. Examples of prodrugs include derivatives containing in vivo cleavable alkyl or acyl substitutents at the ester or amide group in any one of the Formulae disclosed herein.

As used herein, the term “isomerism” means compounds that have identical molecular formulae but differ in the sequence of bonding of their atoms or in the arrangement of their atoms in space. Isomers that differ in the arrangement of their atoms in space are termed “stereoisomers.” Stereoisomers that are not mirror images of one another are termed “diastereoisomers,” and stereoisomers that are non-superimposable mirror images of each other are termed “enantiomers” or sometimes optical isomers. A mixture containing equal amounts of individual enantiomeric forms of opposite chirality is termed a “racemic mixture.”

As used herein, the term “chiral centre” refers to a carbon atom bonded to four nonidentical substituents.

As used herein, the term “chiral isomer” means a compound with at least one chiral centre. Compounds with more than one chiral centre may exist either as an individual diastereomer or as a mixture of diastereomers, termed “diastereomeric mixture.” When one chiral centre is present, a stereoisomer may be characterised by the absolute configuration (R or S) of that chiral centre. Absolute configuration refers to the arrangement in space of the substituents attached to the chiral centre. The substituents attached to the chiral centre under consideration are ranked in accordance with the Sequence Rule of Cahn, Ingold and Prelog. (Cahn et al., Angew. Chem. Inter. Edit. 1966, 5, 385; errata 511; Cahn et al., Angew. Chem. 1966, 78, 413; Cahn and Ingold, J. Chem. Soc. 1951 (London), 612; Cahn et al., Experientia 1956, 12, 81; Cahn, J. Chem. Educ. 1964, 41, 116).

As used herein, the term “geometric isomer” means the diastereomers that owe their existence to hindered rotation about double bonds or a cycloalkyl linker (e.g., 1,3-cyclobutyl). These configurations are differentiated in their names by the prefixes cis and trans, or Z and E, which indicate that the groups are on the same or opposite side of the double bond in the molecule according to the Cahn-Ingold-Prelog rules.

It is to be understood that the compounds of the present disclosure may be depicted as different chiral isomers or geometric isomers. It is also to be understood that when compounds have chiral isomeric or geometric isomeric forms, all isomeric forms are intended to be included in the scope of the present disclosure, and the naming of the compounds does not exclude any isomeric forms, it being understood that not all isomers may have the same level of activity.

It is to be understood that the structures and other compounds discussed in this disclosure include all atropic isomers thereof. It is also to be understood that not all atropic isomers may have the same level of activity.

As used herein, the term “atropic isomers” are a type of stereoisomer in which the atoms of two isomers are arranged differently in space. Atropic isomers owe their existence to a restricted rotation caused by hindrance of rotation of large groups about a central bond. Such atropic isomers typically exist as a mixture, however as a result of recent advances in chromatography techniques, it has been possible to separate mixtures of two atropic isomers in select cases.

As used herein, the term “tautomer” is one of two or more structural isomers that exist in equilibrium and is readily converted from one isomeric form to another. This conversion results in the formal migration of a hydrogen atom accompanied by a switch of adjacent conjugated double bonds. Tautomers exist as a mixture of a tautomeric set in solution. In solutions where tautomerisation is possible, a chemical equilibrium of the tautomers will be reached. The exact ratio of the tautomers depends on several factors, including temperature, solvent and pH. The concept of tautomers that are interconvertible by tautomerisations is called tautomerism. Of the various types of tautomerism that are possible, two are commonly observed. In keto-enol tautomerism a simultaneous shift of electrons and a hydrogen atom occurs. Ring-chain tautomerism arises as a result of the aldehyde group (—CHO) in a sugar chain molecule reacting with one of the hydroxy groups (—OH) in the same molecule to give it a cyclic (ring-shaped) form as exhibited by glucose.

It is to be understood that the compounds of the present disclosure may be depicted as different tautomers. It should also be understood that when compounds have tautomeric forms, all tautomeric forms are intended to be included in the scope of the present disclosure, and the naming of the compounds does not exclude any tautomer form. It will be understood that certain tautomers may have a higher level of activity than others.

Compounds that have the same molecular formula but differ in the nature or sequence of bonding of their atoms or the arrangement of their atoms in space are termed “isomers”. Isomers that differ in the arrangement of their atoms in space are termed “stereoisomers”. Stereoisomers that are not mirror images of one another are termed “diastereomers” and those that are non-superimposable mirror images of each other are termed “enantiomers”. When a compound has an asymmetric centre, for example, it is bonded to four different groups, a pair of enantiomers is possible. An enantiomer can be characterised by the absolute configuration of its asymmetric centre and is described by the R- and S-sequencing rules of Cahn and Prelog, or by the manner in which the molecule rotates the plane of polarised light and designated as dextrorotatory or levorotatory (i.e., as (+) or (−)-isomers respectively). A chiral compound can exist as either individual enantiomer or as a mixture thereof. A mixture containing equal proportions of the enantiomers is called a “racemic mixture”.

The compounds of this disclosure may possess one or more asymmetric centres; such compounds can therefore be produced as individual (R)- or (S)-stereoisomers or as mixtures thereof. Unless indicated otherwise, the description or naming of a particular compound in the specification and claims is intended to include both individual enantiomers and mixtures, racemic or otherwise, thereof. The methods for the determination of stereochemistry and the separation of stereoisomers are well-known in the art (see discussion in Chapter 4 of “Advanced Organic Chemistry”, 4th edition J. March, John Wiley and Sons, New York, 2001), for example by synthesis from optically active starting materials or by resolution of a racemic form. Some of the compounds of the disclosure may have geometric isomeric centres (E- and Z-isomers).

Accordingly, the present disclosure includes those compounds of any one of the Formulae disclosed herein as defined hereinbefore when made available by organic synthesis and when made available within the human or animal body by way of cleavage of a prodrug thereof. Accordingly, the present disclosure includes those compounds of any one of the Formulae disclosed herein that are produced by organic synthetic means and also such compounds that are produced in the human or animal body by way of metabolism of a precursor compound, that is a compound of any one of the Formulae disclosed herein may be a synthetically-produced compound or a metabolically-produced compound.

A suitable pharmaceutically acceptable prodrug of a compound of any one of the Formulae disclosed herein is one that is based on reasonable medical judgment as being suitable for administration to the subject without undesirable pharmacological activities and without undue toxicity. Various forms of prodrug have been described, for example in the following documents: a) Methods in Enzymology, Vol. 42, p. 309-396, edited by K. Widder, et al. (Academic Press, 1985); b) Design of Pro-drugs, edited by H. Bundgaard, (Elsevier, 1985); c) A Textbook of Drug Design and Development, edited by Krogsgaard-Larsen and H. Bundgaard, Chapter 5 “Design and Application of Pro-drugs”, by H. Bundgaard p. 113-191 (1991); d) H. Bundgaard, Advanced Drug Delivery Reviews, 8, 1-38 (1992); e) H. Bundgaard, et al., Journal of Pharmaceutical Sciences, 77, 285 (1988); f) N. Kakeya, et al., Chem. Pharm. Bull., 32, 692 (1984); g) T. Higuchi and V. Stella, “Pro-Drugs as Novel Delivery Systems”, A.C.S. Symposium Series, Volume 14; and h) E. Roche (editor), “Bioreversible Carriers in Drug Design”, Pergamon Press, 1987.

A suitable pharmaceutically acceptable prodrug of a compound of any one of the Formulae disclosed herein that possesses a hydroxy group is, for example, an in vivo cleavable ester or ether thereof. An in vivo cleavable ester or ether of a compound of any one of the Formulae disclosed herein containing a hydroxy group is, for example, a pharmaceutically acceptable ester or ether which is cleaved in the subject to produce the parent hydroxy compound. Suitable pharmaceutically acceptable ester forming groups for a hydroxy group include inorganic esters such as phosphate esters (including phosphoramidic cyclic esters). Further suitable pharmaceutically acceptable ester forming groups for a hydroxy group include C₁-C₁₀ alkanoyl groups such as acetyl, benzoyl, phenylacetyl and substituted benzoyl and phenylacetyl groups, C₁-C₁₀ alkoxycarbonyl groups such as ethoxycarbonyl, N,N—(C₁-C₆ alkyl)₂carbamoyl, 2-dialkylaminoacetyl and 2-carboxyacetyl groups. Examples of ring substituents on the phenylacetyl and benzoyl groups include aminomethyl, N-alkylaminomethyl, N,N-dialkylaminomethyl, morpholinomethyl, piperazin-1-ylmethyl and 4-(C₁-C₄ alkyl)piperazin-1-ylmethyl. Suitable pharmaceutically acceptable ether forming groups for a hydroxy group include α-acyloxyalkyl groups such as acetoxymethyl and pivaloyloxymethyl groups.

A suitable pharmaceutically acceptable prodrug of a compound of any one of the Formulae disclosed herein that possesses a carboxy group is, for example, an in vivo cleavable amide thereof, for example an amide formed with an amine such as ammonia, a C₁-4alkylamine such as methylamine, a (C₁-C₄ alkyl)₂amine such as dimethylamine, N-ethyl-N-methylamine or diethylamine, a C₁-C₄ alkoxy-C₂-C₄ alkylamine such as 2-methoxyethylamine, a phenyl-C₁-C₄ alkylamine such as benzylamine and amino acids such as glycine or an ester thereof.

A suitable pharmaceutically acceptable prodrug of a compound of any one of the Formulae disclosed herein that possesses an amino group is, for example, an in vivo cleavable amide derivative thereof. Suitable pharmaceutically acceptable amides from an amino group include, for example an amide formed with C₁-C₁₀ alkanoyl groups such as an acetyl, benzoyl, phenylacetyl and substituted benzoyl and phenylacetyl groups. Examples of ring substituents on the phenylacetyl and benzoyl groups include aminomethyl, N-alkylaminomethyl, N,N-dialkylaminomethyl, morpholinomethyl, piperazin-1-ylmethyl and 4-(C₁-C₄ alkyl)piperazin-1-ylmethyl.

The dosage regimen utilising the compounds is selected in accordance with a variety of factors including type, species, age, weight, sex and medical condition of the subject; the severity of the condition to be treated; the route of administration; the renal and hepatic function of the subject; and the particular compound or salt thereof employed. An ordinarily skilled physician or veterinarian can readily determine and prescribe the effective amount of the drug required to prevent, counter, or arrest the progress of the condition. An ordinarily skilled physician or veterinarian can readily determine and prescribe the effective amount of the drug required to counter or arrest the progress of the condition.

Techniques for formulation and administration of the disclosed compounds of the disclosure can be found in Remington: the Science andPractice ofPharmacy, 19^{th} edition, Mack Publishing Co., Easton, PA (1995). In an embodiment, the compounds described herein, and the pharmaceutically acceptable salts thereof, are used in pharmaceutical preparations in combination with a pharmaceutically acceptable carrier or diluent. Suitable pharmaceutically acceptable carriers include inert solid fillers or diluents and sterile aqueous or organic solutions. The compounds will be present in such pharmaceutical compositions in amounts sufficient to provide the desired dosage amount in the range described herein.

All percentages and ratios used herein, unless otherwise indicated, are by weight. Other features and advantages of the present disclosure are apparent from the different examples. The provided examples illustrate different components and methodology useful in practicing the present disclosure. The examples do not limit the claimed disclosure. Based on the present disclosure the skilled artisan can identify and employ other components and methodology useful for practicing the present disclosure.

In the synthetic schemes described herein, compounds may be drawn with one particular configuration for simplicity. Such particular configurations are not to be construed as limiting the disclosure to one or another isomer, tautomer, regioisomer or stereoisomer, nor does it exclude mixtures of isomers, tautomers, regioisomers or stereoisomers; however, it will be understood that a given isomer, tautomer, regioisomer or stereoisomer may have a higher level of activity than another isomer, tautomer, regioisomer or stereoisomer.

All publications and patent documents cited herein are incorporated herein by reference as if each such publication or document was specifically and individually indicated to be incorporated herein by reference. Citation of publications and patent documents is not intended as an admission that any is pertinent prior art, nor does it constitute any admission as to the contents or date of the same. The present disclosure having now been described by way of written description, those of skill in the art will recognize that the present disclosure can be practiced in a variety of embodiments and that the foregoing description and examples below are for purposes of illustration and not limitation of the claims that follow.

As use herein, the phrase “compound of the disclosure” refers to those compounds which are disclosed herein, both generically and specifically.

### Compounds of the Present Disclosure

In some aspects, the present disclosure provides, inter alia, a compound of Formula (I):

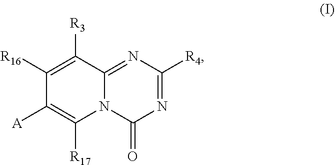

or a pharmaceutically acceptable salt, solvate, or prodrug thereof, wherein:
A is saturated or partially unsaturated mono- or bi-cyclic 4- to 9-membered heterocycloalkyl or NR₁R₂, wherein the heterocycloalkyl comprises 1 or 2 nitrogen ring atoms and is optionally substituted with 1, 2, 3, or 4 R₆;
R₁ is heterocycloalkyl comprising 1 nitrogen ring atom, optionally substituted with 1, 2, 3, or 4 R₆;
R₂ is hydrogen, C₁₋₇alkyl, or C₃₋₈cycloalkyl;
R₃ is H, halo, C₁₋₇alkyl, OR₅, N(R₅)₂, C₃₋₈cycloalkyl, or heterocycloalkyl;
R₄ is aryl or bicyclic 9-membered heteroaryl comprising 2, 3, or 4 heteroatoms independently selected from N, O, and S, wherein R₄ is optionally substituted with 1, 2, or 3 R₇;
each R₅ is independently C₁₋₇alkyl, C₃₋₈cycloalkyl, or heterocycloalkyl;
each R₆ is independently selected from the group consisting of halogen, hydroxy, cyano, —COOH, —C(O)—C₁-C₆alkyl, —C(O)O—C₁-C₆alkyl, C₁-C₇alkyl, C₁-C₈heteroalkyl, C₁₋₇alkoxy-heterocycloalkyl, C₂-C₆alkenyl, C₂-C₆alkynyl, C₁-C₆alkoxy, —(CH₂)₀₋₂—C₃-C₈cycloalkyl, 4-7-membered monocyclic heterocycloalkyl, NH₂, NH(C₁-C₆alkyl), N(C₁-C₆alkyl)₂, —NHC(O)—C₁-C₆alkyl, —N(C₁-C₆alkyl)-C(O)—C₁-C₆alkyl, —C(O)—NH₂, —C(O)—NH(C₁-C₆alkyl), and —C(O)—N(C₁-C₆alkyl)₂, wherein the alkyl, alkenyl, alkynyl, and alkoxy are optionally substituted with one or more halogen, hydroxyl or NH₂, and wherein the cycloalkyl and heterocycloalkyl are optionally substituted with one or more halogen, hydroxyl, C₁-C₆alkyl, C₁-C₆heteroalkyl, C₁-C₆alkoxy, or NH₂; or two R₆ on the same carbon can be taken together as keto (═O); or two R₆ together form C₁₋₇alkylene;
each R₇ is independently halo, cyano, C₁₋₇alkyl, C₁₋₇haloalkyl, C₁₋₇alkoxy, C₁₋₇ haloalkoxy, or C₃₋₈cycloalkyl, wherein the C₁₋₇alkyl is optionally substituted with OH;
R₁₆ is H, halo, C₁₋₇alkyl, OR₅, N(R₅)₂, C₃₋₈cycloalkyl, or heterocycloalkyl; and.
R₁₇ is H, halo, C₁₋₇alkyl, OR₅, N(R₅)₂, C₃₋₈cycloalkyl, or heterocycloalkyl.

In some aspects, the present disclosure provides, inter alia, a compound of Formula (I):

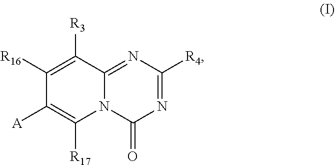

or a pharmaceutically acceptable salt, solvate, or prodrug thereof, wherein:
A is saturated or partially unsaturated mono- or bi-cyclic 4- to 9-membered heterocycloalkyl or NR₁R₂, wherein the heterocycloalkyl comprises 1 or 2 nitrogen ring atoms and is optionally substituted with 1, 2, 3, or 4 R₆;
R₁ is heterocycloalkyl comprising 1 nitrogen ring atom, optionally substituted with 1, 2, 3, or 4 R₆;
R₂ is hydrogen, C₁₋₇alkyl, or C₃₋₈cycloalkyl;
R₃ is H, halo, C₁₋₇alkyl, OR₅, N(R₅)₂, C₃₋₈cycloalkyl, or heterocycloalkyl;
R₄ is aryl or bicyclic 9-membered heteroaryl comprising 2, 3, or 4 heteroatoms independently selected from N, O, and S, wherein R₄ is optionally substituted with 1, 2, or 3 R₇;
each R₅ is independently C₁₋₇alkyl, C₃₋₈cycloalkyl, or heterocycloalkyl;
each R₆ is independently C₁₋₇alkyl, amino, amino-C₁₋₇alkyl, C₃₋₈cycloalkyl, heterocycloalkyl, or C₁₋₇alkoxy-heterocycloalkyl, or two R₆ together form C₁₋₇alkylene;
each R₇ is independently halo, cyano, C₁₋₇alkyl, C₁₋₇haloalkyl, C₁₋₇alkoxy, C₁₋₇ haloalkoxy, or C₃₋₈cycloalkyl, wherein the C₁₋₇alkyl is optionally substituted with OH;
R₁₆ is H, halo, C₁₋₇alkyl, OR₅, N(R₅)₂, C₃₋₈cycloalkyl, or heterocycloalkyl; and.
R₁₇ is H, halo, C₁₋₇alkyl, OR₅, N(R₅)₂, C₃₋₈cycloalkyl, or heterocycloalkyl.

In some aspects, the present disclosure provides, inter alia, a compound of Formula (I).

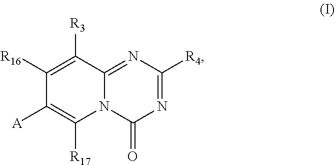

or a pharmaceutically acceptable salt, solvate, or prodrug thereof, wherein:
A is saturated or partially unsaturated mono- or bi-cyclic 4- to 9-membered heterocycloalkyl or NR₁R₂, wherein the heterocycloalkyl comprises 1 or 2 nitrogen ring atoms and is optionally substituted with 1, 2, 3, or 4 R₆;
R₁ is heterocycloalkyl comprising 1 nitrogen ring atom, optionally substituted with 1, 2, 3, or 4 R₆;
R₂ is hydrogen, C₁₋₇alkyl, or C₃₋₈cycloalkyl;
R₃ is H, halo, C₁₋₇alkyl, OR₅, N(R₅)₂, C₃₋₈cycloalkyl, or heterocycloalkyl;
R₄ is aryl or bicyclic 9-membered heteroaryl comprising 2, 3, or 4 heteroatoms independently selected from N, O, and S, wherein R₄ is optionally substituted with 1, 2, or 3 R₇;
each R₅ is independently C₁₋₇alkyl, C₃₋₈cycloalkyl, or heterocycloalkyl;
each R₆ is independently C₁₋₇alkyl, amino, amino-C₁₋₇alkyl, C₃₋₈cycloalkyl, heterocycloalkyl, or C₁₋₇alkoxy-heterocycloalkyl, or two R₆ together form C₁₋₇alkylene;
each R₇ is independently halo, cyano, C₁₋₇alkyl, C₁₋₇haloalkyl, C₁₋₇alkoxy, C₁₋₇ haloalkoxy, or C₃₋₈cycloalkyl, wherein the C₁₋₇alkyl is optionally substituted with OH;
R₁₆ is H; and.
R₁₇ is H.

In some aspects, the present disclosure provides, inter alia, a compound of Formula (I):

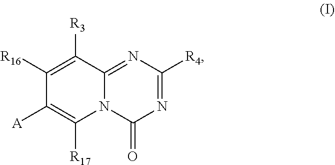

or a pharmaceutically acceptable salt, solvate, or prodrug thereof, wherein:
A is saturated or partially unsaturated mono- or bi-cyclic 4- to 9-membered heterocycloalkyl or NR₁R₂, wherein the heterocycloalkyl comprises 1 or 2 nitrogen ring atoms and is optionally substituted with 1, 2, 3, or 4 R₆;
R₁ is heterocycloalkyl comprising 1 nitrogen ring atom, optionally substituted with 1, 2, 3, or 4 R₆;
R₂ is hydrogen, C₁₋₇alkyl, or C₃₋₈cycloalkyl;
R₃ is H, halo, C₁₋₇alkyl, OR₅, N(R₅)₂, C₃₋₈cycloalkyl, or heterocycloalkyl;
R₄ is aryl or bicyclic 9-membered heteroaryl comprising 2, 3, or 4 heteroatoms independently selected from N, O, and S, wherein R₄ is optionally substituted with 1, 2, or 3 R₇;
each R₅ is independently C₁₋₇alkyl, C₃₋₈cycloalkyl, or heterocycloalkyl;
each R₆ is independently C₁₋₇alkyl, amino, amino-C₁₋₇alkyl, C₃₋₈cycloalkyl, heterocycloalkyl, or C₁₋₇alkoxy-heterocycloalkyl, or two R₆ together form C₁₋₇alkylene;
each R₇ is independently halo, cyano, C₁₋₇alkyl, C₁₋₇haloalkyl, C₁₋₇alkoxy, C₁₋₇ haloalkoxy, or C₃₋₈cycloalkyl, wherein the C₁₋₇alkyl is optionally substituted with OH;
R₁₆ is H, halo, C₁₋₇alkyl, or OR₅; and.
R₁₇ is H, halo, C₁₋₇alkyl, or OR₅.

In some aspects, the present disclosure provides, inter alia, a compound of Formula (I):

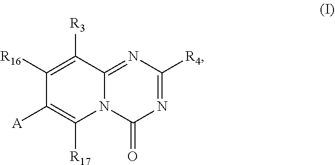

or a pharmaceutically acceptable salt, solvate, or prodrug thereof, wherein:
A is a nitrogen-containing heterocycloalkyl or NR₁R₂, wherein the nitrogen-containing heterocycloalkyl comprises 1 or 2 nitrogen ring atoms and is optionally substituted with 1, 2, 3, or 4 R₆;
R₁ is heterocycloalkyl comprising 1 nitrogen ring atom, optionally substituted with 1, 2, 3, or 4 R₆;
R₂ is hydrogen, C₁₋₇alkyl, or C₃₋₈cycloalkyl;
R₃ is H, C₁₋₇alkyl, OR₅, N(R₅)₂, C₃₋₈cycloalkyl, or heterocycloalkyl;
R₄ is a bicyclic 9-membered heteroaryl comprising 2, 3, or 4 heteroatoms independently selected from N and O, wherein R₄ can be optionally substituted with 1, 2, or 3 R₇;
each R₅ is independently H, C₁₋₇alkyl, C₃₋₈cycloalkyl, or heterocycloalkyl;
each R₆ is independently C₁₋₇alkyl, amino, amino-C₁₋₇alkyl, C₃₋₈cycloalkyl, heterocycloalkyl, and C₁₋₇alkoxy-heterocycloalkyl, or two R₆ together form C₁₋₇alkylene;
each R₇ is independently halo, C₁₋₇alkyl, C₁₋₇haloalkyl, C₁₋₇alkoxy, or C₁₋₇haloalkoxy;
R₁₆ is H, halo, C₁₋₇alkyl, OR₅, or N(R₅)₂; and.
R₁₇ is H, halo, C₁₋₇alkyl, OR₅, or N(R₅)₂.

In some aspects, the present disclosure provides, inter alia, a compound of Formula (I):

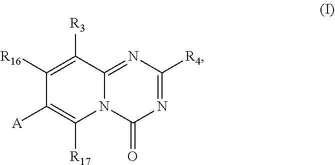

or a pharmaceutically acceptable salt, solvate, or prodrug thereof, wherein:
A is a nitrogen-containing heterocycloalkyl or NR₁R₂, wherein the nitrogen-containing heterocycloalkyl comprises 1 or 2 nitrogen ring atoms and is optionally substituted with 1, 2, 3, or 4 R₆;
R₁ is heterocycloalkyl comprising 1 nitrogen ring atom, optionally substituted with 1, 2, 3, or 4 R₆;
R₂ is hydrogen, C₁₋₇alkyl, or C₃₋₈cycloalkyl;
R₃ is H, C₁₋₇alkyl, OR₅, N(R₅)₂, C₃₋₈cycloalkyl, or heterocycloalkyl;
R₄ is a bicyclic 9-membered heteroaryl comprising 2, 3, or 4 heteroatoms independently selected from N and O, wherein R₄ can be optionally substituted with 1, 2, or 3 R₇;
each R₅ is independently H, C₁₋₇alkyl, C₃₋₈cycloalkyl, or heterocycloalkyl;
each R₆ is independently C₁₋₇alkyl, amino, amino-C₁₋₇alkyl, C₃₋₈cycloalkyl, heterocycloalkyl, and C₁₋₇alkoxy-heterocycloalkyl, or two R₆ together form C₁₋₇alkylene;
each R₇ is independently halo, C₁₋₇alkyl, C₁₋₇haloalkyl, C₁₋₇alkoxy, or C₁₋₇haloalkoxy;
R₁₆ is H; and.
R₁₇ is H.

In some aspects, the present disclosure provides, inter alia, a compound of Formula (I):

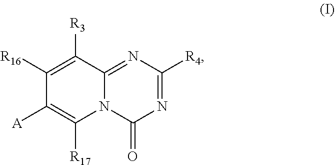

or a pharmaceutically acceptable salt, solvate, or prodrug thereof, wherein:
A is a nitrogen-containing heterocycloalkyl, wherein the nitrogen-containing heterocycloalkyl comprises 1 or 2 nitrogen ring atoms and is optionally substituted with 1, 2, 3, or 4 R₆;
R₃ is H, C₁₋₇alkyl, OR₅, N(R₅)₂, C₃₋₈cycloalkyl, or heterocycloalkyl;
R₄ is a bicyclic 9-membered heteroaryl comprising 2, 3, or 4 heteroatoms independently selected from N and O, wherein R₄ can be optionally substituted with 1, 2, or 3 R₇;
each R₅ is independently H, C₁₋₇alkyl, C₃₋₈cycloalkyl, or heterocycloalkyl;
each R₆ is independently C₁₋₇alkyl, amino, amino-C₁₋₇alkyl, C₃₋₈cycloalkyl, heterocycloalkyl, and C₁₋₇alkoxy-heterocycloalkyl, or two R₆ together form C₁₋₇alkylene;
each R₇ is independently halo, C₁₋₇alkyl, C₁₋₇haloalkyl, C₁₋₇alkoxy, or C₁₋₇haloalkoxy;
R₁₆ is H, halo, C₁₋₇alkyl, or OR₅; and.
R₁₇ is H, halo, C₁₋₇alkyl, or OR₅.

In some aspects, the present disclosure provides, inter alia, a compound of Formula (II):

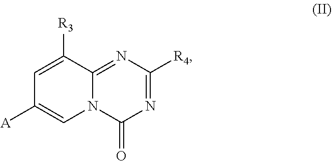

or a pharmaceutically acceptable salt, solvate, or prodrug thereof, wherein:
A is saturated or partially unsaturated mono- or bi-cyclic 4- to 9-membered heterocycloalkyl or NR₁R₂, wherein the heterocycloalkyl comprises 1 or 2 nitrogen ring atoms and is optionally substituted with 1, 2, 3, or 4 R₆;
R₁ is heterocycloalkyl comprising 1 nitrogen ring atom, optionally substituted with 1, 2, 3, or 4 R₆;
R₂ is hydrogen, C₁₋₇alkyl, or C₃₋₈cycloalkyl;
R₃ is H, halo, C₁₋₇alkyl, OR₅, N(R₅)₂, C₃₋₈cycloalkyl, or heterocycloalkyl;
R₄ is aryl or bicyclic 9-membered heteroaryl comprising 2, 3, or 4 heteroatoms independently selected from N, O, and S, wherein R₄ is optionally substituted with 1, 2, or 3 R₇;
each R₅ is independently C₁₋₇alkyl, C₃₋₈cycloalkyl, or heterocycloalkyl;
each R₆ is independently C₁₋₇alkyl, amino, amino-C₁₋₇alkyl, C₃₋₈cycloalkyl, heterocycloalkyl, or C₁₋₇alkoxy-heterocycloalkyl, or two R₆ together form C₁₋₇alkylene; and
each R₇ is independently halo, cyano, C₁₋₇alkyl, C₁₋₇haloalkyl, C₁₋₇alkoxy, C₁₋₇ haloalkoxy, or C₃₋₈cycloalkyl, wherein the C₁₋₇alkyl is optionally substituted with OH.

In some aspects, the present disclosure provides, inter alia, a compound of Formula (II):

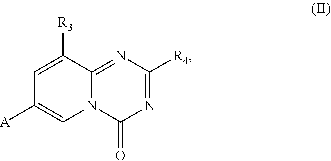

or a pharmaceutically acceptable salt, solvate, or prodrug thereof, wherein:
A is nitrogen-containing heterocycloalkyl or NR₁R₂, wherein the nitrogen-containing heterocycloalkyl comprises 1 or 2 nitrogen ring atoms and is optionally substituted with 1, 2, 3, or 4 R₆;
R₁ is heterocycloalkyl comprising 1 nitrogen ring atom, optionally substituted with 1, 2, 3, or 4 R₆;
R₂ is hydrogen, C₁₋₇alkyl, or C₃₋₈cycloalkyl;
R₃ is H, C₁₋₇alkyl, OR₅, N(R₅)₂, C₃₋₈cycloalkyl, or heterocycloalkyl;
R₄ is a bicyclic 9-membered heteroaryl comprising 2, 3, or 4 heteroatoms independently selected from N and O, wherein R₄ is optionally substituted with 1, 2, or 3 R₇;
each R₅ is independently C₁₋₇alkyl, C₃₋₈cycloalkyl, or heterocycloalkyl;
each R₆ is independently C₁₋₇alkyl, amino, amino-C₁₋₇alkyl, C₃₋₈cycloalkyl, heterocycloalkyl, and C₁₋₇alkoxy-heterocycloalkyl, or two R₆ together form C₁₋₇alkylene; and
each R₇ is independently halo, C₁₋₇alkyl, C₁₋₇haloalkyl, C₁₋₇alkoxy, or C₁₋₇haloalkoxy.

In some aspects, the present disclosure provides, inter alia, a compound of Formula (II):

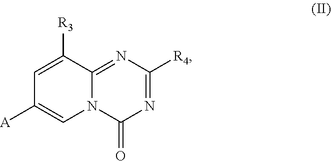

or a pharmaceutically acceptable salt, solvate, or prodrug thereof, wherein:
A is nitrogen-containing heterocycloalkyl, wherein the nitrogen-containing heterocycloalkyl comprises 1 or 2 nitrogen ring atoms and is optionally substituted with 1, 2, 3, or 4 R₆;
R₃ is H, C₁₋₇alkyl, OR₅, N(R₅)₂, C₃₋₈cycloalkyl, or heterocycloalkyl;
R₄ is a bicyclic 9-membered heteroaryl comprising 2, 3, or 4 heteroatoms independently selected from N and O, wherein R₄ is optionally substituted with 1, 2, or 3 R₇;
each R₅ is independently C₁₋₇alkyl, C₃₋₈cycloalkyl, or heterocycloalkyl;
each R₆ is independently C₁₋₇alkyl, amino, amino-C₁₋₇alkyl, C₃₋₈cycloalkyl, heterocycloalkyl, and C₁₋₇alkoxy-heterocycloalkyl, or two R₆ together form C₁₋₇alkylene; and
each R₇ is independently halo or C₁₋₇alkyl.

In some aspects, the present disclosure provides, inter alia, a compound of Formula (II):

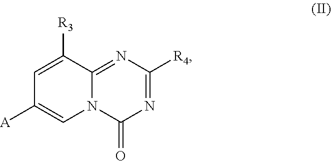

or a pharmaceutically acceptable salt, solvate, or prodrug thereof, wherein:
A is nitrogen-containing heterocycloalkyl, wherein the nitrogen-containing heterocycloalkyl comprises 1 or 2 nitrogen ring atoms and is optionally substituted with 1, 2, 3, or 4 R₆;
R₃ is H, C₁₋₇alkyl, OR₅, N(R₅)₂, C₃₋₈cycloalkyl, or heterocycloalkyl;
R₄ is a bicyclic 9-membered heteroaryl comprising 2, 3, or 4 heteroatoms independently selected from N and O, wherein R₄ is optionally substituted with 1, 2, or 3 R₇;
each R₅ is independently C₁₋₇alkyl, C₃₋₈cycloalkyl, or heterocycloalkyl;
each R₆ is independently C₁₋₇alkyl, amino, amino-C₁₋₇alkyl, C₃₋₈cycloalkyl, heterocycloalkyl, and C₁₋₇alkoxy-heterocycloalkyl, or two R₆ together form C₁₋₇alkylene; and
each R₇ is independently halo or C₁₋₇alkyl.

It is understood that, for a compound of Formula (I) or (II), A, R₁, R₂, R₃, R₄, R₅, R₆, and R₇ can each be, where applicable, selected from the groups described herein, and any group described herein for any of A, R₁, R₂, R₃, R₄, R₅, R₆, and R₇ can be combined, where applicable, with any group described herein for one or more of the remainder of A, R₁, R₂, R₃, R₄, R₅, R₆, and R₇.

In some embodiments, A is nitrogen-containing heterocycloalkyl, wherein the nitrogen-containing heterocycloalkyl comprises 1 or 2 nitrogen ring atoms and is optionally substituted with 1, 2, 3, or 4 R₆, and is bonded to formula (I) or (II) by one of its nitrogen atoms wherein each R₆ is independently C₁₋₇alkyl, amino, amino-C₁₋₇alkyl, C₃₋₈cycloalkyl, heterocycloalkyl, or C₁₋₇alkoxy-heterocycloalkyl or two R₆ together form C₁₋₇alkylene.

In some embodiments each R₆ is independently CI-7 alkyl, heterocycloalkyl, or C₁₋₇alkoxy-heterocycloalkyl or two R₆ together form C₁—. In some embodiments each R₆ is independently methyl, ethyl, isopropyl, methoxy-azetidinyl, or pyrrolidinyl, or two R₆ together form ethylene or propylene. In some embodiments, two R₆ together form C₁₋₇alkylene.

In some embodiments, A is saturated or partially unsaturated mono- or bi-cyclic 4- to 9-membered heterocycloalkyl or NR₁R₂, wherein the heterocycloalkyl comprises 1 or 2 nitrogen ring atoms and is optionally substituted with 1, 2, 3, or 4 R₆.

In some embodiments, A is saturated or partially unsaturated mono- or bi-cyclic 4- to 9-membered heterocycloalkyl, wherein the heterocycloalkyl comprises 1 or 2 nitrogen ring atoms and is optionally substituted with 1, 2, 3, or 4 R₆.

In some embodiments, A is saturated or partially unsaturated mono- or bi-cyclic 4- to 9-membered heterocycloalkyl, wherein the heterocycloalkyl comprises 1 or 2 nitrogen ring atoms.

In some embodiments, A is saturated mono- or bi-cyclic 4- to 9-membered heterocycloalkyl, wherein the heterocycloalkyl comprises 1 or 2 nitrogen ring atoms and is optionally substituted with 1, 2, 3, or 4 R₆.

In some embodiments, A is saturated mono- or bi-cyclic 4- to 9-membered heterocycloalkyl, wherein the heterocycloalkyl comprises 1 or 2 nitrogen ring atoms.

In some embodiments, A is partially unsaturated mono- or bi-cyclic 4- to 9-membered heterocycloalkyl, wherein the heterocycloalkyl comprises 1 or 2 nitrogen ring atoms and is optionally substituted with 1, 2, 3, or 4 R₆.

In some embodiments, A is partially unsaturated mono- or bi-cyclic 4- to 9-membered heterocycloalkyl, wherein the heterocycloalkyl comprises 1 or 2 nitrogen ring atoms.

In some embodiments, A is saturated monocyclic 4- to 9-membered heterocycloalkyl, wherein the heterocycloalkyl comprises 1 or 2 nitrogen ring atoms and is optionally substituted with 1, 2, 3, or 4 R₆.

In some embodiments, A is saturated monocyclic 4- to 9-membered heterocycloalkyl, wherein the heterocycloalkyl comprises 1 or 2 nitrogen ring atoms.

In some embodiments, A is partially unsaturated monocyclic 4- to 9-membered heterocycloalkyl, wherein the heterocycloalkyl comprises 1 or 2 nitrogen ring atoms and is optionally substituted with 1, 2, 3, or 4 R₆.

In some embodiments, A is partially unsaturated monocyclic 4- to 9-membered heterocycloalkyl, wherein the heterocycloalkyl comprises 1 or 2 nitrogen ring atoms.

In some embodiments, A is saturated bicyclic 4- to 9-membered heterocycloalkyl, wherein the heterocycloalkyl comprises 1 or 2 nitrogen ring atoms and is optionally substituted with 1, 2, 3, or 4 R₆.

In some embodiments, A is saturated bicyclic 4- to 9-membered heterocycloalkyl, wherein the heterocycloalkyl comprises 1 or 2 nitrogen ring atoms.

In some embodiments, A is partially unsaturated bicyclic 4- to 9-membered heterocycloalkyl, wherein the heterocycloalkyl comprises 1 or 2 nitrogen ring atoms and is optionally substituted with 1, 2, 3, or 4 R₆.

In some embodiments, A is partially unsaturated bicyclic 4- to 9-membered heterocycloalkyl, wherein the heterocycloalkyl comprises 1 or 2 nitrogen ring atoms.

In some embodiments, A is saturated or partially unsaturated mono- or bicyclic 4- to 8-membered heterocycloalkyl, wherein the heterocycloalkyl comprises 1 or 2 nitrogen ring atoms and is optionally substituted with 1, 2, 3, or 4 R₆.

In some embodiments, A is saturated or partially unsaturated mono- or bi-cyclic 4- to 8-membered heterocycloalkyl, wherein the heterocycloalkyl comprises 1 or 2 nitrogen ring atoms.

In some embodiments, A is saturated or partially unsaturated mono- or bi-cyclic 4- to 7-membered heterocycloalkyl, wherein the heterocycloalkyl comprises 1 or 2 nitrogen ring atoms and is optionally substituted with 1, 2, 3, or 4 R₆.

In some embodiments, A is saturated or partially unsaturated mono- or bi-cyclic 4- to 7-membered heterocycloalkyl, wherein the heterocycloalkyl comprises 1 or 2 nitrogen ring atoms.

In some embodiments, A is saturated or partially unsaturated mono- or bi-cyclic 4- to 6-membered heterocycloalkyl, wherein the heterocycloalkyl comprises 1 or 2 nitrogen ring atoms and is optionally substituted with 1, 2, 3, or 4 R₆.

In some embodiments, A is saturated or partially unsaturated mono- or bi-cyclic 4- to 6-membered heterocycloalkyl, wherein the heterocycloalkyl comprises 1 or 2 nitrogen ring atoms.

In some embodiments, A is saturated or partially unsaturated mono- or bi-cyclic 4- to 5-membered heterocycloalkyl, wherein the heterocycloalkyl comprises 1 or 2 nitrogen ring atoms and is optionally substituted with 1, 2, 3, or 4 R₆.

In some embodiments, A is saturated or partially unsaturated mono- or bi-cyclic 4- to 5-membered heterocycloalkyl, wherein the heterocycloalkyl comprises 1 or 2 nitrogen ring atoms.

In some embodiments, A is saturated or partially unsaturated mono- or bi-cyclic 5- to 9-membered heterocycloalkyl, wherein the heterocycloalkyl comprises 1 or 2 nitrogen ring atoms and is optionally substituted with 1, 2, 3, or 4 R₆.

In some embodiments, A is saturated or partially unsaturated mono- or bi-cyclic 5- to 9-membered heterocycloalkyl, wherein the heterocycloalkyl comprises 1 or 2 nitrogen ring atoms.

In some embodiments, A is saturated or partially unsaturated mono- or bi-cyclic 5- to 8-membered heterocycloalkyl, wherein the heterocycloalkyl comprises 1 or 2 nitrogen ring atoms and is optionally substituted with 1, 2, 3, or 4 R₆.

In some embodiments, A is saturated or partially unsaturated mono- or bi-cyclic 5- to 8-membered heterocycloalkyl, wherein the heterocycloalkyl comprises 1 or 2 nitrogen ring atoms.

In some embodiments, A is saturated or partially unsaturated mono- or bi-cyclic 5- to 7-membered heterocycloalkyl, wherein the heterocycloalkyl comprises 1 or 2 nitrogen ring atoms and is optionally substituted with 1, 2, 3, or 4 R₆.

In some embodiments, A is saturated or partially unsaturated mono- or bi-cyclic 5- to 7-membered heterocycloalkyl, wherein the heterocycloalkyl comprises 1 or 2 nitrogen ring atoms.

In some embodiments, A is saturated or partially unsaturated mono- or bi-cyclic 6- to 9-membered heterocycloalkyl, wherein the heterocycloalkyl comprises 1 or 2 nitrogen ring atoms and is optionally substituted with 1, 2, 3, or 4 R₆.

In some embodiments, A is saturated or partially unsaturated mono- or bi-cyclic 6- to 9-membered heterocycloalkyl, wherein the heterocycloalkyl comprises 1 or 2 nitrogen ring atoms.

In some embodiments, A is saturated or partially unsaturated mono- or bi-cyclic 6- to 8-membered heterocycloalkyl, wherein the heterocycloalkyl comprises 1 or 2 nitrogen ring atoms and is optionally substituted with 1, 2, 3, or 4 R₆.

In some embodiments, A is saturated or partially unsaturated mono- or bi-cyclic 6- to 8-membered heterocycloalkyl, wherein the heterocycloalkyl comprises 1 or 2 nitrogen ring atoms.

In some embodiments, A is saturated or partially unsaturated mono- or bi-cyclic 6- to 7-membered heterocycloalkyl, wherein the heterocycloalkyl comprises 1 or 2 nitrogen ring atoms and is optionally substituted with 1, 2, 3, or 4 R₆.

In some embodiments, A is saturated or partially unsaturated mono- or bi-cyclic 6- to 7-membered heterocycloalkyl, wherein the heterocycloalkyl comprises 1 or 2 nitrogen ring atoms.

In some embodiments, A is saturated or partially unsaturated mono- or bi-cyclic 7- to 9-membered heterocycloalkyl, wherein the heterocycloalkyl comprises 1 or 2 nitrogen ring atoms and is optionally substituted with 1, 2, 3, or 4 R₆.

In some embodiments, A is saturated or partially unsaturated mono- or bi-cyclic 7- to 9-membered heterocycloalkyl, wherein the heterocycloalkyl comprises 1 or 2 nitrogen ring atoms.

In some embodiments, A is saturated or partially unsaturated mono- or bi-cyclic 7- to 8-membered heterocycloalkyl, wherein the heterocycloalkyl comprises 1 or 2 nitrogen ring atoms and is optionally substituted with 1, 2, 3, or 4 R₆.

In some embodiments, A is saturated or partially unsaturated mono- or bi-cyclic 7- to 8-membered heterocycloalkyl, wherein the heterocycloalkyl comprises 1 or 2 nitrogen ring atoms.

In some embodiments, A is saturated or partially unsaturated mono- or bi-cyclic 9-membered heterocycloalkyl, wherein the heterocycloalkyl comprises 1 or 2 nitrogen ring atoms and is optionally substituted with 1, 2, 3, or 4 R₆.

In some embodiments, A is saturated or partially unsaturated mono- or bi-cyclic 9-membered heterocycloalkyl, wherein the heterocycloalkyl comprises 1 or 2 nitrogen ring atoms.

In some embodiments, A is saturated or partially unsaturated mono- or bi-cyclic 8-membered heterocycloalkyl, wherein the heterocycloalkyl comprises 1 or 2 nitrogen ring atoms and is optionally substituted with 1, 2, 3, or 4 R₆.

In some embodiments, A is saturated or partially unsaturated mono- or bi-cyclic 8-membered heterocycloalkyl, wherein the heterocycloalkyl comprises 1 or 2 nitrogen ring atoms.

In some embodiments, A is saturated or partially unsaturated mono- or bi-cyclic 7-membered heterocycloalkyl, wherein the heterocycloalkyl comprises 1 or 2 nitrogen ring atoms and is optionally substituted with 1, 2, 3, or 4 R₆.

In some embodiments, A is saturated or partially unsaturated mono- or bi-cyclic 7-membered heterocycloalkyl, wherein the heterocycloalkyl comprises 1 or 2 nitrogen ring atoms.

In some embodiments, A is saturated or partially unsaturated mono- or bi-cyclic 6-membered heterocycloalkyl, wherein the heterocycloalkyl comprises 1 or 2 nitrogen ring atoms and is optionally substituted with 1, 2, 3, or 4 R₆.

In some embodiments, A is saturated or partially unsaturated mono- or bi-cyclic 6-membered heterocycloalkyl, wherein the heterocycloalkyl comprises 1 or 2 nitrogen ring atoms.

In some embodiments, A is saturated or partially unsaturated mono- or bi-cyclic 5-membered heterocycloalkyl, wherein the heterocycloalkyl comprises 1 or 2 nitrogen ring atoms and is optionally substituted with 1, 2, 3, or 4 R₆.

In some embodiments, A is saturated or partially unsaturated mono- or bi-cyclic 5-membered heterocycloalkyl, wherein the heterocycloalkyl comprises 1 or 2 nitrogen ring atoms.

In some embodiments, A is saturated or partially unsaturated mono- or bi-cyclic 4-membered heterocycloalkyl, wherein the heterocycloalkyl comprises 1 or 2 nitrogen ring atoms and is optionally substituted with 1, 2, 3, or 4 R₆.

In some embodiments, A is saturated or partially unsaturated mono- or bi-cyclic 4-membered heterocycloalkyl, wherein the heterocycloalkyl comprises 1 or 2 nitrogen ring atoms.

In some embodiments, A is saturated or partially unsaturated mono- or bi-cyclic 4- to 9-membered heterocycloalkyl, wherein the heterocycloalkyl comprises 1 or 2 nitrogen ring atoms and is substituted with 1, 2, 3, or 4 R₆.

In some embodiments, A is saturated or partially unsaturated mono- or bi-cyclic 4- to 9-membered heterocycloalkyl, wherein the heterocycloalkyl comprises 1 or 2 nitrogen ring atoms and is substituted with 1, 2, or 3 R₆.

In some embodiments, A is saturated or partially unsaturated mono- or bi-cyclic 4- to 9-membered heterocycloalkyl, wherein the heterocycloalkyl comprises 1 or 2 nitrogen ring atoms and is substituted with 1 R₆.

In some embodiments, A is saturated or partially unsaturated mono- or bi-cyclic 4- to 9-membered heterocycloalkyl, wherein the heterocycloalkyl comprises 1 or 2 nitrogen ring atoms and is substituted with 2 R₆.

In some embodiments, A is saturated or partially unsaturated mono- or bi-cyclic 4- to 9-membered heterocycloalkyl, wherein the heterocycloalkyl comprises 1 or 2 nitrogen ring atoms and is substituted with 3 R₆.

In some embodiments, A is saturated or partially unsaturated mono- or bi-cyclic 4- to 9-membered heterocycloalkyl, wherein the heterocycloalkyl comprises 1 nitrogen ring atom.

In some embodiments, A is saturated or partially unsaturated mono- or bi-cyclic 4- to 9-membered heterocycloalkyl, wherein the heterocycloalkyl comprises 2 nitrogen ring atoms.

In some embodiments, A is saturated or partially unsaturated mono- or bi-cyclic 4- to 9-membered heterocycloalkyl, wherein the heterocycloalkyl comprises 1 nitrogen ring atom and is optionally substituted with 1, 2, 3, or 4 R₆.

In some embodiments, A is saturated or partially unsaturated mono- or bi-cyclic 4- to 9-membered heterocycloalkyl, wherein the heterocycloalkyl comprises 2 nitrogen ring atoms and is optionally substituted with 1, 2, or 3 R₆.

In some embodiments, A is saturated or partially unsaturated mono- or bi-cyclic 4- to 9-membered heterocycloalkyl, wherein the heterocycloalkyl comprises 1 nitrogen ring atom and is substituted with 1, 2, 3, or 4 R₆.

In some embodiments, A is saturated or partially unsaturated mono- or bi-cyclic 4- to 9-membered heterocycloalkyl, wherein the heterocycloalkyl comprises 2 nitrogen ring atoms and is substituted with 1, 2, or 3 R₆.

In some embodiments, A is piperazinyl, diazepanyl, octahydropyrrolopyrazinyl, diazaspirooctanyl, pyrrolidinyl, octahydropyrrolopyrroyl, diazaspirononanyl, diazaspiroheptanyl, or diazabicyclooctanyl, each optionally substituted with 1, 2, 3, or 4 R₆.

In some embodiments, A is

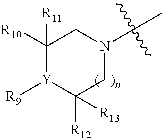

wherein,
Y is absent, N or CH;
R₉ is hydrogen, C₁₋₇alkyl, or (CH₂)ₘ—NR₁₄R₁₅;
R₁₀ is hydrogen or C₁₋₇alkyl;
R₁₁ is hydrogen or C₁₋₇alkyl;
R₁₂ is hydrogen or C₁₋₇alkyl;
R₁₃ is hydrogen or C₁₋₇alkyl;
each R₁₄ and R₁₅ are independently hydrogen, C₁₋₇alkyl and C₃₋₈cycloalkyl;
n is 0, 1 or 2;
m is 0, 1 or 2;
or R₉ and R₁₀ together form C₁₋₇alkylene;
or R₉ and R₁₂ together form C₁₋₇alkylene;
or R₁₀ and R₁₁ together form C₂₋₇alkylene or 4- to 6-membered heterocycloalkyl optionally substituted with C₁₋₇alkyl;
or R₁₀ and R₁₂ together form C₁₋₇alkylene or 4- to 6-membered heterocycloalkyl optionally substituted with C₁₋₇alkyl;
or R₁₀ and R₁₄ together form C₁₋₇alkylene;
or R₁₂ and R₁₃ together form C₂₋₇alkylene;
or R₁₂ and R₁₄ together form C₁₋₇alkylene;
or R₁₄ and R₁₅ together form C₂₋₇alkylene which is optionally substituted with C₁₋₇alkoxy.

In some embodiments, Y is N or CH. In some embodiments, Y is absent. In some embodiments, Y is N. In some embodiments, Y is CH.

In some embodiments, R₉ is hydrogen, C₁₋₇alkyl, or (CH₂)ₘ—NR₁₄R₁₅. In some embodiments, R₉ is hydrogen. In some embodiments, R₉ is C₁₋₇alkyl. In some embodiments, R₉ is (CH₂)ₘ—NR₁₄R₁₅.

In some embodiments, R₁₀ is hydrogen or C₁₋₇alkyl. In some embodiments, R₁₀ is hydrogen. In some embodiments, C₁₋₇alkyl.

In some embodiments, R₁ is hydrogen or C₁₋₇alkyl. In some embodiments, R₁₁ is hydrogen. In some embodiments, C₁₋₇alkyl.

In some embodiments, R₁₂ is hydrogen or C₁₋₇alkyl. In some embodiments, R₁₂ is hydrogen. In some embodiments, C₁₋₇alkyl.

In some embodiments, R₁₃ is hydrogen or C₁₋₇alkyl. In some embodiments, R₁₃ is hydrogen. In some embodiments, C₁₋₇alkyl.

In some embodiments, R₁₄ is hydrogen, C₁₋₇alkyl, or C₃₋₈cycloalkyl. In some embodiments, R₁₄ is hydrogen. In some embodiments, R₁₄ is C₁₋₇alkyl. In some embodiments, R₁₄ is C₃₋₈cycloalkyl.

In some embodiments, R₁₅ is hydrogen, C₁₋₇alkyl, or C₃₋₈cycloalkyl. In some embodiments, R₁₅ is hydrogen. In some embodiments, R₁₅ is C₁₋₇alkyl. In some embodiments, R₁₅ is C₃₋₈cycloalkyl.

In some embodiments, n is 0, 1, or 2. In some embodiments, n is 0. In some embodiments, n is 1. In some embodiments, n is 2.

In some embodiments, m is 0, 1, or 2. In some embodiments, m is 0. In some embodiments, m is 1. In some embodiments, m is 2.

In some embodiments, R₉ and R₁₀ together form C₁₋₇alkylene.

In some embodiments, R₉ and R₁₂ together form C₁₋₇alkylene.

In some embodiments, R₁₀ and R₁₁ together form C₂₋₇alkylene or 4- to 6-membered heterocycloalkyl optionally substituted with C₁₋₇alkyl. In some embodiments, R₁₀ and R₁₁ together form C₂₋₇alkylene.

In some embodiments, R₁₀ and R₁₁ together form 4- to 6-membered heterocycloalkyl optionally substituted with C₁₋₇alkyl. In some embodiments, R₁₀ and R₁₁ together form 4-membered heterocycloalkyl optionally substituted with C₁₋₇alkyl. In some embodiments, R₁₀ and R₁₁ together form 5-membered heterocycloalkyl optionally substituted with C₁₋₇alkyl. In some embodiments, R₁₀ and R₁₁ together form 6-membered heterocycloalkyl optionally substituted with C₁₋₇alkyl.

In some embodiments, R₁₀ and R₁₁ together form 4- to 6-membered heterocycloalkyl substituted with C₁₋₇alkyl. In some embodiments, R₁₀ and R₁₁ together form 4-membered heterocycloalkyl substituted with C₁₋₇alkyl. In some embodiments, R₁₀ and R₁₁ together form 5-membered heterocycloalkyl substituted with C₁₋₇alkyl. In some embodiments, R₁₀ and Ru together form 6-membered heterocycloalkyl substituted with C₁₋₇alkyl.

In some embodiments, R₁₀ and R₁₂ together form C₁₋₇alkylene or 4- to 6-membered heterocycloalkyl optionally substituted with C₁₋₇alkyl. In some embodiments, R₁₀ and R₁₂ together form C₁₋₇alkylene.

In some embodiments, R₁₀ and R₁₂ together form 4- to 6-membered heterocycloalkyl optionally substituted with C₁₋₇alkyl. In some embodiments, R₁₀ and R₁₂ together form 4-membered heterocycloalkyl optionally substituted with C₁₋₇alkyl. In some embodiments, R₁₀ and R₁₂ together form 5-membered heterocycloalkyl optionally substituted with C₁₋₇alkyl. In some embodiments, R₁₀ and R₁₂ together form 6-membered heterocycloalkyl optionally substituted with C₁₋₇alkyl.

In some embodiments, R₁₀ and R₁₂ together form 4- to 6-membered heterocycloalkyl substituted with C₁₋₇alkyl. In some embodiments, R₁₀ and R₁₂ together form 4-membered heterocycloalkyl substituted with C₁₋₇alkyl. In some embodiments, R₁₀ and R₁₂ together form 5-membered heterocycloalkyl substituted with C₁₋₇alkyl. In some embodiments, R₁₀ and R₁₂ together form 6-membered heterocycloalkyl substituted with C₁₋₇alkyl.

In some embodiments, R₁₀ and R₁₄ together form C₁₋₇alkylene.

In some embodiments, R₁₂ and R₁₃ together form C₂₋₇alkylene.

In some embodiments, R₁₂ and R₁₄ together form C₁₋₇alkylene.

In some embodiments, R₁₄ and R₁₅ together form C₂₋₇alkylene.

In some embodiments, R₁₄ and R₁₅ together form C₂₋₇alkylene which is optionally substituted with C₁₋₇alkoxy.

In some embodiments, R₁₄ and R₁₅ together form C₂₋₇alkylene which is substituted with C₁₋₇alkoxy.

In some embodiments, Y is CH and R₉ is (CH₂)ₘ—NR₁₄R₁₅.

In some embodiments, R₉ is hydrogen, pyrrolidinyl, or methoxy-azetidinyl.

In some embodiments, R₁₀ is hydrogen, methyl, ethyl or isopropyl.

In some embodiments, R₁₁ is hydrogen or methyl.

In some embodiments, R₁₂ is hydrogen or methyl.

In some embodiments, R₉ and R₁₀ together form propylene.

In some embodiments, R₁₀ and R₁₁ together form ethylene.

In some embodiments, R₁₄ and R₁₅ together form propylene or butylene.

In some embodiments, A is

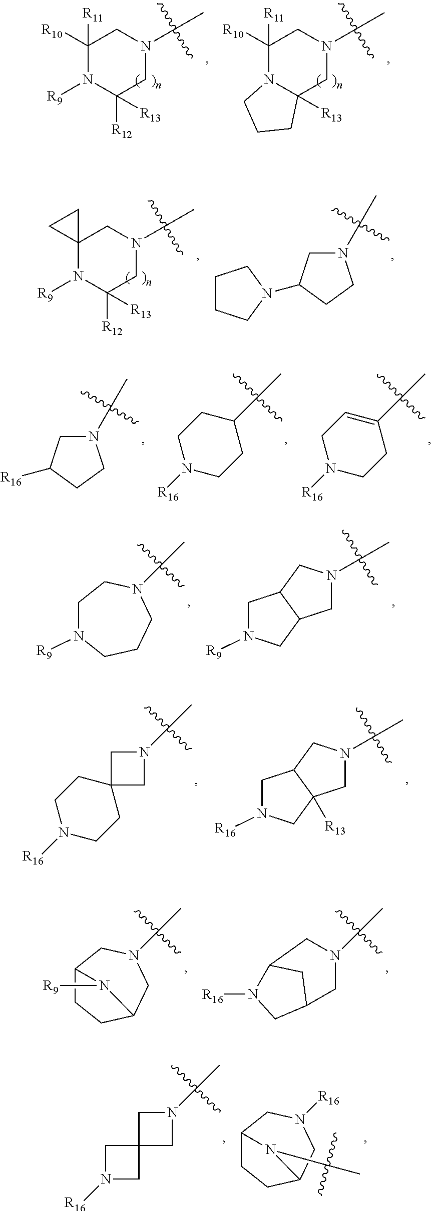

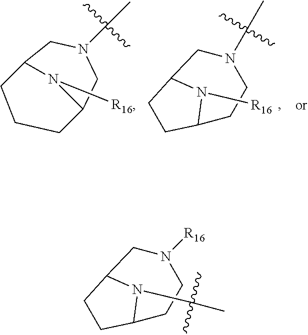

wherein R₉, R₁₀, R₁₁, R₁₂, and R₁₃ are as defined in any one of the preceding claims, and R₁₆ is hydrogen or C₁₋₇alkyl.

In some embodiments, A is

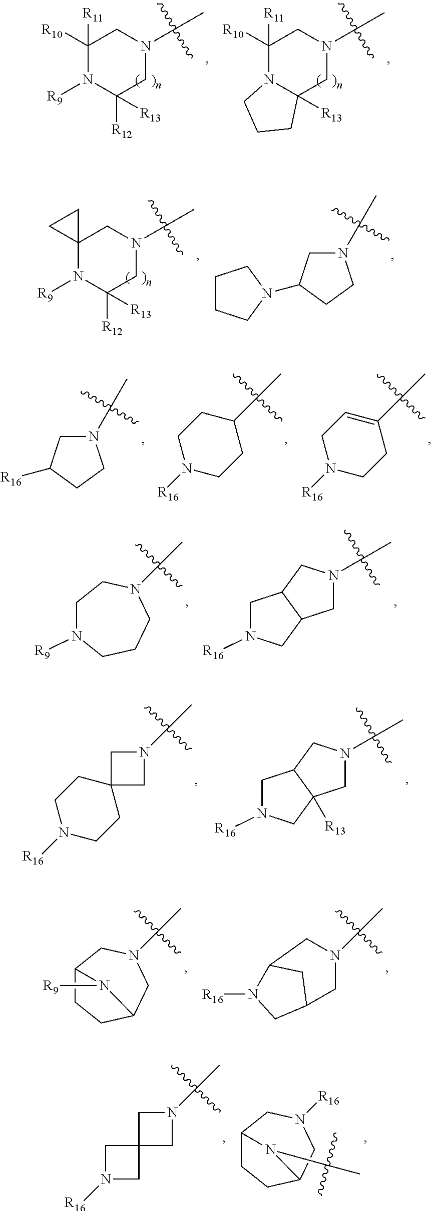

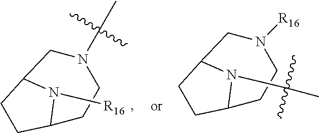

wherein R₉, R₁₀, R₁₁, R₁₂, and R₁₃ are as defined in any one of the preceding claims, and R₁₆ is hydrogen or C₁₋₇alkyl.

In some embodiments, R₁₆ is hydrogen or C₁₋₇alkyl. In some embodiments, R₁₆ is hydrogen. In some embodiments, R₁₆ is C₁₋₇alkyl.

In some embodiments A is

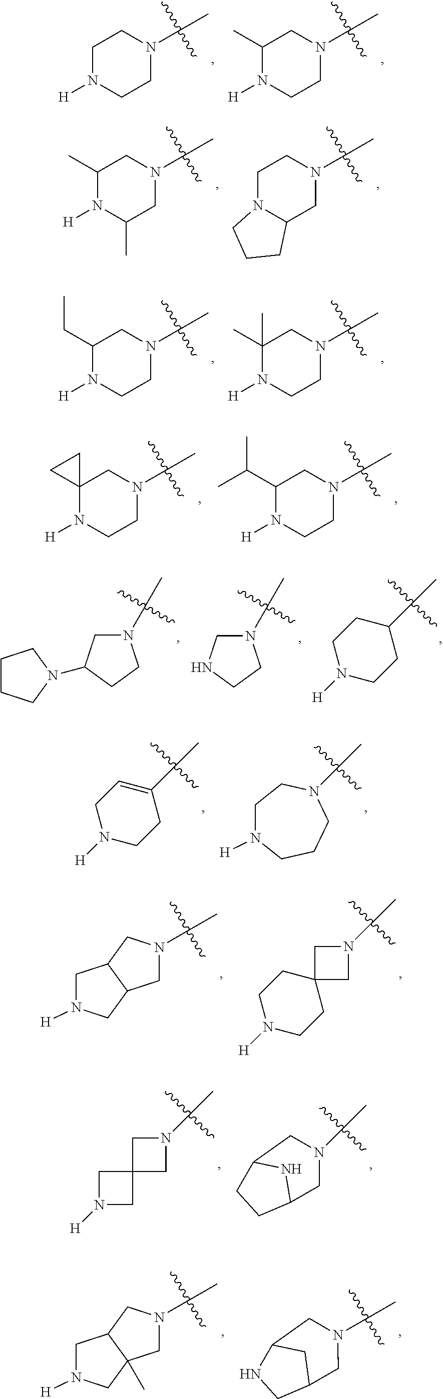

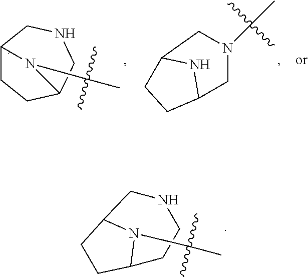 [SMILES: *N1C2CCC1CNC2 ; *N1CC2CCC(C1)N2 ; *N1C2CCNCC1CC2]

In some embodiments A is

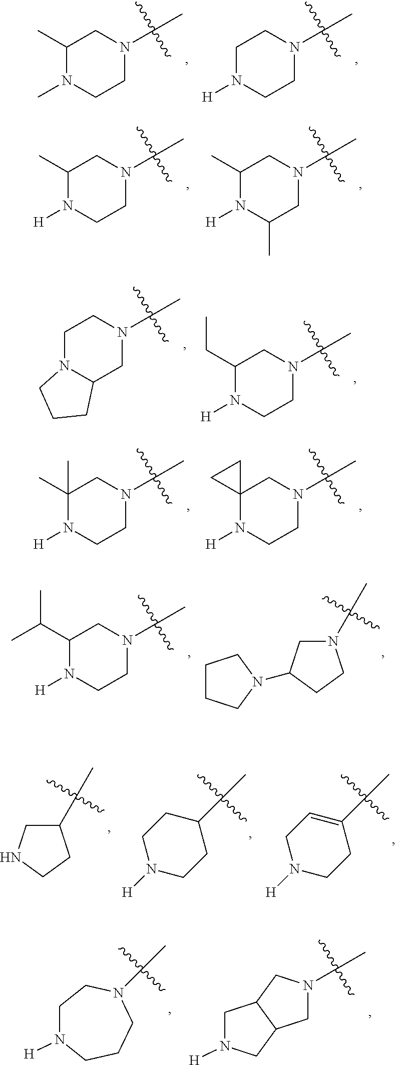

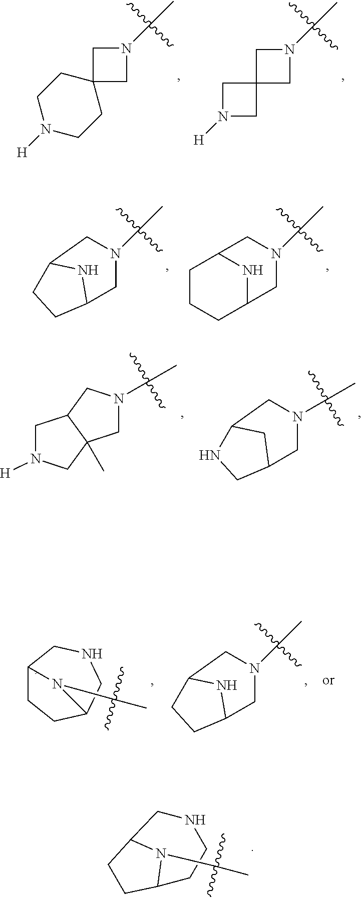

In some embodiments, A is NR₁R₂.

In some embodiments, R₁ is heterocycloalkyl comprising 1 nitrogen ring atom.

In some embodiments, R₁ is heterocycloalkyl comprising 1 nitrogen ring atom, wherein the heterocycloalkyl is optionally substituted with 1, 2, 3, or 4 R₆.

In some embodiments, R₁ is heterocycloalkyl comprising 1 nitrogen ring atom, wherein the heterocycloalkyl is substituted with 1, 2, 3, or 4 R₆.

In some embodiments, R₁ is heterocycloalkyl comprising 1 nitrogen ring atom, wherein the heterocycloalkyl is substituted with 1, 2, or 3 R₆.

In some embodiments, R₁ is heterocycloalkyl comprising 1 nitrogen ring atom, wherein the heterocycloalkyl is substituted with 1 R₆.

In some embodiments, R₁ is heterocycloalkyl comprising 1 nitrogen ring atom, wherein the heterocycloalkyl is substituted with 2 R₆.

In some embodiments, R₁ is heterocycloalkyl comprising 1 nitrogen ring atom, wherein the heterocycloalkyl is substituted with 3 R₆.

In some embodiments, R₁ is heterocycloalkyl comprising 1 nitrogen ring atom, wherein the heterocycloalkyl is substituted with 4 R₆.

In some embodiments, R₂ is hydrogen, C₁₋₇alkyl, or C₃₋₈cycloalkyl.

In some embodiments, R₂ is hydrogen or C₁₋₇alkyl.

In some embodiments, R₂ is hydrogen.

In some embodiments, R₂ is C₁₋₇alkyl. In some embodiments, R₂ is methyl. In some embodiments, R₂ is ethyl. In some embodiments, R₂ is propyl. In some embodiments, R₂ is butyl. In some embodiments, R₂ is pentyl. In some embodiments, R₂ is hexyl. In some embodiments, R₂ is heptyl. In some embodiments, R₂ is isopropyl. In some embodiments, R₂ is isobutyl. In some embodiments, R₂ is isopentyl. In some embodiments, R₂ is isohexyl. In some embodiments, R₂ is secbutyl. In some embodiments, R₂ is secpentyl. In some embodiments, R₂ is sechexyl. In some embodiments, R₂ is tertbutyl.

In some embodiments, R₂ is C₃₋₈cycloalkyl. In some embodiments, R₂ is cyclopropyl, cyclobutyl, cyclopentyl, cyclohexyl, cycloheptyl, or cyclooctyl.

In some embodiments, R₃ is H, halo, C₁₋₇alkyl, —OR₅, —N(R₅)₂, C₃₋₈cycloalkyl, or heterocycloalkyl.

In some embodiments, R₃ is H, C₁₋₇alkyl, —OR₅, —N(R₅)₂, C₃₋₈cycloalkyl, or heterocycloalkyl.

In some embodiments, R₃ is H or C₁₋₇alkyl.

In some embodiments, R₃ is H.

In some embodiments, R₃ is halo. In some embodiments, R₃ is F, Cl, Br, or I. In some embodiments, R₃ is F, Cl, or Br. In some embodiments, R₃ is F or Cl. In some embodiments, R₃ is F. In some embodiments, R₃ is Cl. In some embodiments, R₃ is Br. In some embodiments, R₃ is I.

In some embodiments, R₃ is C₁₋₇alkyl. In some embodiments, R₃ is methyl. In some embodiments, R₃ is ethyl. In some embodiments, R₃ is propyl. In some embodiments, R₃ is butyl. In some embodiments, R₃ is pentyl. In some embodiments, R₃ is hexyl. In some embodiments, R₃ is heptyl. In some embodiments, R₃ is isopropyl. In some embodiments, R₃ is isobutyl. In some embodiments, R₃ is isopentyl. In some embodiments, R₃ is isohexyl. In some embodiments, R₃ is secbutyl. In some embodiments, R₃ is secpentyl. In some embodiments, R₃ is sechexyl. In some embodiments, R₃ is tertbutyl.

In some embodiments, R₃ is —OR₅ or —N(R₅)₂. In some embodiments, R₃ is —OR₅. In some embodiments, R₃ is —N(R₅)₂.

In some embodiments, R₃ is C₃₋₈cycloalkyl or heterocycloalkyl.

In some embodiments, R₃ is C₃₋₈cycloalkyl. In some embodiments, R₃ is cyclopropyl, cyclobutyl, cyclopentyl, cyclohexyl, cycloheptyl, or cyclooctyl.

In some embodiments, R₃ is heterocycloalkyl.

In some embodiments, R₄ is aryl or bicyclic 9-membered heteroaryl comprising 2, 3, or 4 heteroatoms independently selected from N, O, or S.

In some embodiments, R₄ is aryl or bicyclic 9-membered heteroaryl comprising 2, 3, or 4 heteroatoms independently selected from N or O.

In some embodiments, R₄ is aryl.

In some embodiments, R₄ is aryl optionally substituted with one, two, or three R₇.

In some embodiments, R₄ is aryl substituted with one, two, or three R₇. In some embodiments, R₄ is aryl substituted with one R₇. In some embodiments, R₄ is aryl substituted with two R₇. In some embodiments, R₄ is aryl substituted with three R₇.

In some embodiments, R₄ is phenyl.

In some embodiments, R₄ is phenyl optionally substituted with one, two, or three R₇.

In some embodiments, R₄ is phenyl substituted with one, two, or three R₇. In some embodiments, R₄ is phenyl substituted with one R₇. In some embodiments, R₄ is phenyl substituted with two R₇. In some embodiments, R₄ is phenyl substituted with three R₇.

In some embodiments, R₄ is a bicyclic 9-membered heteroaryl comprising 2, 3, or 4 heteroatoms independently selected from N, O, and S.

In some embodiments, R₄ is a bicyclic 9-membered heteroaryl comprising 2, 3, or 4 heteroatoms independently selected from N and O.

In some embodiments, R₄ is a bicyclic 9-membered heteroaryl comprising 2 or 3 heteroatoms independently selected from N, O, and S.

In some embodiments, R₄ is a bicyclic 9-membered heteroaryl comprising 2 or 3 heteroatoms independently selected from N and O.

In some embodiments, R₄ is a bicyclic 9-membered heteroaryl comprising 2, 3, or 4 heteroatoms independently selected from N, O, and S, wherein R₄ is optionally substituted with 1, 2, or 3 R₇.

In some embodiments, R₄ is a bicyclic 9-membered heteroaryl comprising 2, 3, or 4 heteroatoms independently selected from N and O, wherein R₄ is optionally substituted 1, 2, or 3 R₇.

In some embodiments, R₄ is a bicyclic 9-membered heteroaryl comprising 2 or 3 heteroatoms independently selected from N, O, and S, wherein R₄ is optionally substituted with 1, 2, or 3 R₇.

In some embodiments, R₄ is a bicyclic 9-membered heteroaryl comprising 2 or 3 heteroatoms independently selected from N and O, wherein R₄ is optionally substituted with 1, 2, or 3 R₇.

In some embodiments, R₄ is a bicyclic 9-membered heteroaryl comprising 2, 3, or 4 heteroatoms independently selected from N and O, wherein R₄ is substituted with 1, 2, or 3 R₇.

In some embodiments, R₄ is a bicyclic 9-membered heteroaryl comprising 2 or 3 heteroatoms independently selected from N and O, wherein R₄ is substituted with 1, 2, or 3 R₇.

In some embodiments, R₄ is a bicyclic 9-membered heteroaryl comprising 2, 3, or 4 heteroatoms independently selected from N and O, wherein R₄ is substituted with 1 R₇.

In some embodiments, R₄ is a bicyclic 9-membered heteroaryl comprising 2 or 3 heteroatoms independently selected from N and O, wherein R₄ is substituted with 1 R₇.

In some embodiments, R₄ is a bicyclic 9-membered heteroaryl comprising 2, 3, or 4 heteroatoms independently selected from N and O, wherein R₄ is substituted with 2 R₇.

In some embodiments, R₄ is a bicyclic 9-membered heteroaryl comprising 2 or 3 heteroatoms independently selected from N and O, wherein R₄ is substituted with 2 R₇.

In some embodiments, R₄ is a bicyclic 9-membered heteroaryl comprising 2, 3, or 4 heteroatoms independently selected from N and O, wherein R₄ is substituted with 3 R₇.

In some embodiments, R₄ is a bicyclic 9-membered heteroaryl comprising 2 or 3 heteroatoms independently selected from N and O, wherein R₄ is substituted with 3 R₇.

In some embodiments, R₄ is a bicyclic 9-membered heteroaryl comprising 2 heteroatoms independently selected from N and O, wherein R₄ is optionally substituted with 1, 2, or 3 R₇.

In some embodiments, R₄ is a bicyclic 9-membered heteroaryl comprising two heteroatoms independently selected from N and O, wherein R₄ is substituted with 1, 2, or 3 R₇.

In some embodiments, R₄ is a bicyclic 9-membered heteroaryl comprising 2 heteroatoms independently selected from N and O, wherein R₄ is substituted with 1 R₇.

In some embodiments, R₄ is a bicyclic 9-membered heteroaryl comprising 2 heteroatoms independently selected from N and O, wherein R₄ is substituted with 2 R₇.

In some embodiments, R₄ is a bicyclic 9-membered heteroaryl comprising 2 heteroatoms independently selected from N and O, wherein R₄ is substituted with 3 R₇.

In some embodiments, R₄ is a bicyclic 9-membered heteroaryl comprising 3 heteroatoms independently selected from N and O, wherein R₄ is optionally substituted with 1, 2, or 3 R₇.

In some embodiments, R₄ is a bicyclic 9-membered heteroaryl comprising 3 heteroatoms independently selected from N and O, wherein R₄ is substituted with 1, 2, or 3 R₇.

In some embodiments, R₄ is a bicyclic 9-membered heteroaryl comprising 3 heteroatoms independently selected from N and O, wherein R₄ is substituted with 1 R₇.

In some embodiments, R₄ is a bicyclic 9-membered heteroaryl comprising 3 heteroatoms independently selected from N and O, wherein R₄ is substituted with 2 R₇.

In some embodiments, R₄ is a bicyclic 9-membered heteroaryl comprising 3 heteroatoms independently selected from N and O, wherein R₄ is substituted with 3 R₇.

In some embodiments, R₄ is a bicyclic 9-membered heteroaryl comprising four heteroatoms independently selected from N and O, wherein R₄ is optionally substituted with 1, 2, or 3 R₇.

In some embodiments, R₄ is a bicyclic 9-membered heteroaryl comprising 4 heteroatoms independently selected from N and O, wherein R₄ is substituted with 1, 2, or 3 R₇.

In some embodiments, R₄ is a bicyclic 9-membered heteroaryl comprising 4 heteroatoms independently selected from N and O, wherein R₄ is substituted with 1 R₇.

In some embodiments, R₄ is a bicyclic 9-membered heteroaryl comprising 4 heteroatoms independently selected from N and O, wherein R₄ is substituted with 2 R₇.

In some embodiments, R₄ is a bicyclic 9-membered heteroaryl comprising 4 heteroatoms independently selected from N and O, wherein R₄ is substituted with 3 R₇.

In some embodiments, R₄ is imidazo[1,2-a]pyrazine, benzo[d]oxazole, triazolo-pyridazinyl, or imidazo[1,2-a]pyrazine each optionally substituted with 1, 2, 3, or 4 R₇.

In some embodiments, R₄ is

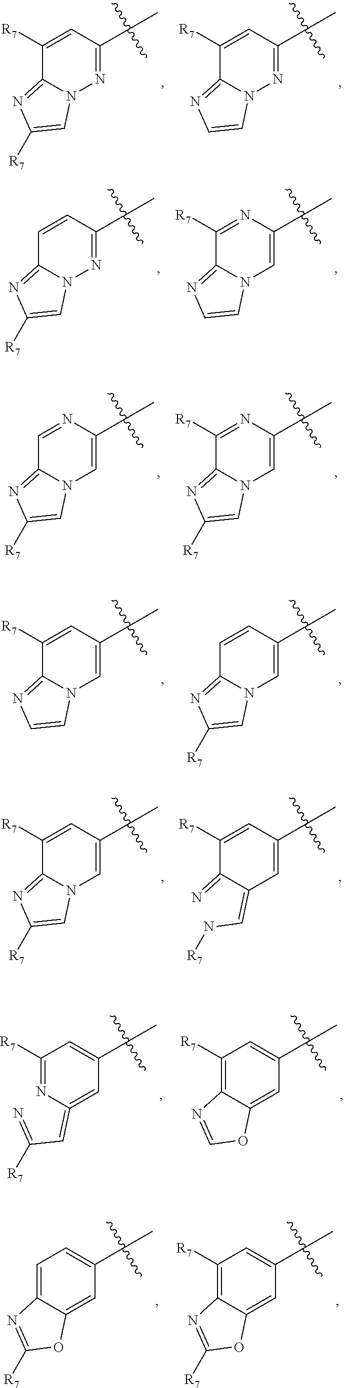

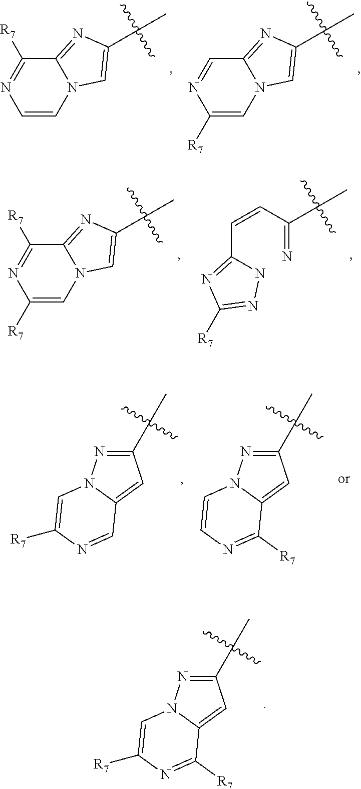

In some embodiments, R₄ is

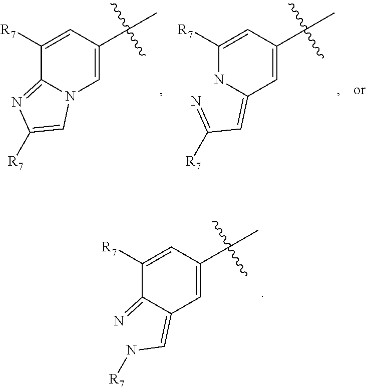

In some embodiments, R₄ is

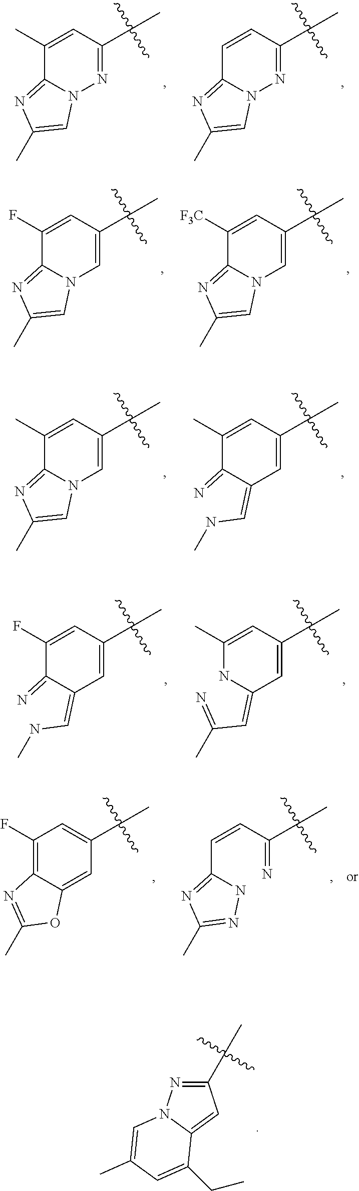

In some embodiments, R₄ is

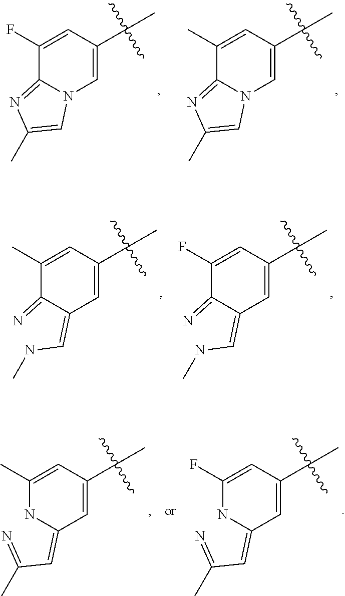 [SMILES: *c1cc(F)c2nc(C)cn2c1 ; *c1cc(C)c2nc(C)cn2c1 ; *C1=C/C(=C/NC)C(=N)C(C)=C1 ; *C1=C/C(=C/NC)C(=N)C(F)=C1 ; *C1=C/C(=C/C(C)=N)NC(C)=C1 ; *C1=C/C(=C/C(C)=N)NC(F)=C1]

In some embodiments, each R₅ is independently C₁₋₇alkyl, C₃₋₈cycloalkyl, or heterocycloalkyl.

In some embodiments, each R₅ is independently C₁₋₇alkyl. In some embodiments, each R₅ is independently methyl. In some embodiments, each R₅ is independently ethyl. In some embodiments, each R₅ is independently propyl. In some embodiments, each R₅ is independently butyl. In some embodiments, each R₅ is independently pentyl. In some embodiments, each R₅ is independently hexyl. In some embodiments, each R₅ is independently heptyl. In some embodiments, each R₅ is independently isopropyl. In some embodiments, each R₅ is independently isobutyl. In some embodiments, each R₅ is independently isopentyl. In some embodiments, each R₅ is independently isohexyl. In some embodiments, each R₅ is independently secbutyl. In some embodiments, each R₅ is independently secpentyl. In some embodiments, each R₅ is independently sechexyl. In some embodiments, each R₅ is independently tertbutyl.

In some embodiments, each R₅ is independently C₃₋₈cycloalkyl or heterocycloalkyl.

In some embodiments, each R₅ is independently C₃₋₈cycloalkyl. In some embodiments, R₅ is cyclopropyl, cyclobutyl, cyclopentyl, cyclohexyl, cycloheptyl, or cyclooctyl.

In some embodiments, each R₅ is independently heterocycloalkyl.

In some embodiments, each R₆ is independently C₁₋₇alkyl, amino, amino-C₁₋₇alkyl, C₃₋₈cycloalkyl, heterocycloalkyl and C₁₋₇alkoxy-heterocycloalkyl or two R₆ together form C₁₋₇alkylene.

In some embodiments, each R₆ is independently C₁₋₇alkyl, amino, amino-C₁₋₇alkyl, C₃₋₈cycloalkyl, heterocycloalkyl and C₁₋₇alkoxy-heterocycloalkyl.

In some embodiments, each R₆ is independently C₁₋₇alkyl. In some embodiments, each R₆ is independently methyl. In some embodiments, each R₆ is independently ethyl. In some embodiments, each R₆ is independently propyl. In some embodiments, each R₆ is independently butyl. In some embodiments, each R₆ is independently pentyl. In some embodiments, each R₆ is independently hexyl. In some embodiments, each R₆ is independently heptyl. In some embodiments, each R₆ is independently isopropyl. In some embodiments, each R₆ is independently isobutyl. In some embodiments, each R₆ is independently isopentyl. In some embodiments, each R₆ is independently isohexyl. In some embodiments, each R₆ is independently secbutyl. In some embodiments, each R₆ is independently secpentyl. In some embodiments, each R₆ is independently sechexyl. In some embodiments, each R₆ is independently tertbutyl.

In some embodiments, each R₆ is independently amino or amino-C₁₋₇alkyl.

In some embodiments, each R₆ is independently amino. In some embodiments, R₆ is independently amino-C₁₋₇alkyl.

In some embodiments, each R₆ is independently C₃₋₈cycloalkyl, heterocycloalkyl and C₁₋₇alkoxy-heterocycloalkyl.

In some embodiments, each R₆ is independently C₃₋₈cycloalkyl.

In some embodiments, each R₆ is independently heterocycloalkyl and C₁₋₇alkoxy-heterocycloalkyl.

In some embodiments, each R₆ is independently heterocycloalkyl. In some embodiments, each R₆ is independently C₁₋₇alkoxy-heterocycloalkyl.

In some embodiments, two R₆ together form C₁₋₇alkylene.

In some embodiments, each R₇ is independently halo, cyano, C₁₋₇alkyl, C₁₋₇haloalkyl, C₁₋₇alkoxy C₁₋₇haloalkoxy, or C₃₋₈cycloalkyl.

In some embodiments, each R₇ is independently halo or cyano.

In some embodiments, each R₇ is independently halo. In some embodiments, each R₇ is independently from F, Cl, Br, and I. In some embodiments, each R₇ is independently from F, Cl, or Br. In some embodiments, each R₇ is independently F. In some embodiments, each R₇ is independently Cl. In some embodiments, each R₇ is independently Br. In some embodiments, each R₇ is independently I.

In some embodiments, each R₇ is independently cyano.

In some embodiments, each R₇ is independently from C₁₋₇alkyl, C₁₋₇haloalkyl, C₁₋₇alkoxy C₁₋₇haloalkoxy, or C₃₋₈cycloalkyl.

In some embodiments, each R₇ is independently from C₁₋₇alkyl, C₁₋₇haloalkyl, C₁₋₇alkoxy, or C₁₋₇haloalkoxy.

In some embodiments, each R₇ is independently C₁₋₇alkyl. In some embodiments, each R₇ is independently methyl. In some embodiments, each R₇ is independently ethyl. In some embodiments, each R₇ is independently propyl. In some embodiments, each R₇ is independently butyl. In some embodiments, each R₇ is independently pentyl. In some embodiments, each R₇ is independently hexyl. In some embodiments, each R₇ is independently heptyl. In some embodiments, each R₇ is independently isopropyl. In some embodiments, each R₇ is independently isobutyl. In some embodiments, each R₇ is independently isopentyl. In some embodiments, each R₇ is independently isohexyl. In some embodiments, each R₇ is independently secbutyl. In some embodiments, each R₇ is independently secpentyl. In some embodiments, each R₇ is independently sechexyl. In some embodiments, each R₇ is independently tertbutyl.

In some embodiments, each R₇ is independently C₁₋₇alkyl optionally substituted with OH.

In some embodiments, each R₇ is independently C₁₋₇alkyl substituted with OH. In some embodiments, each R₇ is independently methyl substituted with OH. In some embodiments, each R₇ is independently ethyl substituted with OH. In some embodiments, each R₇ is independently propyl substituted with OH. In some embodiments, each R₇ is independently butyl substituted with OH. In some embodiments, each R₇ is independently pentyl substituted with OH. In some embodiments, each R₇ is independently hexyl substituted with OH. In some embodiments, each R₇ is independently heptyl substituted with OH. In some embodiments, each R₇ is independently isopropyl substituted with OH. In some embodiments, each R₇ is independently isobutyl substituted with OH. In some embodiments, each R₇ is independently isopentyl substituted with OH. In some embodiments, each R₇ is independently isohexyl substituted with OH. In some embodiments, each R₇ is independently secbutyl substituted with OH. In some embodiments, each R₇ is independently secpentyl substituted with OH. In some embodiments, each R₇ is independently sechexyl substituted with OH. In some embodiments, each R₇ is independently tertbutyl substituted with OH.

In some embodiments, each R₇ is independently C₁₋₇haloalkyl, C₁₋₇alkoxy, or C₁₋₇haloalkoxy.

In some embodiments, each R₇ is independently C₁₋₇haloalkyl.

In some embodiments, each R₇ is independently C₁₋₇alkoxy.

In some embodiments, each R₇ is independently C₁₋₇haloalkoxy.

In some embodiments, each R₇ is independently C₃₋₈cycloalkyl.

To clarify, in all embodiments where two variable groups together form an alkylene, those groups along with the atoms they are attached to and possible intervening atoms, form a ring. This ring can be cycloalkyl or heterocyclyl depending on the attachment point of the variables, but the variable groups themselves are alkylene and thus contain no heteroatoms of their own.

In some embodiments, the compound is selected from the compounds described in Table 1 and pharmaceutically acceptable salts, solvates, or prodrugs thereof.

In some embodiments, the compound is selected from the compounds described in Table 1 and pharmaceutically acceptable salts thereof.

In some embodiments, the compound is selected from the prodrugs of compounds described in Table 1 and pharmaceutically acceptable salts thereof.

In some embodiments, the compound is selected from the compounds described in Table 1, or from the disclosure.

In some embodiments, the compound is selected from the compounds described in Table 1.

For the avoidance of doubt it is to be understood that, where in this specification a group is qualified by “described herein”, the said group encompasses the first occurring and broadest definition as well as each and all of the particular definitions for that group.

The various functional groups and substituents making up the compounds of the Formula (I) or (II) are typically chosen such that the molecular weight of the compound does not exceed 1000 daltons. More usually, the molecular weight of the compound will be less than 900, for example less than 800, or less than 750, or less than 700, or less than 650 daltons. More conveniently, the molecular weight is less than 600 and, for example, is 550 daltons or less.

It will be understood that the compounds of any one of the Formulae disclosed herein and any pharmaceutically acceptable salts thereof, comprise stereoisomers and mixtures of stereoisomers of all isomeric forms of said compounds.

It is to be understood that the compounds of any Formula described herein include the compounds themselves, as well as their salts, and their solvates, if applicable.

The in vivo effects of a compound of any one of the Formulae disclosed herein may be exerted in part by one or more metabolites that are formed within the human or animal body after administration of a compound of any one of the Formulae disclosed herein. As stated hereinbefore, the in vivo effects of a compound of any one of the Formulae disclosed herein may also be exerted by way of metabolism of a precursor compound (a prodrug).

Suitably, the present disclosure excludes any individual compounds not possessing the biological activity defined herein.

### Methods of Synthesis

By way of example only, provided is a scheme for preparing the small molecule splicing modulators (SMSMs) described herein.

In some embodiments, a scheme for preparing an SMSM is described herein in Scheme 1:

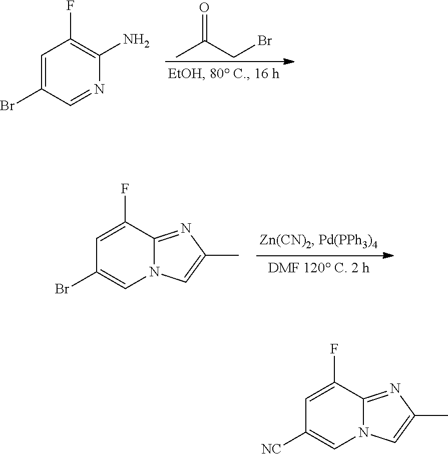

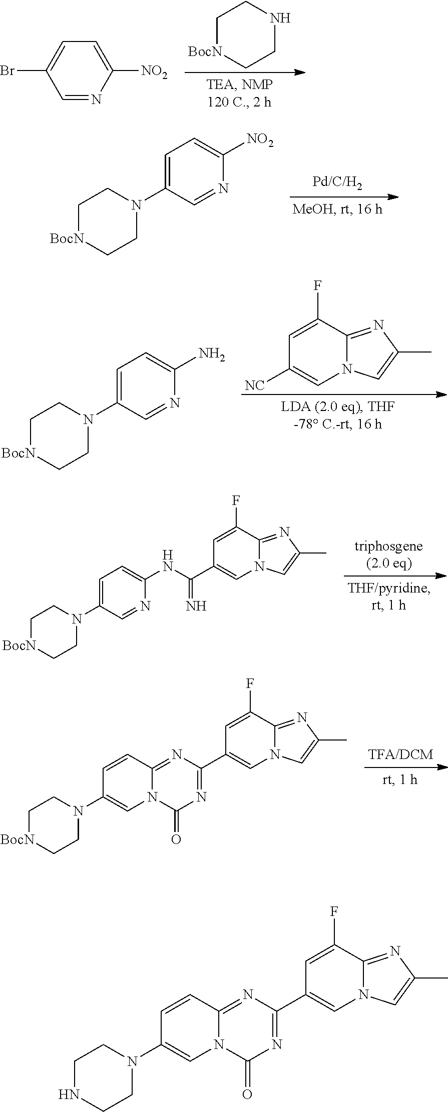

In some aspects, the present disclosure provides a method of preparing a compound of the present disclosure.

In some aspects, the present disclosure provides a method of preparing a compound, comprising one or more steps as described herein.

In some aspects, the present disclosure provides a compound obtainable by, or obtained by, or directly obtained by a method for preparing a compound as described herein.

In some aspects, the present disclosure provides an intermediate as described herein, being suitable for use in a method for preparing a compound as described herein.

The compounds of the present disclosure can be prepared by any suitable technique known in the art. Particular processes for the preparation of these compounds are described further in the accompanying examples.

In the description of the synthetic methods described herein and in any referenced synthetic methods that are used to prepare the starting materials, it is to be understood that all proposed reaction conditions, including choice of solvent, reaction atmosphere, reaction temperature, duration of the experiment and workup procedures, can be selected by a person skilled in the art.

It is understood by one skilled in the art of organic synthesis that the functionality present on various portions of the molecule must be compatible with the reagents and reaction conditions utilized.

It will be appreciated that during the synthesis of the compounds of the disclosure in the processes defined herein, or during the synthesis of certain starting materials, it may be desirable to protect certain substituent groups to prevent their undesired reaction. The skilled chemist will appreciate when such protection is required, and how such protecting groups may be put in place, and later removed. For examples of protecting groups see one of the many general texts on the subject, for example, ‘Protective Groups in Organic Synthesis’ by Theodora Green (publisher: John Wiley & Sons). Protecting groups may be removed by any convenient method described in the literature or known to the skilled chemist as appropriate for the removal of the protecting group in question, such methods being chosen so as to effect removal of the protecting group with the minimum disturbance of groups elsewhere in the molecule. Thus, if reactants include, for example, groups such as amino, carboxy or hydroxy it may be desirable to protect the group in some of the reactions mentioned herein.

By way of example, a suitable protecting group for an amino or alkylamino group is, for example, an acyl group, for example an alkanoyl group such as acetyl, an alkoxycarbonyl group, for example a methoxycarbonyl, ethoxycarbonyl, or t-butoxycarbonyl group, an arylmethoxycarbonyl group, for example benzyloxycarbonyl, or an aroyl group, for example benzoyl. The deprotection conditions for the above protecting groups necessarily vary with the choice of protecting group. Thus, for example, an acyl group such as an alkanoyl or alkoxycarbonyl group or an aroyl group may be removed by, for example, hydrolysis with a suitable base such as an alkali metal hydroxide, for example lithium or sodium hydroxide. Alternatively an acyl group such as a tert-butoxycarbonyl group may be removed, for example, by treatment with a suitable acid as hydrochloric, sulphuric or phosphoric acid or trifluoroacetic acid and an arylmethoxycarbonyl group such as a benzyloxycarbonyl group may be removed, for example, by hydrogenation over a catalyst such as palladium on carbon, or by treatment with a Lewis acid for example boron tris(trifluoroacetate). A suitable alternative protecting group for a primary amino group is, for example, a phthaloyl group which may be removed by treatment with an alkylamine, for example dimethylaminopropylamine, or with hydrazine.

A suitable protecting group for a hydroxy group is, for example, an acyl group, for example an alkanoyl group such as acetyl, an aroyl group, for example benzoyl, or an arylmethyl group, for example benzyl. The deprotection conditions for the above protecting groups will necessarily vary with the choice of protecting group. Thus, for example, an acyl group such as an alkanoyl or an aroyl group may be removed, for example, by hydrolysis with a suitable base such as an alkali metal hydroxide, for example lithium, sodium hydroxide or ammonia. Alternatively an arylmethyl group such as a benzyl group may be removed, for example, by hydrogenation over a catalyst such as palladium on carbon.

A suitable protecting group for a carboxy group is, for example, an esterifying group, for example a methyl or an ethyl group which may be removed, for example, by hydrolysis with a base such as sodium hydroxide, or for example a tert-butyl group which may be removed, for example, by treatment with an acid, for example an organic acid such as trifluoroacetic acid, or for example a benzyl group which may be removed, for example, by hydrogenation over a catalyst such as palladium on carbon.

Once a compound of Formula (I) or (II) has been synthesised by any one of the processes defined herein, the processes may then further comprise the additional steps of: (i) removing any protecting groups present; (ii) converting the compound of Formula (I) into another compound of Formula (I) or (II); (iii) forming a pharmaceutically acceptable salt, hydrate or solvate thereof, and/or (iv) forming a prodrug thereof.

The resultant compounds of Formula (I) or (II) can be isolated and purified using techniques well known in the art.

Conveniently, the reaction of the compounds is carried out in the presence of a suitable solvent, which is preferably inert under the respective reaction conditions. Examples of suitable solvents comprise but are not limited to hydrocarbons, such as hexane, petroleum ether, benzene, toluene or xylene; chlorinated hydrocarbons, such as trichlorethylene, 1,2-dichloroethane, tetrachloromethane, chloroform or dichloromethane; alcohols, such as methanol, ethanol, isopropanol, n-propanol, n-butanol or tert-butanol; ethers, such as diethyl ether, diisopropyl ether, tetrahydrofuran (THF), 2-methyltetrahydrofuran, cyclopentylmethyl ether (CPME), methyl tert-butyl ether (MTBE) or dioxane; glycol ethers, such as ethylene glycol monomethyl or monoethyl ether or ethylene glycol dimethyl ether (diglyme); ketones, such as acetone, methylisobutylketone (MIBK) or butanone; amides, such as acetamide, dimethylacetamide, dimethylformamide (DMF) or N-methylpyrrolidinone (NMP); nitriles, such as acetonitrile; sulphoxides, such as dimethyl sulphoxide (DMSO); nitro compounds, such as nitromethane or nitrobenzene; esters, such as ethyl acetate or methyl acetate, or mixtures of the said solvents or mixtures with water.

The reaction temperature is suitably between about −100° C. and 300° C., depending on the reaction step and the conditions used.

Reaction times are generally in the range between a fraction of a minute and several days, depending on the reactivity of the respective compounds and the respective reaction conditions. Suitable reaction times are readily determinable by methods known in the art, for example reaction monitoring. Based on the reaction temperatures given above, suitable reaction times generally lie in the range between 10 minutes and 48 hours.

Moreover, by utilising the procedures described herein, in conjunction with ordinary skills in the art, additional compounds of the present disclosure can be readily prepared. Those skilled in the art will readily understand that known variations of the conditions and processes of the following preparative procedures can be used to prepare these compounds. As will be understood by the person skilled in the art of organic synthesis, compounds of the present disclosure are readily accessible by various synthetic routes, some of which are exemplified in the accompanying examples. The skilled person will easily recognise which kind of reagents and reactions conditions are to be used and how they are to be applied and adapted in any particular instance—wherever necessary or useful—in order to obtain the compounds of the present disclosure. Furthermore, some of the compounds of the present disclosure can readily be synthesised by reacting other compounds of the present disclosure under suitable conditions, for instance, by converting one particular functional group being present in a compound of the present disclosure, or a suitable precursor molecule thereof, into another one by applying standard synthetic methods, like reduction, oxidation, addition or substitution reactions; those methods are well known to the skilled person. Likewise, the skilled person will apply—whenever necessary or useful—synthetic protecting (or protective) groups; suitable protecting groups as well as methods for introducing and removing them are well-known to the person skilled in the art of chemical synthesis and are described, in more detail, in, e.g., P. G. M. Wuts, T. W. Greene, “Greene's Protective Groups in Organic Synthesis”, 4th edition (2006) (John Wiley & Sons).

General routes for the preparation of a compound of the application are described herein.

### Biological Assays

Compounds designed, selected and/or optimised by methods described above, once produced, can be characterised using a variety of assays known to those skilled in the art to determine whether the compounds have biological activity. For example, the molecules can be characterised by conventional assays, including but not limited to those assays described below, to determine whether they have a predicted activity, binding activity and/or binding specificity.

### Cell Proliferation Assay

Various cancer cell lines including K562, HL60, COLO201, COL0205 can be treated with the compounds of the present disclosure to assess their effect on inhibiting cell proliferation.

Test compounds may be diluted in duplicates and dispensed into an assay plate. Positive and negative controls may be used (e.g. staurosporine). Frozen cell line stock is prepared and the cell suspension is dispensed into the assay plate. After incubation, the luminescence (RLU) signals in each well may be recorded and converted to percentage inhibition values using the signals in negative and positive control standards. IC₅₀ values may be determined by curve fitting in Levenberg-Marquardt algorithm.

### Pharmaceutical Compositions

In some aspects, the present disclosure provides a pharmaceutical composition comprising a compound of the present disclosure as an active ingredient. In some embodiments, the present disclosure provides a pharmaceutical composition comprising at least one compound of each of the formulae described herein, or a pharmaceutically acceptable salt or solvate thereof, and one or more pharmaceutically acceptable carriers or excipients. In some embodiments, the present disclosure provides a pharmaceutical composition comprising at least one compound selected from Table 1, or from the disclosure. In some embodiments, the present disclosure provides a pharmaceutical composition comprising at least one compound selected from Table 1.

As used herein, the term “composition” is intended to encompass a product comprising the specified ingredients in the specified amounts, as well as any product which results, directly or indirectly, from combination of the specified ingredients in the specified amounts.

The compounds of present disclosure can be formulated for oral administration in forms such as tablets, capsules (each of which includes sustained release or timed release formulations), pills, powders, granules, elixirs, tinctures, suspensions, syrups and emulsions. The compounds of present disclosure on can also be formulated for intravenous (bolus or in-fusion), intraperitoneal, topical, subcutaneous, intramuscular or transdermal (e.g., patch) administration, all using forms well known to those of ordinary skill in the pharmaceutical arts.

The formulation of the present disclosure may be in the form of an aqueous solution comprising an aqueous vehicle. The aqueous vehicle component may comprise water and at least one pharmaceutically acceptable excipient. Suitable acceptable excipients include those selected from the group consisting of a solubility enhancing agent, chelating agent, preservative, tonicity agent, viscosity/suspending agent, buffer, and pH modifying agent, and a mixture thereof.

Any suitable solubility enhancing agent can be used. Examples of a solubility enhancing agent include cyclodextrin, such as those selected from the group consisting of hydroxypropyl-β-cyclodextrin, methyl-β-cyclodextrin, randomly methylated-β-cyclodextrin, ethylated-β-cyclodextrin, triacetyl-β-cyclodextrin, peracetylated-β-cyclodextrin, carboxymethyl-β-cyclodextrin, hydroxyethyl-β-cyclodextrin, 2-hydroxy-3-(trimethylammonio)propyl-β-cyclodextrin, glucosyl-β-cyclodextrin, sulphated β-cyclodextrin (S-β-CD), maltosyl-β-cyclodextrin, β-cyclodextrin sulphobutyl ether, branched-β-cyclodextrin, hydroxypropyl-γ-cyclodextrin, randomly methylated-γ-cyclodextrin, and trimethyl-γ-cyclodextrin, and mixtures thereof.

Any suitable chelating agent can be used. Examples of a suitable chelating agent include those selected from the group consisting of ethylenediaminetetraacetic acid and metal salts thereof, disodium edetate, trisodium edetate, and tetrasodium edetate, and mixtures thereof.

Any suitable preservative can be used. Examples of a preservative include those selected from the group consisting of quaternary ammonium salts such as benzalkonium halides (preferably benzalkonium chloride), chlorhexidine gluconate, benzethonium chloride, cetyl pyridinium chloride, benzyl bromide, phenylmercury nitrate, phenylmercury acetate, phenylmercury neodecanoate, merthiolate, methylparaben, propylparaben, sorbic acid, potassium sorbate, sodium benzoate, sodium propionate, ethyl p-hydroxybenzoate, propylaminopropyl biguanide, and butyl-p-hydroxybenzoate, and sorbic acid, and mixtures thereof.

The aqueous vehicle may also include a tonicity agent to adjust the tonicity (osmotic pressure). The tonicity agent can be selected from the group consisting of a glycol (such as propylene glycol, diethylene glycol, triethylene glycol), glycerol, dextrose, glycerin, mannitol, potassium chloride, and sodium chloride, and a mixture thereof.

The aqueous vehicle may also contain a viscosity/suspending agent. Suitable viscosity/suspending agents include those selected from the group consisting of cellulose derivatives, such as methyl cellulose, ethyl cellulose, hydroxyethylcellulose, polyethylene glycols (such as polyethylene glycol 300, polyethylene glycol 400), carboxymethyl cellulose, hydroxypropylmethyl cellulose, and cross-linked acrylic acid polymers (carbomers), such as polymers of acrylic acid cross-linked with polyalkenyl ethers or divinyl glycol (Carbopols—such as Carbopol 934, Carbopol 934P, Carbopol 971, Carbopol 974 and Carbopol 974P), and a mixture thereof.

In order to adjust the formulation to an acceptable pH (typically a pH range of about 5.0 to about 9.0, more preferably about 5.5 to about 8.5, particularly about 6.0 to about 8.5, about 7.0 to about 8.5, about 7.2 to about 7.7, about 7.1 to about 7.9, or about 7.5 to about 8.0), the formulation may contain a pH modifying agent. The pH modifying agent is typically a mineral acid or metal hydroxide base, selected from potassium hydroxide, sodium hydroxide, and hydrochloric acid, and mixtures thereof, and preferably sodium hydroxide and/or hydrochloric acid. These acidic and/or basic pH modifying agents are added to adjust the formulation to the target acceptable pH range. Hence it may not be necessary to use both acid and base—depending on the formulation, the addition of one of the acid or base may be sufficient to bring the mixture to the desired pH range.

The aqueous vehicle may also contain a buffering agent to stabilise the pH. When used, the buffer is selected from the group consisting of a phosphate buffer (such as sodium dihydrogen phosphate and disodium hydrogen phosphate), a borate buffer (such as boric acid, or salts thereof including disodium tetraborate), a citrate buffer (such as citric acid, or salts thereof including sodium citrate), and F-aminocaproic acid, and mixtures thereof.

The formulation may further comprise a wetting agent. Suitable classes of wetting agents include those selected from the group consisting of polyoxypropylene-polyoxyethylene block copolymers (poloxamers), polyethoxylated ethers of castor oils, polyoxyethylenated sorbitan esters (polysorbates), polymers of oxyethylated octyl phenol (Tyloxapol), polyoxyl 40 stearate, fatty acid glycol esters, fatty acid glyceryl esters, sucrose fatty esters, and polyoxyethylene fatty esters, and mixtures thereof.

According to a further aspect of the disclosure there is provided a pharmaceutical composition which comprises a compound of the disclosure as defined hereinbefore, or a pharmaceutically acceptable salt, hydrate or solvate thereof, in association with a pharmaceutically acceptable diluent or carrier.

The compositions of the disclosure may be in a form suitable for oral use (for example as tablets, lozenges, hard or soft capsules, aqueous or oily suspensions, emulsions, dispersible powders or granules, syrups or elixirs), for topical use (for example as creams, ointments, gels, or aqueous or oily solutions or suspensions), for administration by inhalation (for example as a finely divided powder or a liquid aerosol), for administration by insufflation (for example as a finely divided powder) or for parenteral administration (for example as a sterile aqueous or oily solution for intravenous, subcutaneous, intramuscular, intraperitoneal or intramuscular dosing or as a suppository for rectal dosing).

The compositions of the disclosure may be obtained by conventional procedures using conventional pharmaceutical excipients, well known in the art. Thus, compositions intended for oral use may contain, for example, one or more colouring, sweetening, flavouring and/or preservative agents.

An effective amount of a compound of the present disclosure for use in therapy is an amount sufficient to treat or prevent a disease or disorder referred to herein, slow its progression and/or reduce the symptoms associated with the condition.

An effective amount of a compound of the present disclosure for use in therapy is an amount sufficient to treat a disease or disorder referred to herein, slow its progression and/or reduce the symptoms associated with the condition.

The size of the dose for therapeutic or prophylactic purposes of a compound of Formula (I) or (II) will naturally vary according to the nature and severity of the conditions, the age and sex of the animal or subject and the route of administration, according to well-known principles of medicine.

### Methods of Use

In one aspect, the present disclosure provides compounds of the present disclosure or their pharmaceutically acceptable salts for the use in the treatment or prevention of cancer.

In one aspect, the present disclosure provides a method for the treatment or prevention of cancer in a subject in need thereof, which method comprises administering to the subject a pharmaceutically effective amount of a compound or pharmaceutical composition of the present disclosure. In some embodiments, the cancer is a liquid cancer. In some embodiments, the cancer is a leukemic cancer. In some embodiments, the cancer is a leukemia or lymphoma. In some embodiments, the cancer is colorectal cancer, breast cancer, or prostate cancer. In some embodiments, the cancer is a leukemia, acute myeloid leukemia, colon cancer, gastric cancer, acute monocytic leukemia, breast cancer, hepatocellular carcinoma, alveolar soft part sarcoma, myeloma, skin melanoma, pancreatic cancer, adenocarcinoma, adenoiditis, adenoid cystic carcinoma, gastrointestinal stromal tumor, sarcoma, prostate adenocarcinoma, Hodgkin's lymphoma, ovarian cancer, non-Hodgkin's lymphoma, multiple myeloma, chronic myeloid leukemia, acute lymphoblastic leukemia, renal cell carcinoma, transitional cell carcinoma, colorectal cancer, chronic lymphocytic leukemia, anaplastic large cell lymphoma, kidney cancer, breast cancer, or cervical cancer.

In some embodiments, the cancer is a solid cancer or solid tumor. In some embodiments, the tumor is selected from the group consisting of adenocarcinoma, melanoma (e.g., metastatic melanoma), liver cancer {e.g., hepatocellular carcinoma, hepatoblastoma, liver carcinoma), prostate cancer (e.g., prostate adenocarcinoma, androgen-independent prostate cancer, androgen-dependent prostate cancer, prostate carcinoma), sarcoma (e.g., leiomyosarcoma, rhabdomyosarcoma), brain cancer (e.g., glioma, a malignant glioma, astrocytoma, brain stem glioma, ependymoma, oligodendroglioma, nonglial tumor, acoustic neurinoma, craniopharyngioma, medulloblastoma, meningioma, pineocytoma, pineoblastoma, primary brain lymphoma, anaplastic astrocytoma, juvenile pilocytic astrocytoma, a mixture of oligodendroglioma and astrocytoma elements), breast cancer (e.g., triple negative breast cancer, metastatic breast cancer, breast carcinoma, breast sarcoma, adenocarcinoma, lobular (small cell) carcinoma, intraductal carcinoma, medullary breast cancer, mucinous breast cancer, tubular breast cancer, papillary breast cancer, inflammatory breast cancer), Paget's disease, juvenile Paget's disease, lung cancer (e.g., KRAS-mutated non-small cell lung cancer, non-small cell lung cancer, squamous cell carcinoma (epidermoid carcinoma), adenocarcinoma, large-cell carcinoma, small cell lung cancer, lung carcinoma), pancreatic cancer (e.g., insulinoma, gastrinoma, glucagonoma, vipoma, somatostatin-secreting tumor, carcinoid tumor, islet cell tumor, pancreas carcinoma), skin cancer (e.g., skin melanoma, basal cell carcinoma, squamous cell carcinoma, melanoma, superficial spreading melanoma, nodular melanoma, lentigo malignant melanoma, acrallentiginous melanoma, skin carcinoma), cervical cancer (e.g., squamous cell carcinoma, adenocarcinoma, cervical carcinoma), ovarian cancer (e.g., ovarian epithelial carcinoma, borderline tumor, germ cell tumor, stromal tumor, ovarian carcinoma), cancer of the mouth, cancer of the nervous system (e.g., cancer of the central nervous system, a CNS germ cell tumor), goblet cell metaplasia, kidney cancer (e.g., renal cell cancer, adenocarcinoma, hypernephroma, Wilms' tumor, fibrosarcoma, transitional cell cancer (renal pelvis and/or uterer), renal cell carcinoma, renal carcinoma), bladder cancer (e.g., transitional cell carcinoma, squamous cell cancer, carcinosarcoma), stomach cancer (e.g., fungating (polypoid), ulcerating, superficial spreading, diffusely spreading, liposarcoma, fibrosarcoma, carcinosarcoma), uterine cancer (e.g., endometrial cancer, endometrial carcinoma, uterine sarcoma), cancer of the esophagus (e.g., squamous cancer, adenocarcinoma, adenoid cyctic carcinoma, mucoepidermoid carcinoma, adenosquamous carcinoma, sarcoma, melanoma, plasmacytoma, verrucous carcinoma, and oat cell (small cell) carcinoma, esophageal carcinomas), colon cancer (e.g., colon carcinoma), cancer of the rectum (e.g., rectal cancers), colorectal cancer (e.g., colorectal carcinoma, metastatic colorectal cancer, hereditary nonpolyposis colorectal cancer, KRAS mutated colorectal cancer), gallbladder cancer (e.g., adenocarcinoma, cholangiocarcinoma, papillary cholangiocarcinoma, nodular cholangiocarcinoma, diffuse cholangiocarcinoma), testicular cancer (e.g., germinal tumor, seminoma, anaplastic testicular cancer, classic (typical) testicular cancer, spermatocyte testicular cancer, nonseminoma testicular cancer), embryonal carcinoma (e.g., teratoma carcinoma, choriocarcinoma (yolk-sac tumor)), gastric cancer (e.g., gastrointestinal stromal tumor, cancer of other gastrointestinal tract organs, gastric carcinomas), bone cancer (e.g., connective tissue sarcoma, bone sarcoma, cholesteatoma-induced bone osteosarcoma, Paget's disease of bone, osteosarcoma, chondrosarcoma, Ewing's sarcoma, malignant giant cell tumor, fibrosarcoma of bone, chordoma, periosteal sarcoma, soft-tissue sarcoma, angiosarcoma (hemangiosarcoma), fibrosarcoma, Kaposi's sarcoma, leiomyosarcoma, alveolar soft part sarcoma), liposarcoma, lymphangiosarcoma, neurilemmoma, rhabdomyosarcoma, synovial sarcoma, cancer of the lymph node (e.g., lymphangioendotheliosarcoma), adenoid cystic carcinoma, vaginal cancer (e.g., squamous cell carcinoma, adenocarcinoma, melanoma), vulvar cancer (e.g., squamous cell carcinoma, melanoma, adenocarcinoma, sarcoma, Paget's disease), cancer of other reproductive organs, thyroid cancer (e.g., papillary thyroid cancer, follicular thyroid cancer, medullary thyroid cancer, anaplastic thyroid cancer, thyroid carcinoma), salivary gland cancer (e.g., adenocarcinoma, mucoepidermoid carcinoma), eye cancer (e.g., ocular melanoma, iris melanoma, choroidal melanoma, cilliary body melanoma, retinoblastoma), penal cancers, oral cancer (e.g. squamous cell carcinoma, basal cancer), pharynx cancer (e.g., squamous cell cancer, verrucous pharynx cancer), cancer of the head, cancer of the neck, cancer of the throat, cancer of the chest, cancer of the spleen, cancer of skeletal muscle, cancer of subcutaneous tissue, adrenal cancer, pheochromocytoma, adrenocortical carcinoma, pituitary cancer, Cushing's disease, prolactin-secreting tumor, acromegaly, diabetes insipidus, myxosarcoma, osteogenic sarcoma, endotheliosarcoma, mesothelioma, synovioma, hemangioblastoma, epithelial carcinoma, cystadenocarcinoma, bronchogenic carcinoma, sweat gland carcinoma, sebaceous gland carcinoma, papillary carcinoma, papillary adenocarcinomas, ependyoma, optic nerve glioma, primitive neuroectodermal tumor, rhabdoid tumor, renal cancer, glioblastoma multiforme, neurofibroma, neurofibromatosis, pediatric cancer, neuroblastoma, malignant melanoma, carcinoma of the epidermis, polycythemia vera, Waldenstrom's macroglobulinemia, monoclonal gammopathy of undetermined significance, benign monoclonal gammopathy, heavy chain disease, pediatric solid tumor, Ewing's sarcoma, Wilms tumor, carcinoma of the epidermis, HIV-related Kaposi's sarcoma, rhabdomyosarcoma, thecomas, arrhenoblastomas, endometrial carcinoma, endometrial hyperplasia, endometriosis, fibrosarcomas, choriocarcinoma, nasopharyngeal carcinoma, laryngeal carcinoma, hepatoblastoma, Kaposi's sarcoma, hemangioma, cavernous hemangioma, hemangioblastoma, retinoblastoma, glioblastoma, Schwannoma, neuroblastoma, rhabdomyosarcoma, osteogenic sarcoma, leiomyosarcoma, urinary tract carcinoma, abnormal vascular proliferation associated with phakomatoses, edema (such as that associated with brain tumors), Meigs' syndrome, pituitary adenoma, primitive neuroectodermal tumor, medullblastoma, and acoustic neuroma.

A method of treating, preventing, or delaying cancer can comprise administering a therapeutically effective amount of a compound described herein or a pharmaceutically acceptable salt thereof to a subject with basal cell carcinoma, goblet cell metaplasia, or a malignant glioma. A method of treating, preventing, or delaying cancer can comprise administering a therapeutically effective amount of a compound described herein or a pharmaceutically acceptable salt thereof to a subject with a cancer of the liver, breast, lung, prostate, cervix, uterus, colon, pancreas, kidney, stomach, bladder, ovary, or brain.

A method of treating, preventing, or delaying cancer can comprise administering a therapeutically effective amount of a compound described herein or a pharmaceutically acceptable salt thereof to a subject with a cancer of the head, neck, eye, mouth, throat, esophagus, esophagus, chest, bone, lung, kidney, colon, rectum or other gastrointestinal tract organs, stomach, spleen, skeletal muscle, subcutaneous tissue, prostate, breast, ovaries, testicles or other reproductive organs, skin, thyroid, blood, lymph nodes, kidney, liver, pancreas, and brain or central nervous system.

### Routes of Administration

The compounds of the disclosure or pharmaceutical compositions comprising these compounds may be administered to a subject by any convenient route of administration, whether systemically/peripherally or topically (i.e., at the site of desired action).

Routes of administration include, but are not limited to, oral (e.g. by ingestion); buccal; sublingual; transdermal (including, e.g., by a patch, plaster, etc.); transmucosal (including, e.g., by a patch, plaster, etc.); intranasal (e.g., by nasal spray); ocular (e.g., by eye drops); pulmonary (e.g., by inhalation or insufflation therapy using, e.g., via an aerosol, e.g., through the mouth or nose); rectal (e.g., by suppository or enema); vaginal (e.g., by pessary); parenteral, for example, by injection, including subcutaneous, intradermal, intramuscular, intravenous, intra-arterial, intracardiac, intrathecal, intraspinal, intracapsular, subcapsular, intraorbital, intraperitoneal, intratracheal, subcuticular, intraarticular, subarachnoid, and intrasternal; by implant of a depot or reservoir, for example, subcutaneously or intramuscularly.

### EXAMPLES

For exemplary purpose, neutral compounds of Formula (I) or (II) are synthesized and tested in the examples. It is understood that the neutral compounds of Formula (I) or (II) may be converted to the corresponding pharmaceutically acceptable salts of the compounds using routine techniques in the art (e.g., by saponification of an ester to the carboxylic acid salt, or by hydrolyzing an amide to form a corresponding carboxylic acid and then converting the carboxylic acid to a carboxylic acid salt).

Nuclear magnetic resonance (NMR) spectra were recorded at 400 MHz or 300 MHz as stated; the chemical shifts (6) are reported in parts per million (ppm). Spectra were recorded using a Bruker or Varian instrument with 8, 16 or 32 scans.

LC-MS chromatograms and spectra were recorded using an Agilent 1200 or Shimadzu LC-20 AD&MS 2020 instrument using a C-18 column such as C18 2.1×30 mm, unless otherwise stated. Injection volumes were 0.7-8.0 μl and the flow rates were typically 0.8 or 1.2 ml/min. Detection methods were diode array (DAD) or evaporative light scattering (ELSD) as well as positive ion electrospray ionisation. MS range was 100-1000 Da. Solvents were gradients of water and acetonitrile both containing a modifier (typically 0.01-0.04%) such as trifluoroacetic acid or ammonium carbonate.

### ABBREVIATIONS

ACN Acetonitrile
BINAP 2,2′-bis(diphenylphosphino)-1,1′-binaphthyl
CDCl₃ Chloroform-d
DCM Dichloromethane
DMF N,N-dimethylformamide
DMSO dimethylsulphoxide
DMSO-d₆ Hexadeuterodimethylsulphoxide
DP desired product
eq. Equivalents
ESI Electrospray ionisation
EA ethyl acetate
FCC flash column chromatography
h hour(s)
¹H NMR Proton nuclear magnetic resonance spectroscopy
HPLC high performance liquid chromatography
LC-MS Liquid chromatography-mass spectrometry
MeOD Methanol-d₄
MeOH Methanol
min minute(s)
NaOAc Sodium acetate
NMP N-Methyl-2-Pyrrolidone
PE petroleum ether
ppm parts per million
RM reaction mixture
rt room temperature
SM starting material
TEA Triethylamine
TFA trifluoroacetic acid
THF Tetrahydrofuran
TLC thin layer chromatography
Y Yield

### Example 1. Synthesis of 2-{8-fluoro-2-methylimidazo[1,2-a]pyridin-6-yl}-7-(piperazin-1-yl)-4H-pyrido[1,2-a][1,3,5]triazin-4-one (Compound 99)

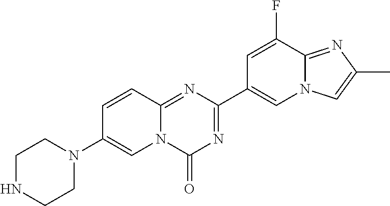 [SMILES: Cc1cn2cc(-c3nc(=O)n4cc(N5CCNCC5)ccc4n3)cc(F)c2n1]

### Step 1: Synthesis of 6-bromo-8-fluoro-2-methylimidazo[1,2-a]pyridine

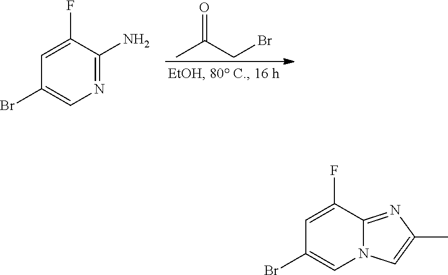

A mixture of 5-bromo-3-fluoropyridin-2-amine (10 g, 52.6 mmol) and 1-bromopropan-2-one (17.9 g, 131.6 mmol) in EtOH (80 mL) was heated to 80° C. for 16 h. LCMS showed 15% of SM was left and 50% of DP was formed. The reaction was stopped and cooled to rt. The solvent was removed under vacuum and the residue was purified by silica gel column chromatography (PE/EA=4:1) to give 6-bromo-8-fluoro-2-methylimidazo[1,2-a]pyridine (5.0 g, Y: 41.7%) as light pink solid. ESI-MS (M+H)⁺: 229.1, 231.1. ¹H NMR (400 MHz, CDCl₃) δ 8.03 (d, J=1.4 Hz, 1H), 7.38 (d, J=1.4 Hz, 1H), 6.97 (dd, J=9.6, 1.5 Hz, 1H), 2.47 (s, 3H).

### Step 2: Synthesis of 8-fluoro-2-methylimidazo[1,2-a]pyridine-6-carbonitrile

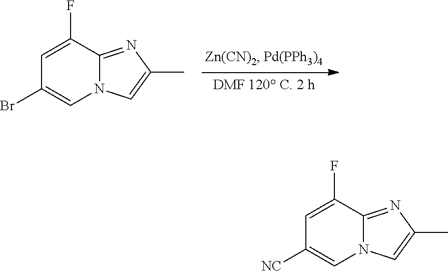

To a mixture of 6-bromo-8-fluoro-2-methylimidazo[1,2-a]pyridine (230 mg, 1.01 mmol) in DMF (10 mL) was added Zn (CN)₂ (230 mg, 2.02 mmol), Pd(PPh₃)₄ (115 mg, 0.1 mmol), and the reaction mixture was stirred for 2 h at 120° C. After cooling to rt, the mixture was diluted with water (80 mL) and 100 mL of EA. The EA layer was separated, washed with brine and water once (100 mL×1), dried over Na₂SO₄, concentrated to dryness. The crude was purified by silica gel column (10˜60% EA in PE). The product was obtained as off-white solid (120 mg, Y: 68%). ¹H NMR (400 MHz, CDCl₃) δ 8.34 (d, J=1.2 Hz, 1H), 7.52 (dd, J=3.0, 0.7 Hz, 1H), 6.98 (dd, J=9.6, 1.3 Hz, 1H), 2.51 (d, J=0.8 Hz, 3H).

### Step 3: Synthesis of tert-butyl 4-(6-nitropyridin-3-yl)piperazine-1-carboxylate

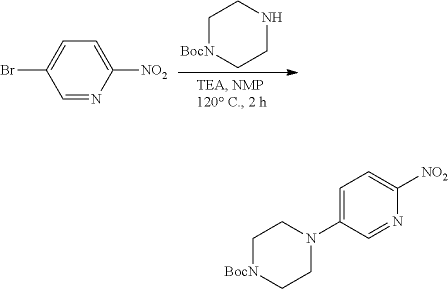 [SMILES: O=[N+]([O-])c1ccc(Br)cn1 ; CC(C)(C)OC(=O)N1CCNCC1 ; CC(C)(C)OC(=O)N1CCN(c2ccc([N+](=O)[O-])nc2)CC1]

To a suspension of 5-bromo-2-nitropyridine (5.0 g, 25.0 mmol) and tert-butyl piperazine-1-carboxylate (9.3 g, 50.0 mmol) in NMP (80 mL) was added TEA (7.5 g, 75.0 mmol), and the reaction mixture was stirred for 3 h at 120° C. After cooling to rt and diluting with water, the mixture was extracted with EA. The combined organics were washed with brine and water, dried over Na₂SO₄ and concentrated to dryness. The crude was purified by silica gel column (10˜100% EA in PE). The product was obtained as off-white solid (5 g, Y: 68%). ¹H NMR (400 MHz, DMSO-d₆) δ 8.24 (d, J=3.0 Hz, 1H), 8.17 (d, J=9.2 Hz, 1H), 7.47 (dd, J=9.3, 3.1 Hz, 1H), 3.55-3.46 (m, 8H), 1.43 (s, 9H).

### Step 4: Synthesis of tert-butyl 4-(6-aminopyridin-3-yl)piperazine-1-carboxylate

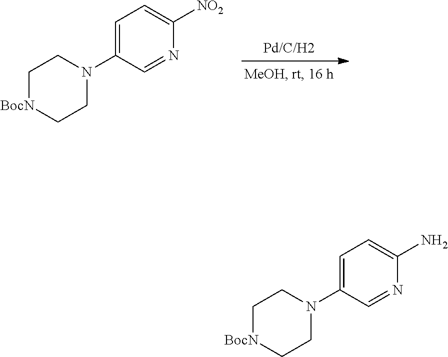 [SMILES: CC(C)(C)OC(=O)N1CCN(c2ccc([N+](=O)[O-])nc2)CC1 ; CC(C)(C)OC(=O)N1CCN(c2ccc(N)nc2)CC1]

To a mixture of tert-butyl 4-(6-nitropyridin-3-yl)piperazine-1-carboxylate (5.5 g, 17.86 mmol) in MeOH (100 mL) was added Pd/C (1.1 g). The reaction mixture was stirred for 16 h at rt under hydrogen atmosphere (balloon pressure). The solvent was filtered, and the filter cake was washed with MeOH (50 mL). The filtrate was concentrated to give desired product as a purple semi-solid (4.9 g, crude yield: 97%), which was used in the next step without further purification. ESI-MS: [M+H]⁺ 279.2.

### Step 5: Synthesis of tert-butyl 4-(6-(8-fluoro-2-methylimidazo[1,2-a]pyridine-6-carboximidamido)pyridin-3-yl)piperazine-1-carboxylate

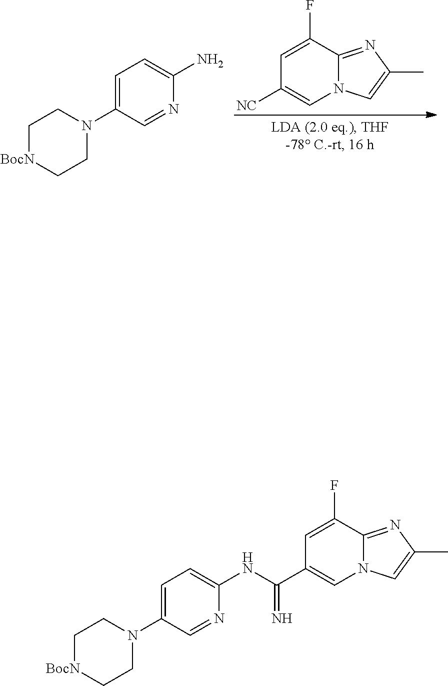 [SMILES: CC(C)(C)OC(=O)N1CCN(c2ccc(N)nc2)CC1 ; Cc1cn2cc(C#N)cc(F)c2n1 ; Cc1cn2cc(C(=N)Nc3ccc(N4CCN(C(=O)OC(C)(C)C)CC4)cn3)cc(F)c2n1]

To a mixture of tert-butyl 4-(6-aminopyridin-3-yl)piperazine-1-carboxylate (280 mg, 1.0 mmol) in THE was added LDA (1 mL, 2.0 mmol, 2M in THF) at −78° C. The mixture was stirred at this temperature for 30 min. Compound 8-fluoro-2-methylimidazo[1,2-a]pyridine-6-carbonitrile (150 mg, 0.9 mmol) was added. The mixture was allowed to warm to rt and stirred for 16 h. After concentration, the crude as purified by silica gel column (DCM/MeOH=10:1) to give desired product (120 mg, Y: 29%) as a brown solid. ESI-MS: [M+H]⁺ 454.1.

### Step 6: Synthesis of tert-butyl 4-(2-(8-fluoro-2-methylimidazo[1,2-a]pyridin-6-yl)-4-oxo-4H-pyrido[1,2-a][1,3,5]triazin-7-yl)piperazine-1-carboxylate

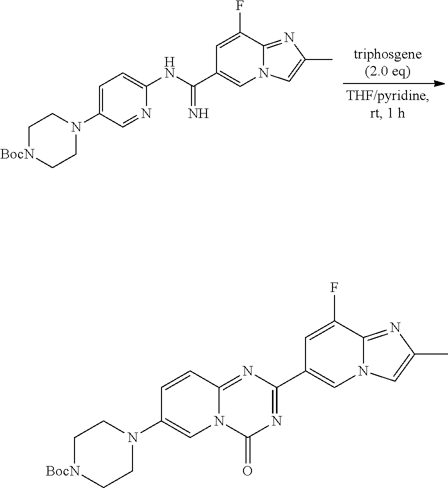 [SMILES: Cc1cn2cc(C(=N)Nc3ccc(N4CCN(C(=O)OC(C)(C)C)CC4)cn3)cc(F)c2n1 ; Cc1cn2cc(-c3nc(=O)n4cc(N5CCN(C(=O)OC(C)(C)C)CC5)ccc4n3)cc(F)c2n1]

To a solution of tert-butyl 4-(6-(8-fluoro-2-methylimidazo[1,2-a]pyridine-6-carboximidamido)pyridin-3-yl)piperazine-1-carboxylate (45 mg, 0.1 mmol) in THE (5 mL) was added triphosgene (59 mg, 0.2 mmol) and pyridine (1 mL). The reaction mixture was stirred for 1 h at rt, After concentration, the residue was purified by silica gel column (1˜10% MeOH in DCM). The product was obtained as a white solid (20 mg, yield: 41%) ESI-MS: [M+H]⁺ 480.1.

### Step 7: Synthesis of 2-{8-fluoro-2-methylimidazo[1,2-a]pyridin-6-yl}-7-(piperazin-1-yl)-4H-pyrido[1,2-a][1,3,5]triazin-4-one

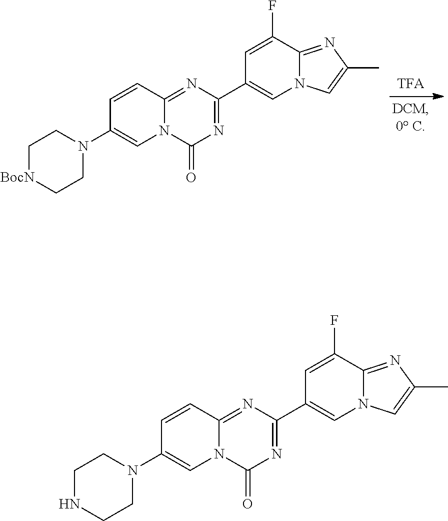 [SMILES: Cc1cn2cc(-c3nc(=O)n4cc(N5CCN(C(=O)OC(C)(C)C)CC5)ccc4n3)cc(F)c2n1 ; Cc1cn2cc(-c3nc(=O)n4cc(N5CCNCC5)ccc4n3)cc(F)c2n1]

To a solution of tert-butyl 4-(2-(8-fluoro-2-methylimidazo[1,2-a]pyridin-6-yl)-4-oxo-4H-pyrido[1,2-a][1,3,5]triazin-7-yl)piperazine-1-carboxylate (20 mg, 0.042 mmol) in DCM (3 mL) was added TFA (0.5 ml) at 0° C. The reaction mixture was warmed to rt and stirred for 1 h. After concentration, the residue was purified by prep-HPLC (MeCN/0.05% NH₃·H₂O in water) to give 2-(8-fluoro-2-methylimidazo[1,2-a]pyridin-6-yl)-7-(piperazin-1-yl)-4H-pyrido[1,2-a][1,3,5]triazin-4-one as white solid (2 mg, yield: 13%). ESI-MS: [M+H]+ 380.2. ¹H NMR (400 MHz, CD₃OD) δ 9.28 (d, J=1.3 Hz, 1H), 8.38 (d, J=2.8 Hz, 1H), 8.25 (dd, J=9.4, 2.6 Hz, 1H), 7.94 (dd, J=12.0, 1.3 Hz, 1H), 7.82 (d, J=2.1 Hz, 1H), 7.72 (d, J=9.6 Hz, 1H), 4.58 (br s, 4H), 3.07-3.01 (m, 4H), 2.45 (d, J=0.7 Hz, 3H).

### Example 2. Synthesis of tert-butyl 3-(6-aminopyridin-3-yl)-3,8-diaza bicyclo[3.2.1]octane-8-carboxylate

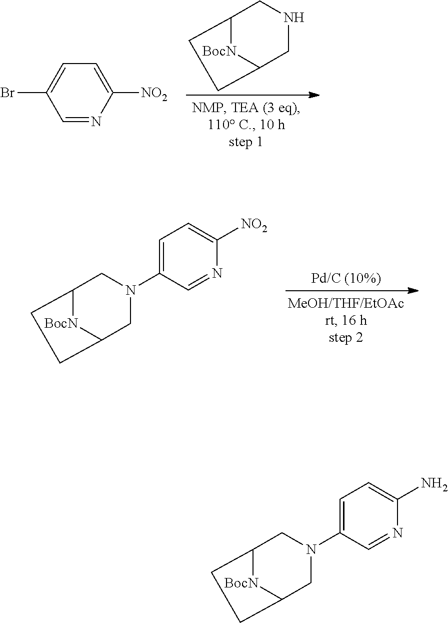 [SMILES: O=[N+]([O-])c1ccc(Br)cn1 ; CC(C)(C)OC(=O)N1C2CCC1CNC2 ; CC(C)(C)OC(=O)N1C2CCC1CN(c1ccc([N+](=O)[O-])nc1)C2 ; CC(C)(C)OC(=O)N1C2CCC1CN(c1ccc(N)nc1)C2]

### Step 1: tert-butyl 3-(6-nitropyridin-3-yl)-3,8-diazabicyclo[3.2.1]octane-8-carboxylate

To a suspension of tert-butyl 3,8-diazabicyclo[3.2.1] octane-8-carboxylate (1.0 g, 4.69 mmol) and 5-bromo-2-nitropyridine (1.44 g, 7.04 mmol) in NMP (10 mL) was added TEA (947 mg, 9.38 mmol), and the reaction mixture was stirred for 10 h at 110° C. After cooling to rt and diluting with water, the mixture was extracted with EA (40 mL×2), the combined organics was washed with brine (60 mL), dried over sodium sulfate and concentrated to dryness. The crude was purified by silica gel column (10˜100% EA in PE) to give title product (1.4 g, yield: 93.3%) as a yellow solid. ¹H NMR (400 MHz, CDCl₃) δ 8.17 (d, J=9.2 Hz, 1H), 8.08 (d, J=3.2 Hz, 1H), 7.15 (dd, J=9.2, 3.2 Hz, 1H), 3.59-3.55 (m, 2H), 3.40-3.36 (m, 1H), 3.30-3.24 (m, 2H), 2.37 (t, J=8.0 Hz, 1H), 2.04-1.98 (m, 2H), 1.83-1.77 (m, 2H), 1.49 (s, 9H).

### Step 2: tert-butyl 3-(6-aminopyridin-3-yl)-3,8-diazabicyclo[3.2.1]octane-8-carboxylate

To a mixture of tert-butyl 3-(6-nitropyridin-3-yl)-3,8-diazabicyclo[3.2.1]octane-8-carboxylate (1.4 g, 4.19 mmol) in MeOH/THF/EtOAc (15 mL/15 mL/15 mL) was added Pd/C (300 mg). The reaction mixture was stirred for 16 h at rt under hydrogen atmosphere (balloon pressure). The solvent was filtered and the filter cake was washed with MeOH (50 mL). The filtrate was concentrated to give title product (1.3 g, crude) as a brown solid. ¹H NMR (400 MHz, CDCl₃) δ 7.69 (d, J=2.8 Hz, 1H), 7.07 (dd, J=8.8, 2.8 Hz, 1H), 6.48 (d, J=8.8 Hz, 1H), 4.32-4.28 (m, 2H), 4.11 (s, 2H), 3.38 (t, J=7.2 Hz, 1H), 3.18 (d, J=10.4 Hz, 2H), 2.91-2.89 (m, 2H), 2.37 (t, J=8.0 Hz, 1H), 2.06-1.96 (m, 2H), 1.47 (s, 9H). ESI-MS: [M+H]⁺ 305.2.

### Example 3. Synthesis of tert-butyl 3-(6-aminopyridin-3-yl)-3,9-diaza bicyclo[3.3.1]nonane-9-carboxylate

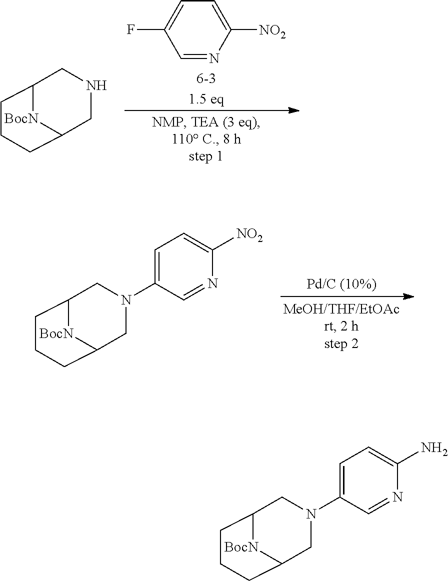 [SMILES: O=[N+]([O-])c1ccc(F)cn1 ; CC(C)(C)OC(=O)N1C2CCCC1CNC2 ; CC(C)(C)OC(=O)N1C2CCCC1CN(c1ccc([N+](=O)[O-])nc1)C2 ; CC(C)(C)OC(=O)N1C2CCCC1CN(c1ccc(N)nc1)C2]

### Step 1: tert-butyl 3-(6-nitropyridin-3-yl)-3,9-diazabicyclo[3.3.1]nonane-9-carboxylate

To a suspension of tert-butyl 3,9-diazabicyclo[3.3.1]nonane-9-carboxylate (400 mg, 1.77 mmol) and 5-fluoro-2-nitropyridine (377 mg, 2.65 mmol) in NMP (10 mL) was added TEA (357 mg, 3.54 mmol), and the reaction mixture was stirred for 6 h at 110° C. After cooling to rt and diluting with water, the mixture was extracted with EA (40 mL×2), the combined organics was washed with brine (60 mL), dried over sodium sulfate and concentrated to dryness. The crude was purified by silica gel column (10-100% EA in PE) to give tert-butyl 3-(6-nitropyridin-3-yl)-3,9-diazabicyclo[3.3.1]nonane-9-carboxylate (350 mg, yield: 56.9%) as a yellow solid. ¹H NMR (400 MHz, CDCl₃) δ 8.19 (d, J=9.2 Hz, 1H), 8.13 (d, J=3.2 Hz, 1H), 7.19 (dd, J=9.2, 3.2 Hz, 1H), 4.52-4.45 (m, 1H), 4.37-4.34 (m, 1H), 3.76 (d, J=12.0 Hz, 2H), 3.31-3.30 (m, 2H), 2.09-2.01 (m, 1H), 1.95-1.87 (m, 2H), 1.81-1.77 (m, 2H), 1.64-1.61 (m, 1H), 1.50 (s, 9H).

### Step 2: tert-butyl 3-(6-aminopyridin-3-yl)-3,9-diazabicyclo[3.3.I]nonane-9-carboxylate

To a mixture of tert-butyl 3-(6-nitropyridin-3-yl)-3,9-diazabicyclo[3.3.1]nonane-9-carboxylate (350 mg, 1.00 mmol) in MeOH/THF (20 mL/10 mL) was added Pd/C (50 mg). The reaction mixture was stirred for 2 h at rt under hydrogen atmosphere (balloon pressure). The solvent was filtered and the filter cake was washed with MeOH (50 mL). The filtrate was concentrated to give tert-butyl 3-(6-aminopyridin-3-yl)-3,9-diazabicyclo[3.3.1]nonane-9-carboxylate (280 mg, yield:87.7%) as a yellow solid. ¹H NMR (400 MHz, CDCl₃) δ 7.71 (d, J=2.8 Hz, 1H), 7.12 (dd, J=8.8, 3.0 Hz, 1H), 6.49 (d, J=8.4 Hz, 1H), 4.33-4.31 (m, 1H), 4.21-4.20 (m, 1H), 4.13-4.11 (m, 2H), 3.37 (t, J=9.8 Hz, 2H), 3.03-2.98 (m, 2H), 1.94-1.83 (m, 2H), 1.80-1.75 (m, 2H), 1.48 (s, 9H). ESI-MS: [M+H]⁺ 319.1.

### Example 4. Synthesis of tert-butyl 4-(6-aminopyridin-3-yl)-2,2-dimethylpiperazine-1-carboxylate

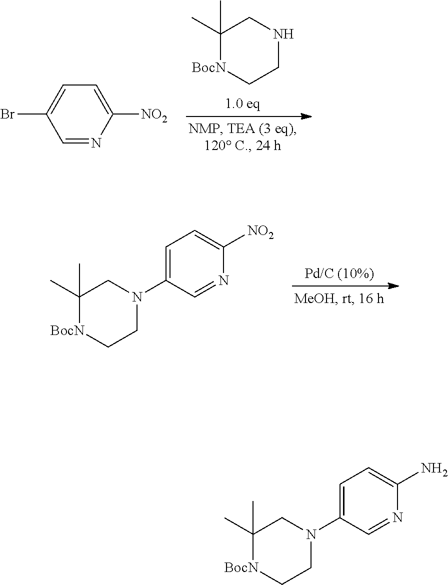 [SMILES: CC(C)(C)OC(=O)N1CCNCC1(C)C ; O=[N+]([O-])c1ccc(Br)cn1 ; CC(C)(C)OC(=O)N1CCN(c2ccc([N+](=O)[O-])nc2)CC1(C)C ; CC(C)(C)OC(=O)N1CCN(c2ccc(N)nc2)CC1(C)C]

### Step 1: tert-butyl 2,2-dimethyl-4-(6-nitropyridin-3-yl) piperazine-1-carboxylate

To a mixture of 5-bromo-2-nitropyridine (300 mg, 1.5 mmol) and tert-butyl 2,2-dimethylpiperazine-1-carboxylate (214 mg, 1 mmol) in NMP (5 mL) was added TEA (303 mg, 3 mmol), the reaction mixture was stirred for 24 h at 120° C. The reaction mixture was diluted with water (20 mL) and extracted with EtOAc (40 mL). The organic layer was washed with brine, dried with Na₂SO₄ and evaporated to give crude title compound. The residue was purified by silica gel column chromatography (PE/EA=6:1 to 1:1) to give title compound (210 mg, 62.3% yield) as a brown solid. ESI-MS (M+H)⁺: 337.3.

### Step 2: tert-butyl 4-(6-aminopyridin-3-yl)-2,2-dimethylpiperazine-1-carboxylate

The compound tert-butyl 4-(6-aminopyridin-3-yl)-2,2-dimethylpiperazine-1-carboxylate (900 mg, 2.67 mmol) was dissolved in MeOH (15 mL) and Pd/C (180 mg, 20%) was added. The mixture was charged with H₂ for three times and stirred at r.t. overnight under H₂ balloon. The mixture was filtered and the filtrate was concentrated to afford the title compound (700 mg, 86% yield) as a grey solid. ESI-MS (M+H)+: 307.3.

### Example 5. Synthesis of tert-butyl 5-(6-nitropyridin-3-yl)hexahydropyrrolo[3,4-c]pyrrole-2(1H)-carboxylate

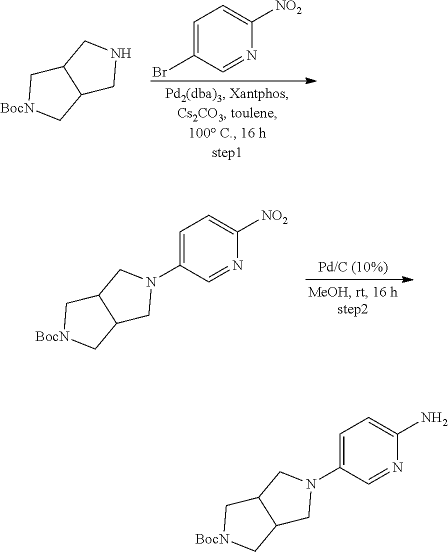 [SMILES: CC(C)(C)OC(=O)N1CC2CNCC2C1 ; O=[N+]([O-])c1ccc(Br)cn1 ; CC(C)(C)OC(=O)N1CC2CN(c3ccc([N+](=O)[O-])nc3)CC2C1 ; CC(C)(C)OC(=O)N1CC2CN(c3ccc(N)nc3)CC2C1]

### Step 1: tert-butyl 5-(6-n Mitropyridin-3-yl)hexahydropyrrolo[3,4-c]pyrrole-2(1H)-carboxylate

To a mixture of 5-bromo-2-nitropyridine (1.28 g, 6.33 mmol) and tert-butyl hexahydropyrrolo[3,4-c]pyrrole-2(1H)-carboxylate (900 mg, 4.2 mmol) in toluene (30 mL) was added Pd₂(dba)₃ (396 mg, 0.42 mmol), Xantphos (495 mg, 0.85 mmol) and Cs₂CO₃ (2.7 g, 8.49 mmol), the reaction mixture was stirred for 16 h at 100° C. The reaction mixture was diluted with water (20 mL) and extracted with EtOAc (40 mL). The organic layer was washed with brine, dried with Na₂SO₄ and evaporated to give crude title compound. The residue was purified by silica gel column chromatography (PE/EA=4:1 to 1:3) to give title compound (700 mg, 50% yield) as a brown solid. ESI-MS (M+H)+: 335.3.

### Step 2: tert-butyl 5-(6-aminopyridin-3-yl)hexahydropyrrolo[3,4-c] pyrrole-2(1H)-carboxylate

To a solution of tert-butyl 5-(6-nitropyridin-3-yl)hexahydropyrrolo[3,4-c]pyrrole-2(1H)-carboxylate (700 mg, 2.09 mmol) in MeOH (15 mL) was added Pd/C (70 mg, 10%), the mixture was charged with H₂ for three times and stirred at r.t. overnight under H₂ balloon. The mixture was filtered and the filtrate was concentrated to afford the title compound (600 mg, 94% yield) as a grey solid. ESI-MS (M+H)+: 305.2. ¹H NMR (400 MHz, CDCl₃) δ 7.50 (d, J=2.7 Hz, 1H), 6.87-6.79 (m, 1H), 6.49 (d, J=8.7 Hz, 1H), 3.64 (br.s, 2H), 3.45-3.25 (m, 4H), 3.17-3.10 (m, 2H), 2.98 (br.s, 2H), 1.45 (s, 9H).

### Example 6. Synthesis of tert-butyl 4-(6-amino-4-methylpyridin-3-yl) piperazine-1-carboxylate

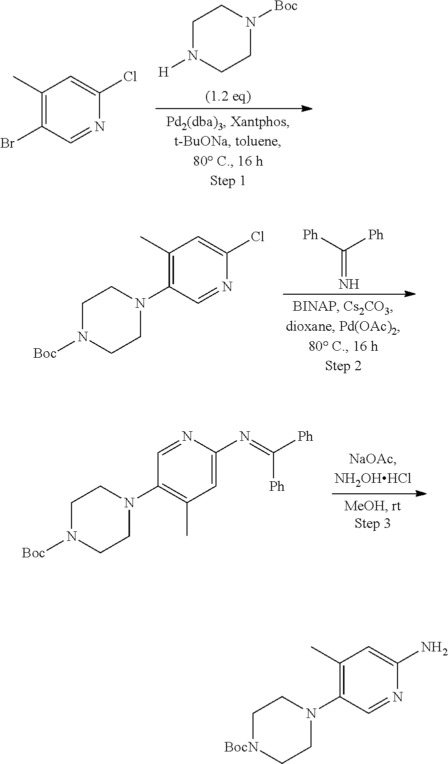

### Step 1: tert-butyl 4-(6-chloro-4-methylpyridin-3-yl)piperazine-1-carboxylate

To a solution of 5-bromo-2-chloro-4-methylpyridine (3.0 g, 14.4 mmol) in toluene (20 mL) were added t-BuONa (1.8 g, 18.6 mmol), Xantphos (333 mg, 0.57 mmol), Pd₂(dba)₃ (394 mg, 0.45 mmol) and tert-butyl piperazine-1-carboxylate (3.3 g, 17.3 mmol), the mixture was stirred at 80° C. overnight. The reaction mixture was concentrated in vacuo, the residue was purified by column chromatography (PE/EA=5:1) to give title product (2.1 g, 52%) as a light yellow solid. ESI-MS (M+H)⁺: 312. ¹H NMR (400 MHz, CDCl₃) δ 7.91 (s, 1H), 7.07 (s, 1H), 3.52-3.49 (m, 4H), 2.84-2.82 (m, 4H), 2.23 (s, 3H), 1.42 (s, 9H).

### Step 2: tert-butyl 4-(6-((diphenylmethylene)amino)-4-methylpyridin-3-yl) piperazine-1-carboxylate

A mixture of tert-butyl 4-(6-chloro-4-methylpyridin-3-yl) piperazine-1-carboxylate (1.7 g, 5.5 mmol) in 1,4-dioxane (20 mL) were added Pd(OAc)₂ (123 mg, 0.55 mmol), Cs₂CO₃ (3.6 g, 11.0 mmol), BINAP (685 mg, 1.1 mmol) and diphenylmethanimine (1.04 g, 5.7 mmol) was stirred at 80° C. overnight. The reaction mixture was concentrated in vacuo, the residue was purified by column chromatography (PE/EA=5:1) to give title product (3.5 g, yield: 60%) as a light yellow solid. ESI-MS (M+H)⁺ 457.3.

### Step 3: tert-butyl 4-(6-amino-4-methylpyridin-3-yl)piperazine-1-carboxylate

To a solution of tert-butyl 4-(6-((diphenylmethylene)amino)-4-methylpyridin-3-yl)piperazine-1-carboxylate (1.64 g, 3.6 mmol) in MeOH (10 mL) was added NaOAc (887 mg, 10.8 mmol) and NH₂OH·HCl (1.3 g, 18 mmol). The mixture was stirred at rt for 2 h. The mixture was diluted with water (20 mL), adjusted to pH=2 with 1M HCl, and extracted with EtOAc(50 mL×2). The aqueous phase was adjusted to pH=9 with NaHCO₃ (aq) and extracted with ethyl acetate (2×100 mL). The organic phases were combined, dried over anhydrous magnesium sulfate and concentrated under reduced pressure. The crude product was washed with tert-butyl methyl ether (10 mL) to give title product (770 mg, crude) as a yellow solid. ESI-MS (M+H)+293.1. ¹H NMR (400 MHz, CDCl₃) δ 7.73 (s, 1H), 6.37 (s, 1H), 4.23 (s, 2H), 3.54-3.52 (m, 4H), 2.83-2.81 (m, 4H), 2.22 (s, 3H), 1.48 (s, 9H).

### Example 7. Synthesis of tert-butyl (S)-4-(6-amino-5-fluoropyridin-3-yl)-2-methylpiperazine-1-carboxylate

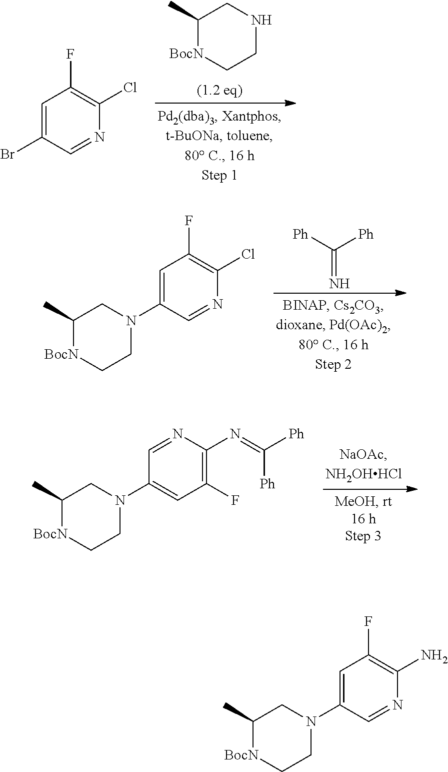 [SMILES: Fc1cc(Br)cnc1Cl ; C[C@H]1CNCCN1C(=O)OC(C)(C)C ; C[C@H]1CN(c2cnc(Cl)c(F)c2)CCN1C(=O)OC(C)(C)C ; N=C(c1ccccc1)c1ccccc1 ; C[C@H]1CN(c2cnc(N=C(c3ccccc3)c3ccccc3)c(F)c2)CCN1C(=O)OC(C)(C)C ; C[C@H]1CN(c2cnc(N)c(F)c2)CCN1C(=O)OC(C)(C)C]

### Step 1: tert-butyl (S)-4-(6-chloro-5-fluoropyridin-3-yl)-2-methylpiperazine-1-carboxylate

To a solution of 5-bromo-2-chloro-3-fluoropyridine (4.0 g, 19.2 mmol) in toluene (20 mL) were added t-BuONa (2.4 g, 2.5 mmol), Xantphos (463 mg, 0.8 mmol), Pd₂(dba)₃ (528 mg, 0.56 mmol), tert-butyl (S)-2-methylpiperazine-1-carboxylate (4.6 g, 23.04 mmol). The mixture was stirred at 80° C. overnight. The reaction mixture was concentrated in vacuum, the residue was purified by column chromatography (PE/EA=5:1) to give title product (5 g, 79.4%) as a light yellow solid. ESI-MS (M+H)+330.2. ¹H NMR (400 MHz, CDCl₃) δ 7.76 (d, J=2.5 Hz, 1H), 6.89 (dd, J=2.5, 0.8 Hz 1H), 4.30 (br.s, 1H), 3.90 (d, J=13.5 Hz, 1H), 3.43 (d, J=11.7 Hz, 1H), 3.31-3.15 (m, 2H), 3.03-3.01 (m, 1H), 2.81-2.79 (m, 1H), 1.42 (s, 9H), 1.20 (d, J=6.8 Hz, 3H).

### Step 2: tert-butyl (S)-4-(6-((diphenylmethylene)amino)-5-fluoropyridin-3-yl)-2-methylpiperazine-1-carboxylate

To a solution of tert-butyl (S)-4-(6-chloro-5-fluoro pyridin-3-yl)-2-methylpiperazine-1-carboxylate (2.0 g, 6 mmol) in 1,4-dioxane (20 mL) were added Pd(OAc)₂ (134 mg, 0.6 mmol), Cs₂CO₃ (3.9 g, 12.0 mmol), BINAP (747 mg, 1.2 mmol) and diphenylmethanimine (1.2 g, 6.4 mmol). The mixture was stirred at 80° C. overnight. The reaction mixture was concentrated in vacuo, the residue was purified by column chromatography (PE/EA=5:1) to give title product (2.1 g yield: 55%) as a light yellow solid. ESI-MS (M+H)+: 475.3.

### Step 3: tert-butyl (S)-4-(6-amino-5-fluoropyridin-3-yl)-2-methylpiperazine-1-carboxylate

To a solution of tert-butyl (S)-4-(6-((diphenylmethylene) amino)-5-fluoropyridin-3-yl)-2-methylpiperazine-1-carboxylate (1.326 g, 2.8 mmol) in MeOH (10 mL) was added NaOAc (688 mg, 8.4 mmol), NH₂OH·HCl (966 mg, 14 mmol) and the mixture was stirred at rt overnight. The mixture was diluted with water (20 mL), adjusted to pH=2 with 1M HCl, and extracted with EtOAc(50 mL×2). The aqueous phase was adjusted to pH=9 with NaHCO₃(aq) and extracted with ethyl acetate (2×100 mL). The organic phases were combined, dried over anhydrous magnesium sulfate and concentrated under reduced pressure. The crude product was purified by C₁₈ flash (0.1% NH₃·H₂O in water/CH₃CN) to give title product (943 mg, yield: 40%) as a yellow solid. ESI-MS (M+H)+: 311.3. ¹H NMR (400 MHz, CDCl₃) δ 7.55 (d, J=2.1 Hz, 1H), 7.01-6.90 (m, 1H), 4.34 (s, 3H), 3.94 (d, J=13.5 Hz, 1H), 3.29-3.17 (m, 2H), 3.10 (d, J=11.6 Hz, 1H), 2.84 (dd, J=11.6, 3.8 Hz, 1H), 2.72-2.59 (m, 1H), 1.48 (s, 9H), 1.31 (d, J=6.8 Hz, 3H).

### Example 8. Synthesis of tert-butyl (S)-4-(6-amino-5-methylpyridin-3-yl)-2-methylpiperazine-1-carboxylate

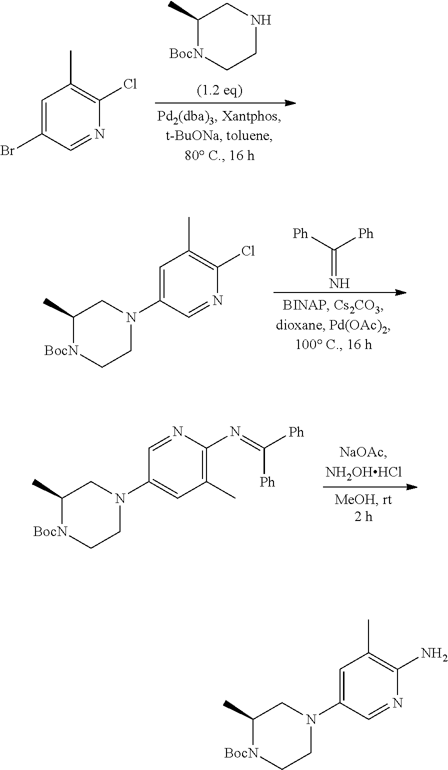

### Step 1: tert-butyl (S)-4-(6-chloro-5-methylpyridin-3-yl)-2-methylpiperazine-1-carboxylate

To a mixture of 5-bromo-2-chloro-3-methylpyridine (20 g, 0.097 mol), tert-butyl (S)-2-methylpiperazine-1-carboxylate (19.4 g, 0.097 mol) in toluene (400 mL) was added t-BuONa (12.1 g, 0.126 mol), Xantphos (2.25 g, 3.88 mmol) and Pd₂(dba)₃ (2.37 g, 2.91 mmol), the mixture was charged with N₂ for three times and stirred at 80° C. for 16 h. The reaction mixture was diluted with water (500 mL) and extracted with EtOAc (500 mL×3). The organic layer was washed with brine, dried over Na₂SO₄ and evaporated to give crude title compound. The crude was purified by silica gel column chromatography (EtOAc:PE=1:5) to give title compound (26 g, 82.4% yield) as a yellow solid. ¹H NMR (400 MHz, CDCl₃) δ 7.84 (d, J=2.9 Hz, 1H), 7.03 (t, J=10.2 Hz, 1H), 4.36 (br.s, 1H), 3.96 (d, J=13.4 Hz, 1H), 3.51-3.41 (m, 1H), 3.34-3.21 (m, 2H), 2.98-2.95 (m, 1H), 2.83-2.75 (m, 1H), 2.33 (s, 3H), 1.48 (s, 9H), 1.29 (d, J=6.8 Hz, 3H). ESI-MS (M+H)⁺: 326.0.

### Step 2: tert-butyl (S)-4-(6-((diphenylmethylene)amino)-5-methyl pyridin-3-yl)-2-methylpiperazine-1-carboxylate

To a mixture of tert-butyl (S)-4-(6-chloro-5-methylpyridin-3-yl)-2-methylpiperazine-1-carboxylate (26 g, 0.08 mol), diphenylmethanimine (15.2 g, 0.08 mol) in 1,4-dioxane (350 mL) was added Cs₂CO₃ (52 g, 0.16 mol), BINAP (9.9 g, 0.016 mol) and Pd(OAc)₂ (1.79 g, 0.008 mol), the mixture was charged with N2 for three times and stirred at 100° C. for 16 h. The reaction mixture was diluted with water (400 mL) and extracted with EtOAc (400 mL×3). The organic layer was washed with brine, dried over Na₂SO₄ and evaporated to give crude title compound. The residue was purified by silica gel column chromatography (EtOAc/PE=1:5) to give title compound (30 g, 80% yield) as a yellow solid. ¹H NMR (400 MHz, DMSO) δ 7.73-7.65 (m, 3H), 7.56-7.53 (m, 1H), 7.47 (t, J=7.4 Hz, 2H), 7.34-7.25 (m, 3H), 7.16-7.05 (m, 3H), 4.16 (br.s, 1H), 3.75 (d, J=13.2 Hz, 1H), 3.46 (d, J=11.9 Hz, 1H), 3.38 (d, J=12.1 Hz, 1H), 3.17-3.05 (m, 1H), 2.71-2.68 (m, 1H), 2.56-2.51 (m, 1H), 2.06 (d, J=8.0 Hz, 3H), 1.45-1.38 (m, 9H), 1.21-1.15 (m, 3H). ESI-MS (M+H)+: 471.2.

### Step 3: tert-butyl (S)-4-(6-amino-5-methylpyridin-3-yl)-2-methylpiperazine-1-carboxylate

To a solution of tert-butyl (S)-4-(6-((diphenylmethylene)amino)-5-methylpyridin-3-yl)-2-methylpiperazine-1-carboxylate (30 g, 0.06 mol) in MeOH (400 mL) was added NaOAc (15.6 g, 0.19 mol) and hydroxylamine hydrochloride (22 g, 0.32 mol), the mixture was stirred for 2 h at RT. The mixture was filtered and the filtrate was concentrated. The crude was purified by C₁₈ flash to give title compound (14 g, 72% yield) as a yellow solid. ESI-MS (M+H)⁺:307.3. ¹H NMR (400 MHz, DMSO-d₆) δ 7.47 (d, J=3.3 Hz, 1H), 7.03 (d, J=2.2 Hz, 1H), 5.22 (s, 2H), 4.17 (br.s, 1H), 3.76 (d, J=13.1 Hz, 1H), 3.29-3.20 (m, 1H), 3.17-3.04 (m, 2H), 2.61 (dd, J=11.7, 3.8 Hz, 1H), 2.48-2.40 (m, 1H), 2.03 (s, 3H), 1.41 (s, 9H), 1.22 (d, J=6.8 Hz, 3H).

### Example 9. Synthesis of tert-butyl 4-(6-amino-2-methylpyridin-3-yl) piperazine-1-carboxylate

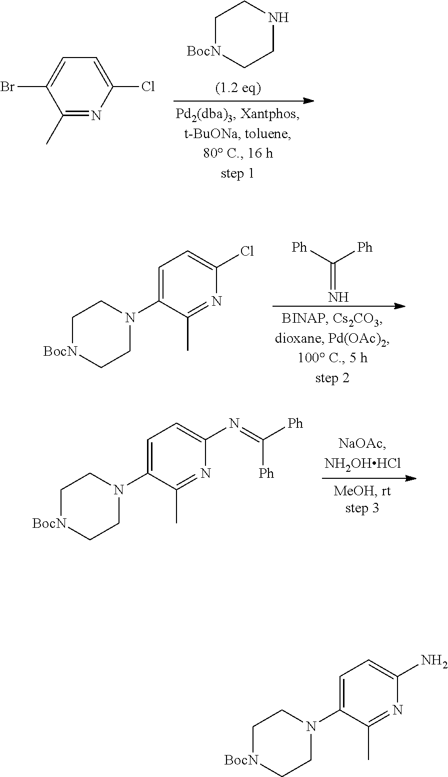

### Step 1: tert-butyl 4-(6-chloro-2-methylpyridin-3-yl)piperazine-1-carboxylate

A mixture of 3-bromo-6-chloro-2-methylpyridine (6.63 g, 32.1 mmol), tert-butyl piperazine-1-carboxylate (8.57 g, 38.5 mmol), Pd₂(dba)₃ (2.94 g, 3.2 mmol), Xantphos (3.7 g, 6.4 mmol) and t-BuONa (4.6 g, 48.2 mmol) in toluene (140 mL) was stirred at 80° C. for 16 h under Ar. After cooling to rt, the mixture was diluted with EA (100 mL) and washed with brine and water, concentrated under reduced pressure. The residue was purified by column chromatography (PE/EA=10:1 to 5:1) to afford title product as a white solid (8.0 g, yield: 80.2%). ¹H NMR (400 MHz, CDCl₃) δ 7.38 (d, J=8.8 Hz, 1H), 6.49 (d, J=8.8 Hz, 1H), 3.76-3.60 (m, 4H), 2.89-2.73 (m, 4H), 2.16 (s, 3H), 1.57 (s, 9H).

### Step 2: tert-butyl 4-(6-((diphenylmethylene)amino)-2-methylpyridin-3-yl)piperazine-1-carboxylate

A mixture of tert-butyl 4-(6-chloro-2-methylpyridin-3-yl) piperazine-1-carboxylate (2.5 g, 8.0 mmol), diphenylmethanimine (1.6 g, 8.8 mmol), Pd(OAc)₂ (180 mg, 0.8 mmol), BINAP (1.0 g, 1.6 mmol) and Cs₂CO₃ (5.2 g, 16.0 mmol) in dioxane (50 mL) was stirred at 100° C. for 5 h under Ar. After cooling to rt, the mixture was filtered and the filtrate was concentrated under reduced pressure. The residue was purified by column chromatography (PE/EA=5:1 to 2:1) to afford title product (1.2 g, yield: 51.3%) as a colorless oil. ¹H NMR (400 MHz, CDCl₃) δ 7.78 (d, J=7.3 Hz, 2H), 7.46-7.29 (m, 3H), 7.26-7.18 (m, 3H), 7.17-7.12 (m, 2H), 7.01 (d, J=8.4 Hz, 1H), 6.26 (d, J=8.4 Hz, 1H), 3.62-3.33 (m, 4H), 2.84-2.60 (m, 4H), 2.44 (s, 3H), 1.47 (s, 9H). ESI-MS: 457.5[M+H]⁺.

### Step 3: tert-butyl 4-(6-amino-2-methylpyridin-3-yl)piperazine-1-carboxylate

A mixture of tert-butyl 4-(6-((diphenylmethylene)amino)-2-methylpyridin-3-yl) piperazine-1-carboxylate (548 mg, 1.2 mmol), NaOAc (296 mg, 3.6 mmol) and NH₂OH—HCl (417 mg, 6.0 mmol) in MeOH (15 mL) was stirred at room temperature for 1 hour. The mixture was diluted with water (50 mL), extracted with EtOAc (30 mL×2). The combined organic layer was washed with brine (50 mL), dried over sodium sulfate, filtered and concentrated in vacuo. The residue was purified by column chromatography (PE/EA=5:1 to 2:1) to give title product as a white solid (280 mg, yield: 83%). ¹H NMR (400 MHz, CDCl₃) δ 7.29 (d, J=8.8 Hz, 1H), 6.40 (d, J=8.8 Hz, 1H), 3.63-3.48 (m, 4H), 2.82-2.66 (m, 4H), 2.41 (s, 3H), 1.48 (s, 9H). ESI-MS: 293.1[M+H]⁺.

### Example 10. Synthesis of tert-butyl 4-(6-amino-2-methoxypyridin-3-yl) piperazine-1-carboxylate

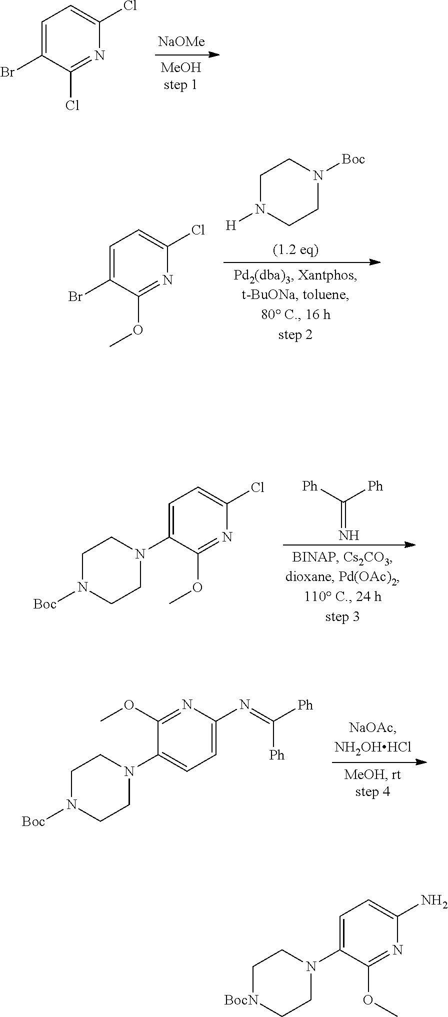

### Step 1: 3-bromo-6-chloro-2-methoxypyridine

To a solution of 3-bromo-2,6-dichloropyridine (554 mg, 2.0 mmol) in MeOH (10 mL) was added sodium methylate (216 mg, 4.0 mmol). The mixture was stirred at 80° C. for 5 h. After cooling to rt, the mixture was diluted with water (50 mL) and extracted with EtOAc (30 mL×2). The combined organic layers were washed with brine (20 mL), dried over anhydrous Na₂SO₄. After filtration, the filtrate was concentrated and used for next step directly without any further purification. ¹H NMR (400 MHz, CDCl₃) δ 7.73 (d, J=8.0 Hz, 1H), 6.82 (d, J=8.0 Hz, 1H), 4.02 (s, 3H).

### Step 2: tert-butyl 4-(6-chloro-2-methoxypyridin-3-yl)piperazine-1-carboxylate

A mixture of 3-bromo-6-chloro-2-methoxypyridine (223 mg, 1.0 mmol), tert-butyl piperazine-1-carboxylate (224 mg, 1.2 mmol), Pd₂(dba)₃ (92 mg, 0.1 mmol), Xantphos (116 mg, 0.2 mmol) and t-BuONa (145 mg, 1.5 mmol) in toluene (10 mL) was stirred at 80° C. for 16 h under Ar. After cooling to rt, the mixture was diluted with EA (80 mL) and washed with brine and water, concentrated under reduced pressure. The residue was purified by column chromatography (PE/EA=10:1 to 5:1) to afford title product as a white solid (100 mg, yield: 30.6%). ¹H NMR (400 MHz, CDCl₃) δ 7.07 (d, J=8.0 Hz, 1H), 6.85 (d, J=8.0 Hz, 1H), 4.01 (s, 3H), 3.66-3.52 (m, 4H), 3.04-2.94 (m, 4H), 1.48 (s, 9H). ESI-MS [M+Na]⁺: 350.2.

### Step 3: tert-butyl 4-(6-((diphenylmethylene)amino)-2-methoxypyridin-3-yl) piperazine-1-carboxylate

A mixture of tert-butyl 4-(6-chloro-2-methoxypyridin-3-yl)piperazine-1-carboxylate (100 mg, 0.3 mmol), diphenylmethanimine (58 mg, 0.32 mmol) Pd(OAc)₂ (7 mg, 0.03 mmol), BINAP (38 mg, 0.06 mmol) and Cs₂CO₃ (196 mg, 0.6 mmol) in dioxane (5 mL) was stirred at 110° C. for 24 h under Ar. After cooling to rt, the mixture was filtered and the filtrate was concentrated under reduced pressure. The residue was purified by column chromatography (PE/EA=5:1 to 2:1) to afford title product as a white solid (100 mg, yield: 69.3%). ESI-MS: 473.2[M+H]⁺.

### Step 4: tert-butyl 4-(6-amino-2-methoxypyridin-3-yl)piperazine-1-carboxylate

A mixture of tert-butyl 4-(6-((diphenylmethylene)amino)-2-methoxypyridin-3-yl)piperazine-1-carboxylate (577 mg, 1.2 mmol), NaOAc (296 mg, 3.6 mmol) and NH₂OH—HCl (417 mg, 6.0 mmol) in MeOH (15 mL) was stirred at room temperature for 1 hour. The mixture was diluted with water (50 mL), extracted with EtOAc (30 mL×2). The combined organic layer was washed with brine (50 mL), dried over sodium sulfate, filtered and concentrated in vacuo. The residue was purified by column chromatography (PE/EA=5:1 to 2:1) to give title product as a white solid (300 mg, yield: 83%). ¹H NMR (400 MHz, CDCl₃) δ 7.19-6.94 (m, 1H), 6.21-5.93 (m, 1H), 4.36 (s, 2H), 3.91 (s, 3H), 3.64-3.47 (m, 4H), 2.97-2.79 (m, 4H), 1.47 (s, 9H). ESI-MS: [M+H]⁺ 309.1.

### Example 11. Synthesis of tert-butyl 4-(6-amino-5-fluoropyridin-3-yl) piperazine-1-carboxylate

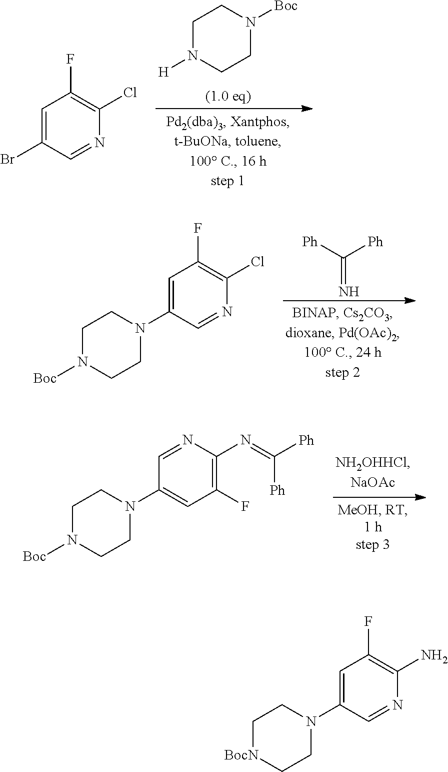

### Step 1: tert-butyl 4-(6-chloro-5-fluoropyridin-3-yl) piperazine-1-carboxylate

To a mixture of 5-bromo-2-chloro-3-fluoropyridine (15 g, 71.5 mmol), tert-butyl piperazine-1-carboxylate (13.3 g, 71.5 mmol) in toluene (250 mL) was added t-BuONa (8.8 g, 92.2 mmol), Xantphos (1.65 g, 2.85 mmol) and Pd₂(dba)₃ (1.95 g, 2.3 mmol), the mixture was charged with N₂ for three times and stirred at 100° C. for 16 h. The reaction mixture was diluted with water (500 mL) and extracted with EtOAc (500 mL×3). The organic layer was washed with brine, dried over Na₂SO₄ and evaporated to give crude title compound. The crude was purified silica gel column chromatography eluted with (EtOAc:PE=3:1) to give title compound (14.7 g, 65.8% yield) as a grey solid, ESI-MS (M+H)⁺:316.0.

### Step 2: tert-butyl 4-(6-((diphenylmethylene)amino)-5-fluoropyridin-3-yl)piperazine-1-carboxylate

To a mixture of tert-butyl 4-(6-chloro-5-fluoropyridin-3-yl)piperazine-1-carboxylate (10.4 g, 33 mmol), diphenylmethanimine (6.27 g, 36 mmol) in 1,4-dioxane (150 mL) was added Cs₂CO₃ (21.5 g, 66 mol), BINAP (3.0 g, 5.0 mmol) and Pd(OAc)₂ (370 mg, 1.65 mmol), the mixture was charged with N₂ for three times and stirred at 100° C. for 24 h. The reaction mixture was diluted with water (400 mL) and extracted with EtOAc (400 mL×3). The organic layer was washed with brine, dried over Na₂SO₄ and evaporated to give crude title compound. The crude was purified by silica gel column chromatography eluted with (EtOAc/PE=3:1) to give title compound (10.2 g, 67% yield) as a brown solid, ESI-MS (M+H)+:461.2.

### Step 3: tert-butyl 4-(6-amino-5-fluoropyridin-3-yl) piperazine-1-carboxylate

To a solution of tert-butyl 4-(6-((diphenylmethylene) amino)-5-fluoropyridin-3-yl)piperazine-1-carboxylate (20.4 g, 44.3 mmol) in MeOH (300 mL) was added NaOAc (10.9 mg, 133 mmol) and hydroxylamine hydrochloride (15.2 g, 221 mmol) at RT, the mixture was stirred for 2 h at RT. The mixture was filtered and the filtrate was concentrated. The crude was purified by silica gel column chromatography (EtOAc/PE=3:1) to give title compound (8.4 g, 65.2% yield) as a grey solid, ESI-MS (M+H)+:297.1.1H NMR (400 MHz, DMSO-d6) δ 7.50 (d, J=1.7 Hz, 1H), 7.22 (dd, J=13.4, 2.4 Hz, 1H), 5.63 (s, 2H), 3.48-3.38 (m, 4H), 2.96-2.84 (m, 4H), 1.41 (s, 9H).

### Example 12. Synthesis of tert-butyl 4-(6-amino-4-methoxypyridin-3-yl) piperazine-1-carboxylate

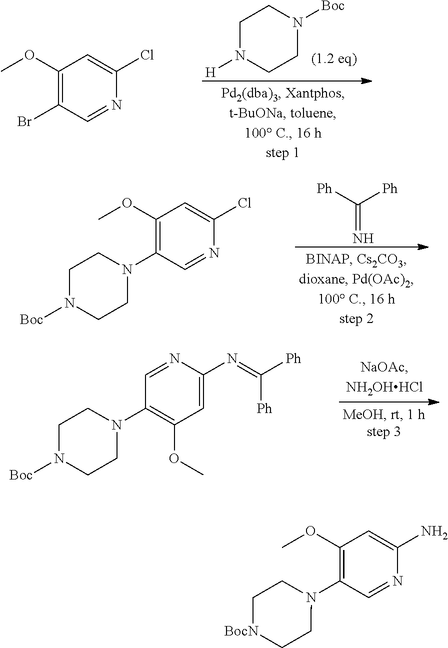

### Step 1: tert-butyl 4-(6-chloro-4-methoxypyridin-3-yl) piperazine-1-carboxylate

To a mixture of 5-bromo-2-chloro-4-methoxypyridine (6 g, 27.1 mmol), tert-butyl piperazine-1-carboxylate (6 g, 32.5 mmol) in toluene (100 mL) was added t-BuONa (5.2 g, 54.2 mmol), Xantphos (3.1 g, 5.42 mmol) and Pd₂(dba)₃ (2.5 g, 2.71 mmol), the mixture was charged with N₂ for three times and stirred at 100° C. for 16 h. The reaction mixture was diluted with water (100 mL) and extracted with EtOAc (100 mL×3). The organic layer was washed with brine, dried over Na₂SO₄ and evaporated to give crude title compound. The crude was purified by silica gel column chromatography (EtOAc:PE=3:1) to give title compound (1.4 g, 16% yield) as a grey solid, ESI-MS (M+H)⁺:328.0

### Step 2: tert-butyl 4-(6-((diphenylmethylene)amino)-4-methoxypyridin-3-yl)piperazine-1-carboxylate

To a mixture of tert-butyl 4-(6-chloro-5-fluoropyridin-3-yl)piperazine-1-carboxylate (1.4 g, 4.28 mmol), diphenylmethanimine (800 mg, 4.50 mmol) in 1,4-dioxane (40 mL) was added Cs₂CO₃ (2.78 g, 8.56 mol), BINAP (532 mg, 0.856 mmol) and Pd(OAc)₂ (96 mg, 0.428 mmol). The mixture was charged with N₂ for three times and stirred at 100° C. for 16 h. The reaction mixture was diluted with water (40 mL) and extracted with EtOAc (40 mL×3). The organic layer was washed with brine, dried over Na₂SO₄ and evaporated to give crude title compound. The crude was purified by silica gel column chromatography eluted with (EtOAc/PE=4:1) to give title compound (800 mg, 40% yield) as a brown solid, ESI-MS (M+H)⁺:473.1.

### Step 3: tert-butyl 4-(6-amino-4-methoxypyridin-3-yl)piperazine-1-carboxylate

To a solution of tert-butyl 4-(6-((diphenylmethylene)amino)-4-methoxypyridin-3-yl)piperazine-1-carboxylate (800 mg, 1.67 mmol) in MeOH (20 mL) was added NaOAc (412 mg, 5.02 mmol) and hydroxylamine hydrochloride (578 mg, 8.37 mmol) at RT, the mixture was stirred for 1 h at RT. The mixture was diluted with water (20 mL), adjusted to pH=2 with 1M HCl, and extracted with EtOAc (50 mL×2). The aqueous phase was adjusted to pH=9 with NaHCO₃(aq) and extracted with ethyl acetate (2×100 mL). The combined organic layers were washed with brine, dried over Na₂SO₄, and filtered filtrate were concentrated to afford title compound (400 mg, 77.8% yield) as a grey solid, ESI-MS (M+H)+: 309.1. ¹H NMR (400 MHz, CDCl₃) δ 7.57 (s, 1H), 6.02 (s, 1H), 4.35 (s, 2H), 3.85 (s, 3H), 3.59-3.56 (m, 4H), 2.94-2.90 (m, 4H), 1.48 (s, 9H).

### Example 13. Synthesis of tert-butyl 4-(6-amino-5-methylpyridin-3-yl)-2,2-dimethylpiperazine-1-carboxylate

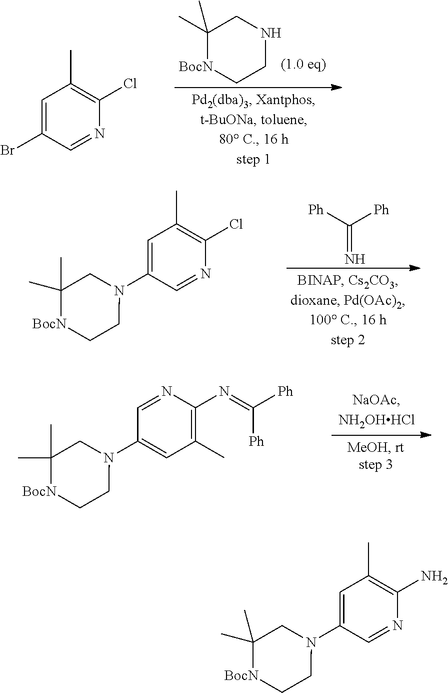

### Step 1: tert-butyl 4-(6-chloro-5-methylpyridin-3-yl)-2,2-dimethylpiperazine-1-carboxylate

To a mixture of 5-bromo-2-chloro-3-methylpyridine (5 g, 25.9 mmol), tert-butyl 2,2-dimethylpiperazine-1-carboxylate (5.32 g, 23.3 mmol) in toluene (100 mL) was added t-BuONa (5 g, 51.8 mmol), Xantphos (3 g, 5.18 mmol) and Pd₂(dba)₃ (2.38 g, 2.5 mmol), the mixture was charged with N₂ for three times and stirred at 80° C. for 16 h. The reaction mixture was diluted with water (100 mL) and extracted with EtOAc (100 mL×3). The organic layer was washed with brine, dried over Na₂SO₄ and evaporated to give crude title compound. The crude was purified silica gel column chromatography eluted with (EtOAc: PE=6:1) to give title compound (4.5 g, 51% yield) as a grey solid, ESI-MS (M+H)⁺:340.2. ¹H NMR (400 MHz, CDCl₃) δ 7.70 (d, J=3.0 Hz, 1H), 6.85 (d, J=2.8 Hz, 1H), 3.87-3.75 (m, 2H), 3.42-3.33 (m, 2H), 3.30 (s, 2H), 2.33 (s, 3H), 1.49 (s, 9H), 1.42 (s, 6H).

### Step 2: tert-butyl 4-(6-((diphenylmethylene)amino)-5-methylpyridin-3-yl)-2,2-dimethylpiperazine-1-carboxylate

To a mixture of tert-butyl 4-(6-chloro-5-methylpyridin-3-yl)-2,2-dimethylpiperazine-1-carboxylate (4.5 g, 13.27 mmol), diphenylmethanimine (2.52 g, 13.93 mmol) in 1,4-dioxane (100 mL) was added Cs₂CO₃ (8.62 g, 26.54 mol), BINAP (1.65 g, 2.65 mmol) and Pd(OAc)₂ (297 mg, 1.32 mmol). The mixture was charged with N₂ for three times and stirred at 100° C. for 16 h. The reaction mixture was diluted with water (40 mL) and extracted with EtOAc (40 mL×3). The organic layer was washed with brine, dried over Na₂SO₄ and evaporated to give crude title compound. The crude was purified by silica gel column chromatography (EtOAc/PE=6:1) to give title compound (4 g, 62% yield) as a brown solid, ESI-MS (M+H)⁺:473.1.

### Step 3: tert-butyl 4-(6-amino-5-methylpyridin-3-yl)-2,2-dimethylpiperazine-1-carboxylate

To a solution of tert-butyl 4-(6-((diphenylmethylene) amino)-5-methylpyridin-3-yl)-2,2-dimethylpiperazine-1-carboxylate (4 g, 8.26 mmol) in MeOH (100 mL) was added NaOAc (2 g, 24.79 mmol) and hydroxylamine hydrochloride (2.8 g, 41.32 mmol) at RT, the mixture was stirred for 1 h at RT. The mixture was diluted with water (20 mL), adjusted to pH=2 with 1M HCl, and extracted with EtOAc(50 mL×2). The aqueous phase was adjusted to pH=9 with NaHCO₃(aq) and extracted with ethyl acetate (2×100 mL). The combined organic layers were washed with brine, dried over Na₂SO₄, and concentrated to afford title compound (1.5 g, 57.7% yield) as a grey solid, ESI-MS (M+H)+: 321.0. ¹H NMR (400 MHz, CDCl₃) δ 7.55 (d, J=2.6 Hz, 1H), 6.90 (d, J=2.6 Hz, 1H), 4.09 (s, 2H), 3.69-3.65 (m, 2H), 3.18-3.13 (m, 2H), 2.97 (s, 2H), 2.13 (s, 3H), 1.49 (s, 9H), 1.44 (s, 6H).

### Example 14. Synthesis of tert-butyl 4-(6-amino-5-methoxypyridin-3-yl) piperazine-1-carboxylate

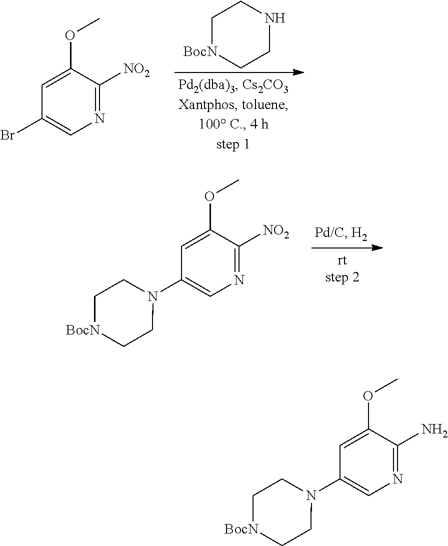 [SMILES: COc1cc(Br)cnc1[N+](=O)[O-] ; CC(C)(C)OC(=O)N1CCNCC1 ; COc1cc(N2CCN(C(=O)OC(C)(C)C)CC2)cnc1[N+](=O)[O-] ; COc1cc(N2CCN(C(=O)OC(C)(C)C)CC2)cnc1N]

### Step 1: tert-butyl 4-(5-methoxy-6-nitropyridin-3-yl) piperazine-1-carboxylate

To a mixture of 5-bromo-3-methoxy-2-nitropyridine (3 g, 12.93 mmol) and tert-butyl piperazine-1-carboxylate (3.61 g, 19.39 mmol) in toluene (30 mL) was added Pd₂(dba)₃ (1.18 g, 1.29 mmol), Xantphos (1.50 g, 2.58 mmol) and Cs₂CO₃ (8.43 g, 25.86 mmol). The reaction mixture was stirred for 4 h at 100° C. The reaction mixture was diluted with water (20 mL) and extracted with EtOAc (40 mL). The organic layer was washed with brine, dried with Na₂SO₄ and evaporated to give crude title compound. The residue was purified by silica gel column chromatography (PE/EA=3:1 to 1:1) to give title compound (600 mg, 13.7% yield) as a brown solid. ESI-MS (M+H)⁺: 339.1. ¹H NMR (400 MHz, CDCl₃) δ 7.72 (d, J=2.4 Hz, 1H), 6.69 (d, J=2.4 Hz, 1H), 3.98 (s, 3H), 3.66-3.63 (m, 4H), 3.44-3.41 (m, 4H), 1.49 (s, 9H).

### Step 2: tert-butyl 4-(6-amino-5-methoxypyridin-3-yl)piperazine-1-carboxylate

To a solution of tert-butyl 5-(6-nitropyridin-3-yl)hexahydropyrrolo[3,4-c]pyrrole-2(1H)-carboxylate (600 mg, 1.77 mmol) in MeOH (15 mL) was added Pd/C (60 mg, 10%). The mixture was charged with H₂ for three times and stirred at r.t. overnight under H₂ balloon. The mixture was filtered and the filtrate was concentrated to afford the title compound (470 mg, 86% yield) as a grey solid. ESI-MS (M+H)⁺: 309.1. ¹H NMR (400 MHz, CDCl₃) δ 7.33 (d, J=2.4 Hz, 1H), 6.70 (d, J=2.4 Hz, 1H), 4.43 (s, 2H), 3.84 (s, 3H), 3.60-3.56 (m, 4H), 3.01-2.94 (m, 4H), 1.48 (s, 9H).

### Example 15. Synthesis of 2,7-dimethylpyrazolo[1,5-a]pyridine-5-carbonitrile

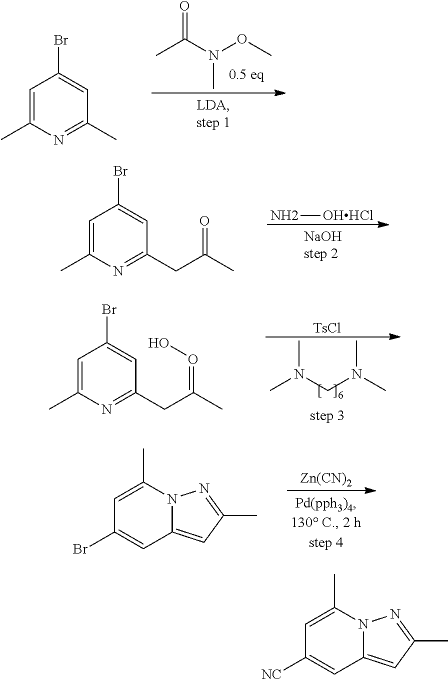

### Step 1: Preparation of 1-(4-bromo-6-methylpyridin-2-yl)propan-2-one

To a solution of 4-bromo-2,6-dimethylpyridine (25 g, 135 mmol) in dry. THE (400 mL) was added LDA (1.0 M, 162 mL, 162 mmol) dropwise at −65° C., the reaction mixture was stirred at this temperature for 4 hours. N-methoxy-N-methylacetamide (6.95 g, 67.5 mmol) in dry. THE (100 mL) was added dropwise at −65° C., the reaction mixture was stirred at this temperature for 1 hour. TLC (PE/EA=5:1) showed the reaction was completed. The mixture was diluted with water (30 mL), extracted with EtOAc (100 mL×2). The organic layer was washed with brine, dried with Na₂SO₄ and evaporated to give crude title compound. The residue was purified by column chromatography (PE/EA=5:1) to afford 1-(4-bromo-6-methylpyridin-2-yl)propan-2-one (16 g, Y:95%) as a yellow oil. 1H NMR (400 MHz, CDCl₃) δ 7.25 (s, 1H), 7.21 (s, 1H), 3.86 (s, 2H), 2.51 (s, 3H), 2.24 (s, 3H).

### Step 2: Preparation of (Z)-1-(4-bromo-6-methylpyridin-2-yl)propan-2-one oxime

To a solution of 1-(4-bromo-6-methylpyridin-2-yl)propan-2-one (14 g, 61.9 mmol) in MeOH (350 mL) were added NH₂OH·HCl (12.8 g, 185.7 mmol) and NaOH (7.4 g, 185.7 mmol), the mixture was stirred 60° C. for 16 h. LCMS showed the reaction was completed. The mixture was concentrated in vacuo. The residue was diluted with water (100 mL), extracted with EtOAc (100 mL×2). The organic layer was washed with brine, dried with Na₂SO₄ and evaporated to give crude title compound. The residue was purified by column chromatography (PE/EA=5:1) to afford (Z)-1-(4-bromo-6-methylpyridin-2-yl)propan-2-one oxime (12.5 g, 83.3% yield) as a yellow solid. ESI-MS (M+H)⁺:245.0.

### Step 3: Preparation of 5-bromo-2,7-dimethylpyrazolo[1,5-a]pyridine

To a solution of (Z)-1-(4-bromo-6-methylpyridin-2-yl)propan-2-one oxime (4.0 g, 16.5 mmol) and N1,N1,N6,N6-tetramethylhexane-1,6-diamine (4 mL, 19.8 mmol) in DCM (140 mL) was added TsC1 (4.0 g, 19.8 mmol) in DCM (20 mL) dropwise at 0° C., the reaction mixture was allowed to warmed to rt and stirred at rt for 16 h. TLC (PE/EA=5:1) showed the reaction was completed. The mixture was diluted with water (100 mL), extracted with DCM (100 mL×2). The organic layer was washed with brine, dried with Na₂SO₄ and evaporated to give crude title compound. The residue was purified by column chromatography (PE/EA=5:1) to 5-bromo-2,7-dimethylpyrazolo[1,5-a]pyridine (1.8 g, 48.6% yield) as a yellow solid. ESI-MS (M+H)⁺:225.2. 1H NMR (400 MHz, DMSO-d₆) δ 7.78 (d, J=1.4 Hz, 1H), 6.90 (d, J=1.4 Hz, 1H), 6.41 (s, 1H), 2.62 (s, 3H), 2.41 (s, 3H).

### Step 4: Preparation of 2,7-dimethylpyrazolo[1,5-a]pyridine-5-carbonitrile

To a solution of 5-bromo-2,7-dimethylpyrazolo[1,5-a]pyridine (2.0 g, 8.93 mmol) and Zn(CN)₂ (2.61 g, 22.32 mmol) in dry·DMF (50 mL) was added Pd(PPh3)₄ (1.03 g, 0.89 mmol), the mixture was charged with Ar for three times and stirred at 130° C. for 2 hours. TLC (PE/EA=5:1) showed the reaction was completed. The mixture was added water (40 mL), extracted with EA (50 mL*2), the combined organic layer was washed with brine (100 mL), dried over sodium sulfate, filtered and concentrated in vacuo, the residue was purified by column chromatography (PE/EA=5:1) to 2,7-dimethylpyrazolo[1,5-a]pyridine-5-carbonitrile (1.4 g, 91.5% yield) as a white solid. 1H NMR (400 MHz, CDCl₃) δ 7.77 (s, 1H), 6.67 (s, 1H), 6.55 (s, 1H), 2.75 (s, 3H), 2.55 (s, 3H).

### Example 16. Synthesis of tert-butyl (S)-4-(6-aminopyridin-3-yl)-2-methylpiperazine-1-carboxylate

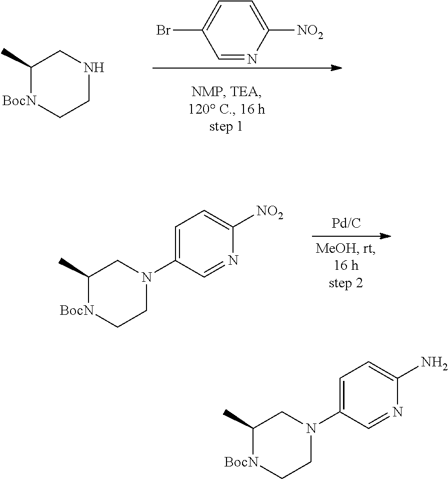 [SMILES: C[C@H]1CNCCN1C(=O)OC(C)(C)C ; O=[N+]([O-])c1ccc(Br)cn1 ; C[C@H]1CN(c2ccc([N+](=O)[O-])nc2)CCN1C(=O)OC(C)(C)C ; C[C@H]1CN(c2ccc(N)nc2)CCN1C(=O)OC(C)(C)C]

### Step 1: Preparation of tert-butyl (S)-2-methyl-4-(6-nitropyridin-3-yl)piperazine-1-carboxylate

To a mixture of tert-butyl (S)-2-methylpiperazine-1-carboxylate (5 g, 0.025 mol), 5-bromo-2-nitropyridine (7.6 g, 0.038 mol) in NMP (40 mL) was added TEA (7.57 g, 0.075 mol), the mixture was stirred at 120° C. for 16 h. The reaction mixture was diluted with water (100 mL) and extracted with EtOAc (80 mL×3). The organic layer was washed with brine, dried over Na₂SO₄ and evaporated to give crude title compound. The crude was purified silica gel column chromatography eluted with (EtOAc:PE=1:1) to give title compound (6 g, 74.5% yield) as a yellow solid. 1H NMR (400 MHz, DMSO-d₆) δ 8.21 (d, J=3.0 Hz, 1H), 8.16 (d, J=9.2 Hz, 1H), 7.46-7.38 (m, 1H), 4.22-4.12 (m, 1H), 3.93-3.75 (m, 3H), 3.42-3.41 (m, 1H), 3.33-3.21 (m, 1H), 3.18-3.10 (m, 1H), 1.42 (s, 9H), 1.11 (d, J=6.6 Hz, 3H). ESI-MS (M+H)+:323.0

### Step 2: Preparation of tert-butyl (S)-4-(6-aminopyridin-3-yl)-2-methylpiperazine-1-carboxylate

To a mixture of tert-butyl (S)-2-methyl-4-(6-nitropyridin-3-yl)piperazine-1-carboxylate (4 g, 0.012 mol) in THE (200 mL) was added Pd/C (400 mg, 10%), the mixture was charged with H₂ for three times and stirred at r.t. overnight. The mixture was filtered and the filtrate was concentrated to afford the title compound (3.5 g, 96.4% yield) as a yellow solid. ESI-MS: (M+H)+:293.1. 1H NMR (400 MHz, DMSO-d₆) δ 7.59 (d, J=2.3 Hz, 1H), 7.16 (dd, J=8.8, 2.6 Hz, 1H), 6.42 (d, J=8.8 Hz, 1H), 5.43 (s, 2H), 4.17-4.16 (m, 1H), 3.77-3.76 (m, 1H), 3.24-3.23 (m, 1H), 3.16-3.06 (m, 2H), 2.62-2.61 (m, 1H), 2.49-2.40 (m, 1H), 1.41 (s, 9H), 1.22 (d, J=6.6 Hz, 3H).

### Example 17. Synthesis of 2-(8-fluoro-2-methylimidazo[1,2-a]pyridin-6-yl)-7-(piperazin-1-yl)-4H-pyrido[1,2-a][1,3,5]triazin-4-one hydrochloride (Compound 100)

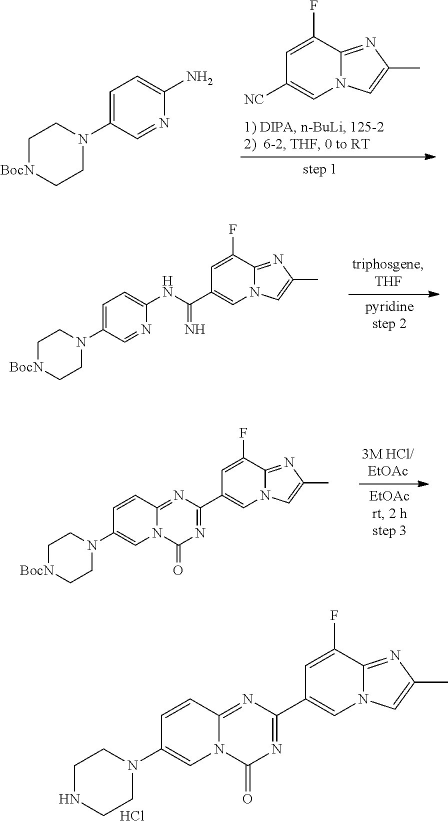 [SMILES: CC(C)(C)OC(=O)N1CCN(c2ccc(N)nc2)CC1 ; Cc1cn2cc(C#N)cc(F)c2n1 ; Cc1cn2cc(C(=N)Nc3ccc(N4CCN(C(=O)OC(C)(C)C)CC4)cn3)cc(F)c2n1 ; Cc1cn2cc(-c3nc(=O)n4cc(N5CCN(C(=O)OC(C)(C)C)CC5)ccc4n3)cc(F)c2n1 ; Cc1cn2cc(-c3nc(=O)n4cc(N5CCNCC5)ccc4n3)cc(F)c2n1 ; Cl]

### Step 1: tert-butyl 4-(6-(8-fluoro-2-methylimidazo[1,2-a]pyridine-6-carboximidamido)pyridin-3-yl)piperazine-1-carboxylate

To a mixture of diisopropylamine (0.6 mL, 4.4 mmol) in dry THE (15 mL) was added n-BuLi (3 mL, 4.8 mmol, 1.6M) dropwise at −65° C. The mixture was stirred at this temperature for 1 hour. Tert-butyl 4-(6-aminopyridin-3-yl)piperazine-1-carboxylate (1.12 g, 4.0 mmol) in dry THE (5 mL) was added. The mixture was allowed to warm to room temperature and stirred for 1 h. 8-fluoro-2-methylimidazo[1,2-a]pyridine-6-carbonitrile (350 mg, 2.0 mmol) in dry THE (10 mL) was added to the mixture at −40° C. and allowed to warm to room temperature and stirred for 16 h. The mixture was diluted with water (30 mL), extracted with EtOAc (30 mL×2). The combined organic layer was washed with brine (50 mL), dried over sodium sulfate, filtered and concentrated in vacuo. The residue was purified by column chromatography (PE/EA=1:1 to 0:1), then slurried with MeOH (30 mL) to give title product (600 mg, yield: 44.4%, three batches) as a yellow solid. ¹H NMR (400 MHz, CDCl₃) δ 8.57 (s, 1H), 8.01 (d, J=2.8 Hz, 1H), 7.47 (d, J=2.0 Hz, 1H), 7.36-7.31 (m, 2H), 7.21 (d, J=8.8 Hz, 1H), 3.62-3.58 (m, 4H), 3.15-3.12 (m, 4H), 2.50 (s, 3H), 1.49 (s, 9H). ESI-MS: [M+H]⁺: 454.3.

### Step 2: tert-butyl 4-(2-(8-fluoro-2-methylimidazo[1,2-a]pyridin-6-yl)-4-oxo-4H-pyrido[1,2-a][1,3,5]triazin-7-yl)piperazine-1-carboxylate

To a solution of tert-butyl 4-(6-(8-fluoro-2-methylimidazo[1,2-a]pyridine-6-carboximidamido) pyridin-3-yl)piperazine-1-carboxylate (600 mg, 1.32 mmol) in THE (60 mL) and pyridine (6 mL) was added triphosgene (1.18 g, 3.97 mmol). The reaction mixture was stirred for 1 h at rt. The mixture was diluted with water (30 mL), extracted with EtOAc (30 mL×2). The combined organic layer was washed with brine (50 mL), dried over sodium sulfate, filtered and concentrated in vacuo. The residue was diluted with MeOH (40 mL) and stirred for 1 hour at rt. The precipitate was filtered and dried in vacuo to give title product (600 mg, yield: 97%). ¹H NMR (400 MHz, CDCl₃) δ 9.21 (s, 1H), 8.48 (d, J=2.4 Hz, 1H), 7.95-7.90 (m, 2H), 7.66 (d, J=9.6 Hz, 1H), 7.48 (d, J=2.0 Hz, 1H), 3.67-3.63 (m, 4H), 3.29-3.26 (m, 4H), 2.50 (s, 3H), 1.50 (s, 9H). ESI-MS: [M+H]⁺: 480.0.

### Step 3: 2-(8-fluoro-2-methylimidazo[1,2-a]pyridin-6-yl)-7-(piperazin-1-yl)-4H-pyrido[1,2-a][1,3,5]triazin-4-one hydrochloride

To a solution of tert-butyl 4-(2-(8-fluoro-2-methylimidazo[1,2-a]pyridin-6-yl)-4-oxo-4H-pyrido[1,2-a][1,3,5]triazin-7-yl)piperazine-1-carboxylate (1.25 g, 2.61 mmol) in EtOAc (15 mL) was added 3M HCl in EtOAc (15 ml) at room temperature. The reaction mixture was stirred for 2 h. The precipitate was filtered and lyophilized to give title product as HCl salt (1.0 g, yield: 86.2%) as a yellow solid. ¹H NMR (400 MHz, MeOD-d₄) δ 9.66 (s, 1H), 8.66 (d, J=11.2 Hz, 1H), 8.60 (d, J=2.4 Hz, 1H), 8.41 (dd, J=9.6, 2.8 Hz, 1H), 8.25 (s, 1H), 7.91 (d, J=9.6 Hz, 1H), 3.71-3.67 (m, 4H), 3.49-3.46 (m, 4H), 2.62 (s, 3H). ESI-MS: [M+H]⁺ 380.2.

### Example 18. Synthesis of (S)-2-(8-fluoro-2-methylimidazo[1,2-a]pyridin-6-yl)-7-(3-methylpiperazin-1-yl-4H-pyrido[1,2-a][1,3,5]triazin-4-one HCl (Compound 101)

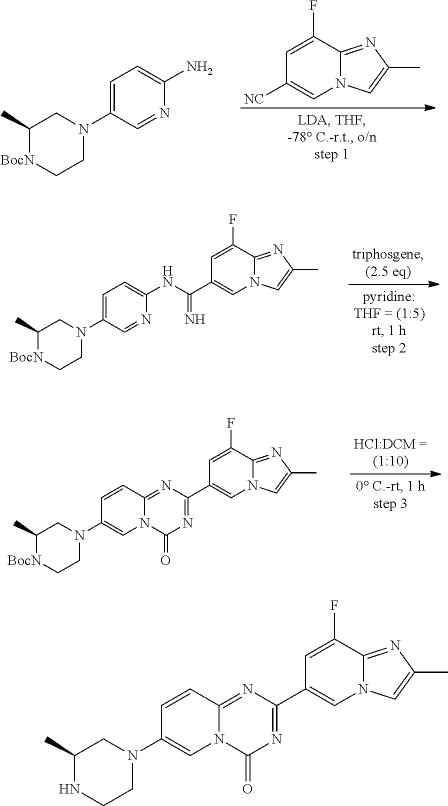

### Step 1: tert-butyl (S)-4-(6-(8-fluoro-2-methylimidazo[1,2-a]pyridine-6-carboximidamido)pyridin-3-yl)-2-methylpiperazine-1-carboxylate

To a mixture of tert-butyl (S)-4-(6-aminopyridin-3-yl)-2-methylpiperazine-1-carboxylate (700 mg, 2.40 mmol) in THF (15 mL) was added LDA (2.8 mL, 1M in THF) at −78° C. under N₂. The resulting mixture was stirred for 1 h at 0° C., The mixture was allowed to cool down to −78° C. and 8-fluoro-2-methylimidazo[1,2-a]pyridine-6-carbonitrile (140 mg, 0.80 mmol) was added. The mixture was stirred overnight at room temperature. After diluting with water, the mixture was extracted with EtOAc (40 mL×3). The organic layers was washed with brine and concentrated. The residue was purified by silica gel column chromatography (DCM:MeOH=20:1) to give title product (130 mg, yield: 35%) as a yellow solid. ESI-MS (M+H)⁺: 468.1 ¹H NMR (400 MHz, CDCl₃) δ 8.59 (s, 1H), 7.97 (d, J=2.9 Hz, 1H), 7.46 (d, J=2.3 Hz, 1H), 7.38-7.33 (m, 1H), 7.29 (dd, J=8.9, 3.0 Hz, 1H), 7.22 (d, J=8.8 Hz, 1H), 4.38 (br s, 1H), 3.98 (d, J=13.0 Hz, 1H), 3.47 (d, J=11.4 Hz, 1H), 3.36-3.22 (m, 2H), 2.97 (dd, J=11.9, 3.7 Hz, 1H), 2.79-2.76 (m, 1H), 2.49 (s, 3H), 1.33 (d, J=6.7 Hz, 3H).

### Step 2: tert-butyl (S)-4-(2-(8-fluoro-2-methylimidazo[1,2-a]pyridin-6-yl)-4-oxo-4H-pyrido[1,2-a][1,3,5]triazin-7-yl)-2-methylpiperazine-1-carboxylate

To a mixture of tert-butyl (S)-4-(6-(8-fluoro-2-methylimidazo[1,2-a]pyridine-6-carboximidamido)pyridin-3-yl)-2-methylpiperazine-1-carboxylate (120 mg, 0.26 mmol) in THE (5 mL) was added triphosgene (193 mg, 0.65 mmol) and pyridine (1 mL). The resulting mixture was stirred for 1 h at room temperature. The mixture was quenched with water and extracted with EtOAc (40 mL×3). The organic layers and washed with brine. The organic layer was concentrated and purified by silica gel column chromatography (DCM:MeOH=20:1) to give title product (120 mg, yield: 94%) as a yellow solid. ESI-MS (M+H)⁺: 494.1 Step 3: (S)-2-(8-fluoro-2-methylimidazo[1,2-a]pyridin-6-yl)-7-(3-methylpiperazin-1-yl)-4H-pyrido[1,2-a][1,3,5]triazin-4-one HCl

To a solution of tert-butyl (S)-4-(2-(8-fluoro-2-methylimidazo[1,2-a]pyridin-6-yl)-4-oxo-4H-pyrido[1,2-a][1,3,5]triazin-7-yl)-2-methylpiperazine-1-carboxylate (110 mg, 0.22 mmol) in DCM (10 mL) was added HCl in dioxane (1 mL) at 0° C. The mixture was stirred for 1 h at rt. After concentration, the residue was stirred with EtOAc. The precipitate was filtered and the solid was dried to give title product (55.68 mg, yield: 64%) as a yellow solid. ESI-MS (M+H)⁺: 394.4 ¹H NMR (400 MHz, MeOD) δ 9.66 (s, 1H), 8.66 (d, J=11.0 Hz, 1H), 8.60 (d, J=2.7 Hz, 1H), 8.43 (dd, J=9.6, 2.8 Hz, 1H), 8.25 (s, 1H), 7.91 (d, J=9.5 Hz, 1H), 4.06 (dd, J=17.7, 14.2 Hz, 2H), 3.64-3.57 (m, 2H), 3.46-3.32 (m, 2H), 3.09 (dd, J=13.3, 10.7 Hz, 1H), 2.63 (s, 3H), 1.47 (d, J=6.6 Hz, 1H).

### Example 19. Synthesis of (R)-2-(8-fluoro-2-methylimidazo[1,2-a]pyridin-6-yl)-7-(3-methylpiperazin-1-yl)-4H-pyrido[1,2-a][1,3,5]triazin-4-one formic acid (Compound 102)

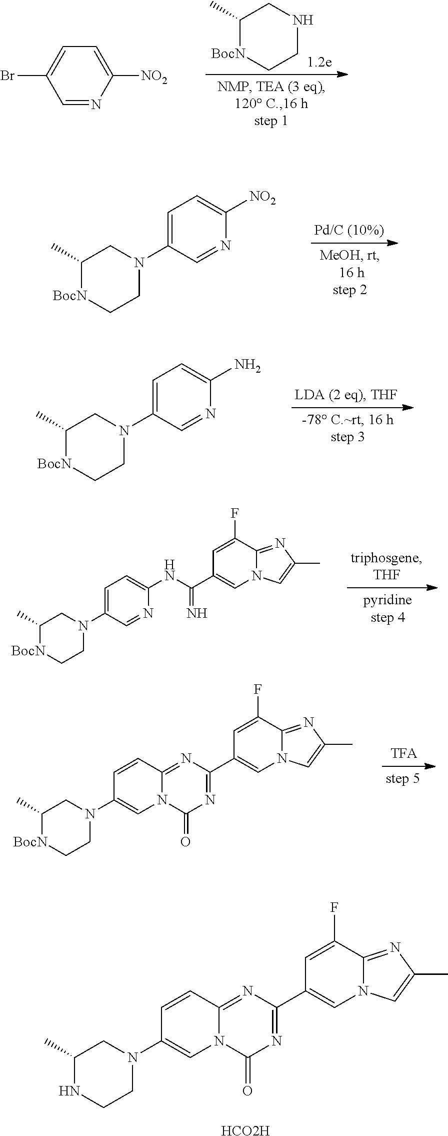

### Step 1: tert-butyl (R)-2-methyl-4-(6-nitropyridin-3-yl)piperazine-1-carboxylate

To a suspension of 5-bromo-2-nitropyridine (2.0 g, 10.0 mmol) and tert-butyl (R)-2-methylpiperazine-1-carboxylate (2.4 g, 12.0 mmol) in NMP (50 mL) was added TEA (3.0 g, 30.0 mmol), and the reaction mixture was stirred for 16 h at 120° C. After cooling to rt, the mixture was diluted with water and extracted with EA. The organic layer was washed with brine, dried over Na₂SO₄, concentrated. The crude was purified by silica gel column (10˜100% EA in PE). The product was obtained as off-white solid (2.5 g, yield: 77%). ESI-MS: [M+H]⁺ 323.1

### Step 2: tert-butyl (R)-4-(6-aminopyridin-3-yl)-2-methylpiperazine-1-carboxylate

To a mixture of tert-butyl (R)-2-methyl-4-(6-nitropyridin-3-yl)piperazine-1-carboxylate (2.5 g, 7.76 mmol) in MeOH (100 mL) was added Pd/C (250 mg). The reaction mixture was stirred for 16 h at rt under hydrogen (balloon pressure). The mixture was filtered and washed with MeOH (50 mL). The filtrate was concentrated to give the desired product as purple semi-solid (2 g, crude yield: 89%), which was used to next step without further purification. ESI-MS: [M+H]⁺ 293.3

### Step 3: tert-butyl (R)-4-(6-(8-fluoro-2-methylimidazo[1,2-a]pyridine-6-carboximidamido)pyridin-3-yl)-2-methylpiperazine-1-carboxylate

To a mixture of N, N-Diisopropylamine (0.15 mL, 1 mmol) in dry THF (5 mL) was added n-BuLi (0.7 mL, 1.6 M in hexane, 1.1 mmol) at −78° C., and the mixture was stirred for 40 min at −70° C. Then tert-butyl (R)-4-(6-aminopyridin-3-yl)-2-methylpiperazine-1-carboxylate (293 mg, 1.0 mmol) was added and the resulting mixture was stirred for 1 h at rt. After cooling to −78° C., 8-fluoro-2-methylimidazo[1,2-a]pyridine-6-carbonitrile (100 mg, 0.57 mmol) was added, the mixture was allowed to warm to rt and stirred for another 16 h. After diluting with water (20 mL), the mixture was extracted with EA (50 ml×2). The combined organics was washed with brine, dried over Na₂SO₄. After concentration, the crude was purified with silica gel (PE: EA=1:2) to give product (70 mg, yield: 26%). ESI-MS: [M+H]⁺ 468.4

### Step 4: tert-butyl (R)-4-(2-(8-fluoro-2-methylimidazo[1,2-a]pyridin-6-yl)-4-oxo-4H-pyrido[1,2-a][1,3,5]triazin-7-yl)-2-methylpiperazine-1-carboxylate

To a solution of tert-butyl (R)-4-(6-(8-fluoro-2-methylimidazo[1,2-a]pyridine-6-carboximidamido)pyridin-3-yl)-2-methylpiperazine-1-carboxylate (70 mg, 0.15 mmol) in THE (8 mL) was added triphosgene (120 mg, 0.75 mmol), the reaction mixture was stirred for 5 min, and pyridine (1.0 mL) was added. The mixture was stirred for 1 h. After concentrated under reduced pressure, the residue was purified by silica gel column (1˜10% MeOH in DCM). The product was obtained as a white solid (40 mg, yield: 53%). ESI-MS: [M+H]⁺ 494.4

### Step 5: (R)-2-(8-fluoro-2-methylimidazo[1,2-a]pyridin-6-yl)-7-(3-methylpiperazin-1-yl)-4H-pyrido[1,2-a][1,3,5]triazin-4-one formic acid

To a solution of tert-butyl (R)-4-(2-(8-fluoro-2-methylimidazo[1,2-a]pyridin-6-yl)-4-oxo-4H-pyrido[1,2-a][1,3,5]triazin-7-yl)-2-methylpiperazine-1-carboxylate (40 mg, 0.08 mmol) in DCM (3 mL) was added TFA (0.5 ml) at 0° C. The reaction mixture was warmed to rt and stirred for 1 h. After concentration, the residue was purified by pre-HPLC (MeCN/0.05% formic acid in water) to give title product as white solid (3.1 mg, yield: 10%). ESI-MS: [M+H]⁺ 394.3. ¹H NMR (400 MHz, MeOD) δ 9.28 (d, J=1.0 Hz, 1H), 8.50 (s, 2H), 8.41 (d, J=2.6 Hz, 1H), 8.27 (dd, J=9.6, 2.7 Hz, 1H), 7.91 (d, J=12.0 Hz, 1H), 7.83 (d, J=2.3 Hz, 1H), 7.74 (d, J=9.6 Hz, 1H), 3.97-3.84 (m, 2H), 3.53-3.43 (m, 2H), 3.26-3.24 (m, 1H), 3.13-3.10 (m, 1H), 2.92-2.84 (m, 1H), 2.45 (s, 3H), 1.40 (d, J=6.5 Hz, 3H).

### Example 20. Synthesis of 2-(8-fluoro-2-methylimidazo[1,2-a]pyridin-6-yl)-7-(4-methylpiperazin-1-yl)-4H-pyrido[1,2-a][1,3,5]triazin-4-one (Compound 103)

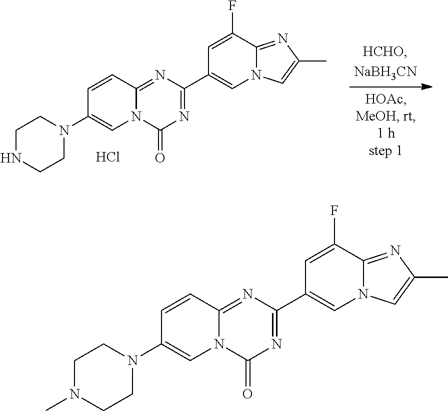

To a solution of 2-(8-fluoro-2-methylimidazo[1,2-a]pyridin-6-yl)-7-(piperazin-1-yl)-4H-pyrido[1,2-a][1,3,5]triazin-4-one hydrochloride (20 mg, 0.048 mmol), HCHO (0.5 mL) and HOAc (0.1 mL) was added NaBH₃CN (10 mg, 0.144 mmol) in MeOH (5 mL) at rt. The reaction mixture was stirred for 1 h at rt. After concentration, the residue was purified by pre-HPLC (MeCN/0.05% NH₃H₂O in water) to give title product (4.8 mg, yield: 25%) as a yellow solid. ESI-MS (M+H)⁺: 394.3. ¹H NMR (400 MHz, DMSO-d₆) δ 9.42 (d, J=1.3 Hz, 1H), 8.35 (dd, J=9.6, 2.9 Hz, 1H), 8.28 (d, J=2.8 Hz, 1H), 8.03 (d, J=2.3 Hz, 1H), 7.82 (dd, J=12.4, 1.3 Hz, 1H), 7.72 (d, J=9.5 Hz, 1H), 3.29 (s, 8H), 2.38 (s, 3H), 2.25 (s, 3H).

### Example 21. Synthesis of 7-(1,4-diazepan-1-yl)-2-(8-fluoro-2-methylimidazo[1,2-a]pyridin-6-yl)-4H-pyrido[1,2-a][1,3,5]triazin-4-one hydrochloride (Compound 104)

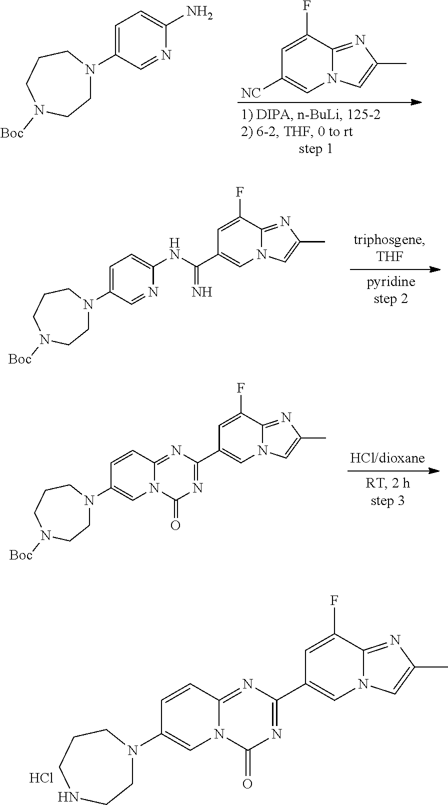 [SMILES: CC(C)(C)OC(=O)N1CCCN(c2ccc(N)nc2)CC1 ; Cc1cn2cc(C#N)cc(F)c2n1 ; Cc1cn2cc(C(=N)Nc3ccc(N4CCCN(C(=O)OC(C)(C)C)CC4)cn3)cc(F)c2n1 ; Cc1cn2cc(-c3nc(=O)n4cc(N5CCCN(C(=O)OC(C)(C)C)CC5)ccc4n3)cc(F)c2n1 ; Cl ; Cc1cn2cc(-c3nc(=O)n4cc(N5CCCNCC5)ccc4n3)cc(F)c2n1]

### Step 1: tert-butyl 4-(6-(8-fluoro-2-methylimidazo[1,2-a]pyridine-6-carboximidamido)pyridin-3-yl)-1,4-diazepane-1-carboxylate

To three-neck flask were added DIPA (220 mg, 2.2 mmol) and THF (6 mL), the mixture was cooling down to −60° C., then 1.6 M n-BuLi (1.5 mL, 2.4 mmol) was dropwise added under N₂ and stirred for 1 h at this temperature. Tert-butyl 4-(6-aminopyridin-3-yl)-1,4-diazepane-1-carboxylate (584 mg, 2 mmol) in THE (2 mL) was added at −60° C., the mixture was stirred for 1.5 h at RT. The mixture was cooling down to −60° C. again. Then 8-fluoro-2-methylimidazo[1,2-a]pyridine-6-carbonitrile (175 mg, 1 mmol) in THE (5 mL) was added slowly. The mixture was stirred at RT overnight. The reaction mixture was diluted with water (30 mL) and extracted with EtOAc (40 mL×3). The organic layer was washed with brine, dried with Na₂SO₄ and evaporated to give crude title compound. The residue was purified by C₁₈ column chromatography to give title compound (160 mg, 34% yield) as a grey solid. ESI-MS (M+H)⁺: 468.3.

### Step 2: tert-butyl 4-(2-(8-fluoro-2-methylimidazo[1,2-a]pyridin-6-yl)-4-oxo-4H-pyrido[1,2-a][1,3,5]triazin-7-yl)-1,4-diazepane-1-carboxylate

To a solution of tert-butyl 4-(6-(8-fluoro-2-methylimidazo[1,2-a]pyridine-6-carboximidamido)pyridin-3-yl)-1,4-diazepane-1-carboxylate (135 mg, 0.29 mmol) was dissolved in THE (15 mL) and pyridine (1 mL) was added triphosgene (301 mg, 1.02 mmol) at 0° C., the mixture was stirred for 2 h at r.t. The reaction mixture was diluted with water (15 mL) and extracted with EtOAc (20 mL×3). The organic layer was washed with brine, dried with Na₂SO₄ and evaporated to give crude title compound. The residue was purified by C18 column chromatography to give title compound (80 mg; 56% yield) as a yellow solid. ESI-MS (M+H)⁺: 494.3.

### Step 3: 7-(1,4-diazepan-1-yl)-2-(8-fluoro-2-methylimidazo[1,2-a]pyridin-6-yl)-4H-pyrido[1,2-a][1,3,5]triazin-4-one hydrochloride

A solution of tert-butyl 4-(2-(8-fluoro-2-methylimidazo[1,2-a]pyridin-6-yl)-4-oxo-4H-pyrido[1,2-a][1,3,5]triazin-7-yl)-1,4-diazepane-1-carboxylate (80 mg, 0.62 mmol) in HCl/EA (3 mL) was stirred for 2 h at RT. The mixture was filtered and the filter cake was washed with EtOAc (1 mL) and dried to afford title compound (50 mg, 78.3% yield) as a yellow solid. ESI-MS (M+H)⁺:394.3. ¹H NMR (400 MHz, DMSO-d₆) δ 9.35 (s, 2H), 9.24 (s, 1H), 8.13 (s, 1H), 8.08 (s, 1H), 7.71 (d, J=9.0 Hz, 1H), 7.49 (d, J=10.6 Hz, 1H), 7.43 (d, J=8.8 Hz, 1H), 3.85 (br.s, 2H), 3.63 (br.s, 2H), 3.26 (br.s, 2H), 3.15 (br.s, 2H), 2.38 (s, 3H), 2.14 (br s, 2H).

### Example 22. Synthesis of 7-(3,8-diazabicyclo[3.2.1]octan-3-yl)-2-(8-fluoro-2-methylimidazo[1,2-a]pyridin-6-yl)-4H-pyrido[1,2-a][1,3,5]triazin-4-one hydrochloride (Compound 105)

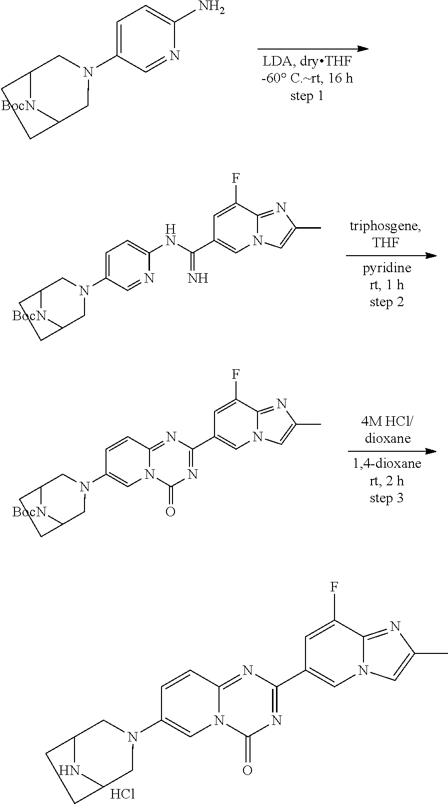 [SMILES: CC(C)(C)OC(=O)N1C2CCC1CN(c1ccc(N)nc1)C2 ; Cc1cn2cc(C(=N)Nc3ccc(N4CC5CCC(C4)N5C(=O)OC(C)(C)C)cn3)cc(F)c2n1 ; Cc1cn2cc(-c3nc(=O)n4cc(N5CC6CCC(C5)N6C(=O)OC(C)(C)C)ccc4n3)cc(F)c2n1 ; Cc1cn2cc(-c3nc(=O)n4cc(N5CC6CCC(C5)N6)ccc4n3)cc(F)c2n1 ; Cl]

### Step 1: tert-butyl 3-(6-(8-fluoro-2-methylimidazo[1,2-a]pyridine-6-carboximidamido)pyridin-3-yl)-3,8-diazabicyclo[3.2.1]octane-8-carboxylate

To a mixture of diisopropylamine (0.3 mL, 2.2 mmol) in dry THE (10 mL) was added n-BuLi (1.5 mL, 2.4 mmol, 1.6M) dropwise at −65° C. The mixture was stirred at this temperature for 1 hour. Tert-butyl 3-(6-aminopyridin-3-yl)-3,8-diazabicyclo[3.2.1]octane-8-carboxylate (608 m, 2.0 mmol) in dry THE (5 mL) was added. The mixture was allowed to warm to room temperature and stirred for 1 h. 8-fluoro-2-methylimidazo[1,2-a]pyridine-6-carbonitrile (175 mg, 1.0 mmol) in dry THE (5 mL) was added to the mixture at −40° C. and allowed to warm to room temperature and stirred for 16 h. The mixture was diluted with water (30 mL), extracted with EtOAc (30 mL×2). The combined organic layer was washed with brine (50 mL), dried over sodium sulfate, filtered and concentrated in vacuo. The residue was purified by column chromatography (PE/EA=1:1 to 0:1) to give title product (170 mg, yield: 35.5%) as a yellow solid. ¹H NMR (400 MHz, CDCl₃) δ 9.15 (s, 1H), 8.07 (s, 1H), 7.75 (s, 2H), 7.16 (s, 2H), 7.06 (d, J=10.9 Hz, 1H), 4.47-4.34 (m, 2H), 3.43 (d, J=10.4 Hz, 2H), 3.11-3.02 (m, 2H), 2.50 (s, 3H), 2.04-1.99 (m, 2H), 1.87 (m, 2H), 1.49 (s, 9H).

### Step 2: tert-butyl 3-(2-(8-fluoro-2-methylimidazo[1,2-a]pyridin-6-yl)-4-oxo-4H-pyrido[1,2-a][1,3,5]triazin-7-yl)-3,8-diazabicyclo[3.2.1]octane-8-carboxylate

To a solution of tert-butyl 3-(6-(8-fluoro-2-methylimidazo[1,2-a]pyridine-6-carboximidamido)pyridine-3-yl)-3,8-diazabicyclo[3.2.1]octane-8-carboxylate (170 mg, 0.35 mmol) in THE (16 mL) and pyridine (2 mL) was added triphosgene (315 mg, 1.05 mmol). The reaction mixture was stirred for 1 h at rt, The mixture was diluted with water (30 mL), extracted with EtOAc (30 mL×2). The combined organic layer was washed with brine (50 mL), dried over sodium sulfate, filtered and concentrated in vacuo. The residue was purified by column chromatography (PE/EA=1:1 to 0:1) to give title product as a brown solid (90 mg, yield:50%). ¹H NMR (400 MHz, CDCl₃) δ 8.84 (s, 1H), 8.07 (d, J=2.8 Hz, 1H), 8.00 (dd, J=11.2, 5.2 Hz, 2H), 7.28 (s, 1H), 6.82 (d, J=9.2 Hz, 1H), 4.44-4.42 (m, 2H), 3.48 (d, J=10.4 Hz, 2H), 3.15-3.12 (m, 2H), 2.47 (s, 3H), 2.03-2.01 (m, 2H), 1.85-1.83 (m, 2H), 1.49 (s, 9H). ESI-MS: [M+H]⁺ 506.2.

### Step 3: 7-(3,8-diazabicyclo[3.2.I]octan-3-yl)-2-(8-fluoro-2-methylimidazo [1,2-a]pyridin-6-yl)-4H-pyrido[1,2-a][1,3,5]triazin-4-one hydrochloride

To a solution of tert-butyl 3-(2-(8-fluoro-2-methylimidazo[1,2-a]pyridin-6-yl)-4-oxo-4H-pyrido[1,2-a][1,3,5]triazin-7-yl)-3,8-diazabicyclo[3.2.1]octane-8-carboxylate (90 mg, 0.18 mmol) in EtOAc (2 mL) was added 3M HCl/EtOAc (2 ml) at room temperature. The reaction mixture was stirred for 2 h. The precipitate was filtered and the solid was dried by lyophilization to give title product (40 mg, yield: 51.3%) as a yellow solid. ¹H NMR (400 MHz, DMSO-d₆) δ 9.61-9.52 (m, 2H), 9.22 (d, J=4.8 Hz, 1H), 8.25 (d, J=2.4 Hz, 1H), 8.08 (s, 1H), 7.76 (dd, J=9.0, 2.8 Hz, 1H), 7.57 (dd, J=9.1, 2.8 Hz, 1H), 7.45-7.41 (m, 1H), 4.18 (br.s, 2H), 3.81 (d, J=12.0 Hz, 2H), 3.30-3.26 (m, 2H), 2.38 (s, 3H), 2.04-1.95 (m, 4H). ESI-MS: [M+H]⁺: 406.1.

### Example 23. Synthesis of 2-(8-fluoro-2-methylimidazo[1,2-a]pyridin-6-yl)-7-(hexahydropyrrolo[3,4-c]pyrrol-2(1H)-yl)-4H-pyrido[1,2-a][1,3,5]triazin-4-one hydrochloride (Compound 106)

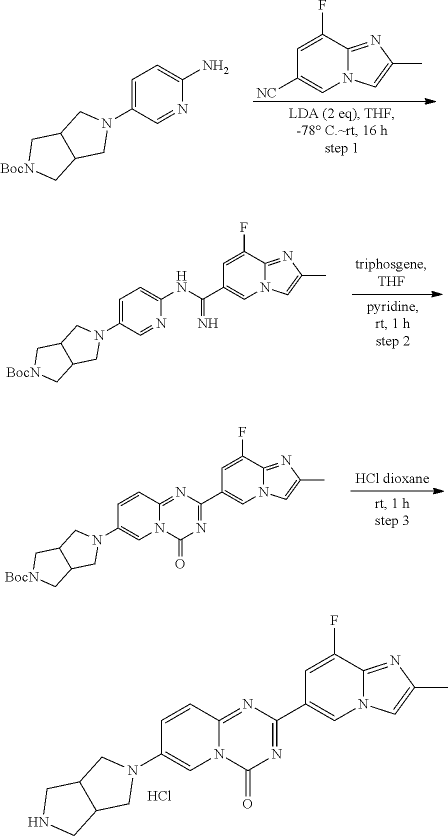 [SMILES: CC(C)(C)OC(=O)N1CC2CN(c3ccc(N)nc3)CC2C1 ; Cc1cn2cc(C#N)cc(F)c2n1 ; Cc1cn2cc(C(=N)Nc3ccc(N4CC5CN(C(=O)OC(C)(C)C)CC5C4)cn3)cc(F)c2n1 ; Cc1cn2cc(-c3nc(=O)n4cc(N5CC6CN(C(=O)OC(C)(C)C)CC6C5)ccc4n3)cc(F)c2n1 ; Cc1cn2cc(-c3nc(=O)n4cc(N5CC6CNCC6C5)ccc4n3)cc(F)c2n1 ; Cl]

### Step 1: tert-butyl 5-(6-(8-fluoro-2-methylimidazo[1,2-a]pyridine-6-carboximidamido)pyridin-3-yl)hexahydropyrrolo[3,4-c]pyrrole-2(1H)-carboxylate

To a solution of DIPA (220 mg, 2.2 mmol) and THF (6 mL) was added n-BuLi (2.4 mmol, 1.5 mL, 1.6M) at −60° C. under N₂. The mixture was stirred for 1 h, then tert-butyl 5-(6-aminopyridin-3-yl)hexahydropyrrolo[3,4-c]pyrrole-2(1H)-carboxylate (608 mg, 2 mmol) in THF (5 mL) was added. The mixture was allowed to warn to rt and stirred for 1.5 h at RT. The mixture was cooling down to −60° C. again. 8-fluoro-2-methylimidazo[1,2-a]pyridine-6-carbonitrile (175 mg, 1 mmol) in THF (5 mL) was added slowly. The mixture was stirred at RT overnight. The reaction mixture was diluted with water (40 mL) and extracted with EtOAc (40 mL×3). The organic layer was washed with brine, dried with Na₂SO₄ and evaporated to give crude title compound. The residue was purified by C₁₈ column chromatography to give title compound (120 mg, 25% yield) as a grey solid. ESI-MS (M+H)⁺: 480.2.

### Step 2: tert-butyl 5-(2-(8-fluoro-2-methylimidazo[1,2-a]pyridin-6-yl)-4-oxo-4H-pyrido[1,2-a][1,3,5]triazin-7-yl)hexahydropyrrolo[3,4-c]pyrrole-2(1H)-carboxylate

To a solution of tert-butyl 5-(6-(8-fluoro-2-methylimidazo[1,2-a]pyridine-6-carboximidamido) pyridine-3-yl)hexahydropyrrolo[3,4-c]pyrrole-2(1H)-carboxylate (120 mg, 0.25 mmol) in THF (8 mL) and pyridine (1 mL) was added triphosgene (222 mg, 0.75 mmol), the mixture was stirred at rt for 1 h. The reaction mixture was diluted with water (15 mL) and extracted with EtOAc (20 mL×3). The organic layer was washed with brine, dried with Na₂SO₄ and evaporated to give crude title compound. The residue was purified by pre-TLC (EtOAc:PE=1:3) to give title compound (50 mg, 47.6% yield) as a yellow solid. ESI-MS (M+H)⁺:506.3.

### Step 3: 2-(8-fluoro-2-methylimidazo[1,2-a]pyridin-6-yl)-7-(hexahydropyrrolo[3,4-c]pyrrol-2(1H)-yl)-4H-pyrido[1,2-a][1,3,5]triazin-4-one hydrochloride

A solution of tert-butyl 5-(2-(8-fluoro-2-methylimidazo[1,2-a]pyridin-6-yl)-4-oxo-4H-pyrido[1,2-a][1,3,5]triazin-7-yl)hexahydropyrrolo[3,4-c]pyrrole-2(1H)-carboxylate (50 mg, 0.099 mmol) in 4M HCl/EA (2 mL) was stirred for 1 h at RT. The precipitate was filtered, washed with EtOAc (1 mL) and dried under vacuum to afford title compound (30 mg, 24.4% yield) as a yellow solid. ESI-MS (M+H)⁺:406.1. ¹H NMR (400 MHz, DMSO-d₆) δ 9.57 (s, 2H), 9.26 (s, 1H), 8.10 (s, 1H), 7.94 (d, J=2.5 Hz, 1H), 7.72 (d, J=8.9 Hz, 1H), 7.48 (d, J=10.5 Hz, 1H), 7.27-7.20 (m, 1H), 3.57 (br.s, 2H), 3.48-3.46 (m, 6H), 3.18 (d, J=2.9 Hz, 2H), 3.14-3.06 (m, 2H), 2.38 (s, 3H).

### Example 24. Synthesis of 7-((3S,5R)-3,5-dimethylpiperazin-1-yl)-2-(8-fluoro-2-methylimidazo[1,2-a]pyridin-6-yl)-4H-pyrido[1,2-a][1,3,5]triazin-4-one hydrochloride (Compound 107)

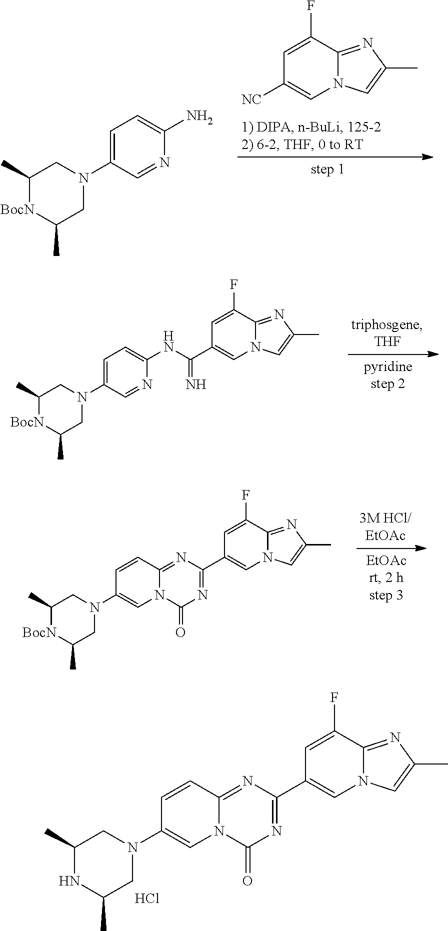

### Step 1: tert-butyl (2S,6R)-4-(6-(8-fluoro-2-methylimidazo[1,2-a]pyridine-6-carboximidamido)pyridin-3-yl)-2,6-dimethylpiperazine-1-carboxylate

To a mixture of diisopropylamine (0.3 mL, 2.2 mmol) in dry THE (10 mL) was added n-BuLi (1.5 mL, 2.4 mmol, 1.6M) dropwise at −65° C. The mixture was stirred at this temperature for 1 hour. Then cis tert-butyl-4-(6-aminopyridin-3-yl)-2,6-dimethylpiperazine-1-carboxylate (612 mg, 2.0 mmol) in dry THE (5 mL) was added. The mixture was allowed to warm to room temperature and stirred for 1 h. 8-fluoro-2-methylimidazo[1,2-a]pyridine-6-carbonitrile (175 mg, 1.0 mmol) in dry THE (5 mL) was added in at −40° C. and the mixture was allowed to warm to room temperature and stirred for 16 h. The mixture was diluted with water (10 mL), extracted with EtOAc (20 mL×2). The combined organic layer was washed with brine (30 mL), dried over sodium sulfate, filtered and concentrated in vacuo. The residue was purified by column C₁₈ column to give title product (100 mg, yield: 20.8%) as a yellow solid. ESI-MS: [M+H]⁺482.3

### Step 2: tert-butyl (2S,6R)-4-(2-(8-fluoro-2-methylimidazo[1,2-a]pyridin-6-yl)-4-oxo-4H-pyrido[1,2-a][1,3,5]triazin-7-yl)-2,6-dimethylpiperazine-1-carboxylate

To a solution of cis tert-butyl-4-(6-(8-fluoro-2-methylimidazo[1,2-a]pyridine-6-carboximidamido) pyridin-3-yl)-2,6-dimethylpiperazine-1-carboxylate (100 mg, 0.208 mmol) in THE (18 mL) and pyridine (0.8 mL) was added triphosgene (185 mg, 0.624 mmol). The reaction mixture was stirred for 1 h at rt, The mixture was diluted with water (10 mL), extracted with EtOAc (20 mL×2). The combined organic layer was washed with brine (30 mL), dried over sodium sulfate, filtered and concentrated in vacuo. The residue was purified by prep-HPLC (0.05% FA in water/CH₃CN) to give title product as a yellow solid (50 mg, 47.6%). ¹H NMR (400 MHz, CDCl₃) δ 9.22 (d, J=1.3 Hz, 1H), 8.51 (d, J=2.7 Hz, 1H), 7.95 (dd, J=11.6, 1.3 Hz, 1H), 7.90 (dd, J=9.6, 2.9 Hz, 1H), 7.67 (d, J=9.5 Hz, 1H), 7.48 (d, J=2.9 Hz, 1H), 4.38-4.33 (m, 2H), 3.47 (d, J=12.1 Hz, 2H), 3.08 (dd, J=12.1, 4.5 Hz, 2H), 2.50 (s, 3H), 1.51 (s, 9H), 1.38 (d, J=6.4 Hz, 6H). ESI-MS: [M+H]⁺: 508.1

### Step 3: 7-((3S,5R)-3,5-dimethylpiperazin-1-yl)-2-(8-fluoro-2-methylimidazo[1,2-a]pyridin-6-yl)-4H-pyrido[1,2-a][1,3,5]triazin-4-one hydrochloride

To a solution of tert-butyl (2S,6R)-4-(2-(8-fluoro-2-methylimidazo[1,2-a]pyridin-6-yl)-4-oxo-4H-pyrido [1,2-a][1,3,5]triazin-7-yl)-2,6-dimethylpiperazine-1-carboxylate (50 mg, 0.099 mmol) in EtOAc (2 mL) was added 3M HCl/EtOAc (3 ml) at room temperature. The reaction mixture was stirred for 2 h. The precipitate was filtered and the solid was dried via lyophilization to give title product (25 mg, yield: 57.3%) as a yellow solid. ¹H NMR (400 MHz, MeOD-d4) δ 9.66 (s, 1H), 8.67 (d, J=11.0 Hz, 1H), 8.58 (s, 1H), 8.42 (d, J=11.8 Hz, 1H), 8.23 (s, 1H), 7.89 (d, J=9.5 Hz, 1H), 4.12 (d, J=11.5 Hz, 2H), 3.65-3.57 (m, 2H), 2.96 (t, J=12.3 Hz, 2H), 2.62 (s, 3H), 1.47 (d, J=6.4 Hz, 6H). ESI-MS: [M+H]⁺ 408.2.

### Example 25. Synthesis of 2-(8-fluoro-2-methylimidazo[1,2-a]pyridin-6-yl)-9-methyl-7-(piperazin-1-yl)-4H-pyrido[1,2-a][1,3,5]triazin-4-one hydrochloride (Compound 108)

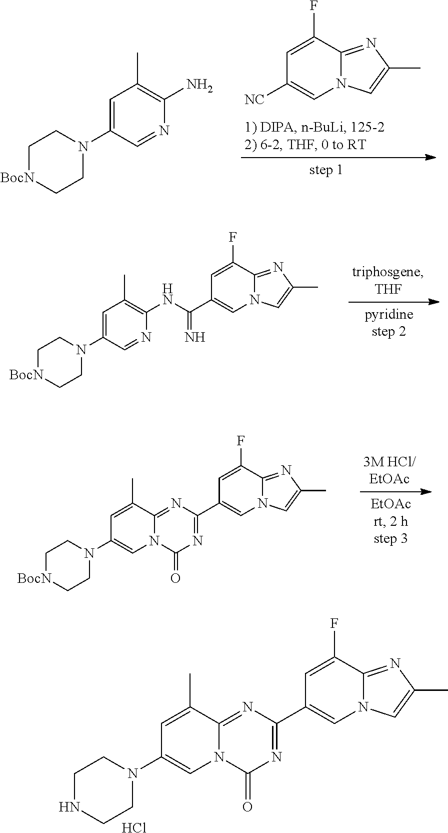 [SMILES: Cc1cc(N2CCN(C(=O)OC(C)(C)C)CC2)cnc1N ; Cc1cn2cc(C#N)cc(F)c2n1 ; Cc1cn2cc(C(=N)Nc3ncc(N4CCN(C(=O)OC(C)(C)C)CC4)cc3C)cc(F)c2n1 ; Cc1cn2cc(-c3nc(=O)n4cc(N5CCN(C(=O)OC(C)(C)C)CC5)cc(C)c4n3)cc(F)c2n1 ; Cc1cn2cc(-c3nc(=O)n4cc(N5CCNCC5)cc(C)c4n3)cc(F)c2n1 ; Cl]

### Step 1: tert-butyl 4-(6-(8-fluoro-2-methylimidazo[1,2-a]pyridine-6-carboximidamido)-5-methylpyridin-3-yl)piperazine-1-carboxylate

To a mixture of diisopropylamine (0.3 mL, 2.2 mmol) in dry THE (10 mL) was added n-BuLi (1.5 mL, 2.4 mmol, 1.6M) dropwise at −65° C. The mixture was stirred at this temperature for 1 hour. Then tert-butyl 4-(6-amino-5-methylpyridin-3-yl)piperazine-1-carboxylate (600 mg, 2.0 mmol) in dry THE (5 mL) was added. The mixture was allowed to warm to room temperature and stirred for 1 h. 8-fluoro-2-methylimidazo[1,2-a]pyridine-6-carbonitrile (175 mg, 1.0 mmol) in dry THE (5 mL) was added to the mixture at −40° C. and the mixture was allowed to warm to room temperature and stirred for 16 h. The mixture was diluted with water (30 mL), extracted with EtOAc (30 mL×2). The combined organic layer was washed with brine (50 mL), dried over sodium sulfate, filtered and concentrated in vacuo. The residue was purified by column chromatography (PE/EA=1:1 to 0:1) to give title product (60 mg, yield: 22.5%) as a yellow solid. ESI-MS: [M+H]⁺: 468.3

### Step 2: tert-butyl 4-(2-(8-fluoro-2-methylimidazo[1,2-a]pyridin-6-yl)-9-methyl-4-oxo-4H-pyrido[1,2-a][1,3,5]triazin-7-yl)piperazine-1-carboxylate

To a solution of tert-butyl 4-(6-(8-fluoro-2-methylimidazo[1,2-a]pyridine-6-carboximidamido)-5-methyl pyridin-3-yl)piperazine-1-carboxylate (60 mg, 0.121 mmol) in THE (6 mL) and pyridine (0.6 mL) was added triphosgene (108 mg, 0.363 mmol). The reaction mixture was stirred for 1 h at rt, The mixture was diluted with water (10 mL), extracted with EtOAc (20 mL×2). The combined organic layer was washed with brine (30 mL), dried over sodium sulfate, filtered and concentrated in vacuo. The residue was purified by prep-HPLC (0.05% FA in water/CH₃CN) to give title product (30 mg, 47.6%). ¹H NMR (400 MHz, CDCl₃) δ 9.22 (d, J=1.3 Hz, 1H), 8.41 (d, J=2.8 Hz, 1H), 7.97 (dd, J=11.6, 1.3 Hz, 1H), 7.76 (dd, J=2.7, 0.9 Hz, 1H), 7.48 s, 1H), 3.66-3.61 (m, 4H), 3.28-3.22 (m, 4H), 2.69 (s, 3H), 2.51 (s, 3H), 1.50 (s, 9H). ESI-MS: [M+H]⁺: 494.3.

### Step 3: 2-(8-fluoro-2-methylimidazo[1,2-a]pyridin-6-yl)-9-methyl-7-(piperazin-1-yl)-4H-pyrido[1,2-a][1,3,5]triazin-4-one hydrochloride

To a solution of tert-butyl 4-(2-(8-fluoro-2-methylimidazo[1,2-a]pyridin-6-yl)-9-methyl-4-oxo-4H-pyrido[1,2-a][1,3,5]triazin-7-yl)piperazine-1-carboxylate (30 mg, 0.061 mmol) in EtOAc (0.5 mL) was added 3M HCl/EtOAc (1 mL) at room temperature. The reaction mixture was stirred for 2 h. The precipitate was filtered and dried via lyophilization to give title product (18 mg, yield: 69.2%) as a yellow solid. ¹H NMR (400 MHz, MeOD-d₄) δ 9.68 (s, 1H), 8.66 (d, J=10.8 Hz, 1H), 8.49 (d, J=2.8 Hz, 1H), 8.31-8.27 (m, 2H), 3.69-3.65 (m, 4H), 3.49-3.45 (m, 4H), 2.75 (s, 3H), 2.63 (s, 3H). ESI-MS: [M+H]⁺: 394.2.

### Example 26. Synthesis of 7-(3,3-dimethylpiperazin-1-yl)-2-(8-fluoro-2-methylimidazo [1,2-a]pyridin-6-yl)-4H-pyrido[1,2-a][1,3,5]triazin-4-one (Compound 109)

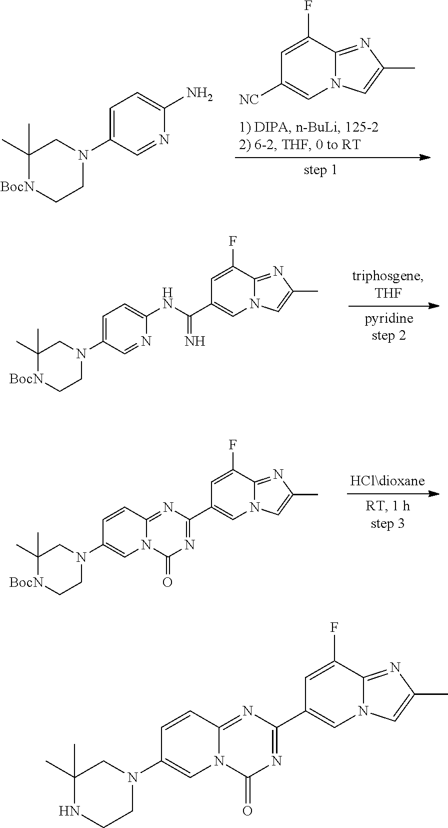

### Step 1: tert-butyl 4-(6-(8-fluoro-2-methylimidazo[1,2-a]pyridine-6-carboximidamido)pyridin-3-yl)-2,2-dimethylpiperazine-1-carboxylate

To a solution of diisopropyl amine (220 mg, 2.2 mmol) in THE (6 mL) was added n-BuLi (1.5 mL, 1.6M) at −60° C. under N₂. The mixture was stirred for 1 h, then tert-butyl 4-(6-aminopyridin-3-yl)-2,2-dimethylpiperazine-1-carboxylate (612 mg, 2 mmol) in THF (5 mL) was added at −60° C. The mixture was allowed to warm to rt and stirred for 1.5 h at RT. The mixture was cooling down to −60° C. again. Then 8-fluoro-2-methylimidazo[1,2-a]pyridine-6-carbonitrile (175 mg, 1 mmol) in THE (5 mL) was added. The mixture was stirred overnight at RT. The reaction mixture was diluted with water (40 mL) and extracted with EtOAc (40 mL×3). The organic layer was washed with brine, dried with Na₂SO₄ and evaporated to give crude title compound. The residue was purified by C18 column chromatography to give title compound (220 mg, 45.7% yield) as a grey solid. ESI-MS (M+H)⁺: 482.3. ¹H NMR (400 MHz, CDCl₃): δ 8.64 (s, 1H), 7.83 (s, 1H), 7.47 (s, 1H), 7.36 (d, J=10.9 Hz, 1H), 7.23 (s, 1H), 7.16-7.05 (m, 1H), 3.81 (d, J=4.9 Hz, 2H), 3.49 (s, 2H), 3.40 (br s, 2H), 2.48 (s, 3H), 1.50 (s, 9H), 1.44 (s, 6H).

### Step 2: tert-butyl 4-(2-(8-fluoro-2-methylimidazo[1,2-a]pyridin-6-yl)-4-oxo-4H-pyrido[1,2-a][1,3,5]triazin-7-yl)-2,2-dimethylpiperazine-1-carboxylate

To a solution of tert-butyl 4-(6-(8-fluoro-2-methylimidazo[1,2-a]pyridine-6-carboximidamido) pyridin-3-yl)-2,2-dimethylpiperazine-1-carboxylate (220 mg, 0.46 mmol) in THE (6 mL) and pyridine (1 mL) was added triphosgene (407 mg, 1.37 mmol). The mixture was stirred at r.t. for 1 h. The reaction mixture was diluted with water (15 mL) and extracted with EtOAc (20 mL×3). The organic layer was washed with brine, dried with Na₂SO₄ and evaporated to give crude title compound. The residue was purified by C18 column chromatography to give title compound (150 mg, 64.6% yield) as a yellow solid. ESI-MS (M+H)⁺:508.7. ¹H NMR (400 MHz, CDCl₃): δ 9.20 (s, 1H), 8.36 (s, 1H), 7.93 (d, J=11.7 Hz, 1H), 7.77 (d, J=9.3 Hz, 1H), 7.67 (d, J=9.5 Hz, 1H), 7.47 (s, 1H), 3.90 (br.s, 2H), 3.52 (br.s, 2H), 3.45 (s, 2H), 2.50 (s, 3H), 1.47 (s, 15H).

### Step 3: 7-(3,3-dimethylpiperazin-1-yl)-2-(8-fluoro-2-methylimidazo [1,2-a]pyridin-6-yl)-4H-pyrido[1,2-a][1,3,5]triazin-4-one

A solution of tert-butyl 4-(2-(8-fluoro-2-methylimidazo[1,2-a]pyridin-6-yl)-4-oxo-4H-pyrido[1,2-a][1,3,5]triazin-7-yl)-2,2-dimethylpiperazine-1-carboxylate (140 mg) in 4M HCl/EA (5 mL) was stirred for 1 h at RT. The precipitate was filtered and the filter cake was further purified by pre-HPLC to afford title compound (30 mg, 24.4% yield) as a yellow solid. ESI-MS (M+H)⁺:408.1. ¹H NMR (400 MHz, CDCl₃): δ 9.22 (s, 1H), 8.46 (s, 1H), 7.95-7.89 (m, 2H), 7.64 (d, J=9.6 Hz, 1H), 7.48 (s, 1H), 3.25-3.17 (m, 2H), 3.17-3.10 (m, 2H), 3.02 (s, 2H), 2.50 (s, 3H), 1.26 (s, 6H).

### Example 27. Synthesis of 9-fluoro-2-(8-fluoro-2-methylimidazo[1,2-a]pyridin-6-yl)-7-(piperazin-1-yl)-4H-pyrido[1,2-a][1,3,5]triazin-4-one hydrochloride (Compound 110)

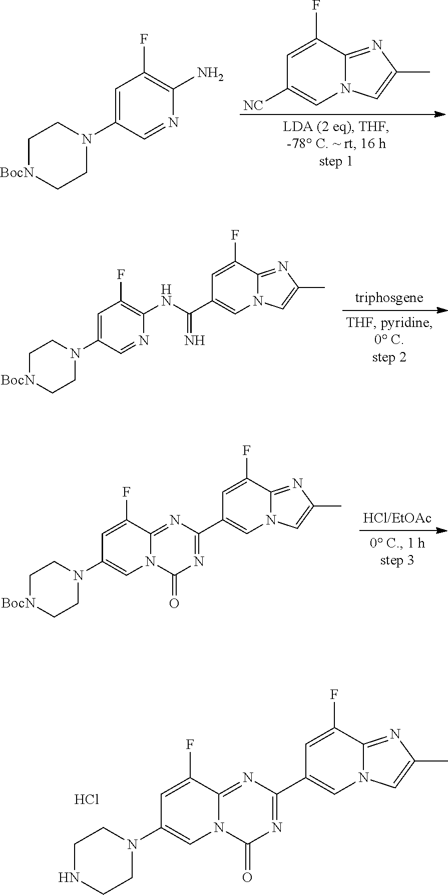 [SMILES: CC(C)(C)OC(=O)N1CCN(c2cnc(N)c(F)c2)CC1 ; Cc1cn2cc(C#N)cc(F)c2n1 ; Cc1cn2cc(C(=N)Nc3ncc(N4CCN(C(=O)OC(C)(C)C)CC4)cc3F)cc(F)c2n1 ; Cc1cn2cc(-c3nc(=O)n4cc(N5CCN(C(=O)OC(C)(C)C)CC5)cc(F)c4n3)cc(F)c2n1 ; Cc1cn2cc(-c3nc(=O)n4cc(N5CCNCC5)cc(F)c4n3)cc(F)c2n1 ; Cl]

### Step 1: tert-butyl 4-(5-fluoro-6-(8-fluoro-2-methylimidazo [1,2-a]pyridine-6-carboximidamido)pyridin-3-yl)piperazine-1-carboxylate

To a solution of diisopropylamine (606 mg, 6 mmol) and THF (10 mL) was added n-BuLi (6.4 mmol, 1.6M, 4 mL) at −60° C. under N₂ and the mixture was stirred for 1 h. Tert-butyl 4-(6-amino-5-fluoropyridin-3-yl)piperazine-1-carboxylate (1.34 g, 4.5 mmol) in THF (10 mL) was added at −60° C., the mixture was allowed to warm to rt and stirred for 1.5 h. The mixture was cooling down to −60° C. again. Then 8-fluoro-2-methylimidazo[1,2-a]pyridine-6-carbonitrile (525 mg, 3 mmol) in THF (15 mL) was added slowly. The mixture was stirred overnight at RT. The reaction mixture was diluted with water (100 mL) and extracted with EtOAc (100 mL×3). The organic layer was washed with brine, dried with Na₂SO₄ and evaporated to give crude title compound. The residue was purified by C18 column chromatography to give title compound (300 mg, 21.2% yield) as a grey solid. ESI-MS (M+H)⁺:472.1.

### Step 2: tert-butyl 4-(9-fluoro-2-(8-fluoro-2-methylimidazo[1,2-a]pyridin-6-yl)-4-oxo-4H-pyrido[1,2-a][1,3,5]triazin-7-yl)piperazine-1carboxylate

To a solution of tert-butyl 4-(5-fluoro-6-(8-fluoro-2-methylimidazo[1,2-a]pyridine-6-carboximidamido)pyridine-3-yl)piperazine-1-carboxylate (300 mg, 0.64 mmol) in THF (15 mL) and pyridine(1.5 mL) was added triphosgene (1.2 g, 2.22 mmol). The mixture was stirred at r.t. for 1 h. The reaction mixture was diluted with water (50 mL) and extracted with EtOAc (50 mL×3). The organic layer was washed with brine, dried over Na₂SO₄ and evaporated to give crude title compound. The residue was purified by pre-HPLC (0.05% FA in water/CH₃CN) to give title compound (130 mg, 41.1% yield) as a yellow solid. ESI-MS (M+H)⁺:498.1.

### Step 3: 9-fluoro-2-(8-fluoro-2-methylimidazo[1,2-a]pyridin-6-yl)-7-(piperazin-1-yl)-4H-pyrido[1,2-a][1,3,5]triazin-4-one hydrochloride

A solution of tert-butyl 4-(9-fluoro-2-(8-fluoro-2-methylimidazo[1,2-a]pyridin-6-yl)-4-oxo-4H-pyrido [1,2-a][1,3,5]triazin-7-yl)piperazine-1-carboxylate (130 mg, 0.26 mmol) in 3M HCl/EtOAc (5 mL) was stirred for 1 h at RT. The mixture was filtered. The solid was washed with EtOAc (1 mL) and dried under vacuum to afford title compound (105 mg, 94.6% yield) as a yellow solid, ESI-MS (M+H)⁺:398.1. ¹H NMR (400 MHz, MeOD-d₄) δ 9.67 (d, J=0.9 Hz, 1H), 8.65 (dd, J=10.9, 1.1 Hz, 1H), 8.44 (d, J=2.0 Hz, 1H), 8.35 (dd, J=11.3, 2.6 Hz, 1H), 8.24 (dd, J=2.1, 1.1 Hz, 1H), 3.72-3.65 (m, 4H), 3.50-3.43 (m, 4H), 2.62 (d, J=0.9 Hz, 3H).

### Example 28. Synthesis of 2-(8-fluoro-2-methylimidazo[1,2-a]pyridin-6-yl)-7-(1,2,3,6-tetrahydropyridin-4-yl)-4H-pyrido[1,2-a][1,3,5]triazin-4-one hydrochloride (Compound 111)

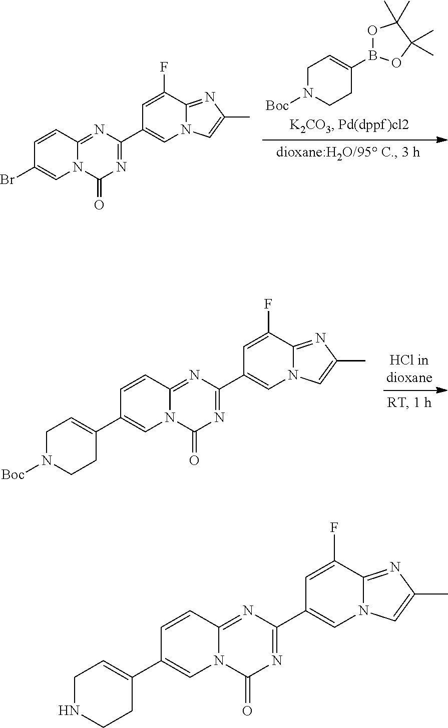

### Step 1: tert-butyl 4-(2-(8-fluoro-2-methylimidazo[1,2-a]pyridin-6-yl)-4-oxo-4H-pyrido[1,2-a][1,3,5]triazin-7-yl)-3,6-dihydropyridine-1(2H)-carboxylate

A mixture of 7-bromo-2-(8-fluoro-2-methylimidazo[1,2-a]pyridin-6-yl)-4H-pyrido[1,2-a][1,3,5]triazin-4-one (100 mg, 0.268 mmol), tert-butyl 4-(4,4,5,5-tetramethyl-1,3,2-dioxaborolan-2-yl)-3,6-dihydropyridine-1(2H)-carboxylate (124 mg, 0.402 mol), K₂CO₃ (111 mg, 0.804 mol) and Pd(dppf)C₁₂ (22 mg, 0.027 mmol) in 1,4-dioxane/H₂O(15 mL/3 mL) was stirred at 95° C. for 3 h. The reaction mixture was diluted with water (15 mL) and extracted with EtOAc (30 mL×3). The organic layer was washed with brine, dried over Na₂SO₄ and evaporated to give crude title compound. The crude was purification by Prep-HPLC (0.05% FA in water/CH₃CN) to give title compound (40 mg, 31.3% yield) as a yellow solid. ESI-MS (M+H)⁺: 477.1.

### Step 2: 2-(8-fluoro-2-methylimidazo[1,2-a]pyridin-6-yl)-7-(1,2,3,6-tetrahydropyridin-4-yl)-4H-pyrido[1,2-a][1,3,5]triazin-4-one hydrochloride

To a solution of tert-butyl 4-(2-(8-fluoro-2-methylimidazo[1,2-a]pyridin-6-yl)-4-oxo-4H-pyrido[1,2-a][1,3,5]triazin-7-yl)-3,6-dihydropyridine-1(2H)-carboxylate (30 mg, 0.063 mmol) in EtOAc (2 mL) was added 3M HCl/EtOAc (2 mL) at room temperature. The reaction mixture was stirred for 1 h. The precipitate was filtered and dried under vacuum to give title product (6 mg, yield: 25.3%) as a white solid. ¹H NMR (400 MHz, DMSO-d₆) δ 9.70-9.65 (m, 1H), 9.36 (s, 2H), 8.89 (s, 1H), 8.60 (d, J=9.2 Hz, 1H), 8.26-8.15 (m, 2H), 7.83 (d, J=9.0 Hz, 1H), 6.61 (s, 1H), 3.85 (s, 2H), 3.40-3.33 (m, 2H), 2.79 (br.s, 2H), 2.48 (s, 3H). ESI-MS: [M+H]⁺=377.1.

### Example 29. Synthesis of 2-(8-fluoro-2-methylimidazo[1,2-a]pyridin-6-yl)-7-(pyrrolidin-3-yl)-4H-pyrido[1,2-a][1,3,5]triazin-4-one hydrochloride (Compound 112)

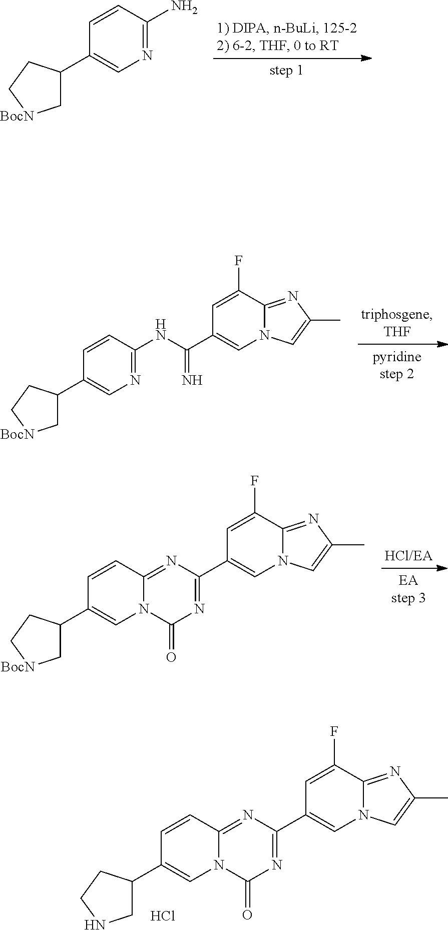 [SMILES: CC(C)(C)OC(=O)N1CCC(c2ccc(N)nc2)C1 ; Cc1cn2cc(C(=N)Nc3ccc(C4CCN(C(=O)OC(C)(C)C)C4)cn3)cc(F)c2n1 ; Cc1cn2cc(-c3nc(=O)n4cc(C5CCN(C(=O)OC(C)(C)C)C5)ccc4n3)cc(F)c2n1 ; Cc1cn2cc(-c3nc(=O)n4cc(C5CCNC5)ccc4n3)cc(F)c2n1 ; Cl]

### Step 1: tert-butyl 3-(6-(8-fluoro-2-methylimidazo[1,2-a]pyridine-6-carboximidamido) pyridine-3-yl)pyrrolidine-1-carboxylate

To a mixture of diisopropylamine (0.3 mL, 2.2 mmol) in dry THE (20 mL) was added n-BuLi (1.5 mL, 2.4 mmol, 1.6M) dropwise at −65° C. The mixture was stirred at this temperature for 1 hour. tert-butyl 3-(6-aminopyridin-3-yl)pyrrolidine-1-carboxylate (512 mg, 2 mmol) in dry THE (7 mL) was added. The mixture was allowed to warm to room temperature and stirred for 1 h. 8-fluoro-2-methylimidazo[1,2-a]pyridine-6-carbonitrile (175 mg, 1 mmol) in dry THE (10 mL) was added to the mixture at −65° C. and allowed to warm to room temperature and stirred for 16 h. The mixture was diluted with water (20 mL), extracted with EtOAc (15 mL×2). The combined organic layer was washed with brine (30 mL), dried over sodium sulfate, filtered and concentrated in vacuo. The residue was purified by C18-flash (0.05% NH₃·H₂O in water/CH₃CN) to give title product (60 mg, yield:13.7%) as a yellow solid. ESI-MS: [M+H]⁺=439.1.

### Step 2: tert-butyl 3-(2-(8-fluoro-2-methylimidazo[1,2-a]pyridin-6-yl)-4-oxo-4H-pyrido [1,2-a][1,3,5]triazin-7-yl)pyrrolidine-1-carboxylate

To a solution of tert-butyl 3-(6-(8-fluoro-2-methylimidazo[1,2-a]pyridine-6-carboximidamido)pyridin-3-yl)pyrrolidine-1-carboxylate (60 mg, 0.14 mmol) in THE (5 mL) and pyridine (1 mL) was added triphosgene (125 mg, 0.42 mmol). The reaction mixture was stirred for 1 h at rt. The mixture was diluted with water (15 mL), extracted with EtOAc (15 mL×2). The combined organic layer was washed with brine (25 mL), dried over sodium sulfate, filtered and concentrated in vacuo. The residue was purified by prep-HPLC (0.05% NH₃·H₂O in water/CH₃CN) to give title product (4 mg, yield: 6.3%). ESI-MS: [M+H]⁺=465.1.

### Step 3: 2-(8-fluoro-2-methylimidazo[1,2-a]pyridin-6-yl)-7-(pyrrolidin-3-yl)-4H-pyrido[1,2-a][1,3,5]triazin-4-one hydrochloride

To a solution of tert-butyl 3-(2-(8-fluoro-2-methylimidazo[1,2-a]pyridin-6-yl)-4-oxo-4H-pyrido[1,2-a][1,3,5]triazin-7-yl)pyrrolidine-1-carboxylate (4 mg, 0.009 mmol) in EtOAc (2 mL) was added 3M HCl/EtOAc (3 mL) at room temperature. The reaction mixture was stirred for 1 h. The precipitate was filtered and dried under vacuum to title product (0.9 mg, yield: 29%) as a white solid. ¹H NMR (400 MHz, MeOD-d₄) δ 9.72 (s, 1H), 9.12 (s, 1H), 8.69 (d, J=10.8 Hz, 1H), 8.46 (d, J=7.0 Hz, 1H), 8.25 (s, 1H), 7.94 (d, J=7.4 Hz, 1H), 3.86 (br.s, 2H), 3.66 (br.s, 1H), 3.40-3.35 (m, 3H), 2.63 (s, 3H), 1.34-1.27 (m, 1H). ESI-MS: [M+H]⁺ 365.1.

### Example 30. Synthesis of (2-(8-fluoro-2-methylimidazo[1,2-a]pyridin-6-yl)-7-(piperidin-4-yl)-4H-pyrido[1,2-a][1,3,5]triazin-4-one hydrochloride (Compound 113)

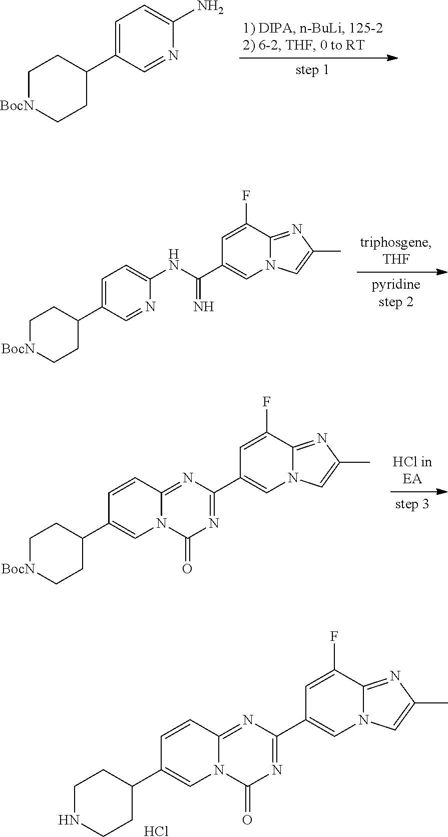 [SMILES: CC(C)(C)OC(=O)N1CCC(c2ccc(N)nc2)CC1 ; Cc1cn2cc(C(=N)Nc3ccc(C4CCN(C(=O)OC(C)(C)C)CC4)cn3)cc(F)c2n1 ; Cc1cn2cc(-c3nc(=O)n4cc(C5CCN(C(=O)OC(C)(C)C)CC5)ccc4n3)cc(F)c2n1 ; Cc1cn2cc(-c3nc(=O)n4cc(C5CCNCC5)ccc4n3)cc(F)c2n1 ; Cl]

### Step 1: tert-butyl 4-(6-(8-fluoro-2-methylindolizine-6-carboximidamido)pyridin-3-yl) piperidine-1-carboxylate

To a mixture of diisopropylamine (0.3 mL, 2.2 mmol) in dry THE (20 mL) was added n-BuLi (1.5 mL, 2.4 mmol, 1.6M) dropwise at −65° C. The mixture was stirred at this temperature for 1 hour. tert-butyl 4-(6-aminopyridin-3-yl)piperidine-1-carboxylate (556 mg, 2 mmol) in dry THE (7 mL) was added. The mixture was allowed to warm to room temperature and stirred for 1 h. 8-fluoro-2-methylimidazo[1,2-a]pyridine-6-carbonitrile (175 mg, 1 mmol) in dry THE (10 mL) was added to the mixture at −65° C. and allowed to warm to room temperature and stirred for 16 h. The mixture was diluted with water (20 mL), extracted with EtOAc (15 mL×2). The combined organic layer was washed with brine (30 mL), dried over sodium sulfate, filtered and concentrated in vacuo. The residue was purified by C18-flash to give title product (150 mg, yield: 33.3%) as a yellow solid. ¹H NMR (400 MHz, DMSO-D6) δ 9.04 (d, J=1.1 Hz, 1H), 8.24 (d, J=2.3 Hz, 1H), 7.94 (d, J=2.5 Hz, 1H), 7.70-7.62 (m, 2H), 7.09 (d, J=8.4 Hz, 1H), 4.14-4.02 (m, 2H), 2.89-2.65 (m, 3H), 2.37 (s, 3H), 1.77-1.74 (m, 2H), 1.57-1.51 (m, 2H), 1.41 (s, 9H). ESI-MS: [M+H]=453.2.

### Step 2: tert-butyl 4-(2-(8-fluoro-2-methylimidazo[1,2-a]pyridin-6-yl)-4-oxo-4H-pyrido [1,2-a][1,3,5]triazin-7-yl)piperidine-1-carboxylate

To a solution of tert-butyl 4-(6-(8-fluoro-2-methylindolizine-6-carboximidamido)pyridin-3-yl)piperidine-1-carboxylate (150 mg, 0.33 mmol) in THE (5 mL) and pyridine (1 mL) was added triphosgene (294 mg, 0.99 mmol). The reaction mixture was stirred for 1 h at rt, The mixture was diluted with water (15 mL), extracted with EtOAc (15 mL×2). The combined organic layer was washed with brine (25 mL), dried over sodium sulfate, filtered and concentrated in vacuo, the residue was purified by prep-HPLC to give title product (60 mg, yield: 38%) as a yellow solid. ESI-MS: [M+H]⁺=479.1.

### Step 3: (2-(8-fluoro-2-methylimidazo[1,2-a]pyridin-6-yl)-7-(piperidin-4-yl)-4H-pyrido[1,2-a][1,3,5]triazin-4-one hydrochloride

To a solution of tert-butyl 4-(2-(8-fluoro-2-methylimidazo[1,2-a]pyridin-6-yl)-4-oxo-4H-pyrido[1,2-a][1,3,5]triazin-7-yl)piperidine-1-carboxylate (60 mg, 0.125 mmol) in EtOAc (2 mL) was added 3M HCl/EtOAc (5 ml) at room temperature. The reaction mixture was stirred for 1 h. The precipitate was filtered and the solid was dried under vacuum to give title product (15 mg, yield: 32%) as a white solid. H NMR (400 MHz, DMSO-d₆) δ 9.71 (s, 1H), 9.17 (s, 2H), 8.85 (s, 1H), 8.32-8.29 (m, 3H), 7.86 (d, J=9.0 Hz, 1H), 3.41-3.39 (m, 2H), 3.19 (br.s, 1H), 3.02 (br.s, 2H), 2.10-1.94 (m, 4H). ESI-MS: [M+H]⁺ 379.2.

### Example 31. Synthesis of 2-(8-fluoro-2-methylimidazo[1,2-a]pyridin-6-yl)-9-methoxy-7-(piperazin-1-yl)-4H-pyrido[1,2-a][1,3,5]triazin-4-one hydrochloride (Compound 114)

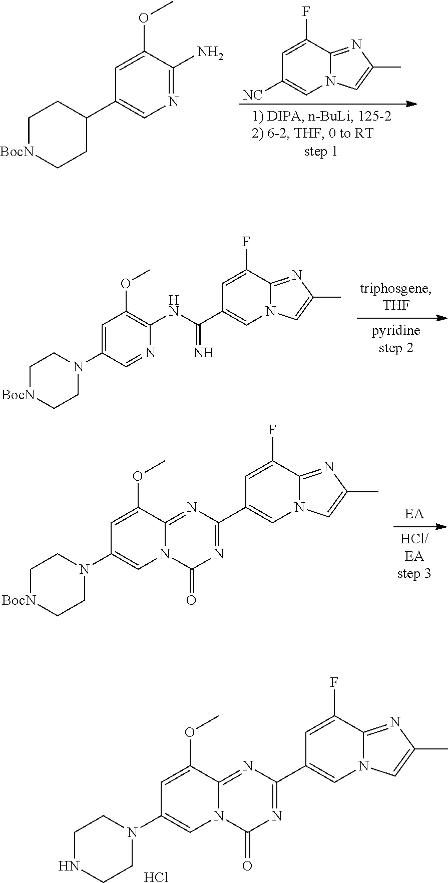 [SMILES: COc1cc(C2CCN(C(=O)OC(C)(C)C)CC2)cnc1N ; Cc1cn2cc(C#N)cc(F)c2n1 ; COc1cc(N2CCN(C(=O)OC(C)(C)C)CC2)cnc1NC(=N)c1cc(F)c2nc(C)cn2c1 ; COc1cc(N2CCN(C(=O)OC(C)(C)C)CC2)cn2c(=O)nc(-c3cc(F)c4nc(C)cn4c3)nc12 ; COc1cc(N2CCNCC2)cn2c(=O)nc(-c3cc(F)c4nc(C)cn4c3)nc12 ; Cl]

### Step 1: tert-butyl 4-(6-(8-fluoro-2-methylimidazo[1,2-a]pyridine-6-carboximidamido)-5-methoxypyridin-3-yl)piperazine-1-carboxylate

To a solution of diisopropylamine (110 mg, 1.1 mmol) in THE (6 mL) was added n-BuLi (0.75 mL, 1.6M) at −60° C. under N₂ and the mixture was stirred for 1 h. Tert-butyl 4-(6-amino-5-methoxypyridin-3-yl)piperazine-1-carboxylate (308 mg, 1 mmol) in THE (5 mL) was added, the mixture was allowed to warm to rt and stirred for 1.5 h. The mixture was cooling down to −60° C. again. 8-fluoro-2-methylimidazo[1,2-a]pyridine-6-carbonitrile (88 mg, 0.5 mmol) in THE (5 mL) was added slowly. The mixture was stirred overnight at RT. The reaction mixture was diluted with water (40 mL) and extracted with EtOAc (40 mL×3). The organic layer was washed with brine, dried with Na₂SO₄ and evaporated to give crude title compound. The residue was purified by C₁₈ column chromatography to give title compound (45 mg, 21.3 yield) as a grey solid. ESI-MS (M+H)⁺: 484.3.

### Step 2: tert-butyl 4-(2-(8-fluoro-2-methylimidazo[1,2-a]pyridin-6-yl)-9-methoxy-4-oxo-4H-pyrido[1,2-a][1,3,5]triazin-7-yl)piperazine-1-carboxylate

To a solution of tert-butyl 4-(6-(8-fluoro-2-methylimidazo[1,2-a]pyridine-6-carboximidamido)-5-methoxy pyridin-3-yl)piperazine-1-carboxylate (45 mg, 0.09 mmol) in THE (8 mL) and pyridine (1 mL) was added triphosgene (83 mg, 0.75 mmol), the mixture was stirred at r.t. for 1 h. The reaction mixture was diluted with water (15 mL) and extracted with EtOAc (20 mL×3). The organic layer was washed with brine, dried with Na₂SO₄ and evaporated to give crude title compound. The residue was suspended in MeOH, and stirred for 1 h, the precipitate was filtered to give title compound (15 mg, 32% yield) as a yellow solid. ESI-MS (M+H)⁺:510.1.

### Step 3: 2-(8-fluoro-2-methylimidazo[1,2-a]pyridin-6-yl)-9-methoxy-7-(piperazin-1-yl)-4H-pyrido[1,2-a][1,3,5]triazin-4-one hydrochloride

A solution of tert-butyl 4-(2-(8-fluoro-2-methylimidazo[1,2-a]pyridin-6-yl)-9-methoxy-4-oxo-4H-pyrido[1,2-a][1,3,5]triazin-7-yl)piperazine-1-carboxylate (15 mg, 0.029 mmol) in 3M HCl/EA (2 mL) was stirred for 1 h at rt. The precipitate was filtered and the solid was dried to afford title compound (9 mg, 81.8% yield) as a yellow solid. ESI-MS (M+H)⁺: 410.2. ¹H NMR (400 MHz, MeOD-d₄) δ 9.66 (s, 1H), 8.68 (d, J=10.8 Hz, 1H), 8.24 (s, 2H), 7.77 (d, J=2.1 Hz, 1H), 4.17 (s, 3H), 3.69-3.65 (m, 4H), 3.49-3.46 (m, 4H), 2.62 (s, 3H).

### Example 32. Synthesis of 2-(8-fluoro-2-methylimidazo[1,2-a]pyridin-6-yl)-8-methyl-7-(piperazin-1-yl)-4H-pyrido[1,2-a][1,3,5]triazin-4-one hydrochloride (Compound 115)

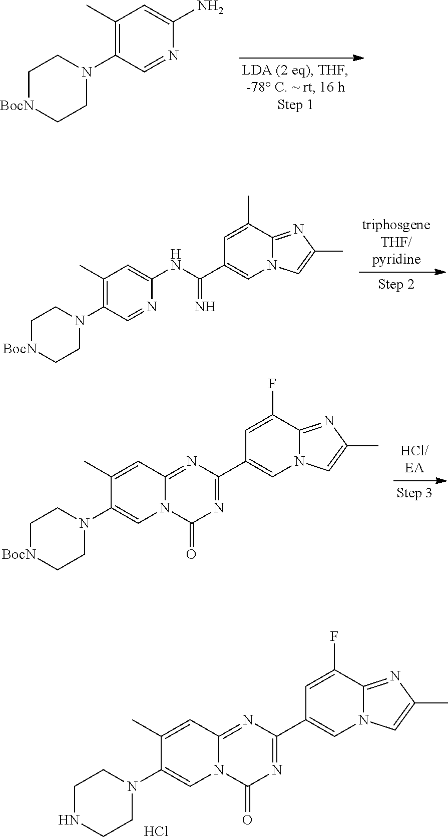 [SMILES: Cc1cc(N)ncc1N1CCN(C(=O)OC(C)(C)C)CC1 ; Cc1cn2cc(C(=N)Nc3cc(C)c(N4CCN(C(=O)OC(C)(C)C)CC4)cn3)cc(C)c2n1 ; Cc1cn2cc(-c3nc(=O)n4cc(N5CCN(C(=O)OC(C)(C)C)CC5)c(C)cc4n3)cc(F)c2n1 ; Cc1cn2cc(-c3nc(=O)n4cc(N5CCNCC5)c(C)cc4n3)cc(F)c2n1 ; Cl]

### Step 1: tert-butyl 4-(6-(8-fluoro-2-methylimidazo[1,2-a]pyridine-6-carboximidamido)-4-methylpyridin-3-yl)piperazine-1-carboxylate

To a mixture of diisopropylamine (0.3 mL, 2.2 mmol) in dry THF (10 mL) was added n-BuLi (1.5 mL, 2.4 mmol, 1.6M) dropwise at −65° C. The mixture was stirred at this temperature for 1 hour. Then tert-butyl 4-(6-amino-4-methylpyridin-3-yl)piperazine-1-carboxylate (438 mg, 1.5 mmol) was added, the mixture was allowed to warm to rt and stirred for 1.5 h at rt. The mixture was cooling down to −60° C. again. Then 8-fluoro-2-methylimidazo[1,2-a]pyridine-6-carbonitrile (175 mg, 1.0 mmol) in THF (5 mL) was added, the mixture was allowed to warm to rt and stirred overnight. The reaction mixture was diluted with water (30 mL) and extracted with EtOAc (40 mL×3). The organic layer was washed with brine, dried with Na₂SO₄ and evaporated to give crude title compound. The residue was purified by C₁₈ column chromatography to give title compound (150 mg, yield: 51.2%) as a grey solid. ESI-MS (M+H)⁺: 468.2.

### Step 2: tert-butyl 4-(2-(8-fluoro-2-methylimidazo[1,2-a]pyridin-6-yl)-8-methyl-4-oxo-4H-pyrido[1,2-a][1,3,5]triazin-7-yl)piperazine-1-carboxylate

To a solution of tert-butyl 4-(6-(8-fluoro-2-methylimidazo[1,2-a]pyridine-6-carboximidamido)-4-methylpyridin-3-yl)piperazine-1-carboxylate (70 mg, 0.15 mmol) in THF (15 mL) and pyridine (1 mL) was added triphosgene (134 mg, 0.45 mmol) at 0° C., the mixture was stirred for 20 min at r.t. The reaction mixture was diluted with water (15 mL) and extracted with EtOAc (20 mL×3). The organic layer was washed with brine, dried with Na₂SO₄ and evaporated to give crude title compound. The residue was purified by C18 column chromatography to give title compound (18 mg, yield: 24.3%) as a yellow solid. ESI-MS (M+H)⁺: 494.1.

### Step 3: 2-(8-fluoro-2-methylimidazo[1,2-a]pyridin-6-yl)-8-methyl-7-(piperazin-1-yl)-4H-pyrido[1,2-a][1,3,5]triazin-4-one hydrochloride

A solution of tert-butyl 4-(2-(8-fluoro-2-methylimidazo[1,2-a]pyridin-6-yl)-8-methyl-4-oxo-4H-pyrido[1,2-a][1,3,5]triazin-7-yl)piperazine-1-carboxylate (18 mg, 0.04 mmol) in HCl/EA (3 mL) was stirred for 1 h at rt. The mixture was filtered and the solid was dried under vacuum to afford title compound (10 mg, 62.5%) as a yellow solid. ESI-MS (M+H)⁺:394.2. ¹H NMR (400 MHz, MeOD-d₄) δ 9.64 (s, 1H), 8.73 (s, 1H), 8.63 (d, J=11.0 Hz, 1H), 8.20 (s, 1H), 7.80 (s, 1H), 3.51-3.47 (m, 4H), 3.39-3.36 (m, 4H), 2.66 (s, 3H), 2.61 (s, 3H).

### Example 33. Synthesis of (S)-9-fluoro-2-(8-fluoro-2-methylimidazo[1,2-a]pyridine-6-yl)-7-(3-methylpiperazin-1-yl)-4H-pyrido[1,2-a][1,3,5]triazin-4-one (Compound 116)

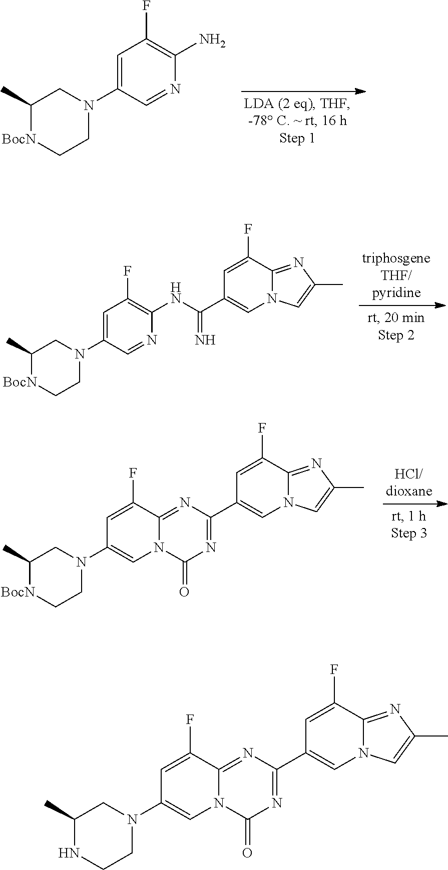

### Step 1: tert-butyl (S)-4-(5-fluoro-6-(8-fluoro-2-methylimidazo[1,2-a]pyridine-6-carboximidamido)pyridin-3-yl)-2-methylpiperazine-1-carboxylate

To a mixture of diisopropylamine (0.3 mL, 2.2 mmol) in dry THF (10 mL) was added n-BuLi (1.5 mL, 2.4 mmol, 1.6M) dropwise at −65° C. The mixture was stirred at this temperature for 1 hour, then tert-butyl (S)-4-(6-amino-5-fluoropyridin-3-yl)-2-methylpiperazine-1-carboxylate (465 mg, 1.56 mmol) in THF (2 mL) was added, the mixture was allowed to warm to rt and stirred for 1.5 h. The mixture was cooling down to −60° C. again. Then 8-fluoro-2-methylimidazo[1,2-a]pyridine-6-carbonitrile (175 mg, 1 mmol) in THE (5 mL) was added slowly, the mixture was stirred overnight at rt. The reaction mixture was diluted with water (30 mL) and extracted with EtOAc (40 mL×3). The organic layer was washed with brine, dried with Na₂SO₄ and evaporated to give crude title compound. The residue was purified by C18 column chromatography to give title compound (150 mg, yield: 30%) as a grey solid. ESI-MS (M+H)⁺: 486.3. ¹H NMR (400 MHz, CDCl₃): δ 8.65 (d, J=1.3 Hz, 1H), 7.78 (d, J=2.6 Hz, 1H), 7.48 (d, J=2.4 Hz, 1H), 7.33 (dd, J=11.1, 1.3 Hz, 1H), 7.04 (dd, J=12.4, 2.6 Hz, 1H), 4.44-4.34 (m, 1H), 3.98 (d, J=13.8 Hz, 1H), 3.48 (d, J=11.9 Hz, 1H), 3.38-3.21 (m, 2H), 3.02-3.00 (m, 1H), 2.83-2.80 (m, 1H), 2.50 (s, 3H), 1.49 (s, 9H), 1.31 (d, J=6.7 Hz, 3H).

### Step 2: tert-butyl (S)-4-(9-fluoro-2-(8-fluoro-2-methylimidazo[1,2-a]pyridin-6-yl)-4-oxo-4H-pyrido[1,2-a][1,3,5]triazin-7-yl)-2-methylpiperazine-1-carboxylate

To a solution of tert-butyl (S)-4-(5-fluoro-6-(8-fluoro-2-methylimidazo[1,2-a]pyridine-6-carboximidamido)pyridin-3-yl)-2-methylpiperazine-1-carboxylate (1.0 g, 2.06 mmol) in THE (100 mL) and pyridine(10 mL) was added triphosgene (1.83 g, 6.18 mmol) at 0° C., the mixture was stirred for 20 min at 0° C. The reaction mixture was diluted with water (50 mL) and extracted with EtOAc (100 mL×3). The organic layer was washed with brine, dried with Na₂SO₄ and evaporated to give crude title compound. The residue was purified by prep-HPLC (0.05% HCl in water/CH₃CN) to give title compound (550 mg yield: 52.6%) as a yellow solid. ESI-MS (M+H)⁺:512.0. ¹H NMR (400 MHz, CDCl₃) δ 9.22 (d, J=1.2 Hz, 1H), 8.31 (d, J=2.0 Hz, 1H), 7.97 (dd, J=11.6, 1.2 Hz, 1H), 7.63 (dd, J=10.5, 2.6 Hz, 1H), 7.49 (d, J=2.1 Hz, 1H), 4.44-4.40 (m, 1H), 4.04 (d, J=13.6 Hz, 1H), 3.59 (d, J=12.0 Hz, 1H), 3.44-3.40 (m, 1H), 3.36-3.29 (m, 1H), 3.21-3.16 (m, 1H), 3.03-2.96 (m, 1H), 2.51 (s, 3H), 1.50 (s, 9H), 1.30 (d, J=6.8 Hz, 3H).

### Step 3: (S)-9-fluoro-2-(8-fluoro-2-methylimidazo[1,2-a]pyridine-6-yl)-7-(3-methylpiperazin-1-yl)-4H-pyrido[1,2-a][1,3,5]triazin-4-one

To a solution of tert-butyl (S)-4-(9-fluoro-2-(8-fluoro-2-methylimidazo[1,2-a]pyridin-6-yl)-4-oxo-4H-pyrido [1,2-a][1,3,5]triazin-7-yl)-2-methylpiperazine-1-carboxylate (1.1 g, 2.15 mmol) in EtOAc (10 mL) was added 3M HCl/EtOAc (15 ml) at room temperature. The reaction mixture was stirred for 1 h. The mixture was concentrated in vacuo, the residue was purified by prep-HPLC (0.05% NH₄₀H in water/CH₃CN) to afford title compound (520 mg, 58%) as a yellow solid. ESI-MS (M+H)⁺:412.1. ¹H NMR (400 MHz, DMSO-d6) δ 9.43 (d, J=1.1 Hz, 1H), 8.43 (dd, J=12.4, 2.5 Hz, 1H), 8.10 (d, J=2.0 Hz, 1H), 8.06 (d, J=2.4 Hz, 1H), 7.78 (dd, J=12.3, 1.0 Hz, 1H), 3.70-3.65 (m, 2H), 3.02-2.99 (m, 1H), 2.81-2.66 (m, 3H), 2.39 (s), 2.37-2.33 (m, 1H), 1.06 (d, J=6.0 Hz, 3H).

### Example 34. Synthesis of (S)-2-(8-fluoro-2-methylimidazo[1,2-a]pyridin-6-yl)-9-methyl-7-(3-methylpiperazin-1-yl)-4H-pyrido[1,2-a][1,3,5]triazin-4-one hydrochloride (Compound 117)

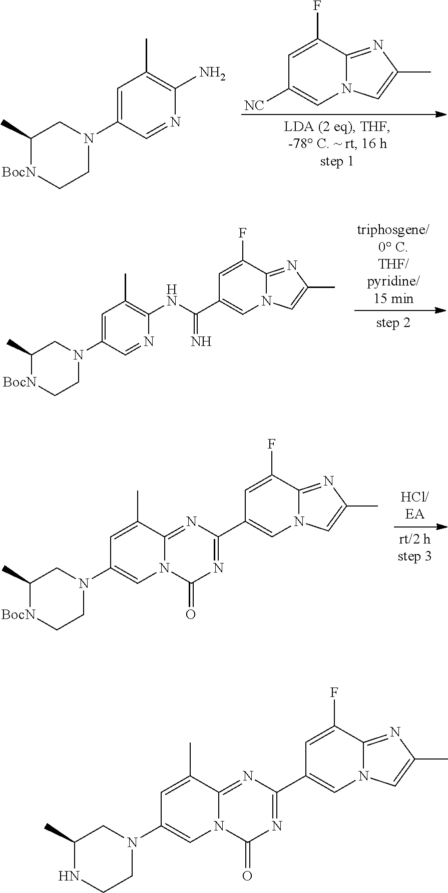

### Step 1: tert-butyl (S)-4-(6-(8-fluoro-2-methylimidazo[1,2-a]pyridine-6-carboximidamido)-5-methylpyridin-3-yl)-2-methylpiperazine-1-carboxylate

To a mixture of diisopropylamine (0.9 mL, 6.6 mmol) in dry THF (25 mL) was added n-BuLi (4.5 mL, 7.2 mmol, 1.6M) dropwise at −65° C. The mixture was stirred at this temperature for 1 hour. tert-butyl (S)-4-(6-amino-5-methylpyridin-3-yl)-2-methylpiperazine-1-carboxylate (1.38 g, 4.5 mmol) in dry THF (7 mL) was added. The mixture was allowed to warm to room temperature and stirred for 1 h. 8-fluoro-2-methylimidazo[1,2-a]pyridine-6-carbonitrile (525 mg, 3.0 mmol) in dry THF (10 mL) was added to the mixture at −65° C. and allowed to warm to room temperature and stirred for 16 h. The mixture was diluted with water (40 mL), extracted with EtOAc (30 mL×2). The combined organic layer was washed with brine (50 mL), dried over sodium sulfate, filtered and concentrated in vacuo. The residue was purified by C18-flash to give title product (600 mg, yield: 41.5%) as a yellow solid. ¹H NMR (400 MHz, MeOD-d₄) δ 8.83 (s, 1H), 7.88 (d, J=2.6 Hz, 1H), 7.79 (d, J=2.1 Hz, 1H), 7.65 (d, J=11.7 Hz, 1H), 7.32 (d, J=2.6 Hz, 1H), 4.35 (br.s, 1H), 3.95 (d, J=13.3 Hz, 1H), 3.56 (d, J=11.5 Hz, 1H), 3.49-3.42 (m, 1H), 3.25 (d, J=3.5 Hz, 1H), 2.89-2.86 (m, 1H), 2.71-2.68 (m, 1H), 2.45 (s, 3H), 2.38 (s, 3H), 1.50 (s, 9H), 1.33 (d, J=6.7 Hz, 3H). ESI-MS: [M+H]⁺=482.1.

### Step 2: tert-butyl (S)-4-(2-(8-fluoro-2-methylimidazo[1,2-a]pyridin-6-yl)-9-methyl-4-oxo-4H-pyrido[1,2-a][1,3,5]triazin-7-yl)-2-methylpiperazine-1-carboxylate

To a solution of tert-butyl (S)-4-(6-(8-fluoro-2-methylimidazo[1,2-a]pyridine-6-carboximidamido)-5-methylpyridin-3-yl)-2-methylpiperazine-1-carboxylate (600 mg, 1.25 mmol) in THE (50 mL) and pyridine (10 mL) was added triphosgene (1.11 g, 3.74 mmol). The reaction mixture was stirred for 15 min at 0° C. The mixture was diluted with water (30 mL), extracted with EtOAc (30 mL×2). The combined organic layer was washed with brine (50 mL), dried over sodium sulfate, filtered and concentrated in vacuo. The residue was mixed with MeOH (40 mL), stirred for 1 hour at rt. The precipitate was filtered and dried in vacuo to give title product (310 mg, yield: 49%) as a yellow solid. ESI-MS: [M+H]=508.2.

### Step 3: (S)-2-(8-fluoro-2-methylimidazo[1,2-a]pyridin-6-yl)-9-methyl-7-(3-methylpiperazin-1-yl)-4H-pyrido[1,2-a][1,3,5]triazin-4-one hydrochloride

To a solution of tert-butyl (S)-4-(2-(8-fluoro-2-methylimidazo[1,2-a]pyridin-6-yl)-9-methyl-4-oxo-4H-pyrido[1,2-a][1,3,5]triazin-7-yl)-2-methylpiperazine-1-carboxylate (80 mg, 0.157 mmol) in EtOAc (2 mL) was added 3M HCl/EtOAc (5 ml) at room temperature. The reaction mixture was stirred for 1 h. The precipitate was filtered and dried under vacuum to give title product (50 mg, yield: 64%) as a yellow solid. ¹H NMR (400 MHz, MeOD-d₄) δ 9.68 (s, 1H), 8.68 (dd, J=10.9, 0.9 Hz, 1H), 8.50 (d, J=2.7 Hz, 1H), 8.31-8.24 (m, 2H), 4.03-3.98 (m, 2H), 3.59-3.56 (m, 2H), 3.38-3.36 (m, 1H), 3.26-3.20 (m, 1H), 3.02-3.00 (m, 1H), 2.76 (s, 3H), 2.62 (s, 3H), 1.45 (d, J=6.7 Hz, 3H). ESI-MS: [M+H]⁺ 408.0.

### Example 35. Synthesis of 7-(3,9-diazabicyclo[3.3.1]nonan-3-yl)-2-(8-fluoro-2-methylimidazo[1,2-a]pyridin-6-yl)-4H-pyrido[1,2-a][1,3,5]triazin-4-one hydrochloride (Compound 118)

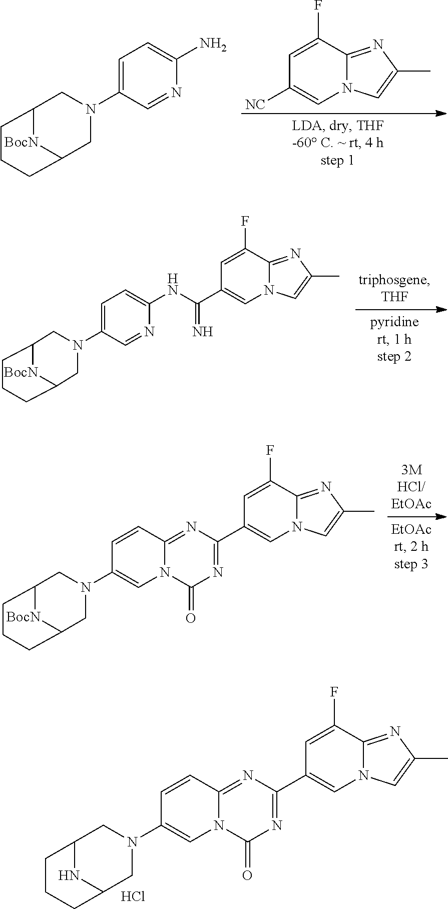 [SMILES: CC(C)(C)OC(=O)N1C2CCCC1CN(c1ccc(N)nc1)C2 ; Cc1cn2cc(C#N)cc(F)c2n1 ; Cc1cn2cc(C(=N)Nc3ccc(N4CC5CCCC(C4)N5C(=O)OC(C)(C)C)cn3)cc(F)c2n1 ; Cc1cn2cc(-c3nc(=O)n4cc(N5CC6CCCC(C5)N6C(=O)OC(C)(C)C)ccc4n3)cc(F)c2n1 ; Cc1cn2cc(-c3nc(=O)n4cc(N5CC6CCCC(C5)N6)ccc4n3)cc(F)c2n1 ; Cl]

### Step 1: tert-butyl 3-(6-(8-fluoro-2-methylimidazo[1,2-a]pyridine-6-carboximidamido)pyridin-3-yl)_{3,9}-diazabicyclo[3.3.I]nonane-9-carboxylate

To a mixture of diisopropylamine (184 mg, 1.06 mmol) in dry THE (10 mL) was added n-BuLi (0.81 mL, 1.30 mmol, 1.6M) dropwise at −65° C. The mixture was stirred at this temperature for 1 hour. Tert-butyl 3-(6-aminopyridin-3-yl)-3,9-diazabicyclo[3.3.1]nonane-9-carboxylate (280 mg, 0.88 mmol) in dry THE (5 mL) was added. The mixture was allowed to warm to room temperature and stirred for 1 h. 8-fluoro-2-methylimidazo[1,2-a]pyridine-6-carbonitrile (100 mg, 0.59 mmol) in dry THE (5 mL) was added to the mixture at −40° C. and allowed to warm to room temperature and stirred for 16 h. The mixture was diluted with water (30 mL), extracted with EtOAc (30 mL×2), the combined organic layer was washed with brine (50 mL), dried over sodium sulfate, filtered and concentrated in vacuo. The residue was purified by prep-HPLC (0.05% HCl in water/CH₃CN) to give title product (20 mg, yield: 7.1%) as a yellow solid. ESI-MS: [M+H]⁺: 494.2.

### Step 2: tert-butyl 3-(2-(8-fluoro-2-methylimidazo[1,2-a]pyridin-6-yl)-4-oxo-4H-pyrido [1,2-a][1,3,5]triazin-7-yl)-3,9-diazabicyclo[3.3.1]nonane-9-carboxylate

To a solution of tert-butyl 3-(6-(8-fluoro-2-methylimidazo[1,2-a]pyridine-6-carboximidamido)pyridin-3-yl)-3,9-diazabicyclo[3.3.1]nonane-9-carboxylate (20 mg, 0.041 mmol) in THE (4 mL) and pyridine (0.4 mL) was added triphosgene (36 mg, 0.122 mmol). The reaction mixture was stirred for 1 h at rt, The mixture was diluted with water (30 mL), extracted with EtOAc (30 mL×2). The combined organic layer was washed with brine (50 mL), dried over sodium sulfate, filtered and concentrated in vacuo. The residue was purified by prep-HPLC (0.05% HCl in water/CH₃CN) to give title product (18 mg, yield:85.7%) as a yellow solid. ¹H NMR (400 MHz, CDCl₃) δ 9.22 (d, J=1.2 Hz, 1H), 8.46 (d, J=2.8 Hz, 1H), 7.96-7.92 (m, 2H), 7.67 (d, J=9.4 Hz, 1H), 7.48 (d, J=2.8 Hz, 1H), 4.49-4.48 (m, 1H), 4.38-4.33 (m, 1H), 3.72-3.69 (m, 1H), 3.63 (d, J=11.6 Hz, 1H), 3.24 (d, J=11.6 Hz, 2H), 2.50 (s, 3H), 2.29-2.19 (m, 2H), 1.97-1.88 (m, 2H), 1.83-1.78 (m, 2H), 1.51 (s, 9H). ESI-MS: [M+H]⁺: 520.0.

### Step 3: 7-(3,9-diazabicyclo[3.3.1]nonan-3-yl)-2-(8-fluoro-2-methylimidazo[1,2-a]pyridin-6-yl)-4H-pyrido[1,2-a][1,3,5]triazin-4-one hydrochloride

To a solution of tert-butyl 3-(2-(8-fluoro-2-methylimidazo[1,2-a]pyridin-6-yl)-4-oxo-4H-pyrido[1,2-a][1,3,5]triazin-7-yl)-3,9-diazabicyclo[3.3.1]nonane-9-carboxylate (18 mg, 0.0347 mmol) in EtOAc (0.5 mL) was added 3M HCl/EtOAc (1 mL) at room temperature. The reaction mixture was stirred for 2 h. The precipitate was filtered and dried under vacuum to give title product (8.08 mg, yield: 50.9%) as a yellow solid. ¹H NMR (400 MHz, MeOD-d4) δ 9.66 (s, 1H), 8.67 (d, J=10.8 Hz, 1H), 8.55 (s, 1H), 8.47 (d, J=10.8 Hz, 1H), 8.24 (s, 1H), 7.91 (d, J=8.8 Hz, 1H), 4.16 (d, J=12.8 Hz, 2H), 3.92-3.86 (m, 2H), 3.54 (d, J=12.4 Hz, 2H), 2.63 (s, 3H), 2.41-2.33 (m, 1H), 2.21-2.23 (m, 4H), 1.74-1.65 (m, 1H). ESI-MS: [M+H]⁺: 420.1.

### Example 36. Synthesis of 2-(8-fluoro-2-methylimidazo[1,2-a]pyridin-6-yl)-8-methoxy-7-(piperazin-1-yl)-4H-pyrido[1,2-a][1,3,5]triazin-4-one (Compound 119)

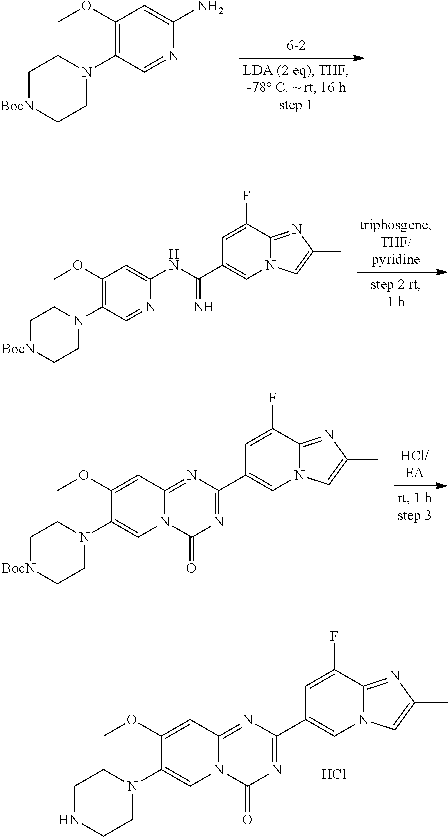 [SMILES: COc1cc(N)ncc1N1CCN(C(=O)OC(C)(C)C)CC1 ; COc1cc(NC(=N)c2cc(F)c3nc(C)cn3c2)ncc1N1CCN(C(=O)OC(C)(C)C)CC1 ; COc1cc2nc(-c3cc(F)c4nc(C)cn4c3)nc(=O)n2cc1N1CCN(C(=O)OC(C)(C)C)CC1 ; COc1cc2nc(-c3cc(F)c4nc(C)cn4c3)nc(=O)n2cc1N1CCNCC1 ; Cl]

### Step 1: tert-butyl 4-(6-(8-fluoro-2-methylimidazo[1,2-a]pyridine-6-carboximidamido)-4-methoxypyridin-3-yl)piperazine-1-carboxylate

To a mixture of diisopropylamine (121 mg, 1.2 mmol) in dry THE (5 mL) was added n-BuLi (0.9 mL, 1.44 mmol, 1.6M) dropwise at −65° C. The mixture was stirred at this temperature for 1 hour. Then tert-butyl 4-(6-amino-4-methoxypyridin-3-yl)piperazine-1-carboxylate (308 mg, 1 mmol) in dry THE (5 mL) was added. The mixture was allowed to warm to room temperature and stirred for 1 h. 8-fluoro-2-methylimidazo[1,2-a]pyridine-6-carbonitrile (110 mg, 0.66 mmol) in dry THE (5 mL) was added to the mixture at −40° C. and allowed to warm to room temperature and stirred for 16 h. The reaction mixture was diluted with water (20 mL) and extracted with EtOAc (20 mL×3). The organic layer was washed with brine, dried with Na₂SO₄ and evaporated to give crude title compound. The residue was purified by C₁₈ column chromatography to give title compound (70 mg, 21.2% yield) as a grey solid. ¹H NMR (400 MHz, CDCl₃) δ 8.57 (s, 1H), 7.82 (s, 1H), 7.46 (s, 1H), 7.36-7.32 (m, 1H), 6.79 (s, 1H), 3.94 (s, 3H), 3.64-3.59 (m, 4H), 3.07-3.00 (m, 4H), 2.50 (s, 3H), 1.49 (s, 9H).

### Step 2: tert-butyl 4-(2-(8-fluoro-2-methylimidazo[1,2-a]pyridin-6-yl)-8-methoxy-4-oxo-4H-pyrido[1,2-a][1,3,5]triazin-7-yl)piperazine-1-carboxylate

To a solution of tert-butyl 4-(6-(8-fluoro-2-methylimidazo[1,2-a]pyridine-6-carboximidamido)-4-methoxypyridin-3-yl)piperazine-1-carboxylate (70 mg, 0.15 mmol) in THE (8 mL) and pyridine (1 mL) was added triphosgene (1.2 g, 2.22 mmol), the mixture was stirred at rt for 1 h. The reaction mixture was diluted with water (30 mL) and extracted with EtOAc (20 mL×3). The organic layer was washed with brine, dried with Na₂SO₄ and evaporated to give crude title compound. The residue was suspended in MeOH, stirred for 1 h. The precipitate was filtered and the solid was dried to give title compound (30 mg, 41% yield) as a yellow solid. ESI-MS (M+H)+:510.2.

### Step 3: 2-(8-fluoro-2-methylimidazo[1,2-a]pyridin-6-yl)-8-methoxy-7-(piperazin-1-yl)-4H-pyrido[1,2-a][1,3,5]triazin-4-one

A solution of tert-butyl 4-(2-(8-fluoro-2-methylimidazo[1,2-a]pyridin-6-yl)-8-methoxy-4-oxo-4H-pyrido[1,2-a][1,3,5]triazin-7-yl)piperazine-1-carboxylate (30 mg, 0.05 mmol) in 3M HCl/EtOAc (4 mL) was stirred for 1 h at RT. The mixture was filtered and the solid was dried under vacuum to afford title compound (9 mg, 45% yield) as a yellow solid. ESI-MS (M+H)⁺:410.0. ¹H NMR (400 MHz, DMSO-d₆) δ 9.71 (s, 2H), 9.56 (s, 1H), 8.34 (s, 1H), 8.18 (s, 1H), 8.08 (d, J=11.8 Hz, 1H), 7.21 (s, 1H), 4.15 (s, 3H), 3.38 (br.s, 4H), 3.25 (br.s, 4H), 2.46 (s, 3H).

### Example 37. Synthesis of 2-(8-fluoro-2-methylimidazo[1,2-a]pyridin-6-yl)-6-methoxy-7-(piperazin-1-yl)-4H-pyrido[1,2-a][1,3,5]triazin-4-one formic acid (Compound 120)

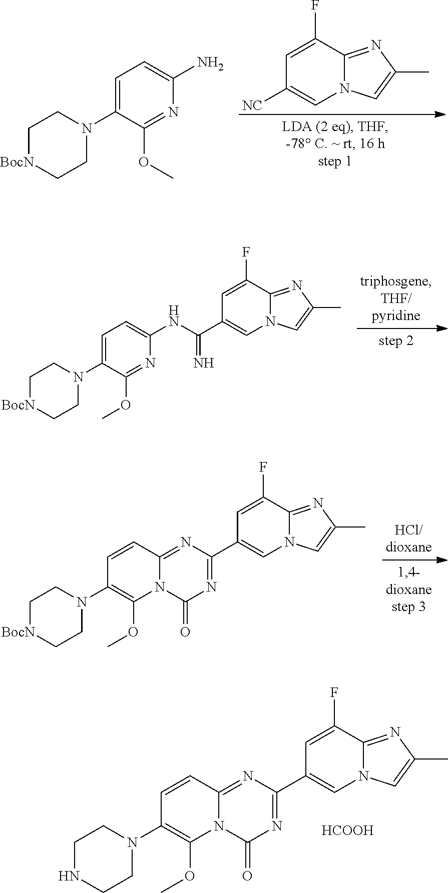 [SMILES: COc1nc(N)ccc1N1CCN(C(=O)OC(C)(C)C)CC1 ; Cc1cn2cc(C#N)cc(F)c2n1 ; COc1nc(NC(=N)c2cc(F)c3nc(C)cn3c2)ccc1N1CCN(C(=O)OC(C)(C)C)CC1 ; COc1c(N2CCN(C(=O)OC(C)(C)C)CC2)ccc2nc(-c3cc(F)c4nc(C)cn4c3)nc(=O)n12 ; COc1c(N2CCNCC2)ccc2nc(-c3cc(F)c4nc(C)cn4c3)nc(=O)n12 ; "HCOOH"]

### Step 1: tert-butyl 4-(6-(8-fluoro-2-methylimidazo[1,2-a]pyridine-6-carboximidamido)-2-methoxypyridin-3-yl)piperazine-1-carboxylate

To a mixture of diisopropylamine (79 mg, 0.78 mmol) in dry THF (15 mL) was added n-BuLi (0.53 mL, 0.85 mmol, 1.6M) dropwise at −65° C. The mixture was stirred at this temperature for 1 hour. tert-butyl 4-(6-amino-2-methoxypyridin-3-yl)piperazine-1-carboxylate (200 mg, 0.65 mmol) in dry THF (2 mL) was added. The mixture was allowed to warm to room temperature and stirred for 1 h. 8-fluoro-2-methylimidazo[1,2-a]pyridine-6-carbonitrile (80 mg, 0.45 mmol) in dry THF (5 mL) was added to the mixture at −65° C. and allowed to warm to room temperature and stirred for 16 h. The mixture was diluted with water (30 mL), extracted with EtOAc (30 mL×2). The combined organic layer was washed with brine (50 mL), dried over sodium sulfate, filtered and concentrated in vacuo. The residue was purified by C18 column to give title product as a white solid (20 mg, yield: 6.4%). ESI-MS: [M+H]⁺484.2.

### Step 2: tert-butyl 4-(2-(8-fluoro-2-methylimidazo[1,2-a]pyridin-6-yl)-6-methoxy-4-oxo-4H-pyrido[1,2-a][1,3,5]triazin-7-yl)piperazine-1-carboxylate

To a solution of tert-butyl 4-(6-(8-fluoro-2-methylimidazo[1,2-a]pyridine-6-carboximidamido)-2-methoxypyridin-3-yl)piperazine-1-carboxylate (150 mg, 0.31 mmol) in THE (10 mL) and pyridine (2 mL) was added triphosgene (276 mg, 0.93 mmol). The reaction mixture was stirred for 1 h at rt, The mixture was diluted with water (30 mL), extracted with EtOAc (30 mL×2). The combined organic layer was washed with brine (50 mL), dried over sodium sulfate, filtered and concentrated in vacuo. The residue was mixed with MeOH (40 mL), stirred for 1 hour at rt. The precipitate was filtered and dried in vacuo to give title product as a white solid (20 mg, 12.6%). ¹H NMR (400 MHz, CDCl₃) δ 8.81 (s, 1H), 8.08-7.96 (m, 1H), 7.71 (d, J=8.2 Hz, 1H), 7.26-7.17 (m, 1H), 6.85 (d, J=9.3 Hz, 1H), 4.04 (s, 3H), 3.65-3.57 (m, 4H), 3.12-3.02 (m, 4H), 2.47 (s, 3H), 1.50 (s, 9H). ESI-MS: [M+H]⁺ 510.2.

### Step 3: 2-(8-fluoro-2-methylimidazo[1,2-a]pyridin-6-yl)-6-methoxy-7-(piperazin-1-yl)-4H-pyrido[1,2-a][1,3,5]triazin-4-one formic acid

To a solution of tert-butyl 4-(2-(8-fluoro-2-methylimidazo[1,2-a]pyridin-6-yl)-6-methoxy-4-oxo-4H-pyrido[1,2-a][1,3,5]triazin-7-yl)piperazine-1-carboxylate (20 mg, 0.039 mmol) in EtOAc (1 mL) was added 3M HCl/EtOAc (2 mL) at room temperature. The reaction mixture was stirred for 2 h. After concentration, the residue was purified by pre-HPLC to give title product (10 mg, yield: 60%) as a yellow solid. ¹H NMR (400 MHz, MeOD-d4) δ ¹H NMR (400 MHz, MeOD) δ 9.23 (s, 1H), 8.47 (s, 1H, HCO2H), 8.07 (s, 1H), 7.51 (dd, J=20.0, 8.2 Hz, 2H), 7.28-7.25 (m, 1H), 4.06 (s, 3H), 3.40 (br s, 8H), 2.45 (s, 3H). ESI-MS: [M+H]⁺: 410.0.

### Example 38. Synthesis of (S)-2-(2,8-dimethylimidazo[1,2-a]pyridin-6-yl)-9-fluoro-7-(3-methyl piperazin-1-yl)-4H-pyrido[1,2-a][1,3,5]triazin-4-one hydrochloride (Compound 121)

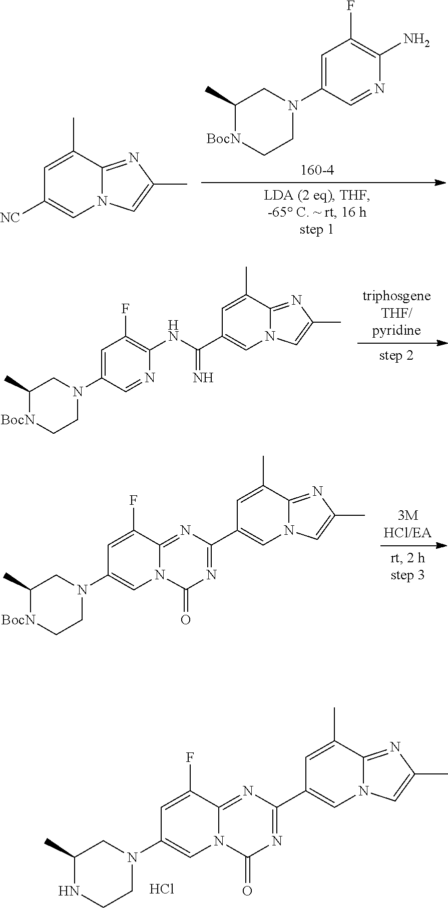

### Step 1: tert-butyl (S)-4-(6-(2,8-dimethylimidazo[1,2-a]pyridine-6-carboximidamido)-5-fluoropyridin-3-yl)-2-methylpiperazine-1-carboxylate

To a mixture of diisopropylamine (0.9 mL, 5.4 mmol) in dry THF (20 mL) was added n-BuLi (4.2 mL, 6.6 mmol, 1.6M) dropwise at −65° C. The mixture was stirred at this temperature for 1 hour. Tert-butyl (S)-4-(6-amino-5-fluoropyridin-3-yl)-2-methylpiperazine-1-carboxylate (1.40 g, 4.5 mmol) in dry THF (10 mL) was added. The mixture was allowed to warm to room temperature and stirred for 1 h. 2,8-dimethylimidazo[1,2-a]pyridine-6-carbonitrile (513 mg, 3.0 mmol) in dry THF (10 mL) was added to the mixture at −40° C. and the mixture was allowed to warm to room temperature and stirred for 16 h. The mixture was diluted with water (30 mL), extracted with EtOAc (30 mL×2). The combined organic layer was washed with brine (50 mL), dried over sodium sulfate, filtered and concentrated in vacuo. The residue was purified by column chromatography (PE/EA=1:1 to 0:1) to title product (133 mg, yield: 10%,) as a yellow solid. ¹H NMR (400 MHz, CDCl₃) δ 8.71 (s, 1H), 7.78 (d, J=2.6 Hz, 1H), 7.40 (s, 1H), 7.36 (s, 1H), 7.06-7.01 (m, 1H), 4.42-4.34 (m, 1H), 3.98-3.96 (m, 1H), 3.47-3.43 (m, 1H), 3.31-3.23 (m, 2H), 3.00-2.96 (m, 1H), 2.85-2.77 (m, 1H), 2.65 (s, 3H), 2.49 (s, 3H), 1.49 (s, 9H), 1.31 (d, J=6.8 Hz, 3H).

### Step 2: tert-butyl (S)-4-(2-(2,8-dimethylimidazo[1,2-a]pyridin-6-yl)-9-fluoro-4-oxo-4H-pyrido[1,2-a][1,3,5]triazin-7-yl)-2-methylpiperazine-1-carboxylate

To a solution of tert-butyl (S)-4-(6-(2,8-dimethylimidazo[1,2-a]pyridine-6-carboximidamido)-5-fluoropyridin-3-yl)-2-methylpiperazine-1-carboxylate (400 mg, 0.83 mmol) in THF (40 mL) and pyridine (4 mL) was added triphosgene (738 mg, 2.50 mmol) at 0° C. The reaction mixture was stirred at 0° C. for 10 min. The mixture was diluted with water (30 mL), extracted with EtOAc (30 mL×2). The combined organic layer was washed with brine (50 mL), dried over sodium sulfate, filtered and concentrated in vacuo. The residue was purified by prep-HPLC (0.05% FA in water/CH₃CN) to give title product (200 mg, 47.5%) as a yellow solid. ESI-MS: [M+H]⁺508.2.

### Step 3: (S)-2-(2,8-dimethylimidazo[1,2-a]pyridin-6-yl)-9-fluoro-7-(3-methyl piperazin-1-yl)-4H-pyrido[1,2-a][1,3,5]triazin-4-one hydrochloride

To a solution of tert-butyl (S)-4-(2-(2,8-dimethylimidazo[1,2-a]pyridin-6-yl)-9-fluoro-4-oxo-4H-pyrido [1,2-a][1,3,5]triazin-7-yl)-2-methylpiperazine-1-carboxylate (200 mg, 0.39 mmol) in EtOAc (10 mL) was added 3M HCl/EtOAc (10 mL) at room temperature. The reaction mixture was stirred for 2 h. The precipitate was filtered and dried under vacuum to give title product (130 mg, yield: 74.5%) as a yellow solid. ¹H NMR (400 MHz, MeOD-d4) δ 9.62 (s, 1H), 8.68 (s, 1H), 8.43 (d, J=2.0 Hz, 1H), 8.38-8.31 (m, 1H), 8.12 (d, J=1.0 Hz, 1H), 4.09-3.96 (m, 2H), 3.64-3.54 (m, 2H), 3.44-3.32 (m, 2H), 3.09-3.05 (m, 1H), 2.73 (s, 3H), 2.61 (s, 3H), 1.46 (d, J=6.4 Hz, 3H). ESI-MS: [M+H]⁺ 408.2.

### Example 39. Synthesis of 2-(8-fluoro-2-methylimidazo[1,2-a]pyridin-6-yl)-6-methyl-7-(piperazin-1-yl)-4H-pyrido[1,2-a][1,3,5]triazin-4-one hydrochloride (Compound 122)

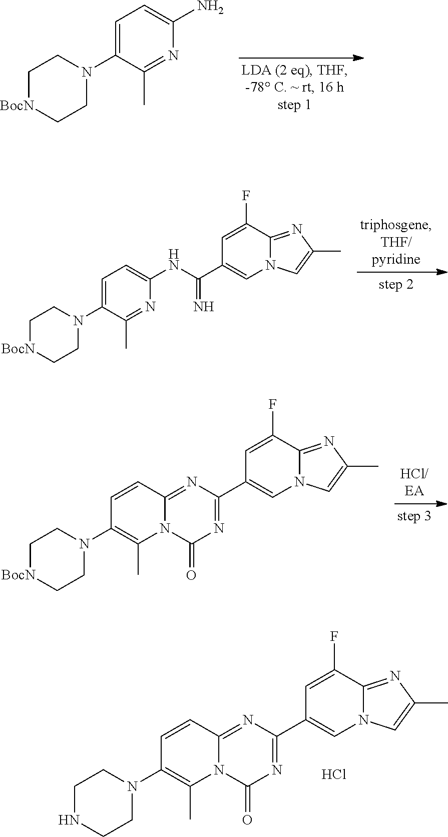 [SMILES: Cc1nc(N)ccc1N1CCN(C(=O)OC(C)(C)C)CC1 ; Cc1cn2cc(C(=N)Nc3ccc(N4CCN(C(=O)OC(C)(C)C)CC4)c(C)n3)cc(F)c2n1 ; Cc1cn2cc(-c3nc(=O)n4c(C)c(N5CCN(C(=O)OC(C)(C)C)CC5)ccc4n3)cc(F)c2n1 ; Cc1cn2cc(-c3nc(=O)n4c(C)c(N5CCNCC5)ccc4n3)cc(F)c2n1 ; Cl]

### Step 1: tert-butyl 4-(6-(8-fluoro-2-methylimidazo[1,2-a]pyridine-6-carboximidamido)-2-methylpyridin-3-yl)piperazine-1-carboxylate

To a mixture of diisopropylamine (300 mg, 3.0 mmol) in dry THF (15 mL) was added n-BuLi (2.0 mL, 3.2 mmol, 1.6M) dropwise at −65° C. The mixture was stirred at this temperature for 1 hour, tert-butyl 4-(6-amino-2-methylpyridin-3-yl)piperazine-1-carboxylate (730 mg, 2.5 mmol) in dry THF (5 mL) was added. The mixture was allowed to warm to room temperature and stirred for 1 h. 8-fluoro-2-methylimidazo[1,2-a]pyridine-6-carbonitrile (297 mg, 1.7 mmol) in dry THF (10 mL) was added to the mixture at −65° C. and allowed to warm to room temperature and stirred for 16 h. The mixture was diluted with water (30 mL), extracted with EtOAc (30 mL×2). The combined organic layer was washed with brine (50 mL), dried over sodium sulfate, filtered and concentrated in vacuo. The residue was purified by C₁₈ column to give title product as a white solid (150 mg, yield: 12.6%). ESI-MS: [M+H]⁺:468.2.

### Step 2: tert-butyl 4-(2-(8-fluoro-2-methylimidazo[1,2-a]pyridin-6-yl)-6-methyl-4-oxo-4H-pyrido[1,2-a][1,3,5]triazin-7-yl)piperazine-1-carboxylate

To a solution of tert-butyl 4-(6-(8-fluoro-2-methylimidazo[1,2-a]pyridine-6-carboximidamido)-2-methylpyridin-3-yl) piperazine-1-carboxylate (100 mg, 0.21 mmol) in THE (10 mL) and pyridine (2 mL) was added triphosgene (187 mg, 0.63 mmol). The reaction mixture was stirred for 1 h at rt, The mixture was diluted with water (30 mL), extracted with EtOAc (30 mL×2). The combined organic layer was washed with brine (50 mL), dried over sodium sulfate, filtered and concentrated in vacuo. The residue was diluted with MeOH (40 mL) and stirred for 1 hour at rt. The precipitate was filtered and the solid was dried in vacuo to give title product as a yellow solid (60 mg, 57.9%). ¹H NMR (400 MHz, CDCl₃) δ 8.60 (d, J=1.2 Hz, 1H), 7.47 (d, J=2.3 Hz, 1H), 7.40-7.33 (m, 2H), 7.09 (d, J=8.5 Hz, 1H), 3.65-3.52 (m, 4H), 2.90-2.81 (m, 4H), 2.52 (s, 3H), 2.49 (s, 3H), 1.49 (s, 9H). ESI-MS: [M+H]+ 494.2.2.

### Step 3: 2-(8-fluoro-2-methylimidazo[1,2-a]pyridin-6-yl)-6-methyl-7-(piperazin-1-yl)-4H-pyrido[1,2-a][1,3,5]triazin-4-one hydrochloride

To a solution of tert-butyl 4-(2-(8-fluoro-2-methylimidazo[1,2-a]pyridin-6-yl)-6-methyl-4-oxo-4H-pyrido[1,2-a][1,3,5]triazin-7-yl)piperazine-1-carboxylate (20 mg, 0.041 mmol) in EtOAc (1 mL) was added 3M HCl/EtOAc (2 mL) at room temperature. The reaction mixture was stirred for 2 h. The precipitate was filtered and the solid was dried under vacuum to give title product (5 mg, yield: 33%) as a yellow solid. ¹H NMR (400 MHz, MeOD-d₄) δ 9.62 (s, 1H), 8.62 (d, J=11.8 Hz, 1H), 8.27-8.20 (m, 2H), 7.72 (d, J=9.2 Hz, 1H), 3.48-3.46 (m, 4H), 3.37-3.35 (m, 4H), 3.03 (s, 3H), 2.62 (s, 3H). ESI-MS: [M+H]⁺ 394.0.

### Example 40. Synthesis of (S)-2-(2,8-dimethylimidazo[1,2-a]pyridin-6-yl)-9-methyl-7-(3-methylpiperazin-1-yl)-4H-pyrido[1,2-a][1,3,5]triazin-4-one hydrochloride (Compound 123)

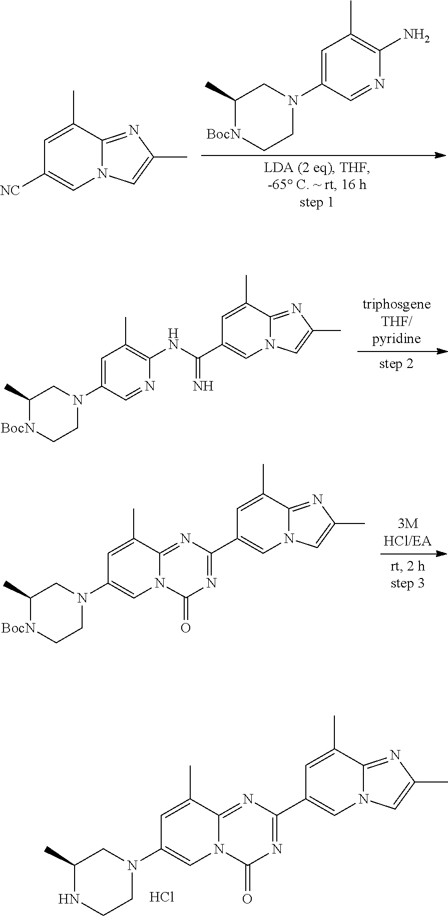

### Step 1: tert-butyl (S)-4-(6-(2,8-dimethylimidazo[1,2-a]pyridine-6-carboximidamido)-5-methylpyridin-3-yl)-2-methylpiperazine-1-carboxylate

To a mixture of diisopropylamine (303 mg, 3.0 mmol) in dry THF (10 mL) was added n-BuLi (2.6 mL, 4.2 mmol, 1.6M) dropwise at −65° C. The mixture was stirred at this temperature for 1 hour. Tert-butyl (S)-4-(6-amino-5-methylpyridin-3-yl)-2-methylpiperazine-1-carboxylate (612 mg, 2.0 mmol) in dry THF (5 mL) was added. The mixture was allowed to warm to room temperature and stirred for 1 h. 2,8-dimethylimidazo[1,2-a]pyridine-6-carbonitrile (171 mg, 1.0 mmol) in dry THF (5 mL) was added to the mixture at −40° C. and allowed to warm to room temperature and stirred for 16 h. The mixture was diluted with water (30 mL), extracted with EtOAc (30 mL×2). The combined organic layer was washed with brine (50 mL), dried over sodium sulfate, filtered and concentrated in vacuo. The residue was purified by column chromatography (PE/EA=1:1 to 0:1) to give title product (90 mg, yield: 18%) as a yellow solid. ¹H NMR (400 MHz, CDCl₃) δ 8.64 (s, 1H), 7.83 (d, J=2.9 Hz, 1H), 7.42-7.40 m, 2H), 7.15 (d, J=2.5 Hz, 1H), 4.40-4.33 (m, 1H), 3.97 (d, J=13.4 Hz, 1H), 3.45 (d, J=10.1 Hz, 1H), 3.34-3.22 (m, 2H), 2.93 (dd, J=11.8, 3.8 Hz, 1H), 2.78-2.72 (m, 1H), 2.65 (s, 3H), 2.50 (s, 3H), 2.49 (s, 3H), 1.49 (s, 9H), 1.33 (d, J=6.8 Hz, 3H). ESI-MS: [M+H]⁺ 478.1.

### Step 2: tert-butyl (S)-4-(2-(2,8-dimethylimidazo[1,2-a]pyridin-6-yl)-9-methyl-4-oxo-4H-pyrido[1,2-a][1,3,5]triazin-7-yl)-2-methylpiperazine-1-carboxylate

To a solution of tert-butyl (S)-4-(6-(2,8-dimethylimidazo[1,2-a]pyridine-6-carboximidamido)-5-methylpyridin-3-yl)-2-methylpiperazine-1-carboxylate (90 mg, 0.18 mmol) in THF (10 mL) and pyridine (1 mL) was added triphosgene (162 mg, 0.55 mmol) at 0° C. The reaction mixture was stirred at 0° C. for 30 min. The mixture was diluted with water (30 mL), extracted with EtOAc (30 mL×2). The combined organic layer was washed with brine (50 mL), dried over sodium sulfate, filtered and concentrated in vacuo. The residue was diluted with MeOH (10 mL), stirred for 1 hour at rt, the precipitate was filtered and dried in vacuo to give title product (70 mg, yield: 73.7%) as a yellow solid. ¹H NMR (400 MHz, CDCl₃) δ 9.25 (d, J=0.9 Hz, 1H), 8.39 (d, J=2.7 Hz, 1H), 8.05 (s, 1H), 7.70 (d, J=1.8 Hz, 1H), 7.42 (s, 1H), 4.45-4.40 (m, 1H), 4.05-4.00 (m, 1H), 3.60-3.56 (m, 1H), 3.44-3.41 (m, 1H), 3.31-3.27 (m, 1H), 3.08 (dd, J=12.0, 4.0 Hz, 1H), 2.92-2.86 (m, 1H), 2.71 (s, 3H), 2.67 (s, 3H), 2.50 (s, 3H), 1.50 (s, 9H), 1.31 (d, J=6.8 Hz, 3H). ESI-MS: [M+H]⁺504.2.

### Step 3: (S)-2-(2,8-dimethylimidazo[1,2-a]pyridin-6-yl)-9-methyl-7-(3-methylpiperazin-1-yl)-4H-pyrido[1,2-a][1,3,5]triazin-4-one hydrochloride

To a solution of tert-butyl (S)-4-(2-(2,8-dimethylimidazo[1,2-a]pyridin-6-yl)-9-methyl-4-oxo-4H-pyrido [1,2-a][1,3,5]triazin-7-yl)-2-methylpiperazine-1-carboxylate (70 mg, 0.139 mmol) in EtOAc (4 mL) was added 3M HCl/EtOAc (2 ml) at room temperature. The reaction mixture was stirred for 2 h. The precipitate was filtered and dried under vacuum to give title product (43 mg, yield: 70.5%) as a yellow solid. ¹H NMR (400 MHz, MeOD-d₄) δ 9.64 (s, 1H), 8.71 (s, 1H), 8.49 (d, J=2.8 Hz, 1H), 8.26 (s, 1H), 8.13 (s, 1H), 4.03 (dd, J=10.6, 7.4 Hz, 2H), 3.61-3.57 (m, 2H), 3.42-3.36 (m, 1H), 3.25-3.18 (m, 1H), 3.02-2.96 (m, 1H), 2.76 (s, 3H), 2.73 (s, 3H), 2.61 (s, 3H), 1.45 (d, J=6.8 Hz, 3H). ESI-MS: [M+H]⁺ 404.1.

### Example 41. Synthesis of (S)-7-(3,4-dimethylpiperazin-1-yl)-2-(8-fluoro-2-methylimidazo[1,2-a]pyridin-6-yl)-9-methyl-4H-pyrido[1,2-a][1,3,5]triazin-4-one (Compound 124)

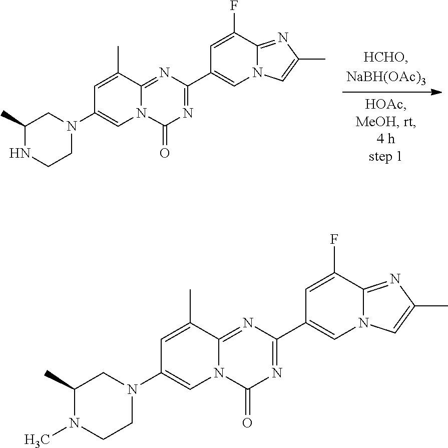

To a mixture of (S)-2-(8-fluoro-2-methylimidazo[1,2-a]pyridin-6-yl)-9-methyl-7-(3-methylpiperazin-1-yl)-4H-pyrido[1,2-a][1,3,5]triazin-4-one hydrochloride (40 mg, 0.09 mmol) in MeOH (5 mL) was added Polyoxymethylene (15 mg, 0.45 mmol), AcOH (cat.) at RT. The mixture was stirred at this temperature for 0.5 hour, NaBH(OAc)₃ (19 mg, 0.27 mmol) was added. The mixture was stirred for 16 h. The mixture was diluted with water (10 mL), extracted with DCM (10 mL×2). The combined organic layer was washed with brine (20 mL), dried over sodium sulfate, filtered and concentrated in vacuo. The residue was purified by prep-HPLC (0.05% NH₃·H₂O in water/CH₃CN) to give title product (30 mg, yield: 78.9%,) as a white solid. ¹H NMR (400 MHz, DMSO-d₆) δ 9.43 (d, J=1.1 Hz, 1H), 8.27 (s, 1H), 8.17 (d, J=2.4 Hz, 1H), 8.03 (d, J=2.2 Hz, 1H), 7.85 (d, J=12.3 Hz, 1H), 3.67 (t, J=10.5 Hz, 2H), 2.89 (t, J=9.6 Hz, 2H), 2.61 (s, 3H), 2.53 (d, J=8.9 Hz, 1H), 2.38 (s, 3H), 2.29 (dd, J=17.8, 7.2 Hz, 1H), 2.24 (s, 3H), 2.18 (d, J=6.3 Hz, 1H), 1.09 (d, J=6.2 Hz, 3H). ESI-MS: [M+H]⁺=422.0.

### Example 42. Synthesis of 7-(3,3-dimethylpiperazin-1-yl)-2-(8-fluoro-2-methylimidazo [1,2-a]pyridin-6-yl)-9-methyl-4H-pyrido[1,2-a][1,3,5]triazin-4-one hydrochloride (Compound 125)

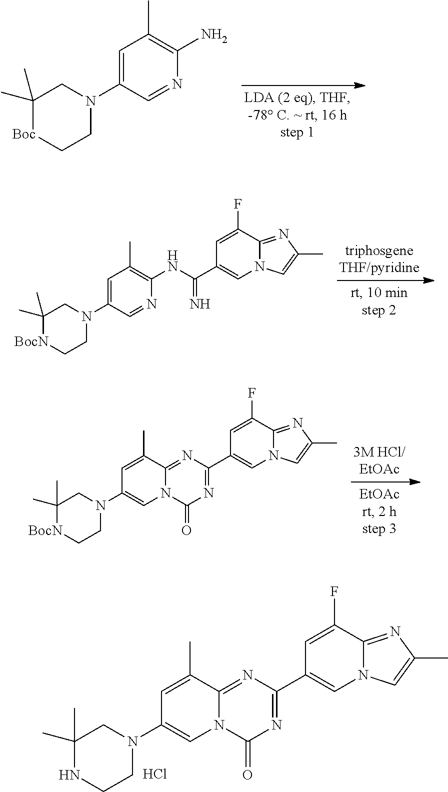

### Step 1: tert-butyl 4-(6-(8-fluoro-2-methylimidazo[1,2-a]pyridine-6-carboximidamido)-5-methylpyridin-3-yl)-2,2-dimethylpiperazine-1-carboxylate

To a mixture of diisopropylamine (666 mg, 6.6 mmo) in dry THF (15 mL) was added n-BuLi (4.5 mL, 7.2 mmol, 1.6M) dropwise at −65° C. The mixture was stirred at this temperature for 1 hour. tert-butyl 4-(6-amino-5-methylpyridin-3-yl)-2,2-dimethylpiperazine-1-carboxylate (1.5 g, 4.5 mmol) in THF (20 mL) was added. The mixture was allowed to warm to room temperature and stirred for 1 h. 8-fluoro-2-methylimidazo[1,2-a]pyridine-6-carbonitrile(525 mg, 3 mmol) in dry THF (5 mL) was added to the mixture at −40° C. and allowed to warm to room temperature and stirred for 16 h. The reaction mixture was diluted with water (100 mL) and extracted with EtOAc (100 mL×3). The organic layer was washed with brine, dried with Na₂SO₄ and evaporated to give crude title compound. The residue was purified by C₁₈ column chromatography to give title compound (400 mg, 30% yield) as a grey solid. ESI-MS (M+H)⁺:496.3. ¹H NMR (400 MHz, CDCl₃) δ 8.51 (d, J=1.1 Hz, 1H), 7.72 (d, J=3.0 Hz, 1H), 7.48-7.45 (m, 2H), 6.98 (d, J=2.9 Hz, 1H), 3.82-3.77 (m, 2H), 3.41-3.36 (m, 2H), 3.27 (s, 2H), 2.49 (s, 3H), 2.48 (s, 3H), 1.50 (s, 9H), 1.44 (s, 6H).

### Step 2: tert-butyl 4-(2-(8-fluoro-2-methylimidazo[1,2-a]pyridin-6-yl)-9-methyl-4-oxo-4H-pyrido[1,2-a][1,3,5]triazin-7-yl)-2,2-dimethylpiperazine-1-carboxylate

The tert-butyl 4-(6-(8-fluoro-2-methylimidazo[1,2-a]pyridine-6-carboximidamido)-5-methylpyridin-3-yl)-2,2-dimethylpiperazine-1-carboxylate (400 mg, 0.808 mmol) was dissolved in THE (20 mL) and pyridine (2 mL) and triphosgene (1.2 g, 2.22 mmol) was added at 0° C. The mixture was stirred at r.t for 1 h. The reaction mixture was diluted with water (30 mL) and extracted with EtOAc (20 mL×3). The organic layer was washed with brine, dried with Na₂SO₄ and evaporated to give crude title compound. The residue was suspended in MeOH, stirred for 1 h. The precipitate was filtered to give title compound (300 mg, 71% yield) as a yellow solid. ESI-MS (M+H)⁺:522.3. ¹H NMR (400 MHz, CDCl₃) δ 9.22-9.17 (m, 1H), 8.31-8.25 (m, 1H), 7.96-7.89 (m, 1H), 7.60-7.57 (m, 1H), 7.49-7.46 (m, 1H), 3.91-3.87 (m, 2H), 3.51-3.49 (m, 2H), 3.41 (s, 2H), 2.71 (s, 3H), 2.50 (s, 3H), 1.51 (s, 9H), 1.46 (s, 6H).

### Step 3: 7-(3,3-dimethylpiperazin-1-yl)-2-(8-fluoro-2-methylimidazo [1,2-a]pyridin-6-yl)-9-methyl-4H-pyrido[1,2-a][1,3,5]triazin-4-one hydrochloride

A solution of tert-butyl 4-(2-(8-fluoro-2-methylimidazo[1,2-a]pyridin-6-yl)-9-methyl-4-oxo-4H-pyrido[1,2-a][1,3,5]triazin-7-yl)-2,2-dimethylpiperazine-1-carboxylate (300 mg, 0.57 mmol) in 3M HCl/EtOAc (10 mL) was stirred for 2 h at RT. The mixture was filtered and the solid washed with EtOAc (3 mL) and dried under vacuum to afford title compound (250 mg, 95.1%) as a yellow solid, ESI-MS (M+H)⁺:422.0. ¹H NMR (400 MHz, MeOD-d4) δ 9.69-9.66 (m, 1H), 8.68-8.64 (m, 1H), 8.53-8.49 (m, 1H), 8.32-8.27 (m, 2H), 3.67-3.62 (m, 2H), 3.53-3.49 (m, 4H), 2.77 (s, 3H), 2.63 (s, 3H), 1.55 (s, 6H).

### Example 43. Synthesis of 2-2,8-dimethylimidazo[1,2-a]pyridin-6-yl-7-(piperazin-1-yl)-4H-pyrido[1,2-a][1,3,5]triazin-4-one hydrochloride (Compound 126)

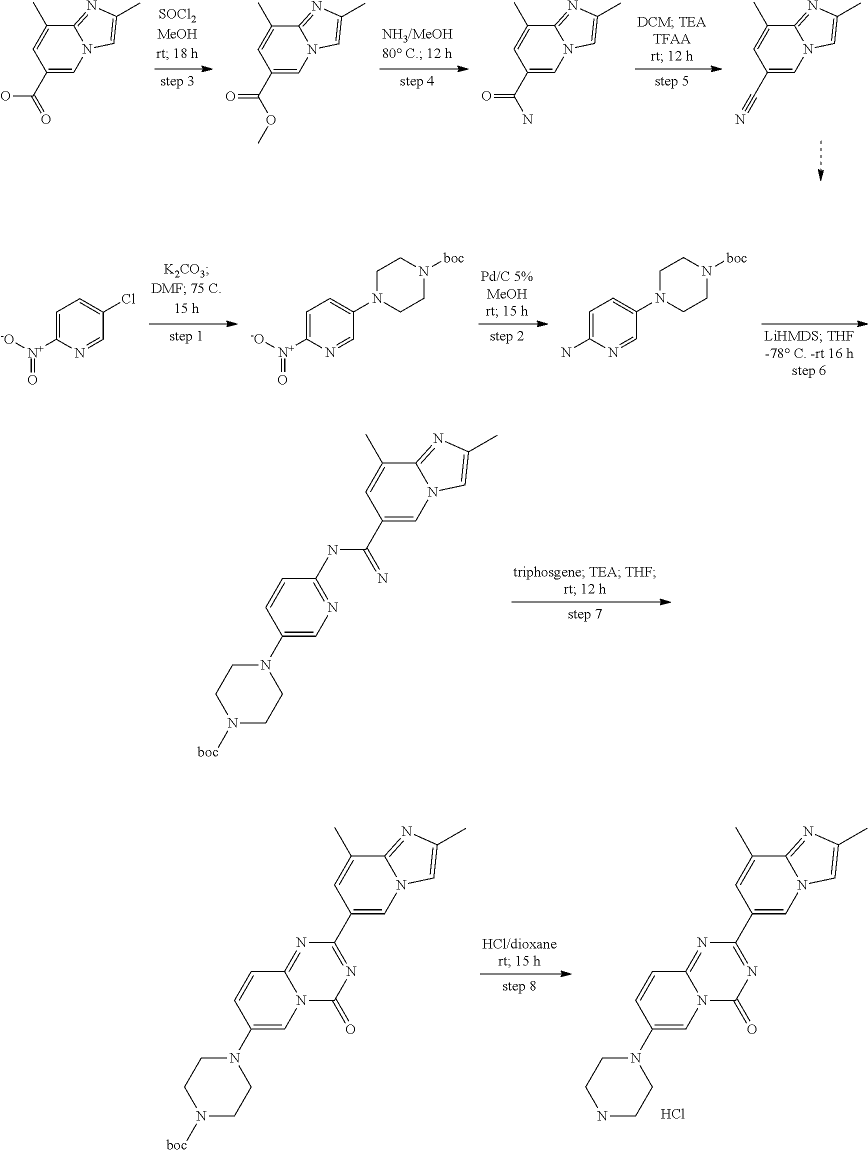

### Step 1: tert-butyl 4-(6-nitropyridin-3-yl)piperazine-1-carboxylate

5-Chloro-2-nitropyridine (150.0 g, 946.12 mmol), tert-butyl piperazine-1-carboxylate (185.03 g, 993.43 mmol) and potassium carbonate (326.9 g, 2.37 mol) were mixed in DMF (1000 mL) The reaction mixture was stirred for 12 h at 75° C. The reaction mixture was diluted with water (4000 mL) and extracted with EtOAc (3×800 mL). The organic layer was washed with brine, dried with Na₂SO₄ and evaporated to give crude title compound. The residue was purified by silica gel column chromatography to give title compound (110 g; 38% yield). ¹H NMR (400 MHz, DMSO-d₆) δ 8.22 (d, J=3.0 Hz, 1H), 8.14 (d, J=9.0 Hz, 1H), 7.44 (dd, J=9.3, 3.0 Hz, 1H), 3.49 (q, J=6.0 Hz, 8H), 1.44-1.38 (s, 9H).

### Step 2: tert-butyl 4-(6-aminopyridin-3-yl)piperazine-1-carboxylate

To a mixture of tert-butyl 4-(6-nitropyridin-3-yl)piperazine-1-carboxylate (9.5 g, 30.81 mmol) in MeOH (200 mL) was added 10% Pd/C (2 g). The reaction mixture was stirred for 16 h at rt under hydrogen atmosphere (balloon pressure). The solvent was filtered and the filter cake was washed with MeOH (50 mL). The filtrate was concentrated to give title product (8 g; 93% yield). ¹H NMR (400 MHz, DMSO-d₆) δ 7.60 (d, J=3.0 Hz, 1H), 7.17 (dd, J=8.8, 3.0 Hz, 1H), 6.40 (d, J=8.8 Hz, 1H), 5.48 (br s, 2H), 3.42 (t, J=4.9 Hz, 4H), 2.84 (t, J=5.0 Hz, 4H), 1.40 (s, 9H). ESI-MS: 279.2 [M+H].

### Step 3: methyl 2,8-dimethylimidazo[1,2-a]pyridine-6-carboxylate

2,8-Dimethylimidazo[1,2-a]pyridine-6-carboxylic acid hydrochloride (10.0 g, 44.11 mmol) was dissolved in MeOH (100 mL) and thionyl chloride (15.74 g, 132.34 mmol, 9.6 ml, 3.0 equiv) was added dropwise and the resulting mixture was stirred 18 h at room temperature. The mixture was then concentrated and DCM (250 mL) was added. Saturated NaHCO₃ (20 mL) was added, and the resulting mixture was stirred at room temperature for 15 minutes. The organic layer was washed with saturated NaHCO₃ (150 mL), water (200 mL), and saturated aqueous NaCl (200 mL), then dried over Na₂SO₄, filtered and concentrated to give methyl 2,8-dimethylimidazo[1,2-a]pyridine-6-carboxylate (8 g, 89% yield). ¹H NMR (400 MHz, DMSO-d₆) δ 9.07 (d, J=1.9 Hz, 1H), 7.80 (s, 1H), 7.41-7.35 (m, 1H), 3.85 (s, 3H), 2.46 (s, 3H), 2.34 (s, 3H). ESI-MS: 205.2 [M+H].

### Step 4: 2,8-dimethylimidazo[1,2-a]pyridine-6-carboxamide

To a solution of methyl 2,8-dimethylimidazo[1,2-a]pyridine-6-carboxylate (5.8 g, 28.4 mmol) in MeOH (60 mL) 25% methanolic ammonia solution (80 mL) was added and the mixture in autoclave and heated at 80° C. for 12 hours. The mixture was concentrated in vacuo to give the product (5 g, 93% yield). ¹H NMR (400 MHz, DMSO-d₆) δ 8.86 (s, 1H), 7.95 (s, 1H), 7.73 (s, 1H), 7.41 (d, J=12.4 Hz, 2H), 2.33 (s, 3H).

### Step 5: 2,8-dimethylimidazo[1,2-a]pyridine-6-carbonitrile

2,8-Dimethylimidazo[1,2-a]pyridine-6-carboxamide (5.0 g, 26.44 mmol) was dissolved in DCM (100 ml). Then triethylamine (10.7 g, 105.75 mmol, 14.74 ml, 4.0 equiv) was added. TFAA (8.33 g, 39.66 mmol, 5.56 ml, 1.5 equiv) was added dropwise at 0 C. The mixture was stirred at this temperature for 20 min and allowed to warm to room temperature and stirred for 16 h. The mixture was diluted with water (150 mL), extracted with DCM (2×50 mL). The combined organic layer was washed with brine (50 mL), dried over sodium sulfate, filtered and concentrated in vacuo The residue was purified by column chromatography (CHCl₃/ACN) to give title product (2.5 g, 55% yield). ESI-MS: 172.1 [M+H].

### Step 6: tert-butyl 4-(6-2,8-dimethylimidazo[1,2-a]pyridine-6-imidamidopyridin-3-yl) piperazine-1-carboxylate

To a mixture of tert-butyl 4-(6-aminopyridin-3-yl)piperazine-1-carboxylate (1.5 g, 5.39 mmol) in dry THE (25 mL) was added lithium bis(trimethylsilyl)amide (1.08 g, 6.47 mmol, 5.39 ml, 1.2 equiv) dropwise at −78° C. The mixture was allowed to warm to −40° C. and stirred for 1 h. 2,8-Dimethylimidazo[1,2-a]pyridine-6-carbonitrile (922.35 mg, 5.39 mmol) in dry THE (10 mL) was added at −78 C. The mixture was allowed to warm to room temperature and stirred for 16 h. The mixture was diluted with water (50 mL), extracted with EtOAc (2×40 mL). The combined organic layer was washed with brine (50 mL), dried over sodium sulfate, filtered and concentrated in vacuo. The residue was purified by column chromatography (ACN/MeOH) to give title product (1.5 g, 62% yield). ESI-MS: 450.2 [M+H].

### Step 7: tert-butyl 4-(2-2,8-dimethylimidazo[1,2-a]pyridin-6-yl-4-oxo-4H-pyrido[1,2-a][1,3,5]triazin-7-yl)piperazine-1-carboxylate

To a mixture of tert-butyl 4-(6-2,8-dimethylimidazo[1,2-a]pyridine-6-imidamidopyridin-3-yl) piperazine-1-carboxylate (502.92 mg, 1.12 mmol) and triethylamine (566.28 mg, 5.6 mmol, 780.0 μl, 5.0 equiv) in THE (15 mL) was added ditrichloromethyl carbonate (331.98 mg, 1.12 mmol). The reaction mixture was stirred for 15 h at rt. The mixture was filtered, washed with THF(15 ml) and water (2×15 ml) and dried in vacuo to give title product (0.2 g, 38% yield). ¹H NMR (400 MHz, DMSO-d₆) δ 9.26 (s, 1H), 8.27 (s, 1H), 8.20 (d, J=9.0 Hz, 1H), 7.85 (s, 1H), 7.78 (s, 1H), 7.64 (d, J=8.7 Hz, 1H), 3.51 (s, 4H), 3.27 (s, 4H), 2.99 (d, J=3.1 Hz, 3H), 2.34 (s, 3H), 1.41 (d, J=5.0 Hz, 9H).

### Step 8: 2-2,8-dimethylimidazo[1,2-a]pyridin-6-yl-7-(piperazin-1-yl)-4H-pyrido[1,2-a][1,3,5]triazin-4-one hydrochloride

Tert-butyl 4-(2-2,8-dimethylimidazo[1,2-a]pyridin-6-yl-4-oxo-4H-pyrido[1,2-a][1,3,5]triazin-7-yl) piperazine-1-carboxylate (99.98 mg, 210.25 μmol) in MeOH (10 mL) was added HCl in dioxane(1 mL) at 0 C. The mixture was stirred for 12 h at rt. After concentration, the residue was stirred with EtOAc. The precipitate was filtered and the solid was dried to give title product (26 mg, 30% yield). ¹H NMR (400 MHz, DMSO-d₆) δ 9.65 (s, 1H), 9.52 (s, 2H), 8.49 (s, 1H), 8.39 (s, 1H), 8.23 (s, 1H), 7.77 (s, 1H), 3.59 (s, 4H), 3.24 (s, 4H), 2.67 (s, 3H). ESI-MS: 376.2 [M+H].

### Example 44. Synthesis of 7-2,6-diazaspiro[3.3]heptan-2-yl-2-8-fluoro-2-methylimidazo[1,2-a]pyridin-6-yl-4H-pyrido[1,2-a][1,3,5]triazin-4-one trifluoroacetic acid (Compound 127)

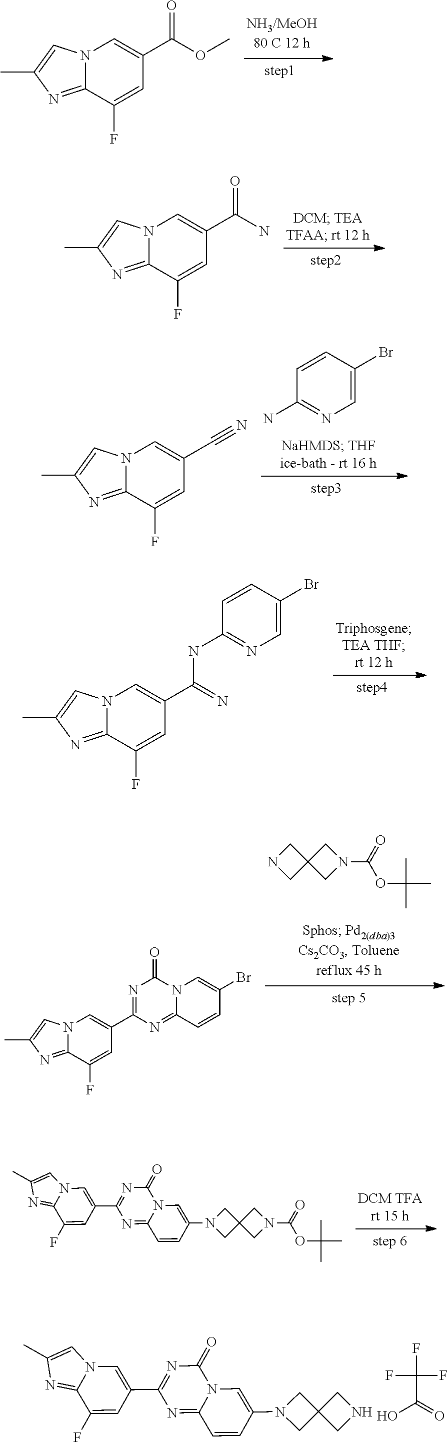

### Step 1: 8-fluoro-2-methylimidazo[1,2-a]pyridine-6-carboxamide

To a solution of methyl 8-fluoro-2-methylimidazo[1,2-a]pyridine-6-carboxylate (8.0 g, 38.43 mmol) in MeOH (60 mL) 25% methanolic ammonia solution (100 mL) was added and the mixture was sealed in autoclave and heated at 80° C. for 12 hours. The mixture was concentrated in vacuo to give the product (5.9 g, 79% yield). ESI-MS: 194.0 [M+H].

### Step 2: 8-fluoro-2-methylimidazo[1,2-a]pyridine-6-carbonitrile

8-Fluoro-2-methylimidazo[1,2-a]pyridine-6-carboxamide (5.9 g, 30.53 mmol) was dissolved in DCM (100 ml). Then triethylamine (9.27 g, 91.58 mmol, 12.76 ml, 3.0 equiv) was added. TFAA (9.62 g, 45.79 mmol, 6.42 ml, 1.5 equiv) was added dropwise at 0° C. The mixture was stirred at this temperature for 20 min and allowed to warm to room temperature and stirred for 16 h. The mixture was diluted with water (150 mL), extracted with DCM (2×50 mL). The combined organic layer was washed with brine (50 mL), dried over sodium sulfate, filtered and concentrated in vacuo. The residue was purified by column chromatography (CHCl3/ACN) to give title product (3 g, 56% yield). ¹H NMR (500 MHz, DMSO-d₆) δ 9.17 (s, 1H), 7.91 (s, 1H), 7.54 (d, J=11.1 Hz, 1H), 2.38 (s, 3H). ESI-MS: 176.2 [M+H].

### Step 3: N-(5-bromopyridin-2-yl)-8-fluoro-2-methylimidazo[1,2-a]pyridine-6-carboximidamide

To a solution of 5-bromopyridin-2-amine (2.96 g, 17.13 mmol) in THE (30 ml) sodium bis(trimethylsilyl)amide (3.74 g, 20.38 mmol, 8.77 ml, 1.19 equiv) was added dropwise while the reaction mixture was magnetically stirred under Ar at ice-bath temperature. After completed addition, the mixture was stirred for 30 min, and 8-fluoro-2-methylimidazo[1,2-a]pyridine-6-carbonitrile (3.0 g, 17.13 mmol) was added in one portion. The resulting mixture was stirred overnight at r.t. The precipitate that had formed was collected by filtration, washing of the precipitate with H₂O and MTBE and drying in vacuo to give title product (4.5 g 75% yield). ¹H NMR (500 MHz, DMSO-d₆) δ 9.77-9.71 (m, 1H), 8.72 (s, 1H), 8.22 (d, J=2.7 Hz, 1H), 7.64 (dd, J=8.6, 2.8 Hz, 1H), 7.49 (d, J=3.1 Hz, 1H), 6.79 (d, J=8.6 Hz, 1H), 6.58 (d, J=13.2 Hz, 1H), 2.18 (s, 3H).

### Step 4: 7-bromo-2-8-fluoro-2-methylimidazo[1,2-a]pyridin-6-yl-4H-pyrido[1,2-a][1,3,5]triazin-4-one

To a solution of N-(5-bromopyridin-2-yl)-8-fluoro-2-methylimidazo[1,2-a]pyridine-6-carboximidamide (4.5 g, 12.92 mmol) in THE (60 mL) and triethylamine (6.54 g, 64.62 mmol, 9.01 ml, 5.0 equiv) was added ditrichloromethyl carbonate (3.84 g, 12.92 mmol). The reaction mixture was stirred for 12 h at rt. The mixture was filtered, washed with THE (2×50 ml) and water (2×45 ml) and dried in vacuo to give title product (3 g, 62% yield). ¹H NMR (500 MHz, DMSO-d₆) δ 9.25 (s, 1H), 8.81 (d, J=2.3 Hz, 1H), 8.33 (dd, J=8.6, 2.5 Hz, 1H), 8.07-7.99 (m, 1H), 7.92 (d, J=8.6 Hz, 1H), 7.37 (d, J=10.6 Hz, 1H), 2.34 (s, 3H). ESI-MS: [M+H]⁺ 376.0.

### Step 5: tert-butyl 6-(2-8-fluoro-2-methylimidazo[1,2-a]pyridin-6-yl-4-oxo-4H-pyrido[1,2-a][1,3,5]triazin-7-yl)-2,6-diazaspiro[3.3]heptane-2-carboxylate

A mixture of 7-bromo-2-8-fluoro-2-methylimidazo[1,2-a]pyridin-6-yl-4H-pyrido[1,2-a][1,3,5]triazin-4-one (997.2 mg, 2.66 mmol), tert-butyl 2,6-diazaspiro[3.3]heptane-2-carboxylate hydrochloride (1.251 g, 5.33 mmol), SPhos (109.4 mg, 266.5 μmol), Pd₂(dba)₃ (244.1 mg, 266.5 μmol), cesium carbonate (2.605 g, 8 mmol) in toluene (25 mL) was stirred at 110° C. under Ar for 45 h. The reaction mixture was diluted with water (30 mL) and extracted with EtOAc (40 mL×2). The organic layer was washed with brine, dried with Na₂SO₄ and evaporated to give crude title compound. The residue was purified by C₁₈ column chromatography to give title compound (20 mg, 1.5% yield). ESI-MS: 492.2 [M+H].

### Step 6: 7-2,6-diazaspiro[3.3]heptan-2-yl-2-8-fluoro-2-methylimidazo[1,2-a]pyridin-6-yl-4H-pyrido[1,2-a][1,3,5]triazin-4-one trifluoroacetic acid

To a solution of tert-butyl 6-(2-8-fluoro-2-methylimidazo[1,2-a]pyridin-6-yl-4-oxo-4H-pyrido[1,2-a][1,3,5]triazin-7-yl)-2,6-diazaspiro[3.3]heptane-2-carboxylate (20.0 mg, 40.69 μmol) in DCM (1 mL) was added TFA (232.37 mg, 2.04 mmol, 160.0 μl, 50.0 equiv) at 0° C. The reaction mixture was warmed to rt and stirred for 15 h. After concentration, the residue was purified by prep-HPLC (MeCN/0.05% formic acid in water) to give title product 7-2,6-diazaspiro[3.3]heptan-2-yl-2-8-fluoro-2-methylimidazo[1,2-a]pyridin-6-yl-4H-pyrido[1,2-a][1,3,5]triazin-4-one; trifluoroacetic acid (6 mg; 29% yield). ESI-MS: 392.2 [M+H].

### Example 45. Synthesis of 7-[(3S)-3-ethylpiperazin-1-yl]-9-fluoro-2-{8-fluoro-2-methylimidazo[1,2-a]pyridin-6-yl}-4H-pyrido[1,2-a][1,3,5]triazin-4-one (Compound 128)

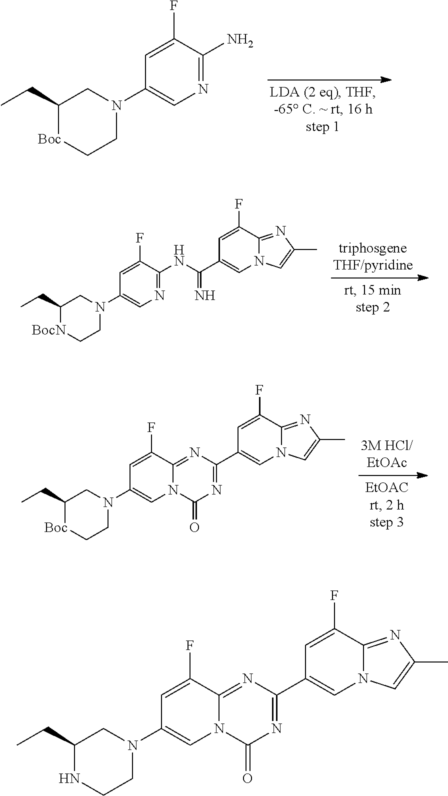

### Step 1: Preparation of tert-butyl (S)-2-ethyl-4-(5-fluoro-6-(8-fluoro-2-methylimidazo[1,2-a]pyridine-6-carboximidamido)pyridin-3-yl)piperazine-1-carboxylate

Fresh prepared LDA (2.16 mmol, THF) was added dropwise to a solution of tert-butyl (S)-4-(6-amino-5-fluoropyridin-3-yl)-2-ethylpiperazine-1-carboxylate (486 mg, 1.5 mmol) in THF (5 mL) at −60° C., the mixture was stirred for 1.5 h at rt. The mixture was cooled down to −60° C. again. Then 8-fluoro-2-methylimidazo[1,2-a]pyridine-6-carbonitrile (175 mg, 1 mmol) in THF (5 mL) was added slowly, the mixture was stirred overnight at rt. The reaction mixture was diluted with water (30 mL) and extracted with EtOAc (40 mL×3). The organic layer was washed with brine, dried with Na2SO4 and evaporated to give crude title compound. The residue was purified by C18 column chromatography eluted with MeCN: H2O=80% to 95% to give title compound (120 mg, Y: 42.1%) as a yellow solid. ESI-MS (M+H)⁺: 500.1.

### Step 2: Preparation of tert-butyl (S)-2-ethyl-4-(9-fluoro-2-(8-fluoro-2-methylimidazo[1,2-a]pyridin-6-yl)-4-oxo-4H-pyrido[1,2-af][1,3,5]triazin-7-yl)piperazine-1-carboxylate

To a solution of tert-butyl (S)-2-ethyl-4-(5-fluoro-6-(8-fluoro-2-methylimidazo[1,2-a]pyridine-6-carboximidamido)pyridin-3-yl)piperazine-1-carboxylate (100 mg, 0.20 mmol) in THE (50 mL) and pyridine (1.0 mL) was added triphosgene (178 mg, 0.60 mmol) at r.t, the mixture was stirred for 15 min at rt. The reaction mixture was diluted with water (500 mL) and extracted with EtOAc (40 mL×3). The organic layer was washed with brine, dried with Na₂SO₄ and evaporated to give crude title compound. The residue was treated with MeOH (10 mL), stirred at room temperature for 1 h. The precipitate was filtered to give title compound (70 mg, Y: 66.7%) as a yellow solid. ESI-MS (M+H)⁺:526.1.

### Step 3: Preparation of (S)-7-(3-ethylpiperazin-1-yl)-9-fluoro-2-(8-fluoro-2-methylimidazo[1,2-a]pyridin-6-yl)-4H-pyrido[1,2-a][1,3,5]triazin-4-one

To a solution of tert-butyl (S)-2-ethyl-4-(9-fluoro-2-(8-fluoro-2-methylimidazo[1,2-a]pyridin-6-yl)-4-oxo-4H-pyrido[1,2-a][1,3,5]triazin-7-yl)piperazine-1-carboxylate (50 mg, 0.095 mmol) in EtOAc (2 mL) was added 3M HCl/EtOAc (2 mL) at room temperature. The reaction mixture was stirred for 2 h. The mixture was concentrated in vacuo and sat. Na2CO3 (10 mL) was added. The resulting mixture was stirred at rt for 30 min, the precipitate was filtered and dried to give title compound (35 mg, 87.5%) as a yellow solid. ESI-MS (M+H)+:426.1. ¹H NMR (400 MHz, CDCl3) δ 9.22 (s, 1H), 8.32 (s, 1H), 7.96 (d, J=11.5 Hz, 1H), 7.68 (d, J=10.5 Hz, 1H), 7.49 (s, 1H), 3.58-3.57 (m, 2H), 3.22-3.21 (m, 1H), 3.02-2.93 (m, 2H), 2.88-2.72 (m, 1H), 2.57-2.56 (m, 1H), 2.50 (s, 3H), 1.57-1.49 (m, 2H), 1.04 (t, J=7.3 Hz, 3H).

### Example 46. Synthesis of 2-{2,7-dimethylpyrazolo[1,5-a]pyridin-5-yl}-9-methyl-7-[(3S)-3-methylpiperazin-1-yl]-4H-pyrido[1,2-a][1,3,5]triazin-4-one (Compound 129)

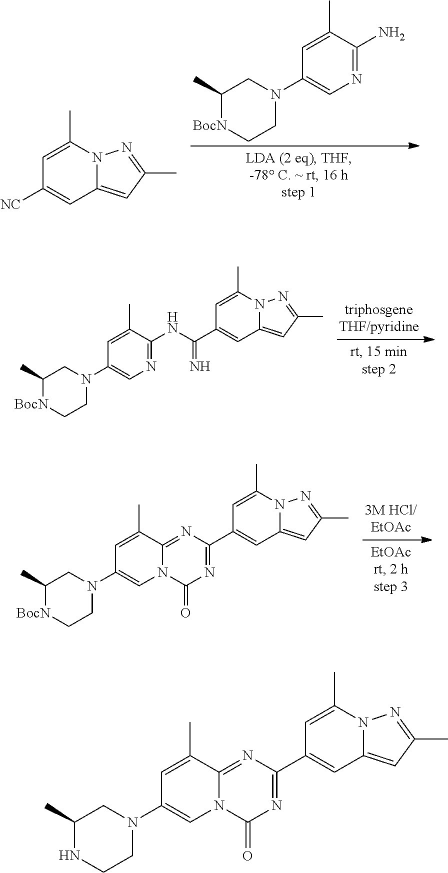

### Step 1: Preparation of tert-butyl (S)-4-(6-(2,7-dimethylpyrazolo[1,5-a]pyridine-5-carboximidamido)-5-methylpyridin-3-yl)-2-methylpiperazine-1-carboxylate

To a clean and dry flask was added DIPA (0.6 mL, 4.8 mmol) and THF(15 mL), the mixture was cooled down to −60° C., then 1.6 M n-Buli (3.3 mL, 5.28 mmol) was added dropwised at −60° C. under N₂ and stirred for 1 h. Tert-butyl (S)-4-(6-amino-5-methylpyridin-3-yl)-2-methylpiperazine-1-carboxylate (1.24 g, 4 mmol) in THF (10 mL) was added dropwise at −60° C., the mixture was stirred for 1.5 h at rt. The mixture was cooled down to −60° C. again, 2,7-dimethylpyrazolo[1,5-a]pyridine-5-carbonitrile (340 mg, 2 mmol) in THF (10 mL) was added slowly, the mixture was stirred overnight at rt. The reaction mixture was diluted with water (30 mL) and extracted with EtOAc (40 mL×3). The organic layer was washed with brine, dried with Na2SO4 and evaporated to give crude title compound. The residue was purified by C18 column chromatography eluted with MeCN: H2O=80% to 95% to give title compound (150 mg, Y: 15.8%) as a yellow solid. ESI-MS (M+H)+: 478.3.

### Step 2: Preparation of tert-butyl (S)-4-(2-(2,7-dimethylpyrazolo[1,5-a]pyridin-5-yl)-9-methyl-4-oxo-4H-pyrido[1,2-a][1,3,5]triazin-7-yl)-2-methylpiperazine-1-carboxylate

To a solution of tert-butyl (S)-4-(6-(2,7-dimethylpyrazolo[1,5-a]pyridine-5-carboximidamido)-5-methylpyridin-3-yl)-2-methylpiperazine-1-carboxylate (130 mg, 0.273 mmol) in THF (60 mL) and pyridine (1.5 mL) was added triphosgene (242 mg, 0.819 mmol) at r.t, the mixture was stirred for 15 min at rt. The reaction mixture was diluted with water (50 mL) and extracted with EtOAc (40 mL×3). The organic layer was washed with brine, dried with Na₂SO₄ and evaporated to give crude title compound. The residue was added MeOH (10 mL), stirred at room temperature for 1 h, the precipitate was filtered to give title compound (100 mg, Y: 65.7%) as a yellow solid. ¹H NMR (400 MHz, CDCl₃) δ 8.68 (s, 1H), 8.39 (d, J=2.7 Hz, 1H), 7.73-7.67 (m, 2H), 6.98 (s, 1H), 6.54 (s, 1H), 4.45-4.44 (m, 1H), 4.05-4.00 (m, 1H), 3.61-3.56 (m, 1H), 3.45-3.41 (m, 1H), 3.34-3.27 (m, 1H), 3.10-3.06 (m, 1H), 2.93-2.86 (m, 1H), 2.80 (s, 3H), 2.72 (s, 3H), 2.56 (s, 3H), 1.50 (s, 9H), 1.32 (d, J=6.7 Hz, 3H).

### Step 3: Preparation of (S)-2-(2,7-dimethylpyrazolo[1,5-a]pyridin-5-yl)-9-methyl-7-(3-methylpiperazin-1-yl)-4H-pyrido[1,2-a][1,3,5]triazin-4-one

To a solution of tert-butyl (S)-4-(2-(2,7-dimethylpyrazolo[1,5-a]pyridin-5-yl)-9-methyl-4-oxo-4H-pyrido[1,2-a][1,3,5]triazin-7-yl)-2-methylpiperazine-1-carboxylate (75 mg, 0.15 mmol) in EtOAc (2 mL) was added 3M HCl/EtOAc (3 ml) at room temperature. The reaction mixture was stirred for 2 h. The mixture was concentrated in vacuo, the residue was added sat. Na2CO3 (10 mL), stirred at rt for 30 min, the precipitate was filtered to give title compound (50 mg, 83.3%) as a yellow solid. ESI-MS (M+H)+:404.2. ¹H NMR (400 MHz, CDCl3) δ 8.68 (s, 1H), 8.40 (d, J=2.7 Hz, 1H), 7.77-7.71 (m, 2H), 6.53 (s, 1H), 3.61-3.54 (m, 2H), 3.19-3.17 (m, 1H), 3.08-2.97 (m, 2H), 2.86-2.85 (m, 1H), 2.81 (s, 3H), 2.71 (s, 3H), 2.56 (s, 3H), 2.51-2.47 (m, 1H), 1.18 (d, J=6.3 Hz, 3H).

### Example 47. Synthesis of 7-(3,3-dimethylpiperazin-1-yl)-9-fluoro-2-{8-fluoro-2-methylimidazo[1,2-a]pyridin-6-yl}-4H-pyrido[1,2-a][1,3,5]triazin-4-one (Compound 130)

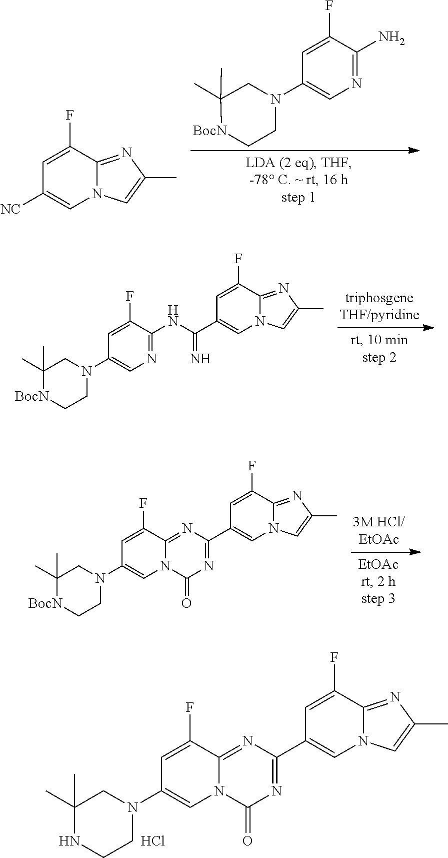

### Step 1: Preparation of tert-butyl 4-(5-fluoro-6-(8-fluoro-2-methylimidazo[1,2-a]pyridine-6-carboximidamido)pyridin-3-yl)-2,2-dimethylpiperazine-1-carboxylate

To a mixture of diisopropylamine (0.34 mL, 2.4 mmol) in dry·THF (10 mL) was added n-BuLi (1.15 mL, 2.88 mmol, 2.5 M) dropwise at −65° C. The mixture was stirred at this temperature for 1 hour, tert-butyl 4-(6-amino-5-fluoropyridin-3-yl)-2,2-dimethylpiperazine-1-carboxylate (620 mg, 2.0 mmol) in dry·THF (4 mL) was added. The mixture was allowed to warm to room temperature and stirred for 1 h. 8-fluoro-2-methyl-3,8a-dihydroimidazo[1,2-a]pyridine-6-carbonitrile (175 mg, 1.0 mmol) in dry·THF (4 mL) was added to the mixture at −65° C. and allowed to warm to room temperature and stirred for 16 h. LCMS showed the reaction was completed. The mixture was diluted with water (30 mL), extracted with EtOAc (80 mL×2). The combined organic layer was washed with brine (40 mL), dried over sodium sulfate, filtered and concentrated in vacuo. The residue was purified by C18-flash (0.1% NH₃·H2O in water/CH₃CN) to give title product (170 mg, Y: 33.9%) as a yellow solid. ESI-MS: [M+H]+:500.2.

### Step 2: Preparation of tert-butyl 4-(9-fluoro-2-(8-fluoro-2-methylimidazo[1,2-a]pyridin-6-yl)-4-oxo-4H-pyrido[1,2-af][1,3,5]triazin-7-yl)-2,2-dimethylpiperazine-1-carboxylate

To a solution of tert-butyl 4-(5-fluoro-6-(8-fluoro-2-methylimidazo[1,2-a]pyridine-6-carboximidamido)pyridin-3-yl)-2,2-dimethylpiperazine-1-carboxylate (170 mg, 0.339 mmol) in THE (18 mL) and pyridine (1.8 mL) was added triphosgene (322 mg, 1.08 mmol). The reaction mixture was stirred for 10 min at rt. LCMS showed the reaction was completed. The mixture was diluted with water (50 mL), extracted with EtOAc (100 mL*2). The combined organic layer was washed with brine (80 mL), dried over sodium sulfate, filtered and concentrated in vacuo. The residue was diluted with MeOH (30 mL), stirred for 1 hour, the precipitate was filtered and dried in vacuo to give title product (80 mg, 44.6%) as a yellow solid. ¹H NMR (400 MHz, CDCl₃) δ 9.21 (d, J=1.3 Hz, 1H), 8.23 (d, J=2.1 Hz, 1H), 7.97 (dd, J=11.6, 1.3 Hz, 1H), 7.56-7.46 (m, 2H), 3.91 (t, J=5.6 Hz, 2H), 3.51 (t, J=5.7 Hz, 2H), 3.44 (s, 2H), 2.50 (s, 3H), 1.51 (s, 9H), 1.47 (s, 6H). ESI-MS: [M+H]⁺:526.3.

### Step 3: 7-(3,3-dimethylpiperazin-1-yl)-9-fluoro-2-(8-fluoro-2-methylimidazo[1,2-a]pyridin-6-yl)-4H-pyrido[1,2-a][1,3,5]triazin-4-one hydrochloride

To a solution of tert-butyl 4-(9-fluoro-2-(8-fluoro-2-methylimidazo[1,2-a]pyridin-6-yl)-4-oxo-4H-pyrido[1,2-a][1,3,5]triazin-7-yl)-2,2-dimethylpiperazine-1-carboxylate (80 mg, 0.15 mmol) in EtOAC (2 mL) was added 3M HCl/EtOAc (3 mL) at room temperature. The reaction mixture was stirred for 2 h. The precipitate was filtered and lyophilizied to give title product (18 mg, yield: 28.1%) as a yellow solid. ¹H NMR (400 MHz, DMSO-d₆) δ 9.60 (d, J=1.0 Hz, 1H), 9.51 (s, 2H), 8.49 (dd, J=12.0, 2.4 Hz, 1H), 8.27 (d, J=2.1 Hz, 1H), 8.22 (s, 1H), 8.12 (d, J=11.9 Hz, 1H), 3.58 (d, J=5.2 Hz, 4H), 3.31 (s, 2H), 2.47 (s, 3H), 1.41 (s, 6H). ESI-MS: [M+H]+:426.1.

### Example 48. Synthesis of 2-{2,7-dimethylpyrazolo[1,5-a]pyridin-5-yl}-9-fluoro-7-[(3S)-3-methylpiperazin-1-yl]-4H-pyrido[1,2-a][1,3,5]triazin-4-one (Compound 131)

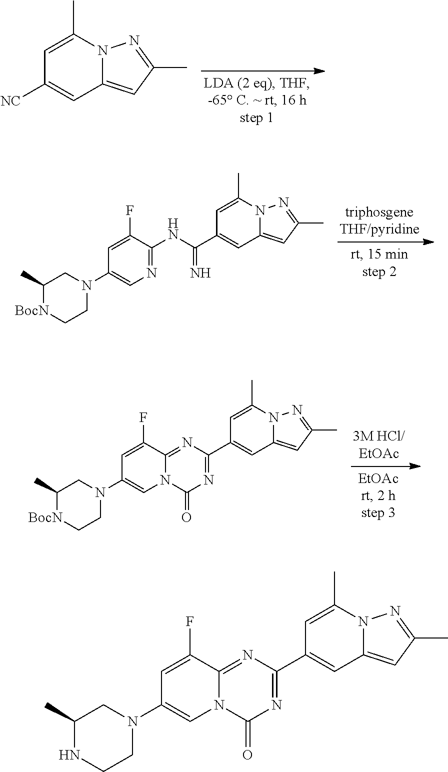

### Step 1: Preparation of tert-butyl (S)-4-(6-(2,7-dimethylpyrazolo[1,5-a]pyridine-5-carboximidamido)-5-fluoropyridin-3-yl)-2-methylpiperazine-1-carboxylate

Fresh prepared LDA was added to tert-butyl (S)-4-(6-amino-5-fluoropyridin-3-yl)-2-methylpiperazine-1-carboxylate (1.82 g, 5.84 mmol) in THF (50 mL) dropwised at −60° C., the mixture was stirred for 1.5 h at rt. The mixture was cool down to −60° C. again. Then 2,7-dimethylpyrazolo[1,5-a]pyridine-5-carbonitrile (500 mg, 2.92 mmol) in THF (10 mL) was added slowly, the mixture was stirred overnight at rt. The reaction mixture was diluted with water (30 mL) and extracted with EtOAc (460 mL×3). The organic layer was washed with brine, dried with Na2SO4 and evaporated to give crude title compound. The residue was purified by C18 column chromatography eluted with MeCN: H2O=80% to 95% to give title compound (200 mg, Y: 28%) as a yellow solid. ESI-MS (M+H)⁺: 482.2. ¹H NMR (400 MHz, CDCl3) δ 7.85 (d, J=1.3 Hz, 1H), 7.79 (d, J=2.6 Hz, 1H), 7.24 (s, 1H), 7.04 (dd, J=12.4, 2.7 Hz, 1H), 6.45 (s, 1H), 4.43-4.33 (m, 1H), 3.98 (d, J=13.3 Hz, 1H), 3.48 (d, J=11.8 Hz, 1H), 3.35-3.24 (m, 2H), 3.01 (dd, J=12.0, 3.8 Hz, 1H), 2.83 (dd, J=11.7, 3.6 Hz, 1H), 2.78 (s, 3H), 2.54 (s, 3H), 1.49 (s, 9H), 1.31 (d, J=6.7 Hz, 3H).

### Step 2: Preparation of tert-butyl (S)-4-(2-(2,7-dimethylpyrazolo[1,5-a]pyridin-5-yl)-9-fluoro-4-oxo-4H-pyrido[1,2-a][1,3,5]triazin-7-yl)-2-methylpiperazine-1-carboxylate

To a solution of tert-butyl (S)-4-(6-(2,7-dimethylpyrazolo[1,5-a]pyridine-5-carboximidamido)-5-fluoropyridin-3-yl)-2-methylpiperazine-1-carboxylate (150 mg, 0.312 mmol) in THE (60 mL) and pyridine(1.5 mL) was added triphosgene (277 mg, 0.936 mmol) at rt, the mixture was stirred for 15 min at rt. The reaction mixture was diluted with water (50 mL) and extracted with EtOAc (80 mL×3). The organic layer was washed with brine, dried with Na2SO4 and evaporated to give crude title compound. The residue was diluted with MeOH (10 mL) and stirred at room temperature for 1 h, the precipitate was filtered and dried to give title compound (180 mg, Y: 85.7%) as a yellow solid. ESI-MS (M+H)⁺:508.2. ¹H NMR (400 MHz, CDCl₃) δ 8.69 (d, J=1.4 Hz, 1H), 8.32 (d, J=1.8 Hz, 1H), 7.73-7.68 (m, 1H), 7.62 (dd, J=10.6, 2.6 Hz, 1H), 6.55 (s, 1H), 4.49-4.37 (m, 1H), 4.09-3.96 (m, 1H), 3.58 (d, J=12.1 Hz, 1H), 3.41 (d, J=12.3 Hz, 1H), 3.35-3.27 (m, 1H), 3.16 (dd, J=12.2, 3.9 Hz, 1H), 3.01-2.94 (m, 1H), 2.80 (s, 3H), 2.56 (s, 3H), 1.50 (s, 9H), 1.30 (d, J=6.7 Hz, 3H).

### Step 3: Preparation of (S)-2-(2,7-dimethylpyrazolo[1,5-a]pyridin-5-yl)-9-fluoro-7-(3-methylpiperazin-1-yl)-4H-pyrido[1,2-a][1,3,5]triazin-4-one

To a solution of tert-butyl (S)-4-(2-(2,7-dimethylpyrazolo[1,5-a]pyridin-5-yl)-9-fluoro-4-oxo-4H-pyrido[1,2-a][1,3,5]triazin-7-yl)-2-methylpiperazine-1-carboxylate (180 mg, 0.35 mmol) in EtOAc (10 mL) was added 3M HCl/EtOAc (15 ml) at room temperature. The reaction mixture was stirred for 2 h. The mixture was concentrated in vacuo, and sat. Na2CO3 (10 mL) was added, stirred at rt for 30 min, the precipitate was filtered to give title compound (100 mg, 69%) as a yellow solid. ESI-MS (M+H)⁺:408.1. ¹H NMR (400 MHz, CDCl₃) δ 8.68 (s, 1H), 8.32 (d, J=1.9 Hz, 1H), 7.71 (s, 1H), 7.66 (dd, J=10.9, 2.6 Hz, 1H), 6.54 (s, 1H), 3.55 (t, J=11.4 Hz, 2H), 3.20 (d, J=12.0 Hz, 1H), 3.08-2.95 (m, 2H), 2.92 (dd, J=11.4, 3.1 Hz, 1H), 2.80 (s, 3H), 2.56 (s, 3H), 2.54-2.51 (m, 1H), 1.19 (d, J=6.3 Hz, 3H).

### Example 49. Synthesis of 7-{4,7-diazaspiro[2.5]octan-7-yl}-2-{8-fluoro-2-methylimidazo[1,2-a]pyridin-6-yl}-9-methyl-4H-pyrido[1,2-a][1,3,5]triazin-4-one (Compound 132)

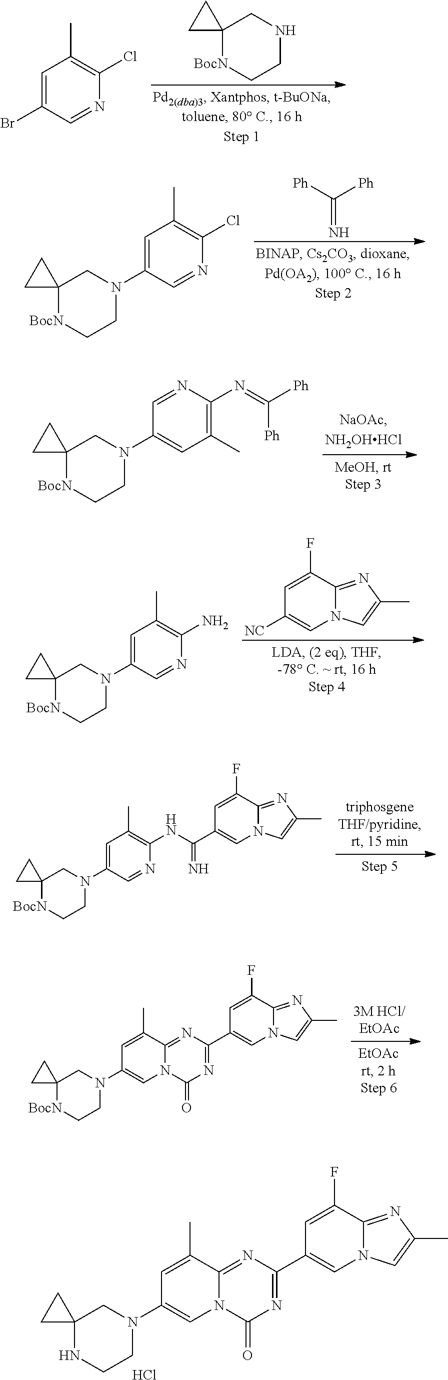

### Step 1: Preparation of tert-butyl 7-(6-chloro-5-methylpyridin-3-yl)-4,7-diazaspiro[2.5]octane-4-carboxylate

To a mixture of 5-bromo-2-chloro-3-methylpyridine (1 g, 4.854 mmol), tert-butyl 4,7-diazaspiro[2.5]octane-4-carboxylate (926 mg, 4.36 mmol) in toluene (20 mL) was added t-BuONa (932 mg, 9.708 mmol), Xantphos (561 mg, 0.9708 mmol) and Pd2(dba)3 (445 mg, 0.485 mmol), the mixture was charged with N₂ for three times and stirred at 80° C. for 16 h. The reaction mixture was diluted with water (100 mL) and extracted with EtOAc (100 mL×3). The organic layer was washed with brine, dried over Na2SO4 and evaporated to give crude title compound. The crude was purified silica gel column chromatography eluted with (EtOAc:PE=1:5) to give title compound (0.9 g, 55% yield) as a grey solid. ESI-MS (M+H)+:338.2

### Step 2: Preparation of tert-butyl 7-(6-((diphenylmethylene)amino)-5-methylpyridin-3-yl)-4,7-diazaspiro[2.5]octane-4-carboxylate

To a mixture of tert-butyl 7-(6-chloro-5-methylpyridin-3-yl)-4,7-diazaspiro[2.5]octane-4-carboxylate (0.9 g, 2.670 mmol), diphenylmethanimine (507 mg, 2.804 mmol) in 1,4-dioxane (40 mL) was added Cs2CO3 (1.74 g, 5.34 mol), BINAP (332 mg, 0.534 mmol) and Pd(OAc)2 (60 mg, 0.267 mmol), the mixture was charged with N2 for three times and stirred at 100° C. for 16 h. The reaction mixture was diluted with water (40 mL) and extracted with EtOAc (40 mL×3). The organic layer was washed with brine, dried over Na2SO4 and evaporated to give crude title compound. The crude was purified by silica gel column chromatography eluted with (EtOAc/PE=4:1) to give title compound (1.2 g, 93% yield) as a brown solid. ESI-MS (M+H)⁺:483.3.

### Step 3: Preparation of tert-butyl 7-(6-amino-5-methylpyridin-3-yl)-4,7-diazaspiro[2.5]octane-4-carboxylate

To a solution of tert-butyl 7-(6-((diphenylmethylene)amino)-5-methylpyridin-3-yl)-4,7-diazaspiro[2.5]octane-4-carboxylate. (1.2 g, 2.489 mmol) in MeOH (20 mL) was added NaOAc (612 mg, 7.469 mmol) and hydroxylamine hydrochloride (858 mg, 12.445 mmol) at RT, the mixture was stirred for 1 h at RT. The mixture was diluted with water (20 mL), acidized to pH=2 by 1N HCl, extracted with EtOAc (100 mL×2). Aqueous phase was based with NaHCO₃(aq) to pH=9, extracted with EtOAc (60 mL×3), the combined organic layers were washed with brine, dried over Na₂SO₄, and filtered filtrate were concentrated to afford title compound (600 mg, 75.8% yield) as a grey solid. ¹H NMR (400 MHz, CDCl₃) δ 7.59 (d, J=2.7 Hz, 1H), 7.03-6.94 (m, 1H), 4.17-4.11 (m, 2H), 3.72-3.67 (m, 2H), 3.03-2.99 (m, 2H), 2.81 (s, 2H), 2.11 (s, 3H), 1.47 (s, 9H), 1.09-1.00 (m, 2H), 0.88-0.82 (m, 2H). ESI-MS (M+H)⁺: 319.2.

### Step 4: Preparation of tert-butyl 7-(6-(8-fluoro-2-methylimidazo[1,2-a]pyridine-6-carboximidamido)-5-methylpyridin-3-yl)-4,7-diazaspiro[2.5]octane-4-carboxylate

Fresh prepared LDA was added to a solution of 6-amino-5-methylpyridin-3-yl)-4,7-diazaspiro[2.5]octane-4-carboxylate (1 g, 3 mmol) in THF (5 mL) dropwised at −60° C., the mixture was stirred for 1.5 at RT. The mixture was cool down to −60° C. Then 8-fluoro-2-methylimidazo[1,2-a]pyridine-6-carbonitrile (350 mg, 2 mmol) in THE (5 mL) was added slowly. The mixture was stirred overnight at RT. The reaction mixture was diluted with water (20 mL) and extracted with EtOAc (5 mL×3). The organic layer was washed with brine, dried with Na2SO4 and evaporated to give crude title compound. The residue was purified by C18 column chromatography eluted with MeCN: H2O=0% to 100% to give title compound (130 mg, 13.1% yield) as an off-white solid. ¹H NMR (400 MHz, CDCl₃) δ 8.55-8.53 (m, 1H), 7.81-7.77 (m, 1H), 7.48-7.43 (m, 2H), 7.14-7.11 (m, 1H), 3.76-3.71 (m, 2H), 3.19-3.15 (m, 2H), 2.98-2.95 (m, 2H), 2.50 (s, 3H), 2.46 (s, 3H), 1.48 (s, 9H), 1.11-1.07 (m, 2H), 0.89-0.86 (m, 2H). ESI-MS (M+H)+:494.2.

### Step 5: Preparation of tert-butyl 7-(2-(8-fluoro-2-methylimidazo[1,2-a]pyridin-6-yl)-9-methyl-4-oxo-4H-pyrido[1,2-a][1,3,5]triazin-7-yl)-4,7-diazaspiro[2.5]octane-4-carboxylate

To a solution of t-butyl 7-(6-(8-fluoro-2-methylimidazo[1,2-a]pyridine-6-carboximidamido)-5-methylpyridin-3-yl)-4,7-diazaspiro[2.5]octane-4-carboxylate (130 mg, 0.15 mmol) in THE (30 mL) and pyridine (3 mL) was added triphosgene (234 mg, 0.791 mmol), the mixture was stirred at rt for 15 min. The reaction mixture was diluted with water (30 mL) and extracted with EtOAc (40 mL×3). The organic layer was washed with brine, dried with Na2SO4 and evaporated to give crude title compound. The residue was purified by pre-HPLC (0.1% NH3·H2O in water/CH₃CN) to afford title compound (60 mg, 44.1% yield) as an off-white solid. ¹H NMR (400 MHz, CDCl₃) δ 9.22 (s, 1H), 8.34 (d, J=2.6 Hz, 1H), 7.96 (d, J=11.6 Hz, 1H), 7.72 (s, 1H), 7.48 (d, J=2.7 Hz, 1H), 3.78-3.73 (m, 2H), 3.31-3.26 (m, 2H), 3.06 (s, 2H), 2.69 (s, 3H), 2.50 (s, 3H), 1.49 (s, 9H), 1.14-1.10 (m, 2H), 0.90-0.87 (m, 2H). ESI-MS (M+H)+:520.1.

### Step 6: Preparation of 2-(8-fluoro-2-methylimidazo[1,2-a]pyridin-6-yl)-9-methyl-7-(4,7-diazaspiro[2.5]octan-7-yl)-4H-pyrido[1,2-a][1,3,5]triazin-4-one HCl salt

A solution of tert-butyl 7-(2-(8-fluoro-2-methylimidazo[1,2-a]pyridin-6-yl)-9-methyl-4-oxo-4H-pyrido[1,2-a][1,3,5]triazin-7-yl)-4,7-diazaspiro[2.5]octane-4-carboxylate (60 mg, 0.116 mmol) in 3M HCl/EtOAc (4 mL) was stirred for 2 h at RT. The mixture was filtered and the filter cake was washed with EtOAc (2 mL) and dried to afford title compound (30 mg, 60% yield) as a yellow solid. ESI-MS (M+H)⁺:420.1. ¹H NMR (400 MHz, MeOD-d4) δ 9.69-9.66 (m, 1H), 8.67-8.63 (m, 1H), 8.49-8.46 (m, 1H), 8.33-8.27 (m, 2H), 3.77-3.71 (m, 2H), 3.61-3.57 (m, 4H), 2.76 (s, 3H), 2.63 (s, 3H), 1.25-1.20 (m, 2H), 1.18-1.14 (m, 2H).

### Example 49. Synthesis of (S)-2-(7-fluoro-2-methyl-2H-indazol-5-yl)-7-(3-methylpiperazin-1-yl)-4H-pyrido[1,2-a][1,3,5]triazin-4-one hydrochloride (Compound 133)

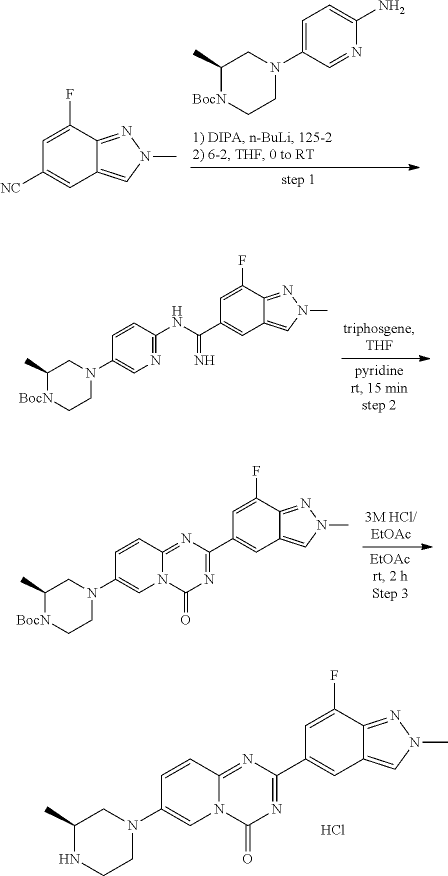

### Step 1: tert-butyl (S)-4-(6-(7-fluoro-2-methyl-2H-indazole-5-carboximidamido)pyridin-3-yl)-2-methylpiperazine-1-carboxylate

To a solution of fresh prepared LDA (2.625 mmol) in dry THE (10 mL) was added dropwise at −65° C. a solution of tert-butyl (S)-4-(6-aminopyridin-3-yl)-2-methylpiperazine-1-carboxylate (523 mg, 1.79 mmol) in dry THE (4 mL). The mixture was allowed to warm to room temperature and stirred for 1 h. 7-fluoro-2-methyl-2H-indazole-5-carbonitrile (210 mg, 1.193 mmol) in dry·THF (4 mL) was added to the mixture at −65° C. and allowed to warm to room temperature and stirred for 16 h. The mixture was diluted with water (80 mL) and extracted with EtOAc (80 mL*2). The combined organic layer was washed with brine (40 mL), dried over sodium sulfate, filtered and concentrated in vacuo. The residue was purified by C18-flash (0.1% NH₃·H₂O in water/CH₃CN) to give tert-butyl (S)-4-(6-(7-fluoro-2-methyl-2H-indazole-5-carboximidamido)pyridin-3-yl)-2-methylpiperazine-1-carboxylate (40 mg, Y: 7.1%) as a yellow solid. ¹H NMR (400 MHz, CDCl₃) δ 8.03 (d, J=2.5 Hz, 1H), 7.98-7.95 (m, 2H), 7.62 (d, J=12.4 Hz, 1H), 7.30 (dd, J=8.9, 3.0 Hz, 1H), 7.25 (s, 1H), 4.37 (br.s, 1H), 4.27 (s, 3H), 3.98 (d, J=13.4 Hz, 1H), 3.46 (d, J=11.9 Hz, 1H), 3.35-3.23 (m, 2H), 2.95 (dd, J=11.9, 3.8 Hz, 1H), 2.81-2.73 (m, 1H), 1.49 (s, 9H), 1.33 (d, J=6.7 Hz, 3H). ESI-MS: [M+H]⁺: 468.3.

### Step 2: tert-butyl (S)-4-(2-(7-fluoro-2-methyl-2H-indazol-5-yl)-4-oxo-4H-pyrido[1,2-a][1,3,5]triazin-7-yl)-2-methylpiperazine-1-carboxylate

To a solution of tert-butyl (S)-4-(6-(7-fluoro-2-methyl-2H-indazole-5-carboximidamido)pyridin-3-yl)-2-methylpiperazine-1-carboxylate (40 mg, 0.085 mmol) in THE (35 mL) and pyridine (3 mL) was added triphosgene (76 mg, 0.257 mmol). The reaction mixture was stirred for 15 min at rt. The mixture was diluted with water (25 mL) and extracted with EtOAc (50 mL*2). The combined organic layer was washed with brine (40 mL), dried over sodium sulfate, filtered and concentrated in vacuo. The residue was diluted with MeOH (40 mL) and stirred for 1 hour at rt. The precipitate was filtered and dried in vacuo to give tert-butyl (S)-4-(2-(7-fluoro-2-methyl-2H-indazol-5-yl)-4-oxo-4H-pyrido[1,2-a][1,3,5]triazin-7-yl)-2-methylpiperazine-1-carboxylate (30 mg, 71.1%) as a brown solid. ESI-MS: [M+H]+:494.6. ¹H NMR (400 MHz, CDCl₃) δ 8.82 (d, J=0.9 Hz, 1H), 8.45 (d, J=2.7 Hz, 1H), 8.12 (dd, J=16.0, 1.8 Hz, 2H), 7.85 (dd, J=9.6, 2.8 Hz, 1H), 7.65 (d, J=9.6 Hz, 1H), 4.44 (br.s, 1H), 4.28 (s, 3H), 4.03 (d, J=13.2 Hz, 1H), 3.59 (d, J=11.4 Hz, 1H), 3.43 (d, J=12.0 Hz, 1H), 3.37-3.27 (m, 1H), 3.11 (dd, J=12.1, 3.8 Hz, 1H), 2.98-2.88 (m, 1H), 1.50 (s, 9H), 1.32 (d, J=6.7 Hz, 3H).

### Step 3: (S)-2-(7-fluoro-2-methyl-2H-indazol-5-yl)-7-(3-methylpiperazin-1-yl)-4H-pyrido[1,2-a][1,3,5]triazin-4-one hydrochloride

To a solution of tert-butyl (S)-4-(2-(7-fluoro-2-methyl-2H-indazol-5-yl)-4-oxo-4H-pyrido[1,2-a][1,3,5]triazin-7-yl)-2-methylpiperazine-1-carboxylate (30 mg, 0.061 mmol) in EtOAC (2 mL) was added 3M HCl/EtOAc (5 ml) at room temperature. The reaction mixture was stirred for 2 h. The precipitate was filtered and dried to give (S)-2-(7-fluoro-2-methyl-2H-indazol-5-yl)-7-(3-methylpiperazin-1-yl)-4H-pyrido[1,2-a][1,3,5]triazin-4-one hydrochloride (20 mg, yield: 83.7%) as a yellow solid. ¹H NMR (400 MHz, DMSO-d₆) δ 9.63 (s, 1H), 9.42 (s, 1H), 8.79-8.67 (m, 2H), 8.35 (d, J=7.4 Hz, 2H), 7.96 (d, J=13.4 Hz, 1H), 7.80 (d, J=10.3 Hz, 1H), 4.25 (s, 3H), 4.00-3.88 (m, 2H), 3.43 (d, J=9.4 Hz, 2H), 3.25-3.12 (m, 2H), 3.02-2.93 (m, 1H), 1.35 (d, J=6.5 Hz, 3H). ESI-MS: [M+H]⁺: 394.0.

### Example 50. Synthesis of (S)-2-(2,7-dimethylpyrazolo[1,5-a]pyridin-5-yl)-7-(3-methylpiperazin-1-yl)-4H-pyrido[1,2-a][1,3,5]triazin-4-one (Compound 134)

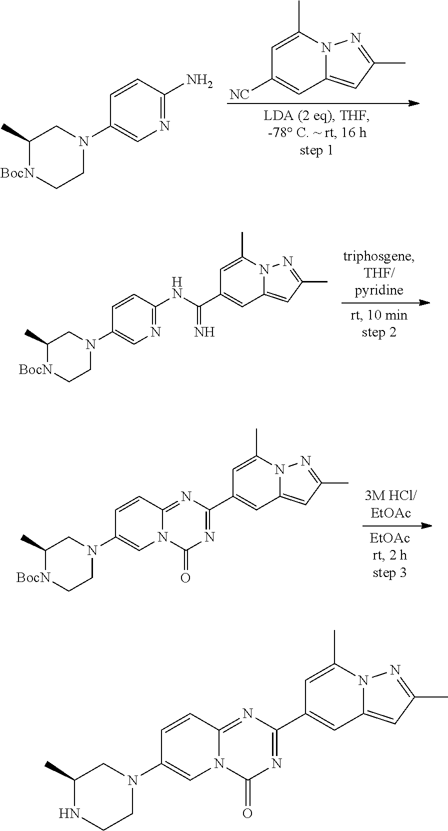

### Step 1: tert-butyl (S)-4-(6-(7-fluoro-2-methyl-2H-indazole-5-carboximidamido)pyridin-3-yl)-2-methylpiperazine-1-carboxylate

To a solution of fresh prepared LDA (2.2 mmol) in dry THF (10 mL) was added dropwise at −65° C. a solution of tert-butyl (S)-4-(6-aminopyridin-3-yl)-2-methylpiperazine-1-carboxylate (438 mg, 1.5 mmol) in dry THF (4 mL). The mixture was allowed to warm to room temperature and stirred for 1 h. 2,7-dimethylpyrazolo[1,5-a]pyridine-5-carbonitrile (171 mg, 1.0 mmol) in dry THF (4 mL) was added to the mixture at −65° C. and allowed to warm to room temperature and stirred for 16 h. The mixture was diluted with water (30 mL), extracted with EtOAc (30 mL*2). The combined organic layer was washed with brine (40 mL), dried over sodium sulfate, filtered and concentrated in vacuo. The residue was purified by C18-flash (0.1% NH3·H2O in water/CH3CN) to give tert-butyl (S)-4-(6-(7-fluoro-2-methyl-2H-indazole-5-carboximidamido)pyridin-3-yl)-2-methylpiperazine-1-carboxylate (120 mg, Y: 17.3%) as a yellow solid. ¹H NMR (400 MHz, DMSO-d6) δ 8.11 (d, J=1.2 Hz, 1H), 8.02 (d, J=3.1 Hz, 1H), 7.46-7.38 (m, 2H), 7.10 (d, J=8.9 Hz, 1H), 6.58 (s, 1H), 4.22-4.21 (m, 1H), 3.82-3.81 (m, 1H), 3.58-3.57 (m, 1H), 3.49-3.48 (m, 1H), 3.23-3.12 (m, 1H), 2.86-2.80 (m, 1H), 2.68 (d, J=5.3 Hz, 3H), 2.67-2.60 (m, 1H), 2.44 (s, 3H), 1.43 (s, 9H), 1.22 (d, J=6.7 Hz, 3H). ESI-MS: [M+H]⁺:464.1.

### Step 2: tert-butyl (S)-4-(2-(2,7-dimethylpyrazolo[1,5-a]pyridin-5-yl)-4-oxo-4H-pyrido[1,2-a][1,3,5]triazin-7-yl)-2-methylpiperazine-1-carboxylate

To a solution of tert-butyl (S)-4-(6-(7-fluoro-2-methyl-2H-indazole-5-carboximidamido)pyridin-3-yl)-2-methylpiperazine-1-carboxylate (120 mg, 0.259 mmol) in THE (10 mL) and pyridine (1.5 mL) was added triphosgene (231 mg, 0.778 mmol). The reaction mixture was stirred for 10 min at rt. The mixture was diluted with water (25 mL), extracted with EtOAc (15 mL*2). The combined organic layer was washed with brine (40 mL), dried over sodium sulfate, filtered and concentrated in vacuo. The residue was diluted with MeOH (30 mL), stirred for 1 hour at rt. The precipitate was filtered and dried in vacuo to give title product (65 mg, 51.3%) as a yellow solid. ESI-MS: [M+H]⁺:490.2. ¹H NMR (400 MHz, DMSO-d₆) δ 8.54 (d, J=1.2 Hz, 1H), 8.33-8.25 (m, 2H), 7.75 (d, J=9.5 Hz, 1H), 7.64-7.61 (m, 1H), 6.69 (s, 1H), 4.27-4.26 (m, 1H), 3.86-3.85 (m, 1H), 3.72-3.71 (m, 1H), 3.65-3.64 (, 1H), 3.26-3.20 (m, 1H), 3.11-3.04 (m, 1H), 2.91-2.82 (m, 1H), 2.72 (s, 3H), 2.46 (s, 3H), 1.44 (s, 9H), 1.21 (d, J=6.7 Hz, 3H).

### Step 3: (S)-2-(2,7-dimethylpyrazolo[1,5-a]pyridin-5-yl)-7-(3-methylpiperazin-1-yl)-4H-pyrido[1,2-a][1,3,5]triazin-4-one

To a solution of tert-butyl (S)-4-(2-(2,7-dimethylpyrazolo[1,5-a]pyridin-5-yl)-4-oxo-4H-pyrido[1,2-a][1,3,5]triazin-7-yl)-2-methylpiperazine-1-carboxylate (65 mg, 0.133 mmol) in EtOAC (2 mL) was added 3M HCl/EtOAc (3 ml) at room temperature. The reaction mixture was stirred for 2 h. The mixture was concentrated in vacuo, to the residue was added sat. NaHCO3 (5 mL), stirred for 30 min. The precipitate was filtered and lyophilizied to give title compound (21 mg, yield: 40.6%) as a yellow solid. ¹H NMR (400 MHz, DMSO-d6) δ 8.55 (s, 1H), 8.34 (d, J=9.5 Hz, 1H), 8.25 (s, 1H), 7.73 (d, J=9.6 Hz, 1H), 7.64 (s, 1H), 6.70 (s, 1H), 3.66-3.65 (m, 2H), 3.02-3.01 (m, 1H), 2.87-2.77 (m, 2H), 2.73 (s, 3H), 2.46 (s, 3H), 2.41-2.25 (m, 2H), 1.07 (d, J=6.3 Hz, 3H). ESI-MS: [M+H]⁺:390.1

### Example 51. Synthesis of 7-(3,3-dimethylpiperazin-1-yl)-2-(2,7-dimethylpyrazolo[1,5-a]pyridin-5-yl)-9-fluoro-4H-pyrido[1,2-a][1,3,5]triazin-4-one (Compound 135)

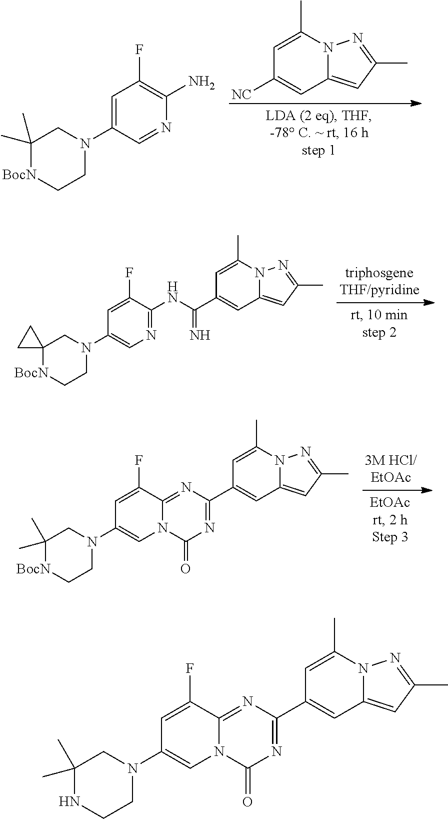

### Step 1: tert-butyl 4-(6-(2,7-dimethylpyrazolo[1,5-a]pyridine-5-carboximidamido)-5-fluoropyridin-3-yl)-2,2-dimethylpiperazine-1-carboxylate

To a solution of fresh prepared LDA (2.2 mmol) in dry THF (10 mL) was added tert-butyl 4-(6-amino-5-fluoropyridin-3-yl)-2,2-dimethylpiperazine-1-carboxylate (486 mg, 1.5 mmol) in dry THF (4 mL) at 0° C. The mixture was allowed to warm to room temperature and stirred for 1 h. 2,7-dimethylpyrazolo[1,5-a]pyridine-5-carbonitrile (171 mg, 1.0 mmol) in dry·THF (4 mL) was added to the mixture at −65° C. and allowed to warm to room temperature and stirred for 16 h. The mixture was diluted with water (30 mL), extracted with EtOAc (30 mL*2). The combined organic layer was washed with brine (40 mL), dried over sodium sulfate, filtered and concentrated in vacuo. The residue was purified by C18-flash (0.1% NH₃·H₂O in water/CH₃CN) to give tert-butyl 4-(6-(2,7-dimethylpyrazolo[1,5-a]pyridine-5-carboximidamido)-5-fluoropyridin-3-yl)-2,2-dimethylpiperazine-1-carboxylate (50 mg, Y: 10.1%) as a yellow solid. ¹H NMR (400 MHz, CDCl₃) δ 7.84 (d, J=1.4 Hz, 1H), 7.66 (d, J=2.7 Hz, 1H), 7.25 (d, J=0.8 Hz, 1H), 6.90-6.85 (m, 1H), 6.44 (s, 1H), 3.86-3.80 (m, 2H), 3.44-3.39 (m, 2H), 3.32 (s, 2H), 2.78 (s, 3H), 2.54 (s, 3H), 1.50 (s, 9H), 1.44 (s, 6H). ESI-MS: [M+H]⁺:496.1.

### Step 2: tert-butyl 4-(2-(2,7-dimethylpyrazolo[1,5-a]pyridin-5-yl)-9-fluoro-4-oxo-4H-pyrido[1,2-a][1,3,5]triazin-7-yl)-2,2-dimethylpiperazine-1-carboxylate

To a solution of tert-butyl 4-(6-(2,7-dimethylpyrazolo[1,5-a]pyridine-5-carboximidamido)-5-fluoropyridin-3-yl)-2,2-dimethylpiperazine-1-carboxylate (90 mg, 0.182 mmol) in THE (10 mL) and pyridine (1.5 mL) was added triphosgene (162 mg, 0.545 mmol). The reaction mixture was stirred for 10 min at rt. The mixture was diluted with water (25 mL), extracted with EtOAc (15 mL*2). The combined organic layer was washed with brine (40 mL), dried over sodium sulfate, filtered and concentrated in vacuo. The residue was diluted with MeOH (30 mL), stirred for 1 hour. The precipitate was filtered and dried in vacuo to give title product (60 mg, 63.4%) as a yellow solid. ESI-MS: [M+H]⁺:522.1. ¹H NMR (400 MHz, CDCl₃) δ 8.67 (d, J=1.2 Hz, 1H), 8.23 (d, J=2.1 Hz, 1H), 7.71 (s, 1H), 7.52 (dd, J=10.8, 2.7 Hz, 1H), 6.54 (s, 1H), 3.93-3.88 (m, 2H), 3.53-3.48 (m, 2H), 3.42 (s, 2H), 2.80 (s, 3H), 2.56 (s, 3H), 1.51 (s, 9H), 1.47 (s, 6H).

### Step 3: 7-(3,3-dimethylpiperazin-1-yl)-2-(2,7-dimethylpyrazolo[1,5-a]pyridin-5-yl)-9-fluoro-4H-pyrido[1,2-a][1,3,5]triazin-4-one

To a solution of tert-butyl 4-(2-(2,7-dimethylpyrazolo[1,5-a]pyridin-5-yl)-9-fluoro-4-oxo-4H-pyrido[1,2-a][1,3,5]triazin-7-yl)-2,2-dimethylpiperazine-1-carboxylate (60 mg, 0.115 mmol) in EtOAC (2 mL) was added 3M HCl/EtOAc (3 ml) at room temperature. The reaction mixture was stirred for 2 h. The mixture was concentrated in vacuo, to the residue was added sat. NaHCO₃ (5 mL), stirred for 30 min. The precipitate was filtered and lyophilized to give title product (19 mg, yield: 39.2%) as a yellow solid. ¹H NMR (400 MHz, DMSO-d₆) δ 8.54 (s, 1H), 8.43-8.37 (m, 1H), 8.09 (s, 1H), 7.61 (s, 1H), 6.71 (s, 1H), 3.19-3.14 (m, 2H), 3.03 (s, 2H), 2.93-2.87 (m, 2H), 2.73 (s, 3H), 2.46 (s, 3H), 1.12 (s, 6H). ESI-MS: [M+H]+: 422.1.

TABLE 1

| Com- | |
|---|---|
| pound | |
| No. | Structure |
| 99 | 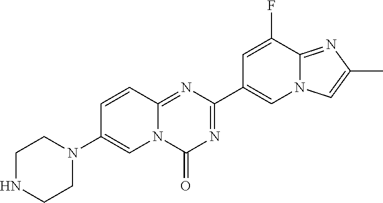 [SMILES: Cc1cn2cc(-c3nc(=O)n4cc(N5CCNCC5)ccc4n3)cc(F)c2n1] |
| 100 | 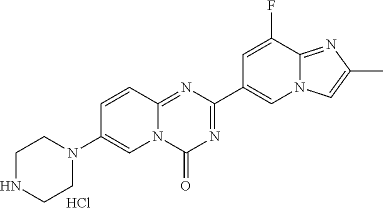 [SMILES: Cc1cn2cc(-c3nc(=O)n4cc(N5CCNCC5)ccc4n3)cc(F)c2n1 ; Cl] |
| 101 | 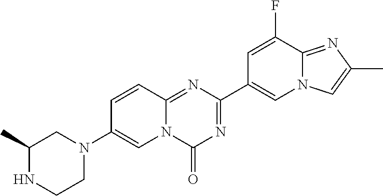 |
| 102 | 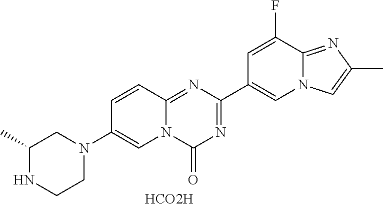 |
| 103 | 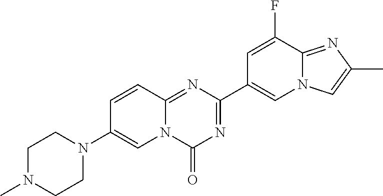 |
| 104 | 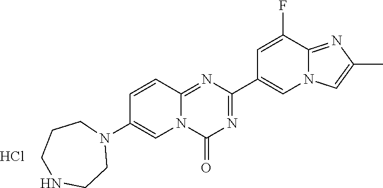 [SMILES: Cl ; Cc1cn2cc(-c3nc(=O)n4cc(N5CCCNCC5)ccc4n3)cc(F)c2n1] |
| 105 | 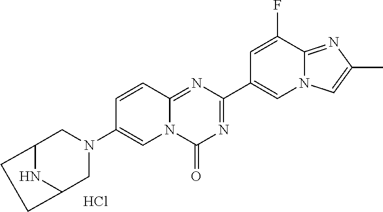 [SMILES: Cc1cn2cc(-c3nc(=O)n4cc(N5CC6CCC(C5)N6)ccc4n3)cc(F)c2n1 ; Cl] |
| 106 | 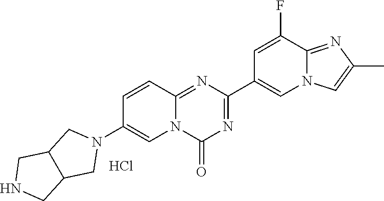 [SMILES: Cc1cn2cc(-c3nc(=O)n4cc(N5CC6CNCC6C5)ccc4n3)cc(F)c2n1 ; Cl] |
| 107 | 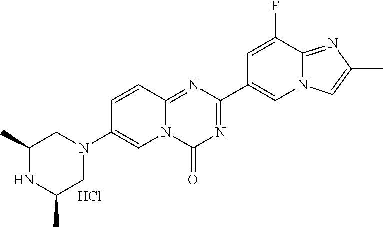 |
| 108 | 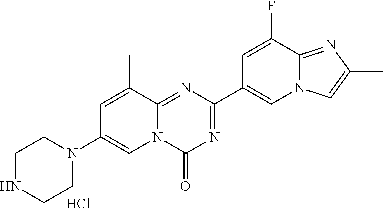 [SMILES: Cc1cn2cc(-c3nc(=O)n4cc(N5CCNCC5)cc(C)c4n3)cc(F)c2n1 ; Cl] |
| 109 | 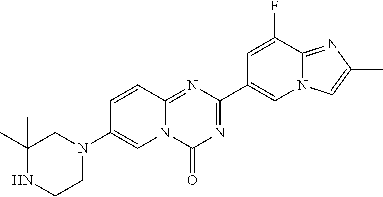 |
| 110 | 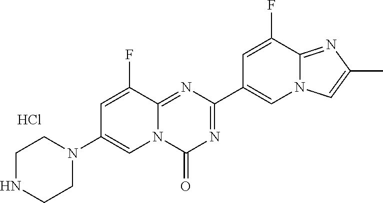 [SMILES: Cc1cn2cc(-c3nc(=O)n4cc(N5CCNCC5)cc(F)c4n3)cc(F)c2n1 ; Cl] |
| 111 | 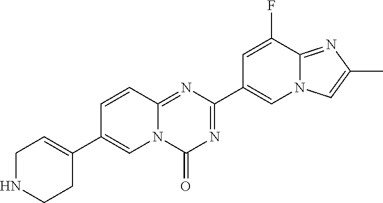 [SMILES: Cc1cn2cc(-c3nc(=O)n4cc(C5=CCNCC5)ccc4n3)cc(F)c2n1] |
| 112 | 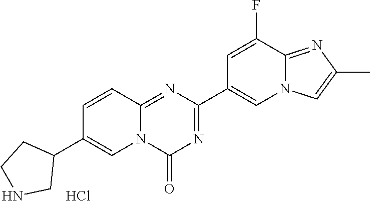 [SMILES: Cc1cn2cc(-c3nc(=O)n4cc(C5CCNC5)ccc4n3)cc(F)c2n1 ; Cl] |
| 113 | 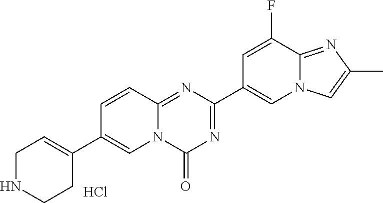 [SMILES: Cc1cn2cc(-c3nc(=O)n4cc(C5=CCNCC5)ccc4n3)cc(F)c2n1 ; Cl] |
| 114 | 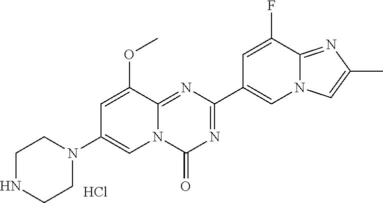 [SMILES: COc1cc(N2CCNCC2)cn2c(=O)nc(-c3cc(F)c4nc(C)cn4c3)nc12 ; Cl] |
| 115 | 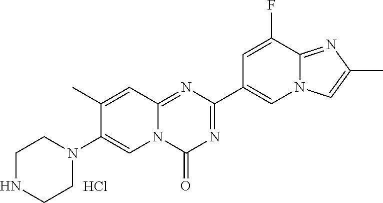 [SMILES: Cc1cn2cc(-c3nc(=O)n4cc(N5CCNCC5)c(C)cc4n3)cc(F)c2n1 ; Cl] |
| 116 | 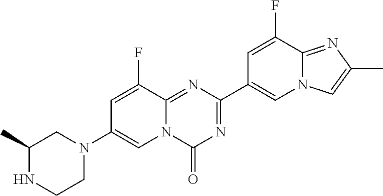 |
| 117 | 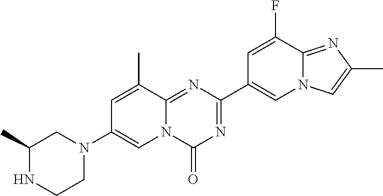 |
| 118 | 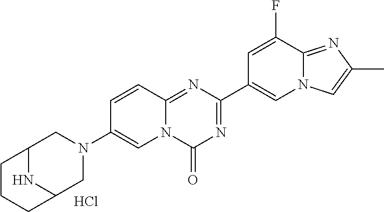 [SMILES: Cc1cn2cc(-c3nc(=O)n4cc(N5CC6CCCC(C5)N6)ccc4n3)cc(F)c2n1 ; Cl] |
| 119 | 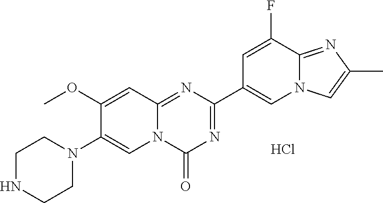 [SMILES: COc1cc2nc(-c3cc(F)c4nc(C)cn4c3)nc(=O)n2cc1N1CCNCC1 ; Cl] |
| 120 | 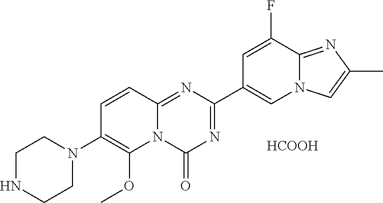 [SMILES: COc1c(N2CCNCC2)ccc2nc(-c3cc(F)c4nc(C)cn4c3)nc(=O)n12 ; "HCOOH"] |
| 121 | 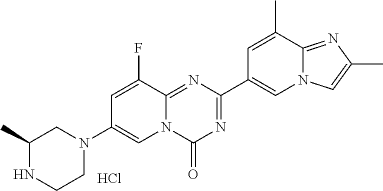 |
| 122 | 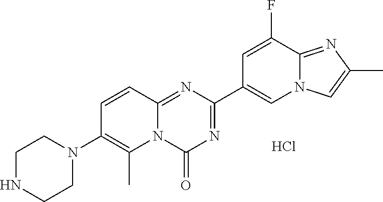 [SMILES: Cc1cn2cc(-c3nc(=O)n4c(C)c(N5CCNCC5)ccc4n3)cc(F)c2n1 ; Cl] |
| 123 | 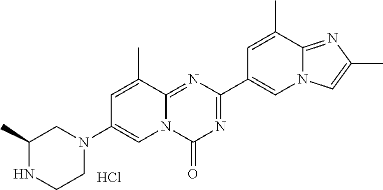 |
| 124 | 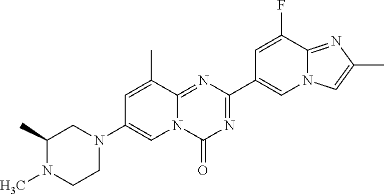 |
| 125 | 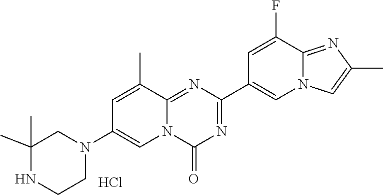 |
| 126 | 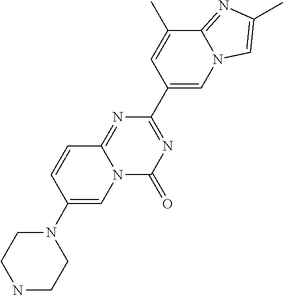 [SMILES: Cc1cn2cc(-c3nc(=O)n4cc(N5CCNCC5)ccc4n3)cc(C)c2n1] |
| 127 | 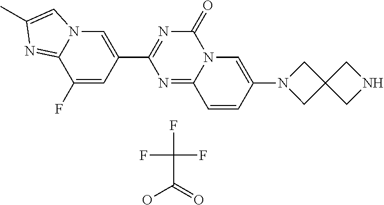 |
| 128 | 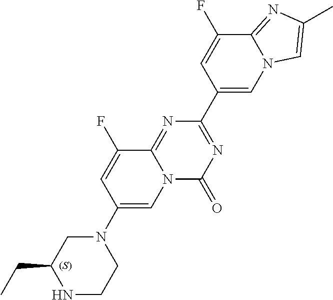 |
| 129 | 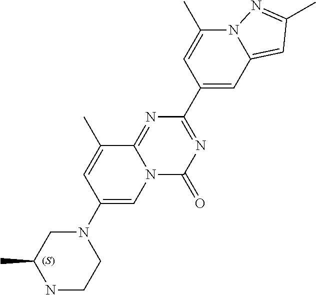 |
| 130 | 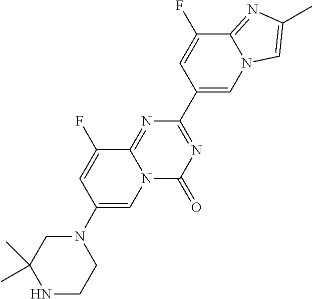 |
| 131 | 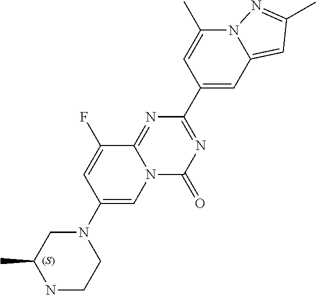 |
| 132 | 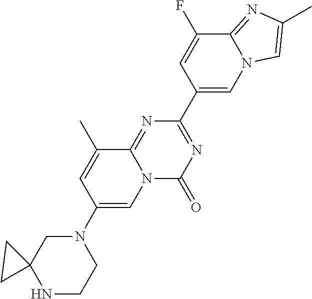 [SMILES: Cc1cn2cc(-c3nc(=O)n4cc(N5CCNC6(CC6)C5)cc(C)c4n3)cc(F)c2n1] |
| 133 | 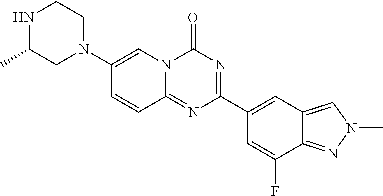 |
| 134 | 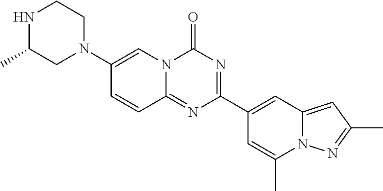 |
| 135 | 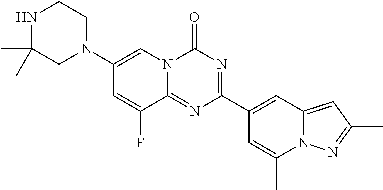 |

### Biological Activity of the Compounds of the Present Disclosure

The biological activity of the compounds of the present disclosure was determined utilizing the assay described herein.

### Example 52. Cell Proliferation Assay

Various cancer cell lines including K562 and COLO201, were treated with the compounds of the present disclosure to assess their effect on inhibiting cell proliferation.

For compound preparation, test compounds were diluted in duplicates from 10 mM stocks in 11-points 1:3 serial dilutions in DMSO. Then 125 nL test compounds at the serial dilutions were dispensed into 384-well assay plate using Echo dispenser. Staurosporine at 1 mM stock (5 μM final concentration) and DMSO (0.500 final concentration) were used as the positive and negative controls respectively.

To prepare cell lines, each frozen cell line stock was resuspended and grown in proper growth medium to a density of 40,000 cells/mL. Then 25 μL of cell suspension were dispensed into the assay plate to give seed cell density at 1,000 cells/well, compounds at 50 μM top concentration with 11-point serial dilutions, and 0.5% o DMSO final concentration. Assay plates were then centrifuged at 300 rpm for 1 minute and then incubated at 37° C., 9500 humidity, 500 CO2 for 72 hours. After the 72 hour incubation, the assay plate and its contents were equilibrated at room temperature for approximately 30 minutes. 25 μL of CellTiterGlo reagent was added to the assay plate and mixed for two minutes on an orbital shaker. Then after 10 minutes of incubation of assay plate at from temperature, the luminescence (RLU) signals in each well were recorded using Envision plate reader, then converted to percentage inhibition values using the signals in staurosporine and DMSO treated wells as the references. IC₅₀ values were determined by curve fitting in Levenberg-Marquardt algorithm.

In Table D below, A indicates a IC₅₀ (μM)<1 μM, B indicates a IC₅₀ (μM) 1 μM to <10 μM, and C indicates a IC₅₀ (μM)≥10 μM.

TABLE D
K562 and COLO201 Assay Data

| Compound | IC₅₀ (μM) | IC₅₀ (μM) |
|---|---|---|
| No. | K562 | COLO201 |
| 99 | A | |
| 100 | A | A |
| 101 | A | A |
| 102 | A | B |
| 103 | B | B |
| 104 | C | C |
| 105 | C | C |
| 106 | C | C |
| 107 | A | A |
| 108 | B | A |
| 109 | A | A |
| 110 | A | A |
| 111 | B | A |
| 112 | B | |
| 113 | B | |
| 114 | B | B |
| 115 | A | B |
| 116 | A | A |
| 117 | A | A |
| 118 | A | B |
| 119 | B | B |
| 120 | C | C |
| 121 | A | A |
| 122 | B | C |
| 123 | B | |
| 124 | B | |
| 125 | B | |
| 126 | B | |
| 127 | B | C |
| 128 | A | |
| 129 | B | |
| 130 | A | |
| 131 | A | A |
| 132 | B | |
| 133 | A | B |
| 134 | B | |
| 135 | B | |

### EQUIVALENTS

The details of one or more embodiments of the disclosure are set forth in the accompanying description above. Although any methods and materials similar or equivalent to those described herein can be used in the practice or testing of the present disclosure, the preferred methods and materials are now described. Other features, objects, and advantages of the disclosure will be apparent from the description and from the claims. In the specification and the appended claims, the singular forms include plural referents unless the context clearly dictates otherwise. Unless defined otherwise, all technical and scientific terms used herein have the same meaning as commonly understood by one of ordinary skill in the art to which this disclosure belongs. All patents and publications cited in this specification are incorporated by reference.

The foregoing description has been presented only for the purposes of illustration and is not intended to limit the disclosure to the precise form disclosed, but by the claims appended hereto.

1. A compound of Formula (I): 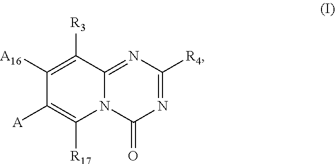
or a pharmaceutically acceptable salt, solvate, or prodrug thereof, wherein:
A is saturated or partially unsaturated mono- or bi-cyclic 4- to 9-membered heterocycloalkyl or NR₁R₂, wherein the heterocycloalkyl comprises 1 or 2 nitrogen ring atoms and is optionally substituted with 1, 2, 3, or 4 R₆;
R₁ is heterocycloalkyl comprising 1 nitrogen ring atom, optionally substituted with 1, 2, 3, or 4 R₆;
R₂ is hydrogen, C₁₋₇alkyl, or C₃₋₈cycloalkyl;
R₃ is H, halo, C₁₋₇alkyl, OR₅, N(R₅)₂, C₃₋₈cycloalkyl, or heterocycloalkyl;
R₄ is aryl or bicyclic 9-membered heteroaryl comprising 2, 3, or 4 heteroatoms independently selected from N, O, and S, wherein R₄ is optionally substituted with 1, 2, or 3 R₇;
each R₅ is independently C₁₋₇alkyl, C₃₋₈cycloalkyl, or heterocycloalkyl;
each R₆ is independently selected from the group consisting of halogen, hydroxy, cyano, —COOH, —C(O)—C₁-C₆alkyl, —C(O)O—C₁-C₆alkyl, C₁-C₇alkyl, C₁-C₈heteroalkyl, C₁₋₇alkoxy-heterocycloalkyl, C₂-C₆alkenyl, C₂-C₆alkynyl, C₁-C₆alkoxy, —(CH₂)₀₋₂-C₃-C₈cycloalkyl, 4-7-membered monocyclic heterocycloalkyl, NH₂, NH(C₁-C₆alkyl), N(C₁-C₆alkyl)₂, —NHC(O)—C₁-C₆alkyl, —N(C₁-C₆alkyl)-C(O)—C₁-C₆alkyl, —C(O)—NH₂, —C(O)—NH(C₁-C₆alkyl), and —C(O)—N(C₁-C₆alkyl)₂, wherein the alkyl, alkenyl, alkynyl, and alkoxy are optionally substituted with one or more halogen, hydroxyl or NH₂, and wherein the cycloalkyl and heterocycloalkyl are optionally substituted with one or more halogen, hydroxyl, C₁-C₆alkyl, C₁-C₆heteroalkyl, C₁-C₆alkoxy, or NH₂; or
two R₆ on the same carbon can be taken together as keto (═O); or
two R₆ together form C₁₋₇alkylene;
each R₇ is independently halo, cyano, C₁₋₇alkyl, C₁₋₇haloalkyl, C₁₋₇alkoxy, C₁₋₇ haloalkoxy, or C₃₋₈cycloalkyl, wherein the C₁₋₇alkyl is optionally substituted with OH;
R₁₆ is H, halo, C₁₋₇alkyl, OR₅, N(R₅)₂, C₃₋₈cycloalkyl, or heterocycloalkyl; and. R₁₇ is H, halo, C₁₋₇alkyl, OR₅, N(R₅)₂, C₃₋₈cycloalkyl, or heterocycloalkyl.

2. A compound of Formula (I): 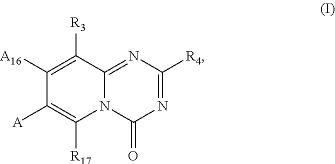
or a pharmaceutically acceptable salt, solvate, or prodrug thereof, wherein:
A is saturated or partially unsaturated mono- or bi-cyclic 4- to 9-membered heterocycloalkyl or NR₁R₂, wherein the heterocycloalkyl comprises 1 or 2 nitrogen ring atoms and is optionally substituted with 1, 2, 3, or 4 R₆;
R₁ is heterocycloalkyl comprising 1 nitrogen ring atom, optionally substituted with 1, 2, 3, or 4 R₆;
R₂ is hydrogen, C₁₋₇alkyl, or C₃₋₈cycloalkyl;
R₃ is H, halo, C₁₋₇alkyl, OR₅, N(R₅)₂, C₃₋₈cycloalkyl, or heterocycloalkyl;
R₄ is aryl or bicyclic 9-membered heteroaryl comprising 2, 3, or 4 heteroatoms independently selected from N, O, and S, wherein R₄ is optionally substituted with 1, 2, or 3 R₇;
each R₅ is independently C₁₋₇alkyl, C₃₋₈cycloalkyl, or heterocycloalkyl;
each R₆ is independently C₁₋₇alkyl, amino, amino-C₁₋₇alkyl, C₃₋₈cycloalkyl, heterocycloalkyl, or C₁₋₇alkoxy-heterocycloalkyl, or two R₆ together form C₁₋₇alkylene;
each R₇ is independently halo, cyano, C₁₋₇alkyl, C₁₋₇haloalkyl, C₁₋₇alkoxy, C₁₋₇ haloalkoxy, or C₃₋₈cycloalkyl, wherein the C₁₋₇alkyl is optionally substituted with OH;
R₁₆ is H, halo, C₁₋₇alkyl, or OR₅; and.
R₁₇ is H, halo, C₁₋₇alkyl, or OR₅.

3. A compound of Formula (I): 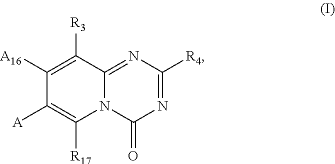
or a pharmaceutically acceptable salt, solvate, or prodrug thereof, wherein:
A is a nitrogen-containing heterocycloalkyl, wherein the nitrogen-containing heterocycloalkyl comprises 1 or 2 nitrogen ring atoms and is optionally substituted with 1, 2, 3, or 4 R₆;
R₃ is H, C₁₋₇alkyl, OR₅, N(R₅)₂, C₃₋₈cycloalkyl, or heterocycloalkyl;
R₄ is a bicyclic 9-membered heteroaryl comprising 2, 3, or 4 heteroatoms independently selected from N and O, wherein R₄ can be optionally substituted with 1, 2, or 3 R₇;
each R₅ is independently H, C₁₋₇alkyl, C₃₋₈cycloalkyl, or heterocycloalkyl;
each R₆ is independently C₁₋₇alkyl, amino, amino-C₁₋₇alkyl, C₃₋₈cycloalkyl, heterocycloalkyl, and C₁₋₇alkoxy-heterocycloalkyl, or two R₆ together form C₁₋₇alkylene;
each R₇ is independently halo, C₁₋₇alkyl, C₁₋₇haloalkyl, C₁₋₇alkoxy, or C₁₋₇haloalkoxy;
R₁₆ is H, halo, C₁₋₇alkyl, or OR₅; and.
R₁₇ is H, halo, C₁₋₇alkyl, or OR₅.

4. A compound of Formula (II): 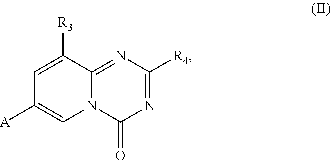
or a pharmaceutically acceptable salt, solvate, or prodrug thereof, wherein:
A is saturated or partially unsaturated mono- or bi-cyclic 4- to 9-membered heterocycloalkyl or NR₁R₂, wherein the heterocycloalkyl comprises 1 or 2 nitrogen ring atoms and is optionally substituted with 1, 2, 3, or 4 R₆;
R₁ is heterocycloalkyl comprising 1 nitrogen ring atom, optionally substituted with 1, 2, 3, or 4 R₆;
R₂ is hydrogen, C₁₋₇alkyl, or C₃₋₈cycloalkyl;
R₃ is H, halo, C₁₋₇alkyl, OR₅, N(R₅)₂, C₃₋₈cycloalkyl, or heterocycloalkyl;
R₄ is aryl or bicyclic 9-membered heteroaryl comprising 2, 3, or 4 heteroatoms independently selected from N, O, and S, wherein R₄ is optionally substituted with 1, 2, or 3 R₇;
each R₅ is independently C₁₋₇alkyl, C₃₋₈cycloalkyl, or heterocycloalkyl;
each R₆ is independently C₁₋₇alkyl, amino, amino-C₁₋₇alkyl, C₃₋₈cycloalkyl, heterocycloalkyl, or C₁₋₇alkoxy-heterocycloalkyl, or two R₆ together form C₁₋₇alkylene; and
each R₇ is independently halo, cyano, C₁₋₇alkyl, C₁₋₇haloalkyl, C₁₋₇alkoxy, C₁₋₇ haloalkoxy, or C₃₋₈cycloalkyl, wherein the C₁₋₇alkyl is optionally substituted with OH.

5. A compound of Formula (II): 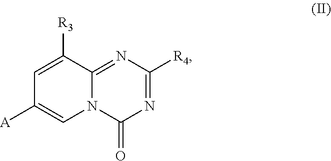
or a pharmaceutically acceptable salt, solvate, or prodrug thereof, wherein:
A is nitrogen-containing heterocycloalkyl, wherein the nitrogen-containing heterocycloalkyl comprises 1 or 2 nitrogen ring atoms and is optionally substituted with 1, 2, 3, or 4 R₆;
R₃ is H, C₁₋₇alkyl, OR₅, N(R₅)₂, C₃₋₈cycloalkyl, or heterocycloalkyl;
R₄ is a bicyclic 9-membered heteroaryl comprising 2, 3, or 4 heteroatoms independently selected from N and O, wherein R₄ can be optionally substituted with 1, 2, or 3 R₇;
each R₅ is independently C₁₋₇alkyl, C₃₋₈cycloalkyl, or heterocycloalkyl;
each R₆ is independently C₁₋₇alkyl, amino, amino-C₁₋₇alkyl, C₃₋₈cycloalkyl, heterocycloalkyl, and C₁₋₇alkoxy-heterocycloalkyl, or two R₆ together form C₁₋₇alkylene; and
each R₇ is independently halo or C₁₋₇alkyl.

6. A compound according to any of claims 1-5, wherein:
A is nitrogen-containing heterocycloalkyl, wherein the nitrogen-containing heterocycloalkyl comprises 1 or 2 nitrogen ring atoms and is optionally substituted with 1, 2, 3, or 4 R₆, and is bonded to formula (I) or (II) by one of its nitrogen atoms; and
each R₆ is independently C₁₋₇alkyl, amino, amino-C₁₋₇alkyl, C₃₋₈cycloalkyl, heterocycloalkyl, or C₁₋₇alkoxy-heterocycloalkyl or two R₆ together form C₁₋₇alkylene.

7. A compound according to any one of claims 1-6, wherein each R₆ is independently C₁₋₇ alkyl, heterocycloalkyl, or C₁₋₇alkoxy-heterocycloalkyl or two R₆ together form C₁₋₇alkylene.

8. A compound according to any one of claims 1-7, wherein each R₆ is independently methyl, ethyl, isopropyl, methoxy-azetidinyl, or pyrrolidinyl, or two R₆ together form ethylene or propylene.

9. A compound according to any one of claims 1-8, wherein
A is 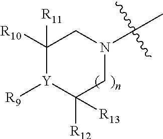
Y is absent, N or CH;
R₉ is hydrogen, C₁₋₇alkyl, or (CH₂)ₘ—NR₁₄R₁₅;
R₁₀ is hydrogen or C₁₋₇alkyl;
R₁₁ is hydrogen or C₁₋₇alkyl;
R₁₂ is hydrogen or C₁₋₇alkyl;
R₁₃ is hydrogen or C₁₋₇alkyl;
each R₁₄ and R₁₅ are independently hydrogen, C₁₋₇alkyl and C₃₋₈cycloalkyl;
n is 0, 1 or 2;
m is 0, 1 or 2;
or R₉ and R₁₀ together form C₁₋₇alkylene;
or R₉ and R₁₂ together form C₁₋₇alkylene;
or R₁₀ and R₁₁ together form C₂₋₇alkylene or 4- to 6-membered heterocycloalkyl optionally substituted with C₁₋₇alkyl;
or R₁₀ and R₁₂ together form C₁₋₇alkylene or 4- to 6-membered heterocycloalkyl optionally substituted with C₁₋₇alkyl;
or R₁₀ and R₁₄ together form C₁₋₇alkylene;
or R₁₂ and R₁₃ together form C₂₋₇alkylene;
or R₁₂ and R₁₄ together form C₁₋₇alkylene;
or R₁₄ and R₁₅ together form C₂₋₇alkylene which is optionally substituted with C₁₋₇alkoxy.

10. A compound according to claim 9, wherein Y is N.

11. A compound according to claim 9, wherein Y is CH and R₉ is (CH₂)ₘ—NR₁₄R₁₅.

12. A compound according to any one of claims 9-11, wherein n is 1.

13. A compound according to any one of claims 9-10 and 12, wherein R₉ is hydrogen, pyrrolidinyl, or methoxy-azetidinyl.

14. A compound according to any one of claims 9-13, wherein R₁₀ is hydrogen, methyl, ethyl or isopropyl.

15. A compound according to any one of claims 9-14, wherein R₁₁ is hydrogen or methyl.

16. A compound according to any one of claims 9-15, wherein R₁₂ is hydrogen or methyl.

17. A compound according to any one of claims 9-16, wherein R₁₃ is hydrogen.

18. A compound according to any one of claims 9-17, wherein R₉ and R₁₀ together form propylene.

19. A compound according to any one of claims 9-17, wherein R₁₀ and R₁₁ together form ethylene.

20. A compound according to any one of claims 9-19, wherein R₁₄ and R₁₅ together form propylene or butylene.

21. A compound according to any one of claims 1-20, wherein A is 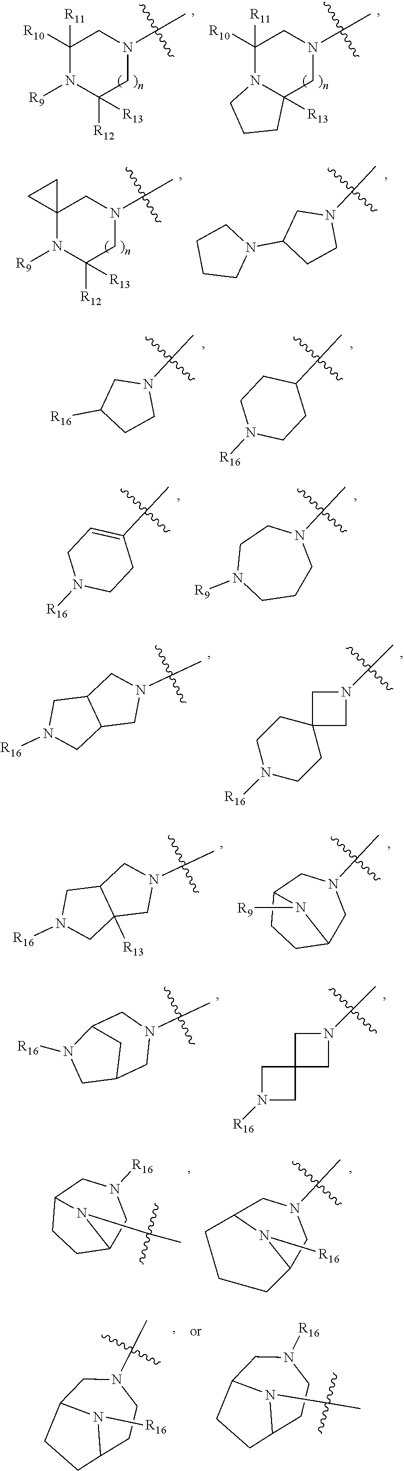
wherein R₉, R₁₀, R₁₁, R₁₂, and R₁₃ are as defined in any one of claims 1-20, and R₁₆ is hydrogen or C₁₋₇alkyl.

22. A compound according to any one of claims 1-21, wherein A is piperazinyl, diazepanyl, octahydropyrrolopyrazinyl, diazaspirooctanyl, pyrrolidinyl, octahydropyrrolopyrroyl, diazaspirononanyl, diazaspiroheptanyl, or diazabicyclooctanyl, wherein piperazinyl, diazepanyl, octahydropyrrolopyrazinyl, diazaspirooctanyl, pyrrolidinyl, octahydropyrrolopyrroyl, diazaspirononanyl, diazaspiroheptanyl, or diazabicyclooctanyl are each optionally substituted with 1, 2, 3, or 4 R₆.

23. A compound according to any one of claims 1-22, wherein A is NR₁R₂.

24. A compound according to any one of claims 1-22, wherein A is

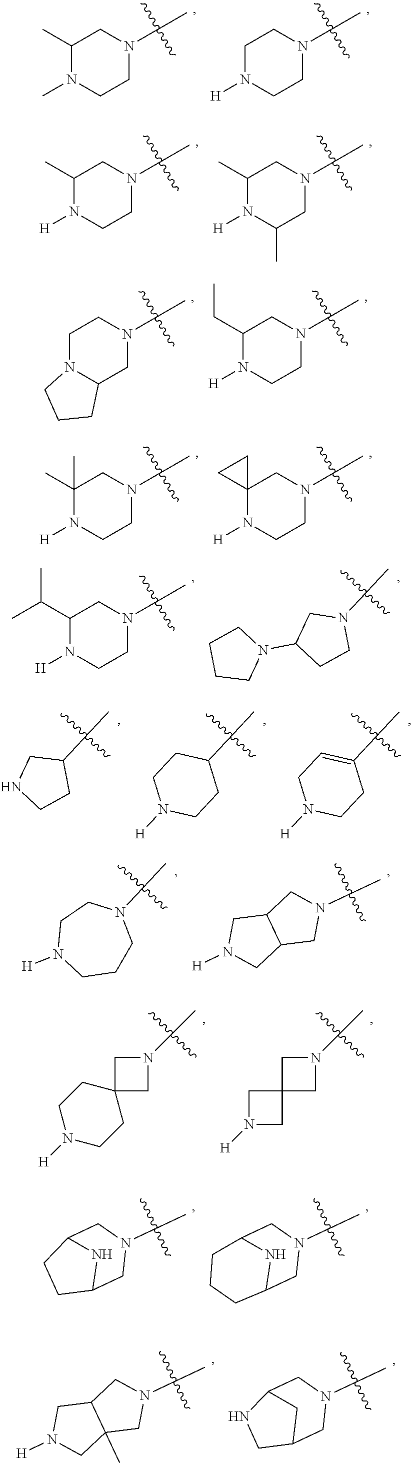

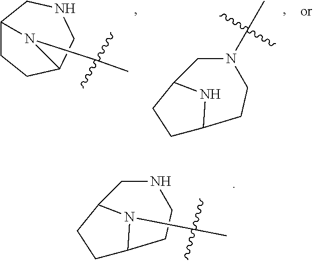 [SMILES: *N1C2CCC1CNC2 ; *N1CCC2CCC(C1)N2 ; *N1C2CCNCC1CC2]

25. A compound according to any one of claims 1-24, wherein R₄ is a bicyclic 9-membered heteroaryl comprising 2 heteroatoms independently selected from N and O, optionally substituted with 1, 2, or 3 R₇.

26. A compound according to any one of claims 1-25, wherein R₄ is a bicyclic 9-membered heteroaryl comprising two heteroatoms independently selected from N and O, substituted with 1 or 2 R₇.

27. A compound according to any one of claims 1-26, wherein R₄ is

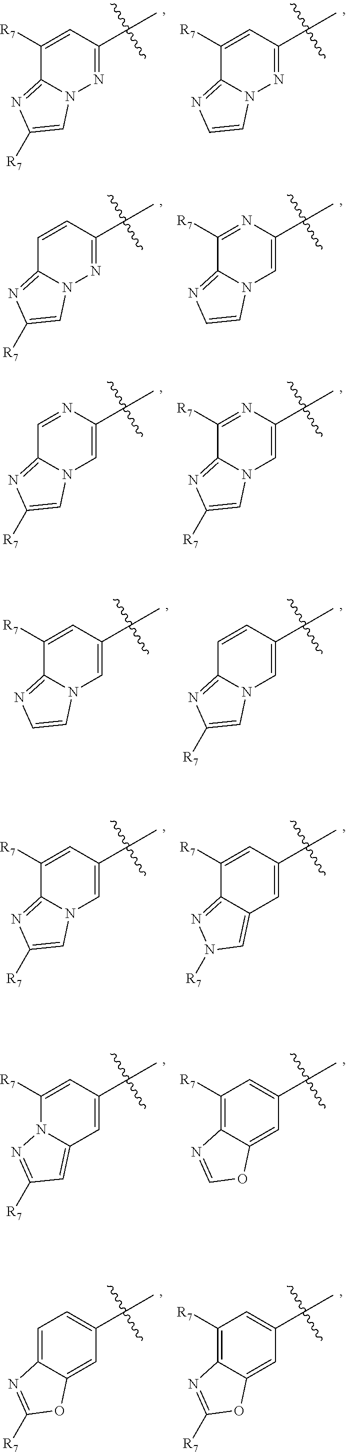

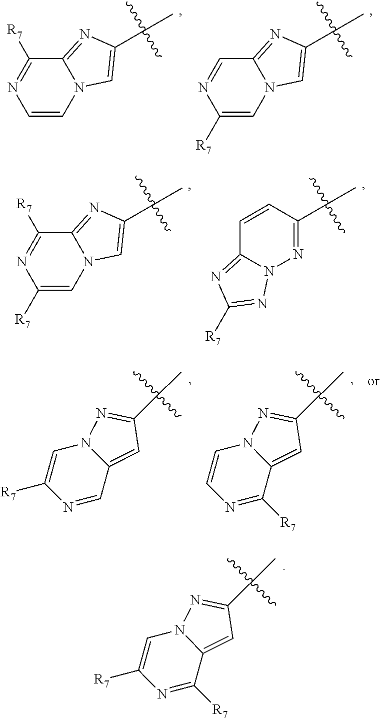

28. A compound according to any one of claims 1-26, wherein R₄ is

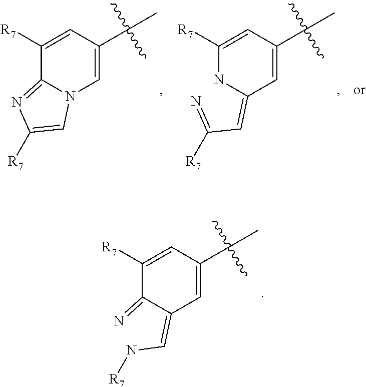

29. A compound according to any one of claims 1-26, wherein R₄ is imidazo[1,2-a]pyrazine, benzo[d]oxazole, triazolo-pyridazinyl, or imidazo[1,2-a]pyrazine, wherein imidazo[1,2-a]pyrazine, benzo[d]oxazole, triazolo-pyridazinyl, or imidazo[1,2-a]pyrazine each are optionally substituted with 1, 2, 3, or 4 R₇.

30. A compound according to any one of claims 1-26, wherein R₄ is

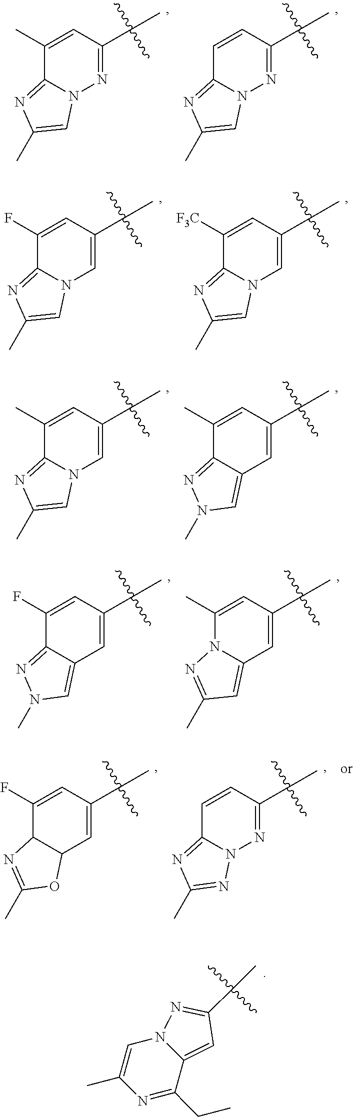

31. A compound according to any one of claims 1-26, wherein R₄ is

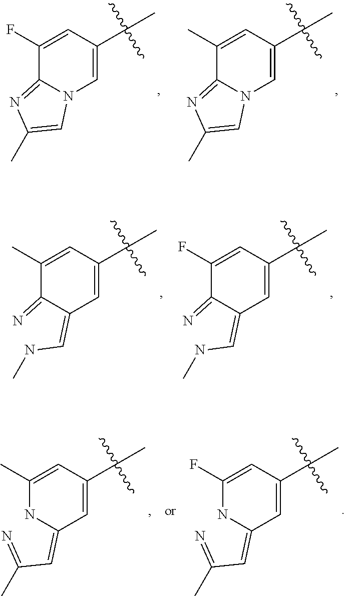 [SMILES: *c1cc(F)c2nc(C)cn2c1 ; *c1cc(C)c2nc(C)cn2c1 ; *C1=C/C(=C/NC)C(=N)C(C)=C1 ; *C1=C/C(=C/NC)C(=N)C(F)=C1 ; *C1=C/C(=C/C(C)=N)NC(C)=C1 ; *C1=C/C(=C/C(C)=N)NC(F)=C1]

32. A compound according to any one of claims 1-31, selected from a compound of Table 1, or from the disclosure.

33. A compound according to any one of claims 1-32, or a pharmaceutically acceptable salt, solvate, or prodrug thereof for use as a therapeutically active substance.

34. A compound according to any one of claims 1-32, or a pharmaceutically acceptable salt, solvate, or prodrug thereof for use as a small molecule splicing modulator.

35. A pharmaceutical composition comprising a compound according to any one of claims 1-34, or a pharmaceutically acceptable salt, solvate, or prodrug thereof and one or more pharmaceutically acceptable excipients.

36. A method of treatment or prevention of cancer in a subject in need thereof, the method comprising administering to the subject a therapeutically effective amount of a compound of claims 1-34 or a pharmaceutical composition of claim 35.

37. The method of claim 36, wherein the cancer is a leukemic cancer.

38. The method of claim 36, wherein the cancer is colorectal cancer, breast cancer, or prostate cancer.

39. with the method of claim 36 wherein the cancer is a liquid cancer.

40. The method of claim 36, wherein the cancer is a leukemia or lymphoma.

41. The method of claim 36, wherein the cancer is a leukemia, acute myeloid leukemia, colon cancer, gastric cancer, acute monocytic leukemia, breast cancer, hepatocellular carcinoma, alveolar soft part sarcoma, myeloma, skin melanoma, pancreatic cancer, adenocarcinoma, adenoiditis, adenoid cystic carcinoma, gastrointestinal stromal tumor, sarcoma, prostate adenocarcinoma, Hodgkin's lymphoma, ovarian cancer, non-Hodgkin's lymphoma, multiple myeloma, chronic myeloid leukemia, acute lymphoblastic leukemia, renal cell carcinoma, transitional cell carcinoma, colorectal cancer, chronic lymphocytic leukemia, anaplastic large cell lymphoma, kidney cancer, breast cancer, or cervical cancer.

42. The method of claim 36, wherein the cancer is a solid cancer or solid tumor.
